(19) United States
(12) Patent Application Publication
(10) Pub. No.: US 2003/0224383 A1
(43) Pub. Date: Dec. 4, 2003

(54) Atherosclerotic phenotype determinative genes and methods for using the same

(76) Inventors: Mike West, Durham, NC (US); Joseph R. Nevins, Chapel Hill, NC (US); Pascal Goldschmidt, Chapel Hill, NC (US)

Correspondence Address: Gregory J. Glover, Ropes & Gray, Suite 800 East, 1301 K Street, NW, Washington, DC, 20005, US

(21) Appl. No.: 10/291,885

(22) Filed: Nov. 12, 2002

Related U.S. Application Data
(60) Provisional application No. 60/374,547, filed on Apr. 23, 2002.
(60) Provisional application No. 60/420,784, filed on Oct. 24, 2002.
(60) Provisional application No. 60/421,043, filed on Oct. 25, 2002.
(60) Provisional application No. 60/424,680, filed on Nov. 8, 2002.

(51) Int. Cl. C12Q 1/68; A61K 31/00; C12M 1/34

(52) U.S. Cl. 435/6; 435/287.2; 514/1

(57) ABSTRACT

Genes whose expression is correlated with and determinant of an atherosclerotic phenotype are provided. Also provided are methods of using the subject atherosclerotic determinant genes in diagnosis and treatment methods, as well as drug screening methods. In addition, reagents and kits thereof that find use in practicing the subject methods are provided. Also provided are methods of determining whether a gene is correlated with a disease phenotype, where correlation is determined using at least one parameter that is not expression level and is preferably determined using a Bayesian analysis.

### FIELD OF THE INVENTION

The field of this invention is atherosclerosis.

### BACKGROUND OF THE INVENTION

Atherosclerosis is a complex trait manifested by chronic inflammation that selectively affects arterial vessels and progressively destroys the structure of the vessel wall, leading to thromboembolic complications. The thromboembolic consequences of atherosclerosis, sudden cardiac death, myocardial infarction, and other ischemic organ damage such as stroke and ischemic renovascular disease, represent the major causes of death, morbidity and disability for developed countries and are spreading rapidly worldwide. In spite of substantial improvement in our understanding of risk for atherosclerosis and thromboembolic complications, improved predictive tools are needed to allow for early prevention in a fashion that is cost-effective.

The sequencing of the entire human genome promises to transform the study of human health by providing an opportunity to develop genomic knowledge that will eventually boost prevention, diagnosis and treatment of disease. Genome research in the post-sequencing era is now faced with massive, multi-disciplinary challenges in order to realize this promise. Most complex illnesses result (i) from the combined action of gene variants that are considered as “normal”, as they do not destroy the function of the gene that they modify; (ii) from factors provided by the environment, and (iii) from a stochastic component that can be best defined as “chance”. The ensemble of genetic modifiers that enhance the impact of environmental factors on health represents the genetic susceptibility to ailments.

Because of the potential benefits of genetic approaches to diagnosis and treatment of atherosclerosis, there is intense interest in the identification of genes whose contribution is relevant to atherosclerosis. Ideally, one would like to test all variants of all genes for their contribution to atherosclerosis. However, such effort would be unacceptably expensive, and even if the resources were to become accessible, our current ability to analyze data would become limiting. Hence, the prioritization of contributory genes has become a necessity. A systematic approach to satisfy this need and provide such prioritization process has been defined and is based on gene expression that correlates with atherosclerosis. Hence, the present invention satisfies this need.

As with most complex illnesses, atherosclerosis results from the combined interaction of a genetic component and environmental factors. However, unlike classical Mendelian disorders, the genetic component is not attributable to single causative genes making it difficult to study by standard genetic and molecular biological approaches. Instead, it is anticipated that combinations of gene variants determine an individual's susceptibility to atherosclerosis by enhancing the impact of environmental factors.

The gene variants are often in the form of single nucleotide polymorphisms (SNPs). SNPs represent subtle variations in a gene's coding sequence or the associated regulatory regions resulting in a mild to moderate impact on the function or concentration of the encoded protein. The inheritance of unique combinations of genetic variants can have a dominant impact that fosters the pathogenesis of atherosclerosis. In principle, we would like to identify all variants of all genes and assay them for their contribution towards the genesis of atherosclerosis. Even if we were able to identify all variants, we would be limited by our ability to assay and analyze such a vast number of SNPs. Practically, one must take an approach that falls somewhere between an analysis restricted to known candidate genes identified on the basis of clinical and biological knowledge (functional candidate genes) and an investigation of the entire genomic complement of genes. See Nussbaum R L MRaWH. Genetics in Medicine. New York: W. B. Saunders Company, 2001. Science 1996; 272:689-93.

Such an approach should involve prioritization based on programmatic qualification mechanisms.

Recent advances in the knowledge of the human genome, coupled with the development of technologies for large scale analysis of gene activity via DNA microarrays, now affords the opportunity to identify genes whose expression implies a role in a phenotype. We have used a unique collection of human aorta samples, which exhibit a progression of atherosclerotic disease, coupled with novel strategies for analyzing gene expression data, to identify genes whose expression closely relates to, and indeed predicts, the extent of fatty streaks and more advanced atherosclerotic lesions. We believe this represents a novel approach to the identification of genes that contribute to atherosclerosis.

### SUMMARY OF THE INVENTION

Genes whose expression is correlated with and determinant of an atherosclerotic phenotype are provided. Also provided are methods of using the subject atherosclerotic determinant genes in diagnosis and treatment methods, as well as drug screening methods. In addition, reagents and kits thereof that find use in practicing the subject methods are provided. Also provided are methods of determining whether a gene is correlated with a disease phenotype, e.g., atherosclerosis, where correlation is determined using at least one parameter that is not expression level and is preferably determined using a Bayesian analysis.

Several predictive models are provided in this invention, which include a linear regression model incorporating Bayesian techniques, as well as predictive tree models incorporating Bayesian techniques. The invention also provides metagenes for atherosclerosis identified by the use of a predictive tree model, that characterize multiple patterns of expression of the genes across the samples.

### BRIEF DESCRIPTION OF THE FIGURES

FIG. 1: Soudan IV staining and morphometric analysis. Three representative samples of an aorta collection are shown. The aorta originated from donors of different age and, accordingly, the extent of atherosclerosis varied from mild (age 20) to severe (age 60). For each sample, Sudan IV staining is shown (left panel), followed by morphometric analysis of Soudan IV positive lesions (MA/S-IV, middle panel), and morphometric analysis of raised plaque (MA/RP, complex lesions). Location of segments I and IV are shown on the top panel. Note that Soudan IV positive lesions in segments I of donors ages 40 and 60 were unusually pronounced for this segment. For the group, Soudan IV positive lesions were rare in segments I, and frequent in segments IV. Inset-d: Lipid deposition is shown within the space that separates media and neointima (oil-red stain). Inset-e: Elastin stain of area that are Soudan IV positive. Note the accumulation of neointima on the lumenal side of the aortic tissue.

FIG. 2. Display of the probabilistic within-sample discrimination, giving estimated classification probabilities that identify tissues as likely “diseased” (segment IV) versus “healthy” (segment I) based on the expression profile of the 83 genes and summarized in terms of the implied “supergene” regression predictor.

FIG. 3. Expression levels of top 83 genes providing discrimination of segment I vs. IV status. Expression levels are depicted by color coding with black representing the lowest level, followed by red, orange, yellow, and then white as the highest level of expression. Each raw represents all 83 genes for an individual aorta sample which are grouped according to segment identity (I versus IV). Each column represents an individual gene, ordered from top to bottom according to regression coefficients. Natural grouping of samples was observed: samples 1-15 were from segments I, whereas samples 16-27 were from the diseased segments IV.

FIG. 4. Display of expression levels (log2 scale) of six selected genes whose patterns are representative of a larger group.

FIG. 5. Linear regressions in which the % area affected by early atherosclerosis (as measured by Soudan IV staining) is predicted by an optimized linear function of expression levels of selected genes. Gene subset selection in this pilot analysis follows that of the binary analysis in selecting genes most highly correlated with the Soudan IV measure. A total of 55 genes were selected this way. Each of the 12 segment IV tissue samples is represented by its measured % scaling, on the horizontal axis, and by the corresponding predicted value from the linear regression model using the 55 genes, on the vertical axis. The line of equality is drawn; a “perfect” model fit to the data would have all 12 circles sitting on this line. The vertical dashed lines represent approximate 95% probability intervals that represent uncertainty in the predictions. The point predictions alone represent a fit to the data with a traditional regression R2 measure of fit of about 0.94, consistent with a statistically significant model.

FIG. 6. Schematic of multi-prong approach for the identification of atherosclerotic phenotype determinative genes, and gene variants, e.g., SNPs, thereof.

FIG. 7: Table 1: 83 genes (+2 genes identified by an Epigenetic protocol) with an atherosclerotic determinative phenotype as determinative phenotype as determined by statistical Analysis and Screening via Binary Regression. Ordered according to Estimated Regression Coelticients.

FIG. 8: Table 2: Genes Identified Using Statistical Analysis and Screening via Binary Regresion that are logically Implicated with an Atherosclerotic Phenotype.

FIG. 9: Table 3: 55 Subset Genes selected using Statistical Analysis and Screening via Binary Regression in which the percentage Area affected by Atherosclerotic scaling (as measured by Sondan IV Staining) is predicted by an optimized linear function of selected genes.

FIG. 10: Table of specific atherosclerotic phenotypic determinative genes identified by a binary factor regression model.

FIG. 11: Aorta Phenotyping and Processing. (a) Diagram showing aortic anatomical landmarks: I—innominate (brachiocephalic) artery, LC—left carotid artery, LS—left subclavian artery;, (b) Sectioning lines were traced on a prototypical aorta sample; note the symmetrical atherosclerotic pattern; (c) Sudan IV staining (d) with automated mapping of sudanophilia and (e) morphometric mapping of raised lesions were obtained for another sample with more advanced lesions.

FIG. 12: Probability of Occurrence Maps. Maps showing frequencies for the presence of fatty streaks (Sudan IV positive) and raised lesions were provided for the aortas of younger (age <35) and older (age≧35) organ donors. Note that the two segments selected based on location and age significantly differ in terms of susceptibility for atherosclerotic lesions. Note also the two separate color scales for sudanophilia and raised lesions.

FIG. 13: Image Plot for Expression Data. Image plots of standardized expression levels were constructed for the 100 genes highlighted by the binary regression analysis.

FIG. 14: Cross-validation Predictions for Aorta Samples. Blue numbers represent resistant samples and the red numbers, the susceptible samples. All tissue samples were correctly reclassified, except for samples 15, 23, and 28 which mapped outside of their respective groups and were therefore misclassified.

FIG. 15: Demographics of Aorla Donors and Penolypic Characteristics

FIG. 16: Top 50 genes with increased expression levels in the high Susceptibility vs low susceptibility Tissue Sections

FIG. 17: Top 36 genes with decreased expression levels in the high susceptibility vs low susceptibility tissue Sections

FIG. 18: Analysis of 1A vs IVB for Atherosclerotic Susceptibility

FIG. 19: Analysis based upon the Biological Measurements of Atherosclerosis

FIG. 20: 395 Metagenes Identified Using Tree Prediction Model

FIG. 21: 99 Genes Identified from the Susceptibility Analysis

FIG. 22: 99 Genes Identified from the Disease Extent Analysis

FIG. 23: List of 18 Genes Common to Genes listed in both FIGS. 21 and 22.

### DETAILED DESCRIPTION OF THE SPECIFIC EMBODIMENTS

Genes whose expression is correlated with and determinant of an atherosclerotic phenotype are provided using various predictive statistical models. Also provided are methods of using the subject atherosclerotic determinant genes in diagnosis and treatment methods, as well as drug screening methods. In addition, reagents and kits thereof that find use in practicing the subject methods are provided. Also provided are methods of determining whether a gene is correlated with a disease phenotype, where correlation is determined using at least one parameter that is not expression level and is preferably determined using a Bayesian analysis.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

As summarized above, the subject invention is directed to a collection of genes whose expression is correlated with atherosclerosis, i.e., that are atherosclerotic phenotype determinative genes, as well as methods for using the collection or subparts thereof in various applications. In further describing the invention, the collection of genes determinative of the atherosclerotic phenotype is described first in greater detail, followed by a review of the various different applications in which the collection finds use, including diagnostic, therapeutic and screening applications. Also reviewed are reagents and kits for use in practicing the subject methods. Finally, a review of various methods of identifying genes whose expression correlates with a given phenotype, such as atherosclerosis, is provided.

### Atherosclerotic Phenotype Determinative Genes

The subject invention provides a collection of atherosclerotic phenotype determinative genes. By atherosclerotic phenotype determinative genes is meant genes whose expression or lack thereof correlates with an atherosclerotic phenotype. Thus, atherosclerotic determinative genes include genes: (a) whose expression is correlated with an atherosclerotic phenotype, i.e., are expressed in cells and tissues thereof that have an atherosclerotic phenotype, and (b) whose lack of expression is correlated with an atherosclerotic phenotype, i.e., are not expressed in cells and tissues thereof that have an atherosclerotic phenotype. A cell is a cell with an atherosclerotic phenotype if it is obtained from vascular tissue that is determined to be atherosclerotic, e.g., by Sudan staining according to the method reported in the experimental section, below. Likewise, tissue is tissue with an atherosclerotic phenotype if it is vascular tissue or obtained from vascular tissue that is determined to be atherosclerotic, e.g., by Sudan staining according to the method reported in the experimental section, below.

The invention claims all collections and subsets thereof of atherosclerotic phenotype determinative genes as well as metagenes disclosed herewith. The subject collections of atherosclerotic phenotype determinative genes may be physical or virtual. Physical collections are those collections that include a population of different nucleic acid molecules, where the atherosclerotic phenotype determinative genes are represented in the population, i.e., there are nucleic acid molecules in the population that correspond in sequence to the genomic, or more typically, coding sequence of the atherosclerotic phenotype determinative genes in the collection. In many embodiments, the nucleic acid molecules are either substantially identical or identical in sequence to the sense strand of the gene to which they correspond, or are complementary to the sense strand to which they correspond, typically to an extent that allows them to hybridize to their corresponding sense strand under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

The nucleic acids that make up the subject physical collections may be single-stranded or double-stranded. In addition, the nucleic acids that make up the physical collections may be linear or circular, and the individual nucleic acid molecules may include, in addition to an atherosclerotic phenotype determinative gene coding sequence, other sequences, e.g., vector sequences. A variety of different nucleic acids may make up the physical collections, e.g., libraries, such as vector libraries, of the subject invention, where examples of different types of nucleic acids include, but are not limited to, DNA, e.g., cDNA, etc., RNA, e.g., mRNA, cRNA, etc. and the like. The nucleic acids of the physical collections may be present in solution or affixed, i.e., attached to, a solid support, such as a substrate as is found in array embodiments, where further description of such diverse embodiments is provided below.

Also provided are virtual collections of the subject atherosclerotic phenotype determinative genes. By virtual collection is meant one or more data files or other computer readable data organizational elements that include the sequence information of the genes of the collection, where the sequence information may be the genomic sequence information but is typically the coding sequence information. The virtual collection may be recorded on any convenient computer or processor readable storage medium. The computer or processor readable storage medium on which the collection data is stored may be any convenient medium, including CD, DAT, floppy disk, RAM, ROM, etc, which medium is capable of being read by a hardware component of the device.

Also provided are databases of expression profiles of atherosclerotic phenotype determinative genes. Such databases will typically comprise expression profiles of various cells/tissues having atherosclerotic phenotypes, such as various stages of atherosclerosis, negative expression profiles, prognostic profiles, etc., where such profiles are further described below.

The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks expression profiles possessing varying degrees of similarity to a reference expression profile. Such presentation provides a skilled artisan with a ranking of similarities and identifies the degree of similarity contained in the test expression profile.

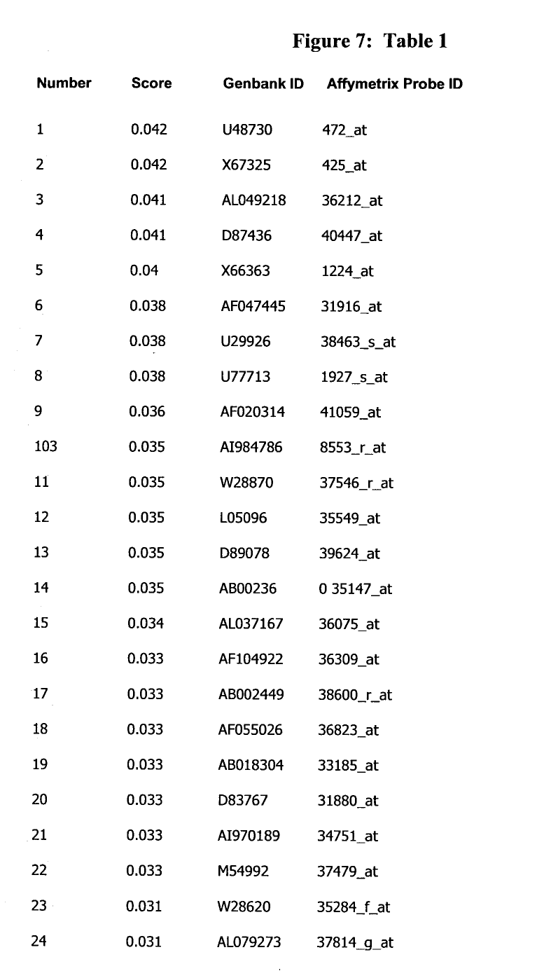

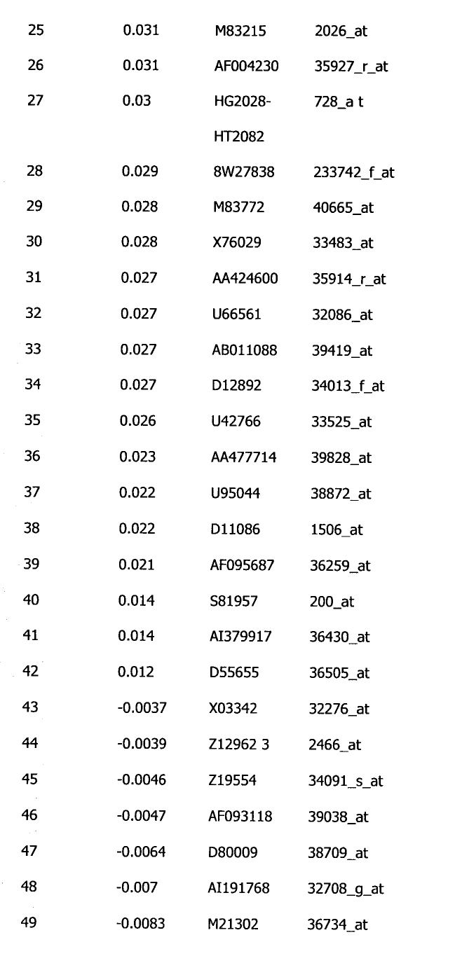

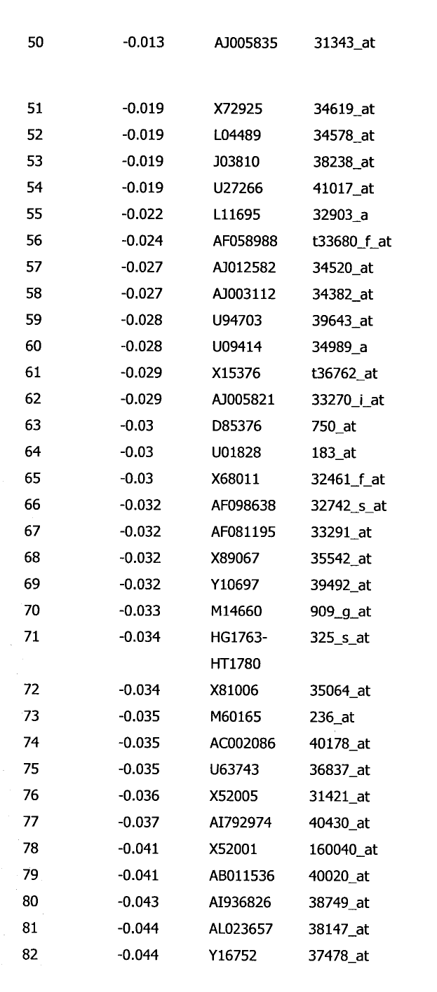

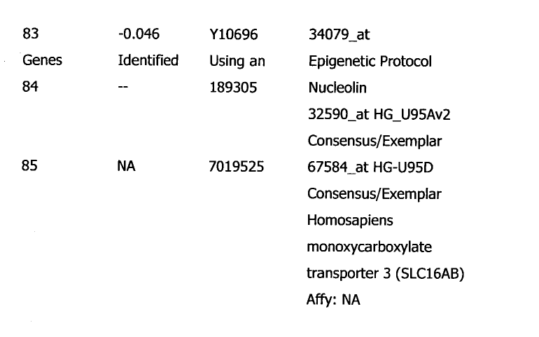

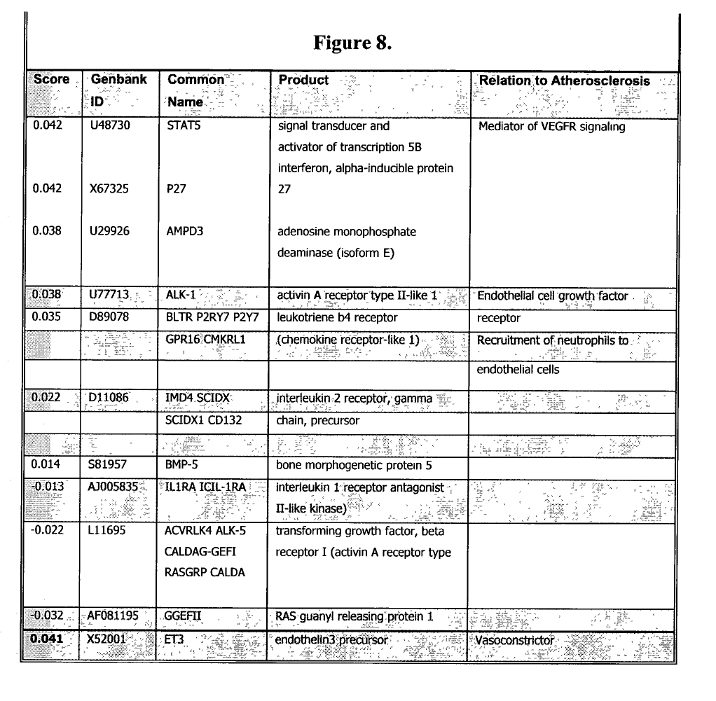

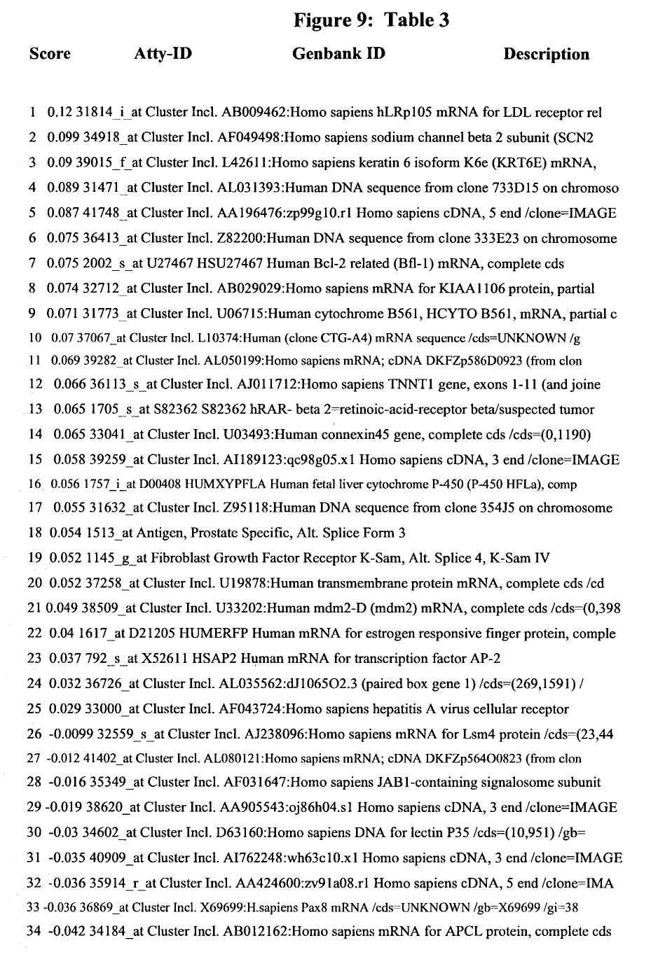

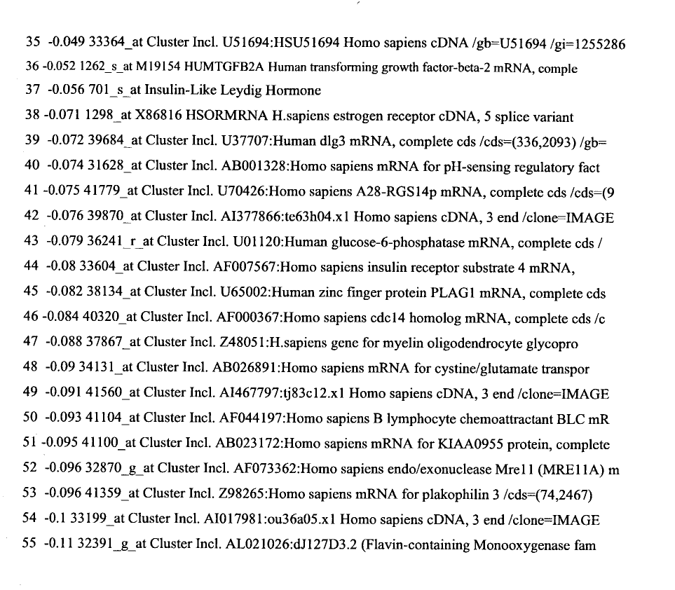

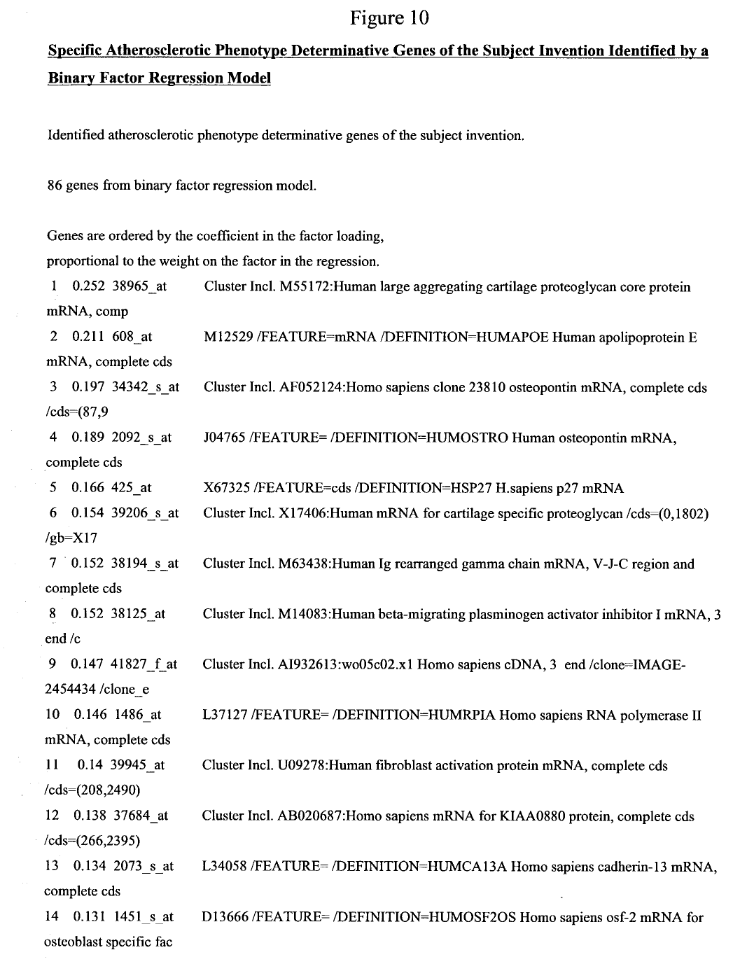

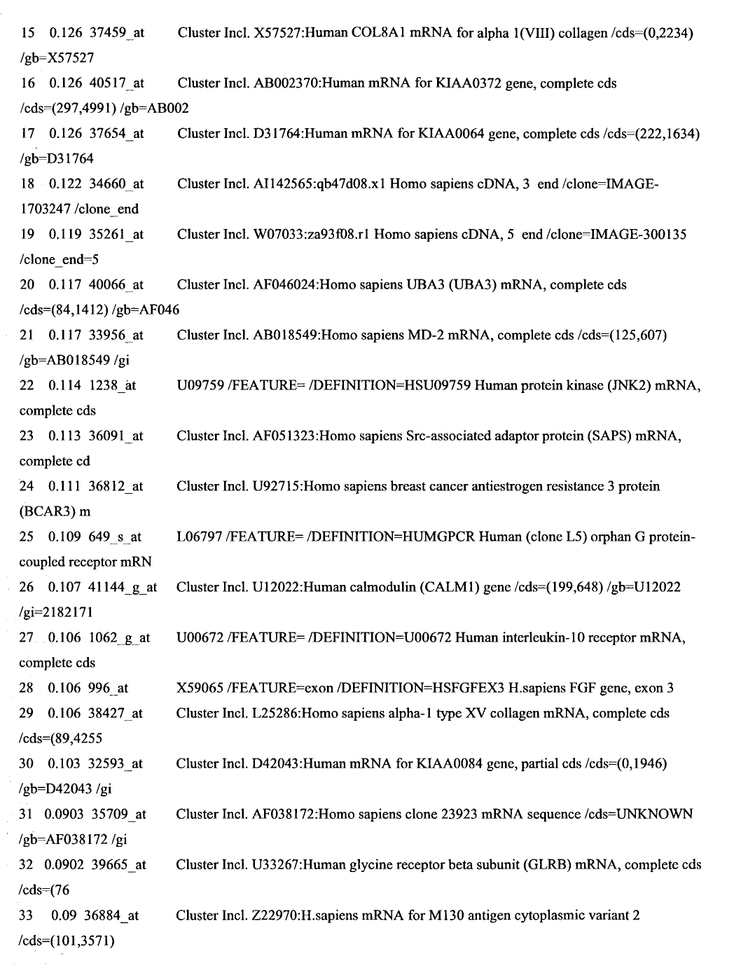

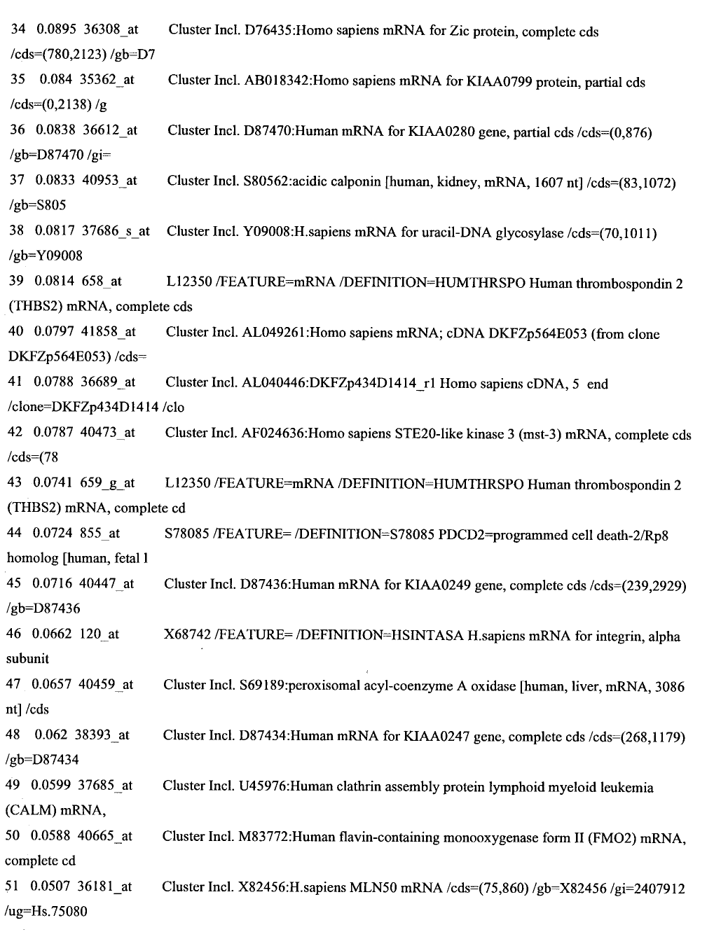

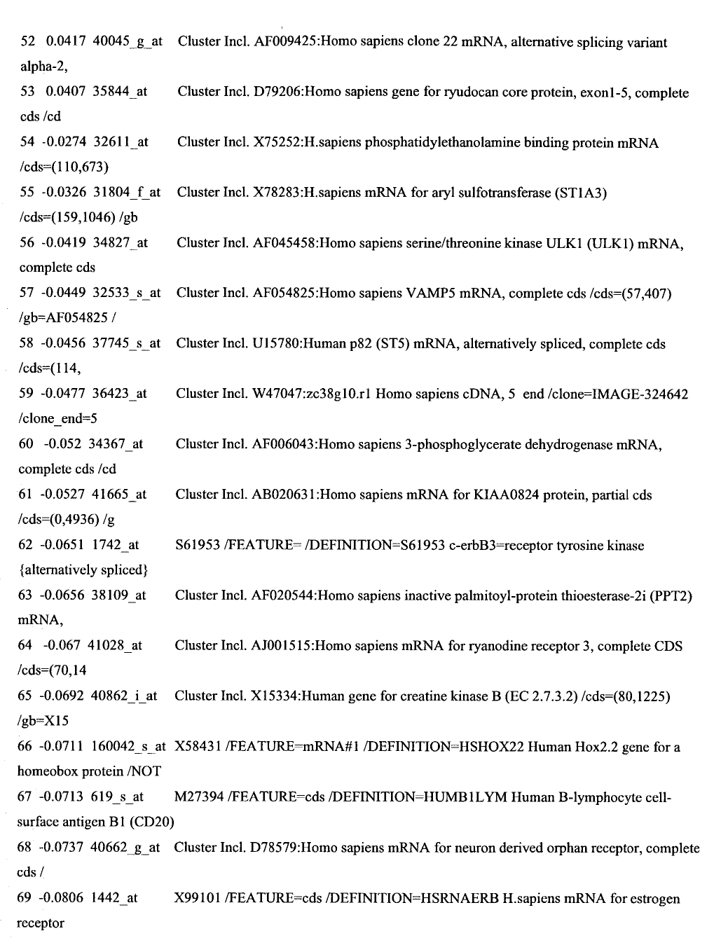

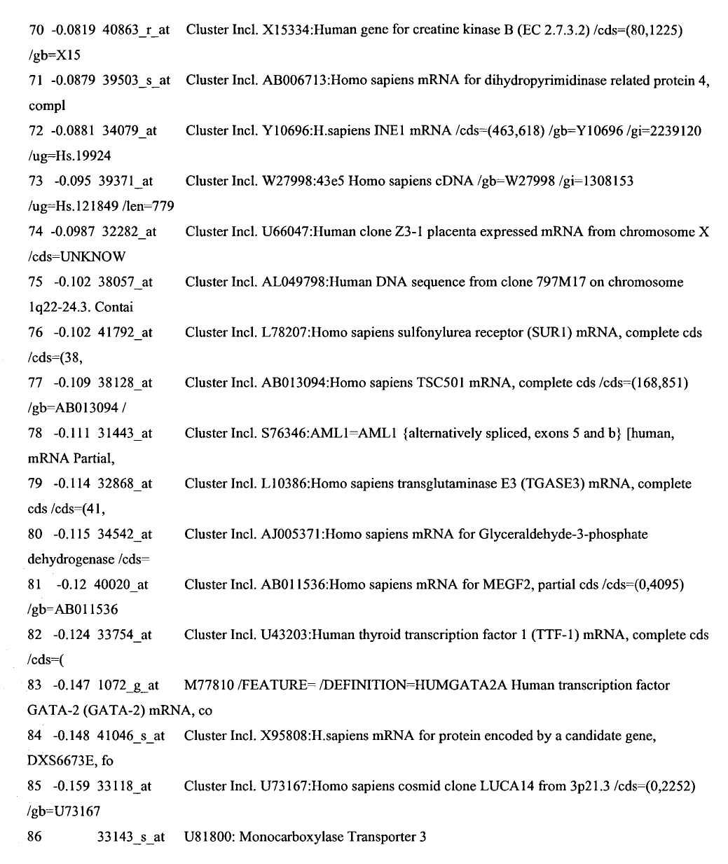

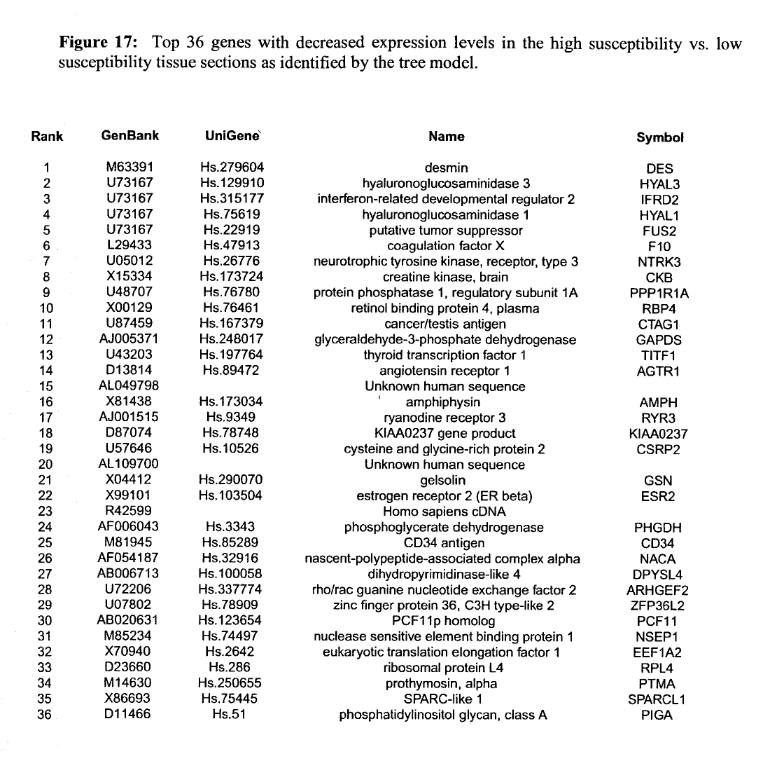

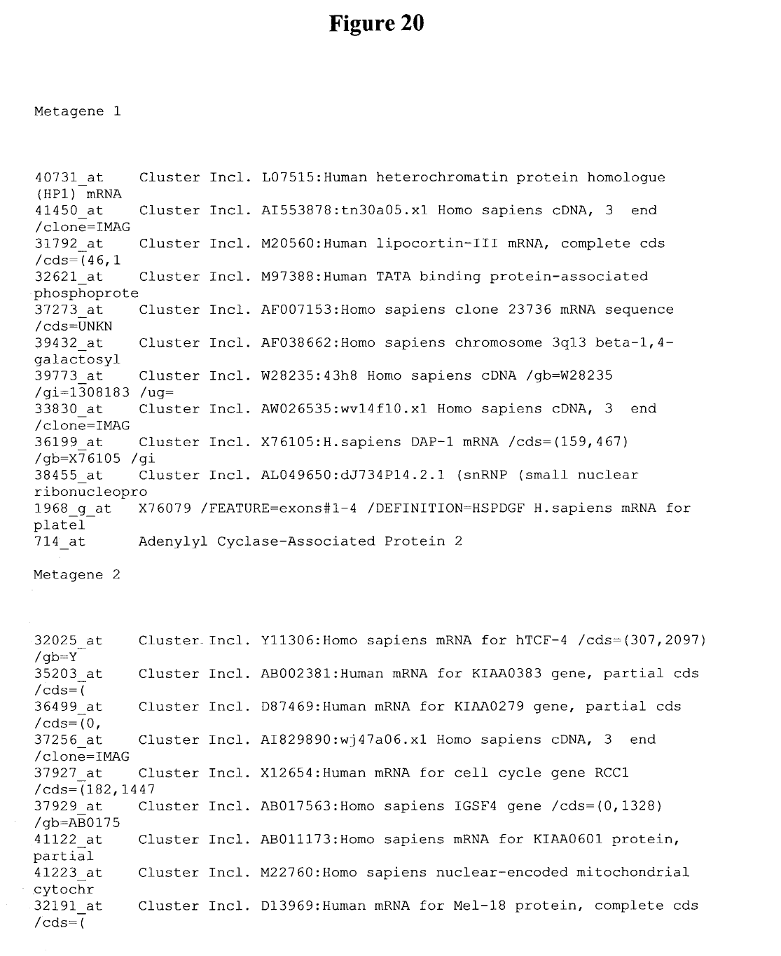

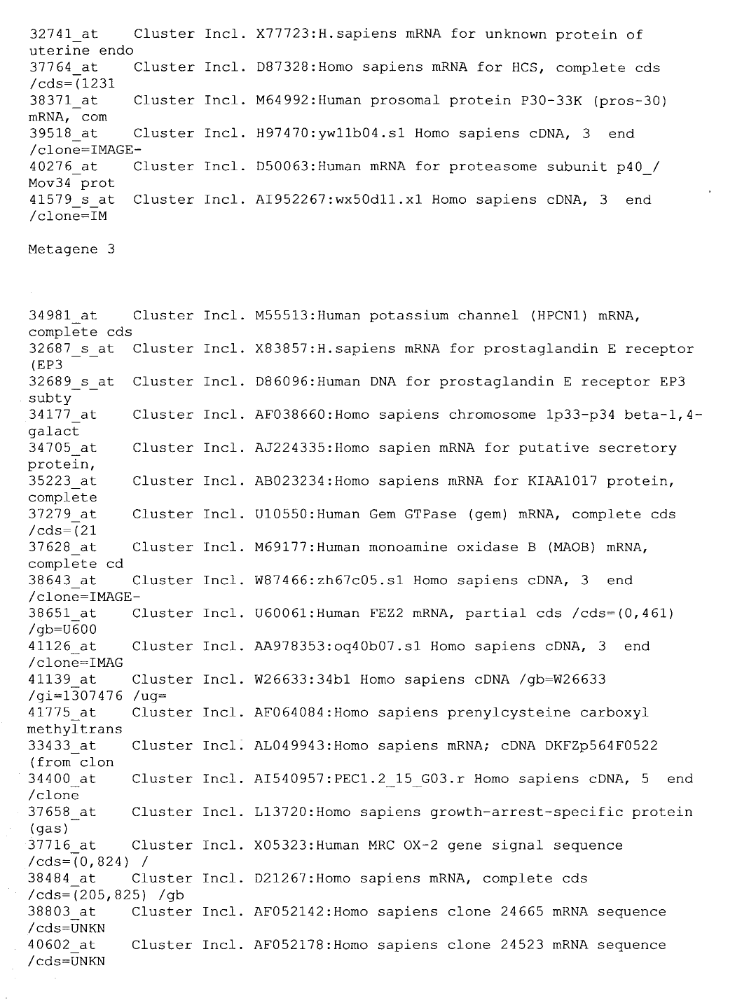

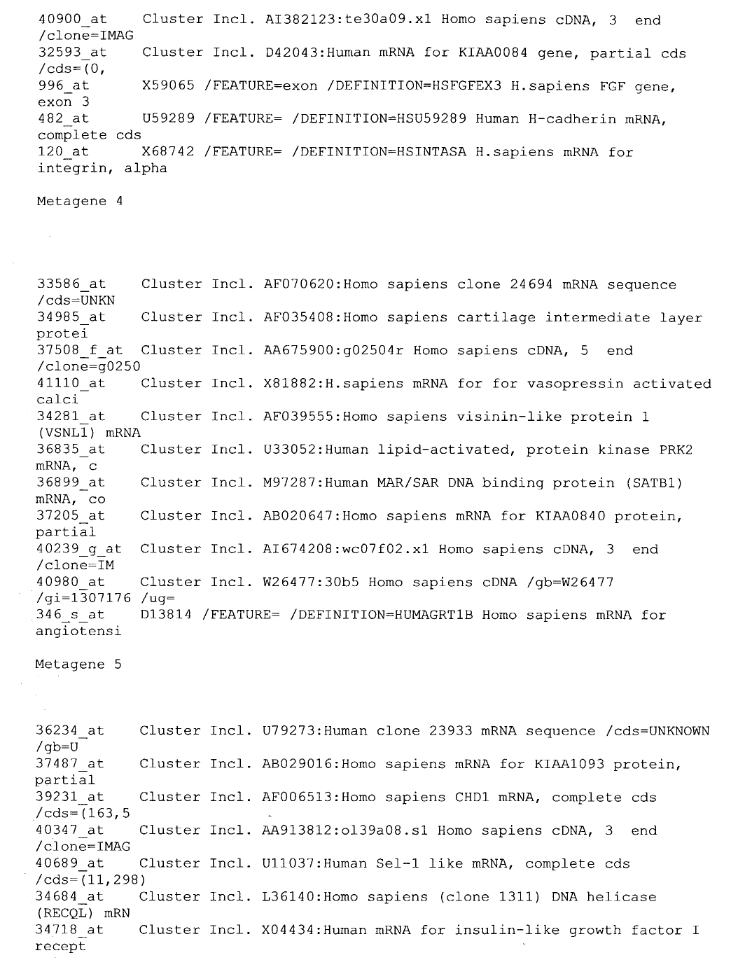

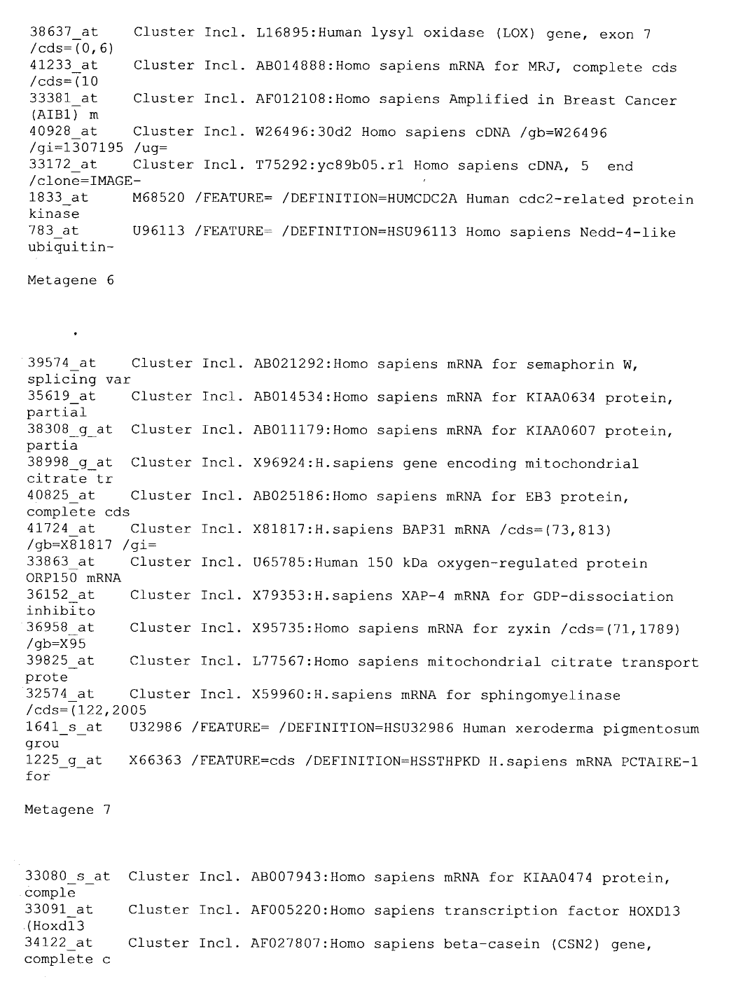

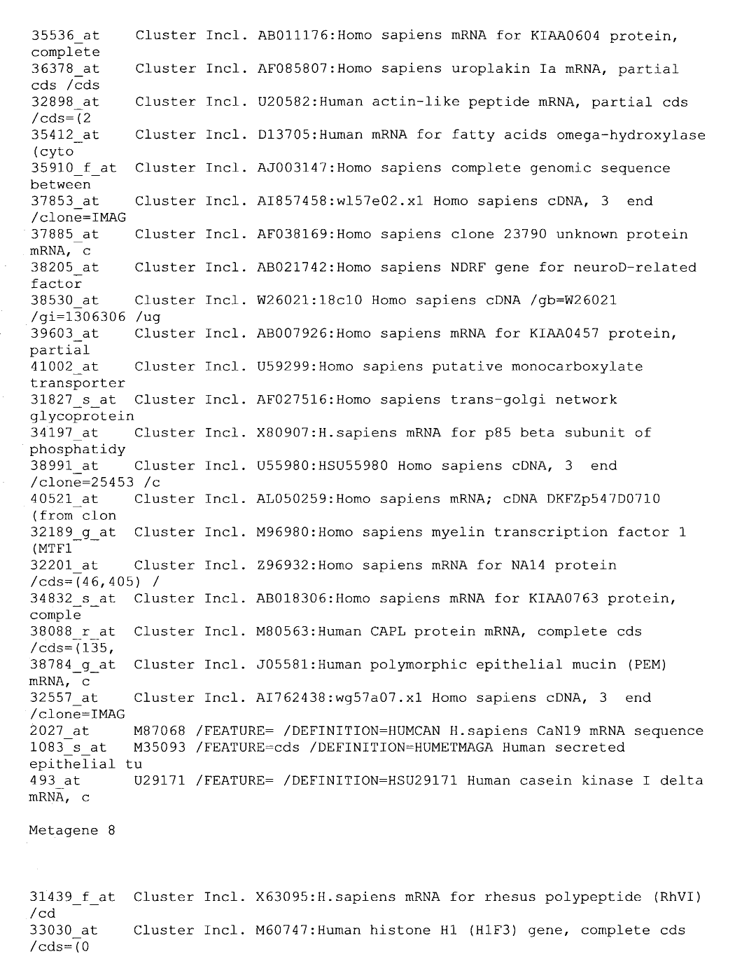

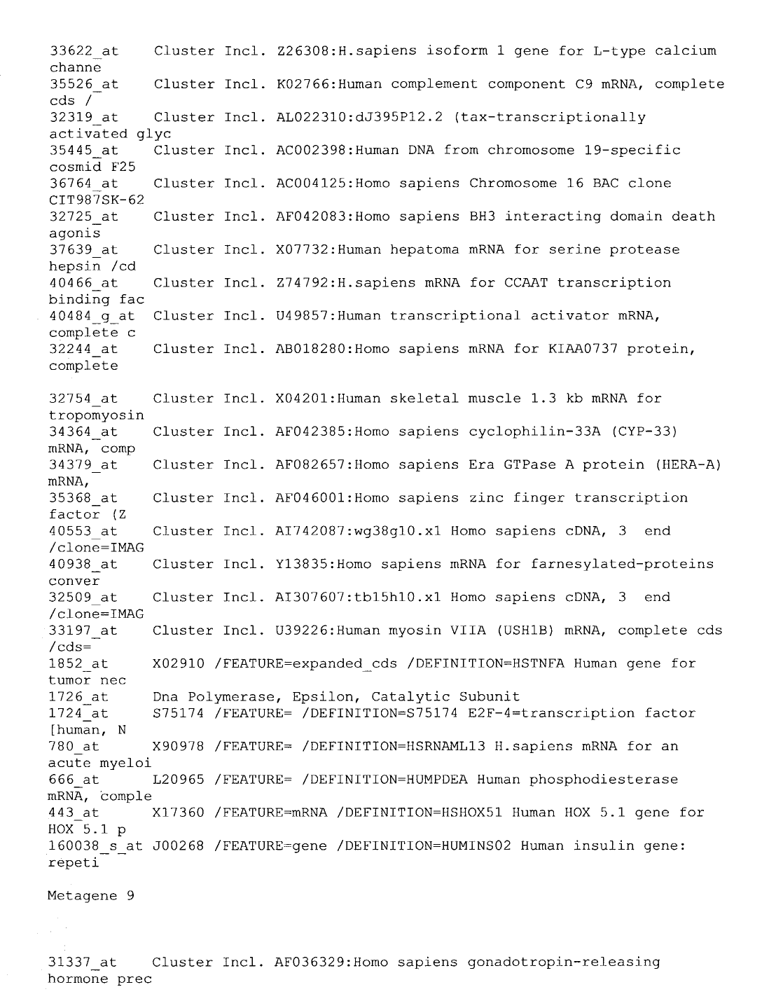

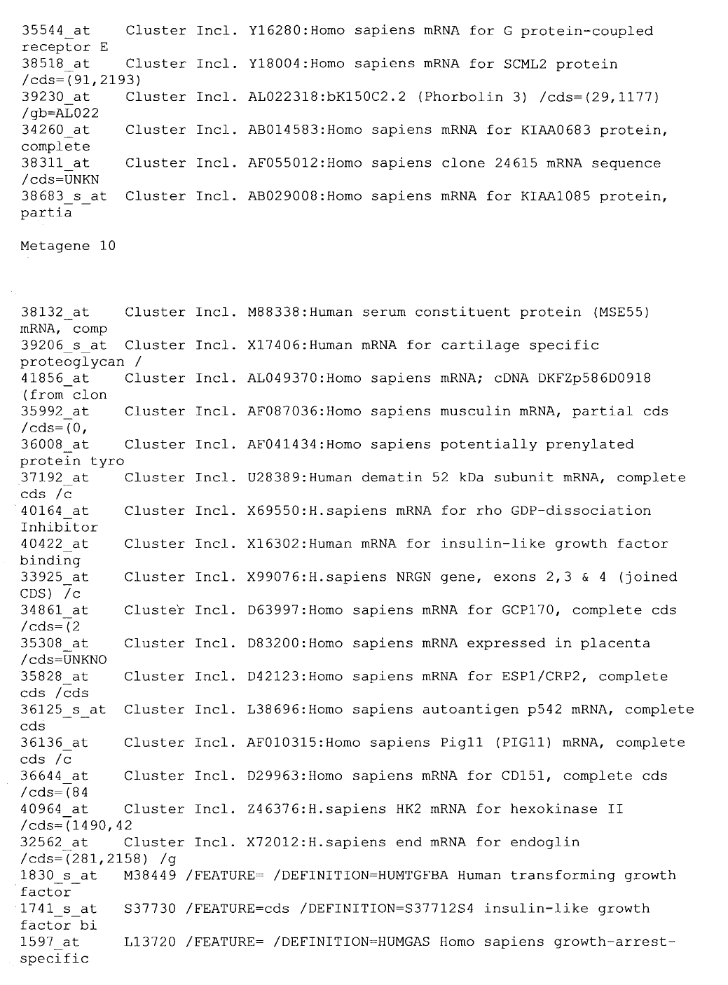

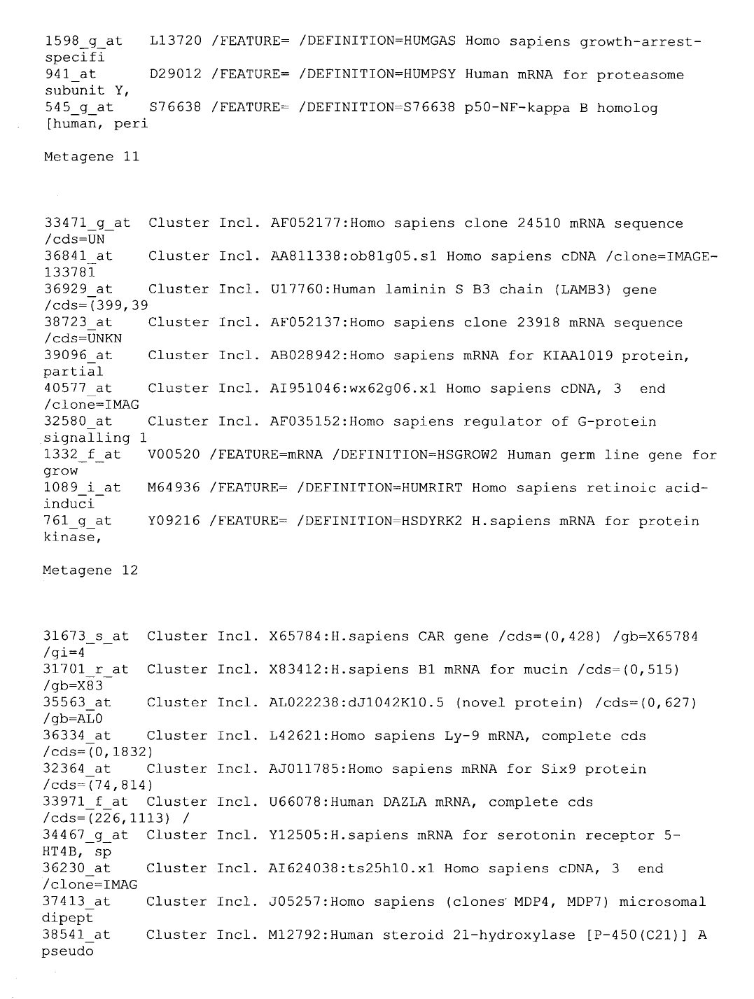

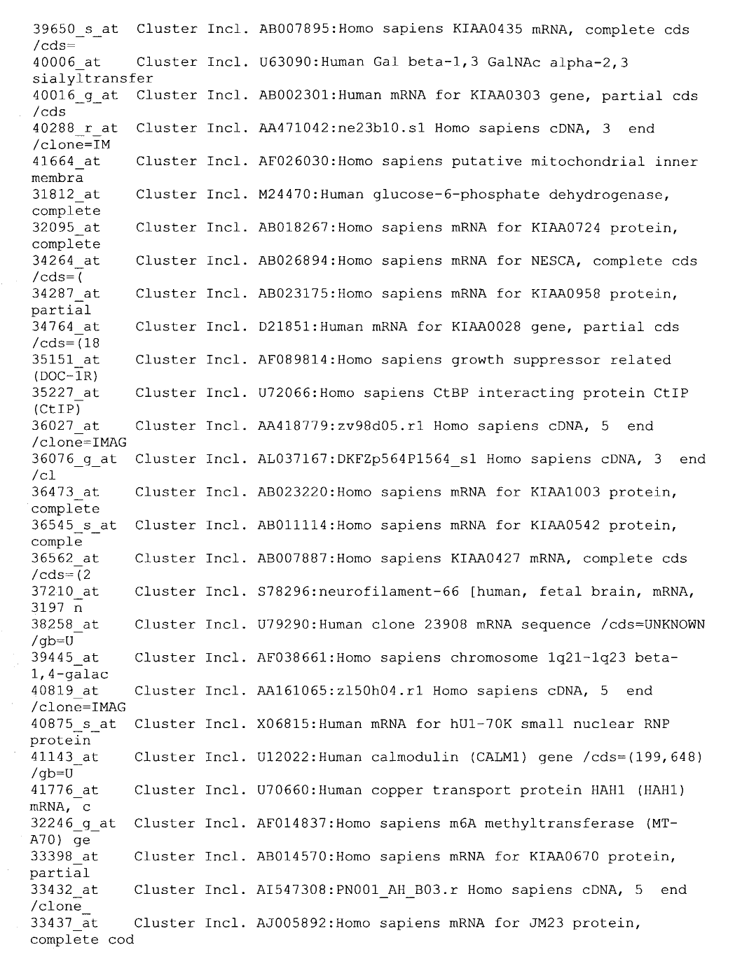

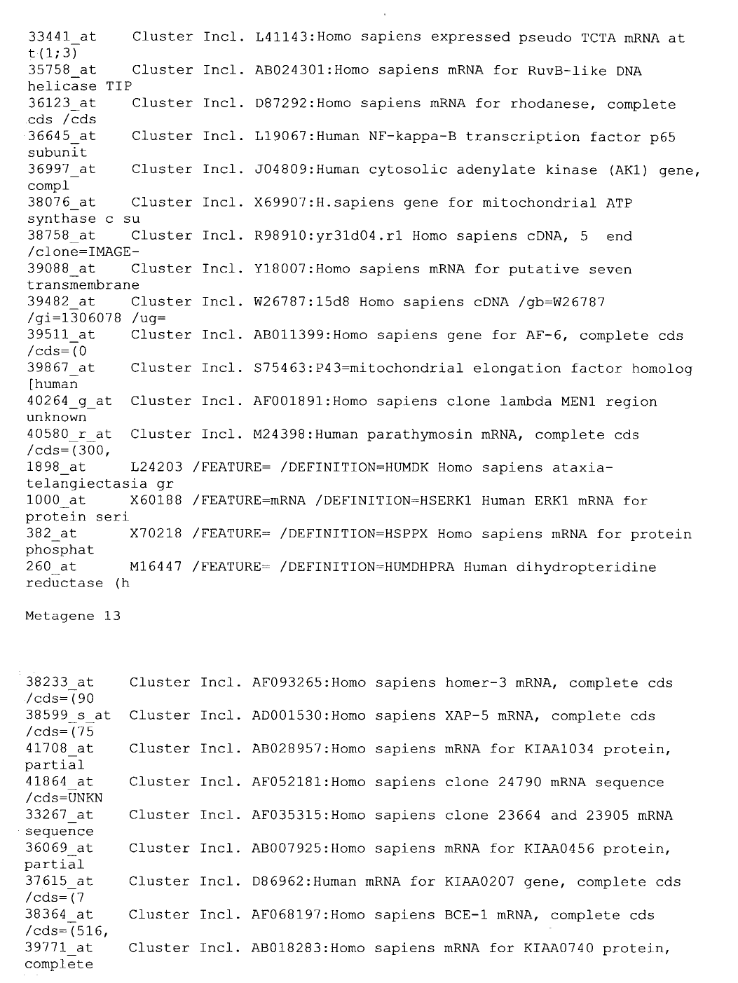

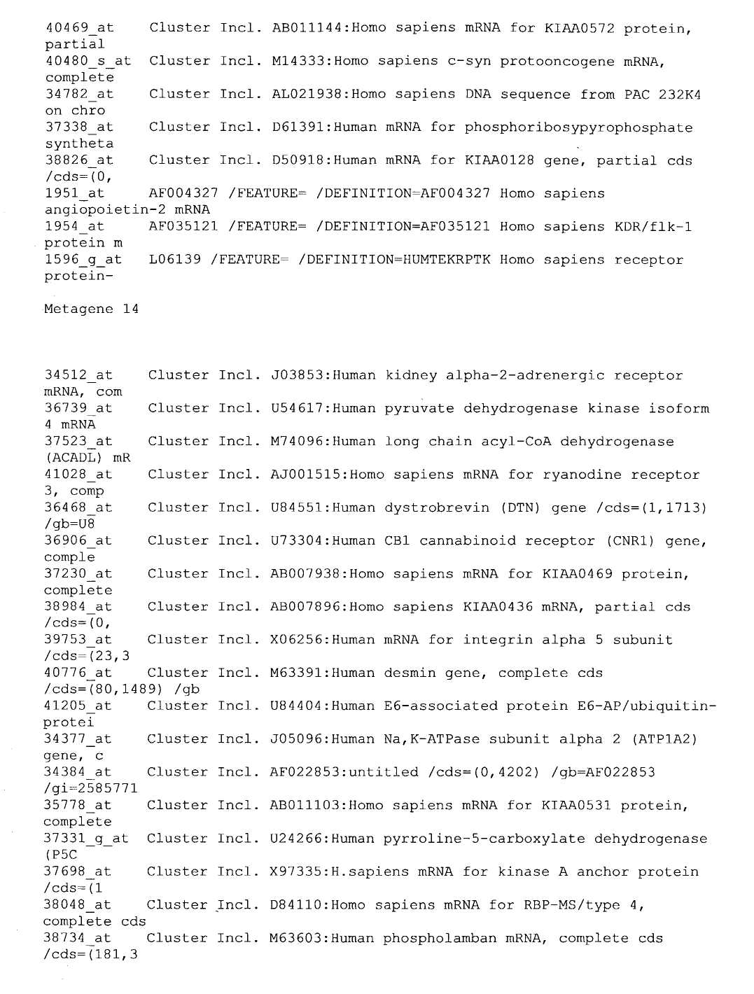

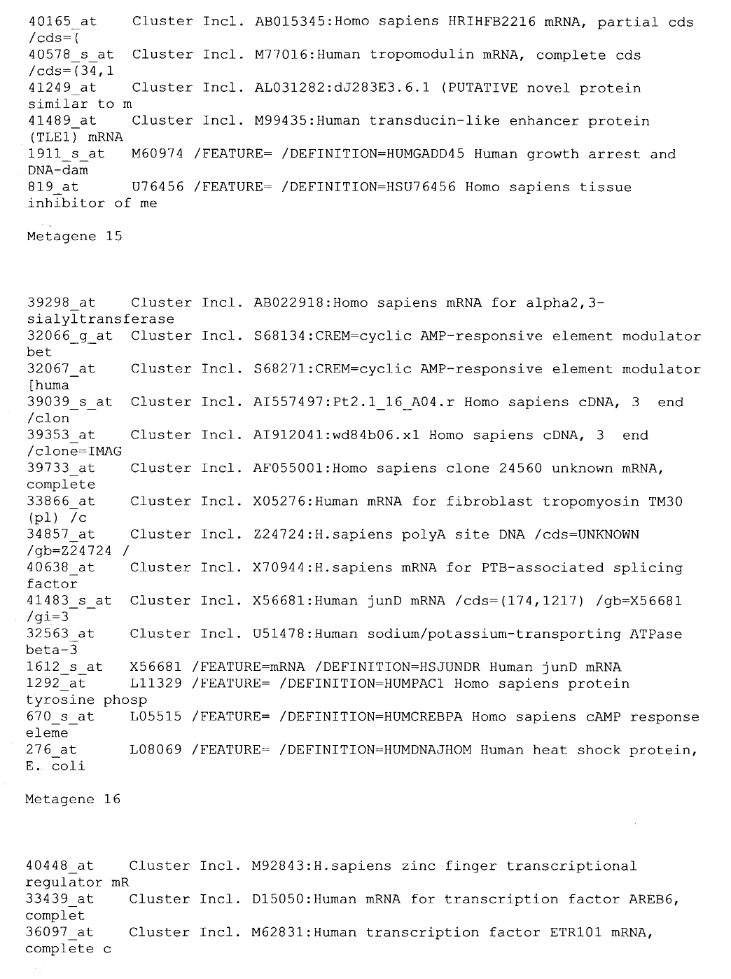

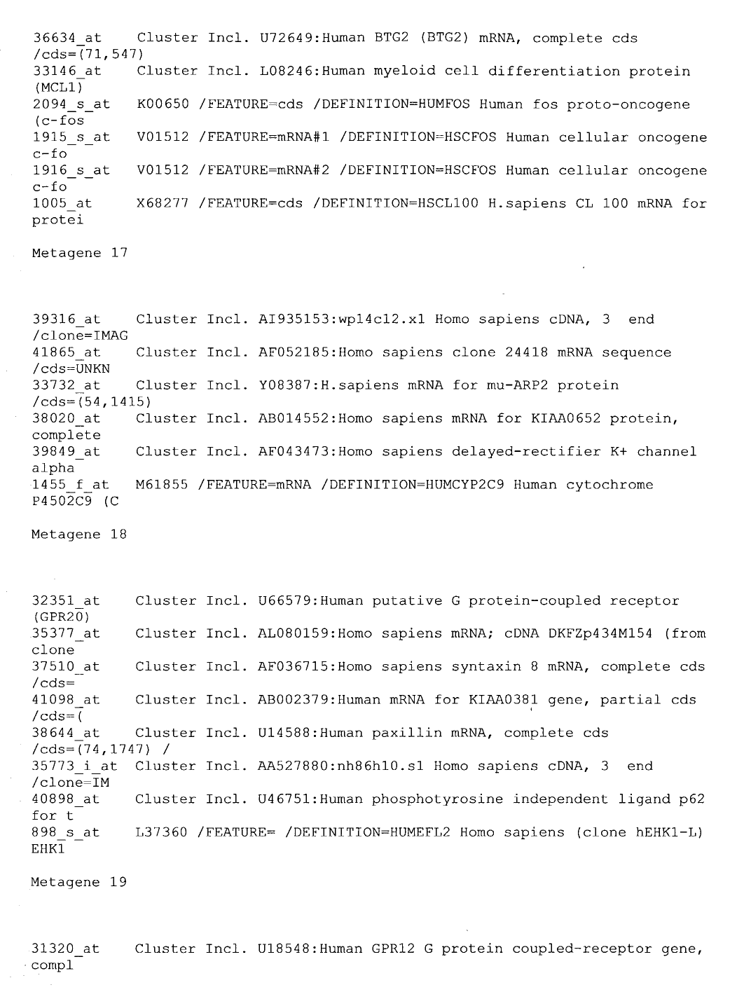

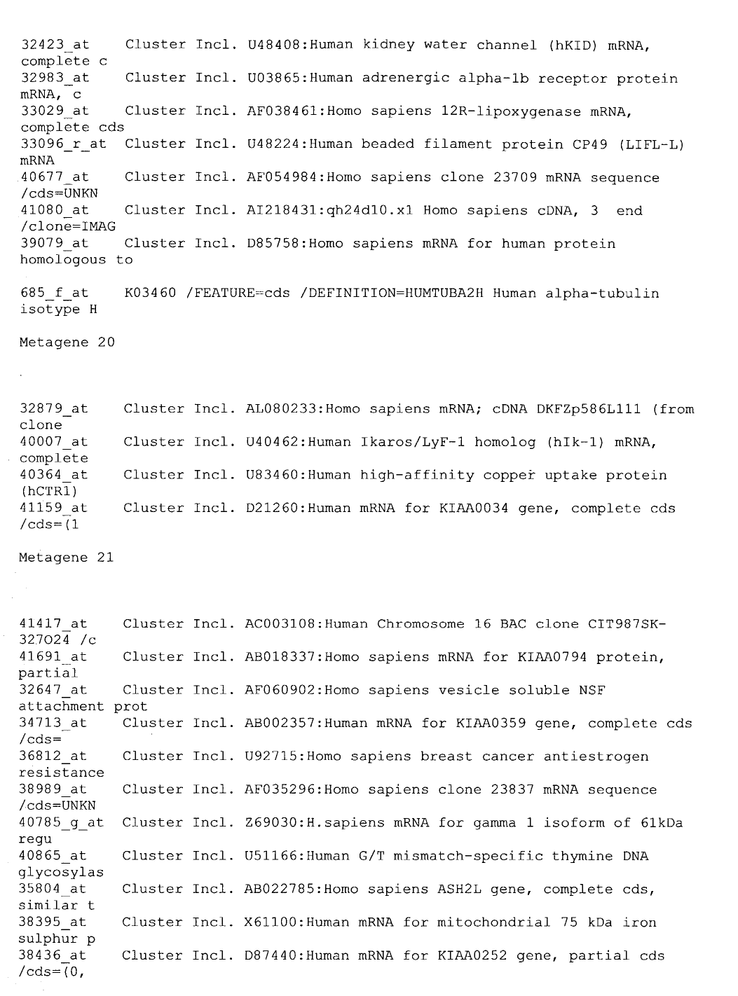

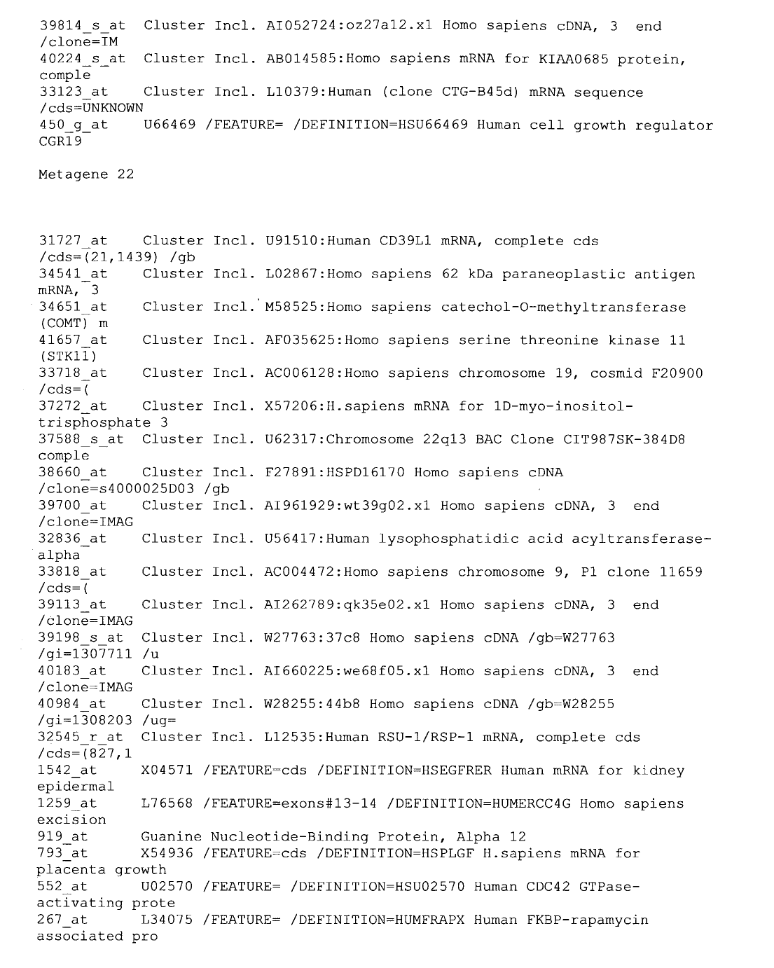

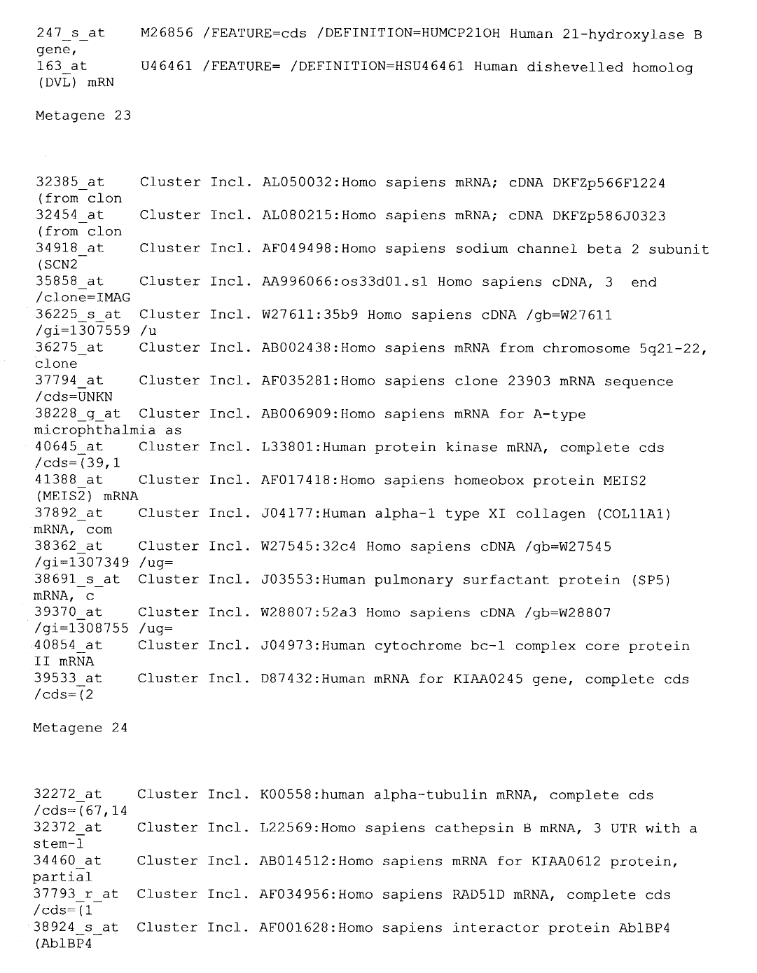

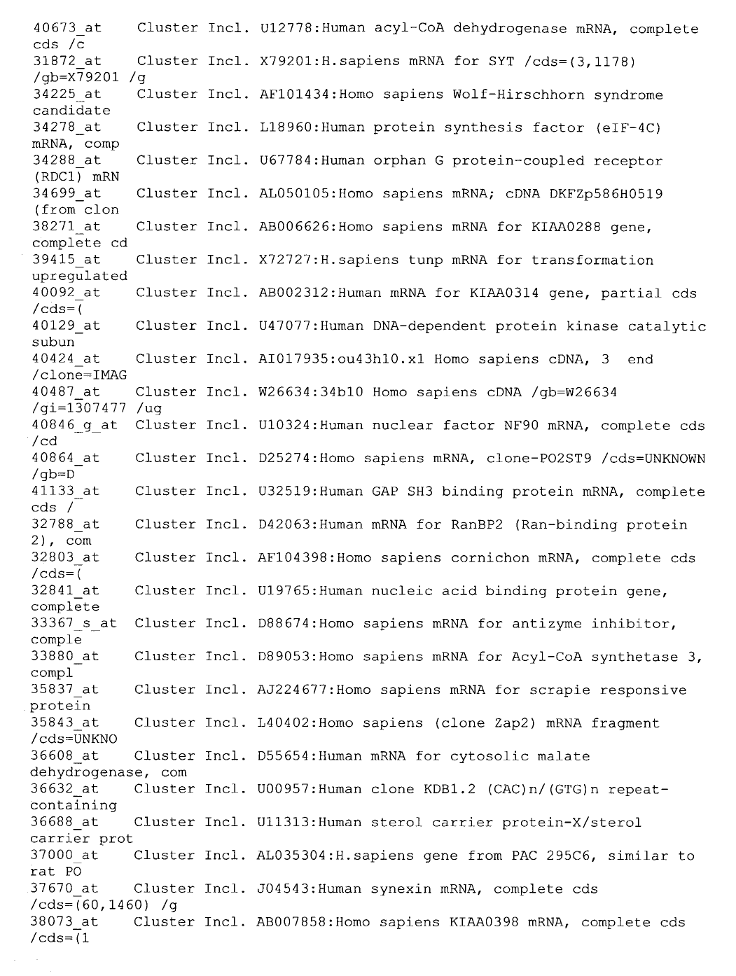

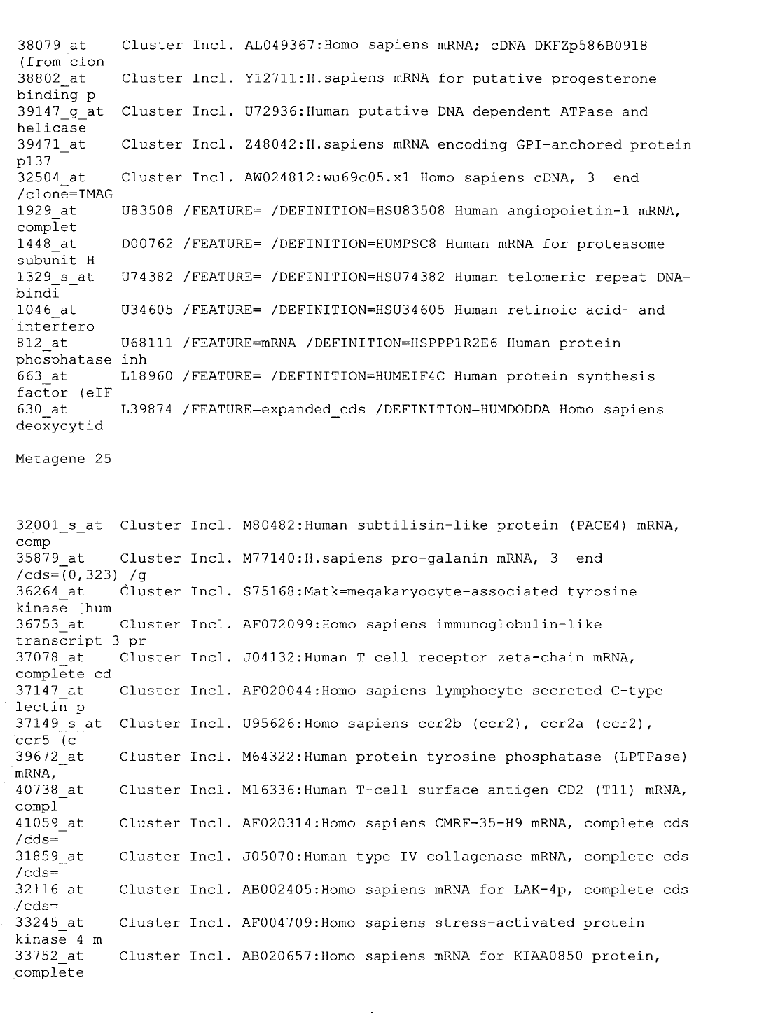

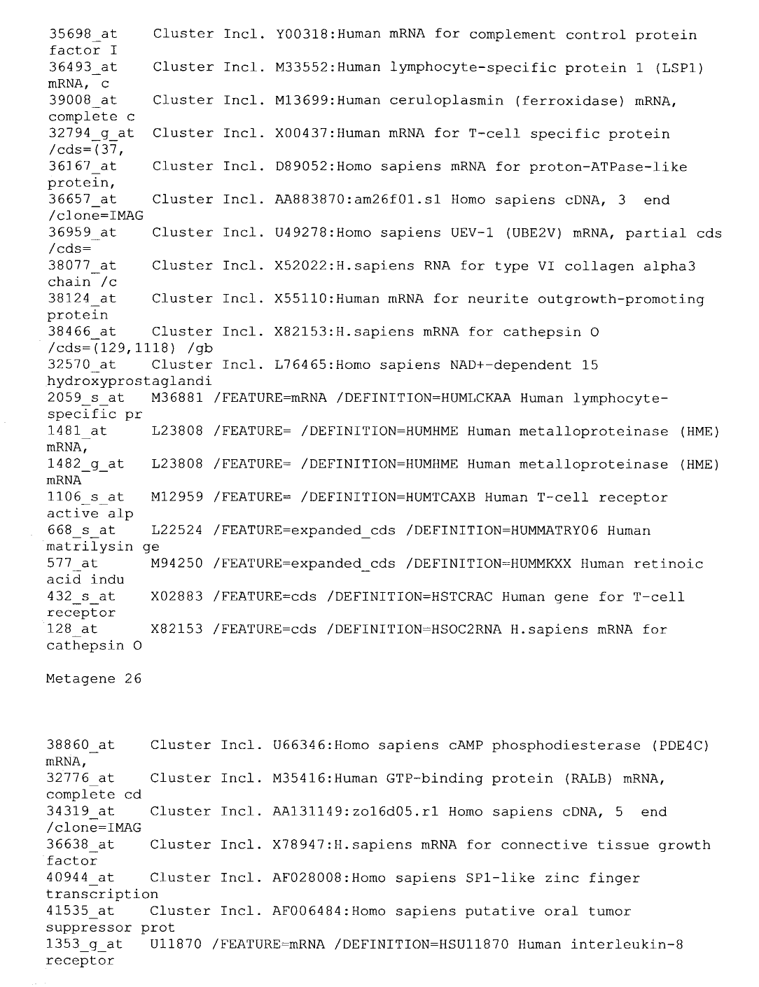

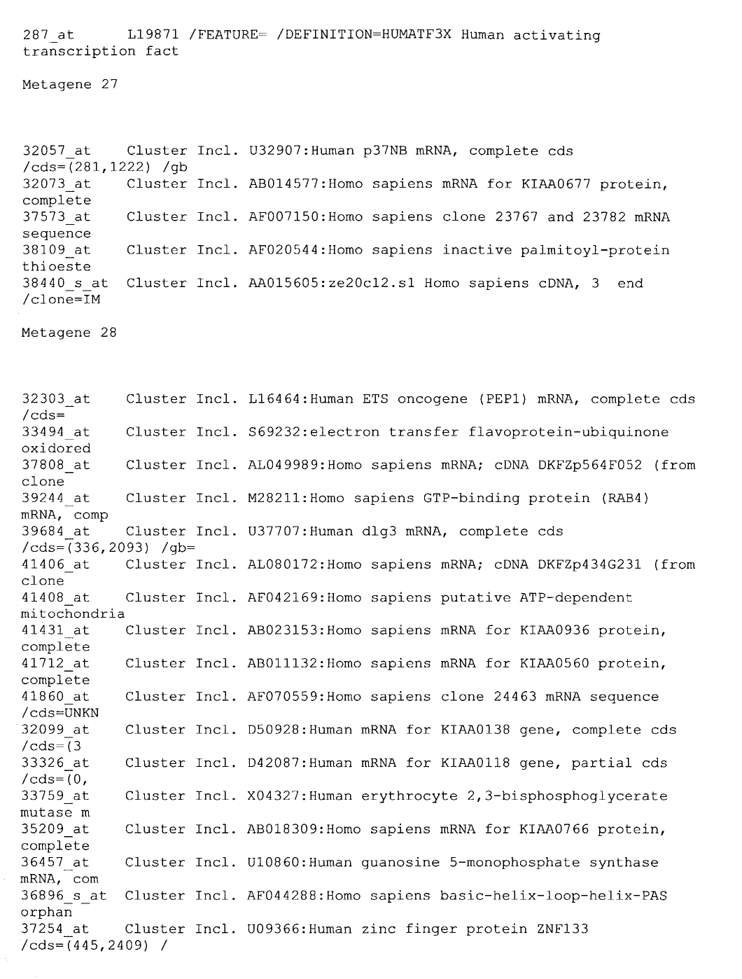

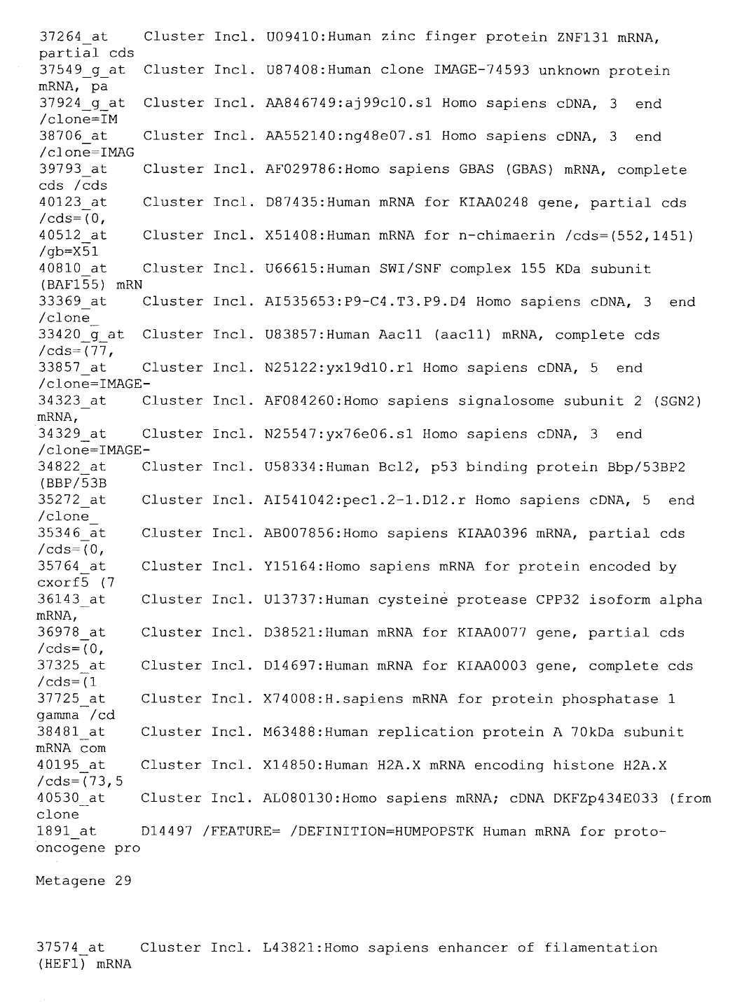

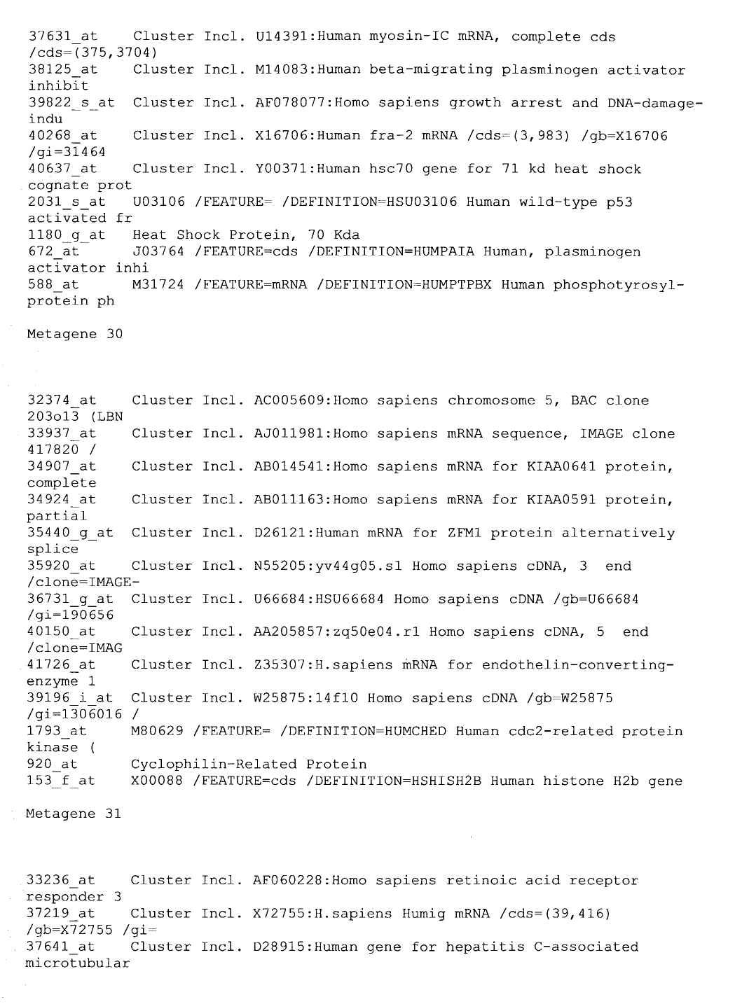

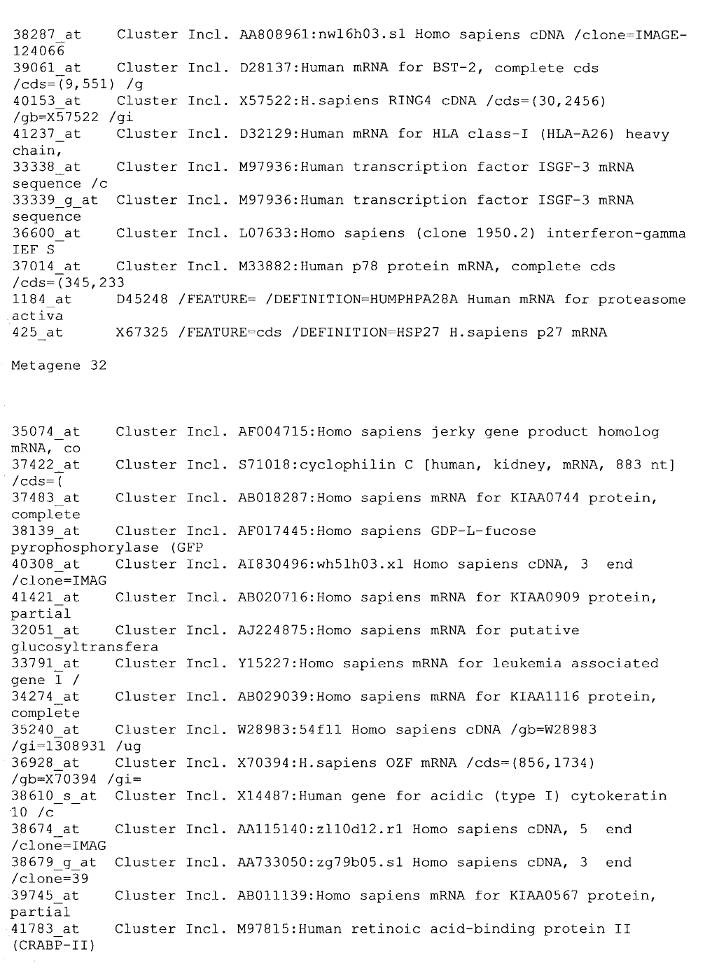

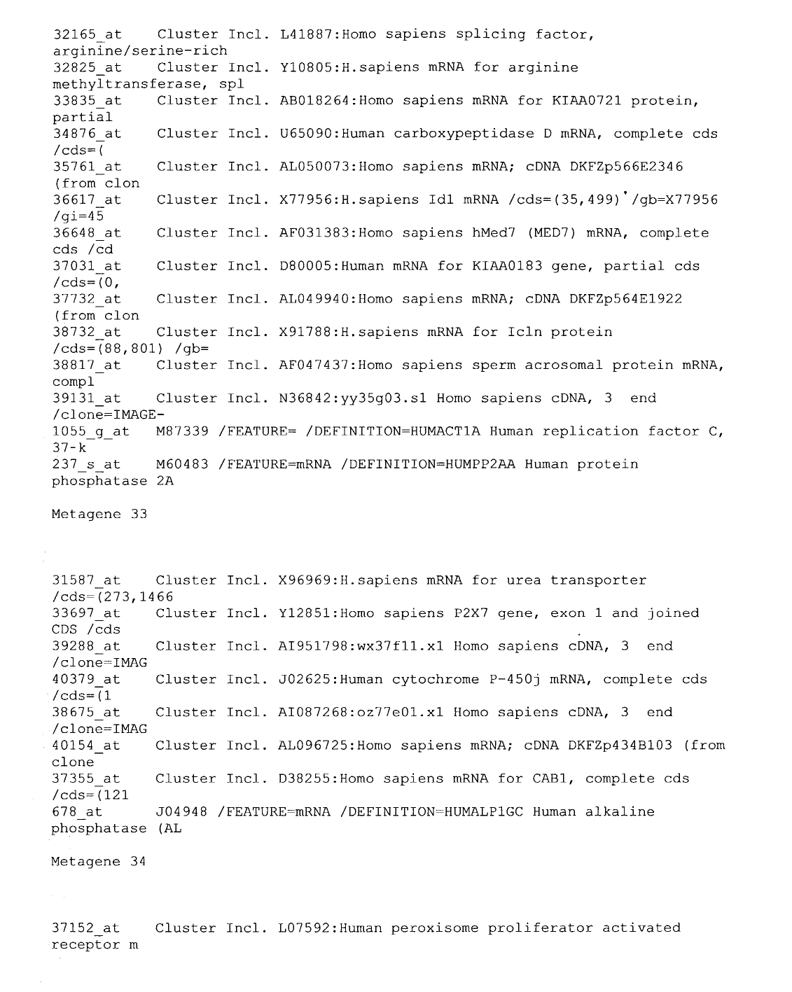

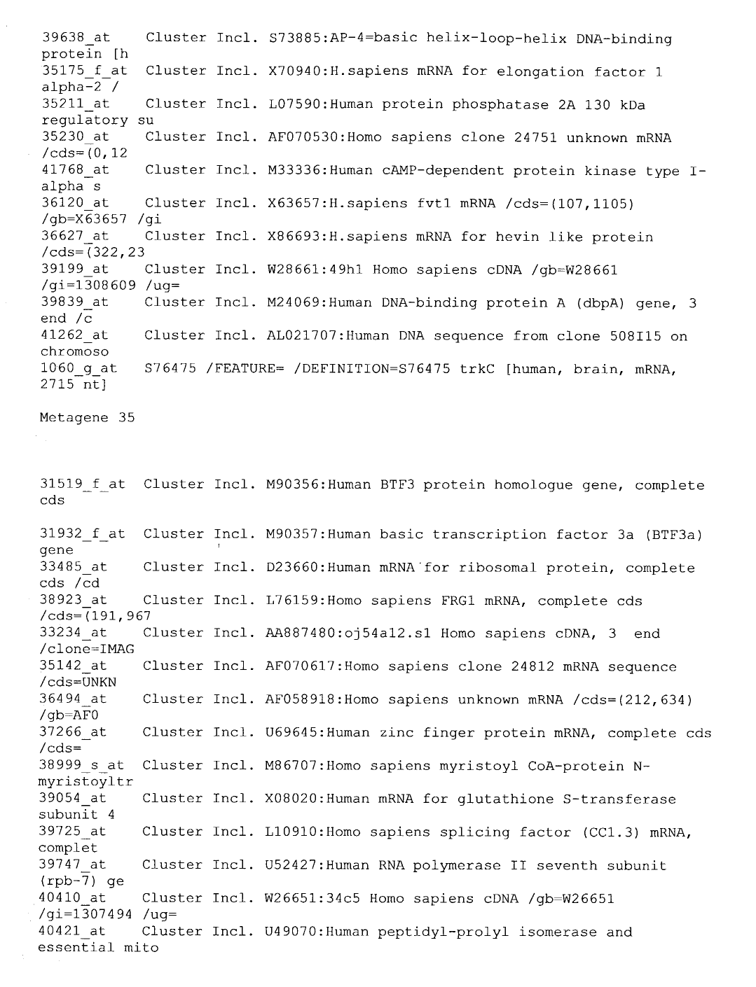

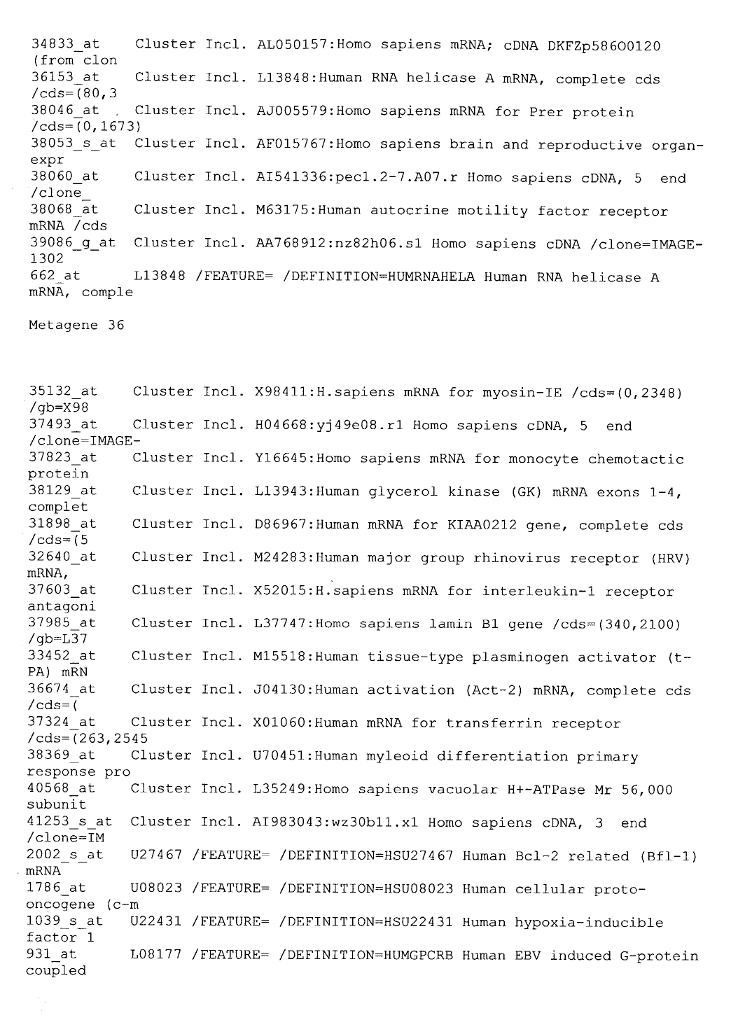

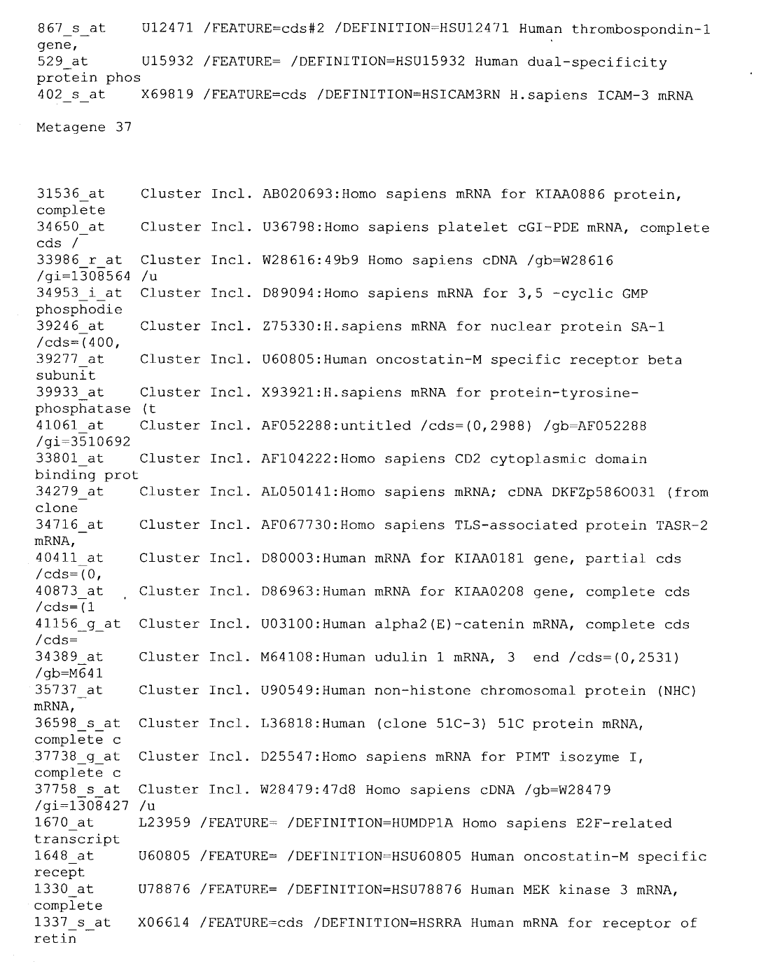

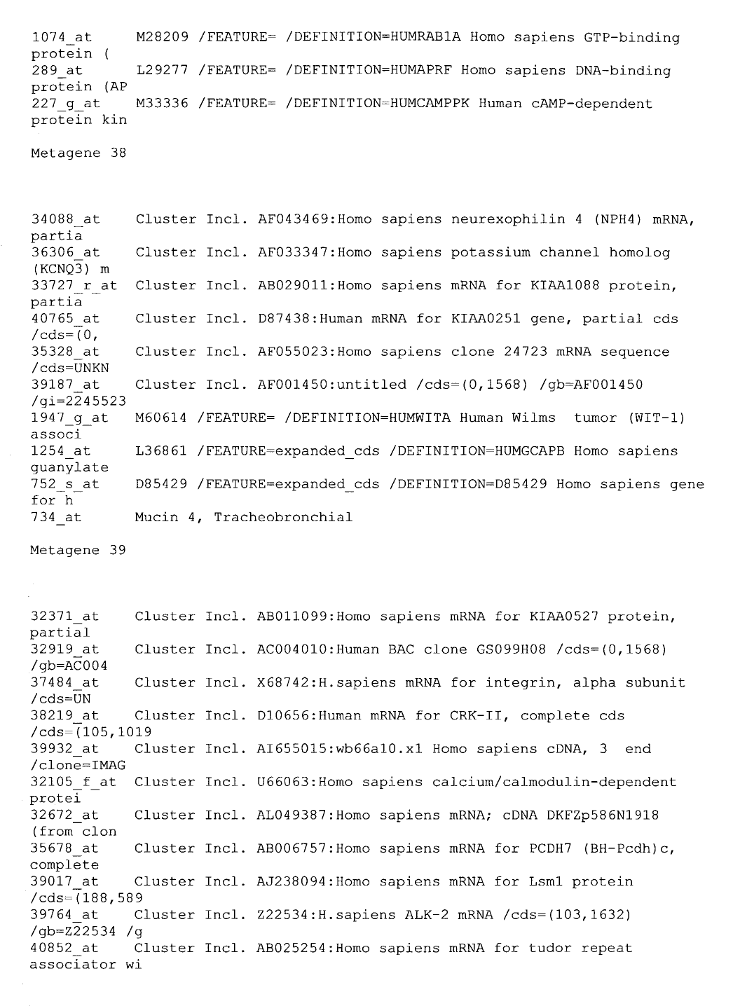

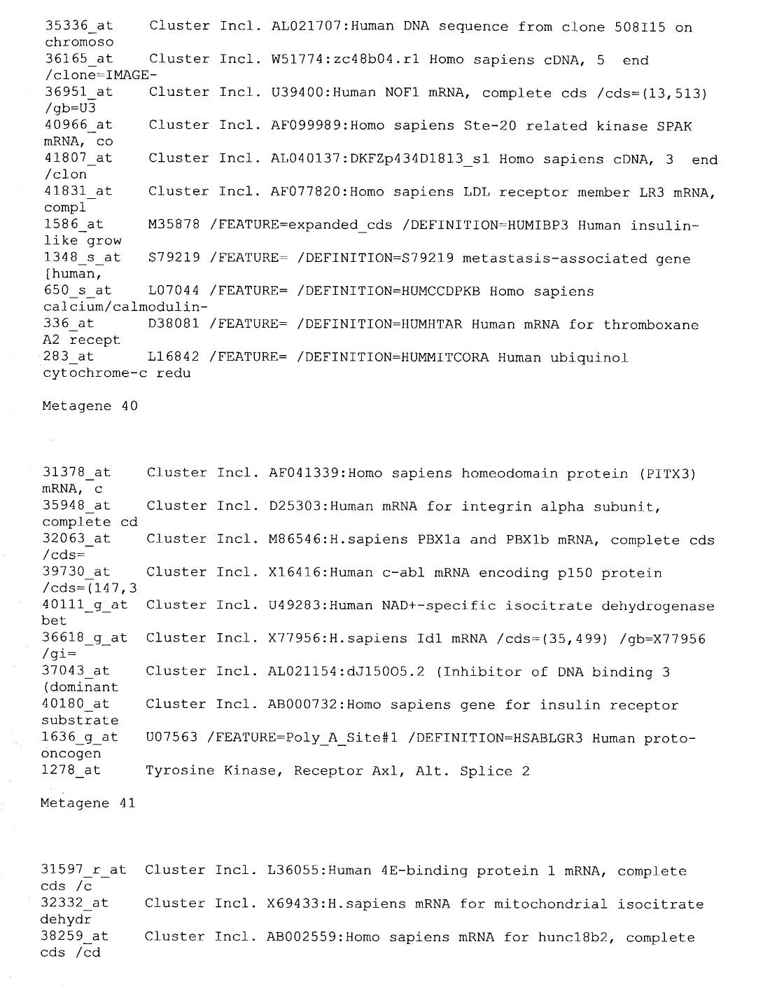

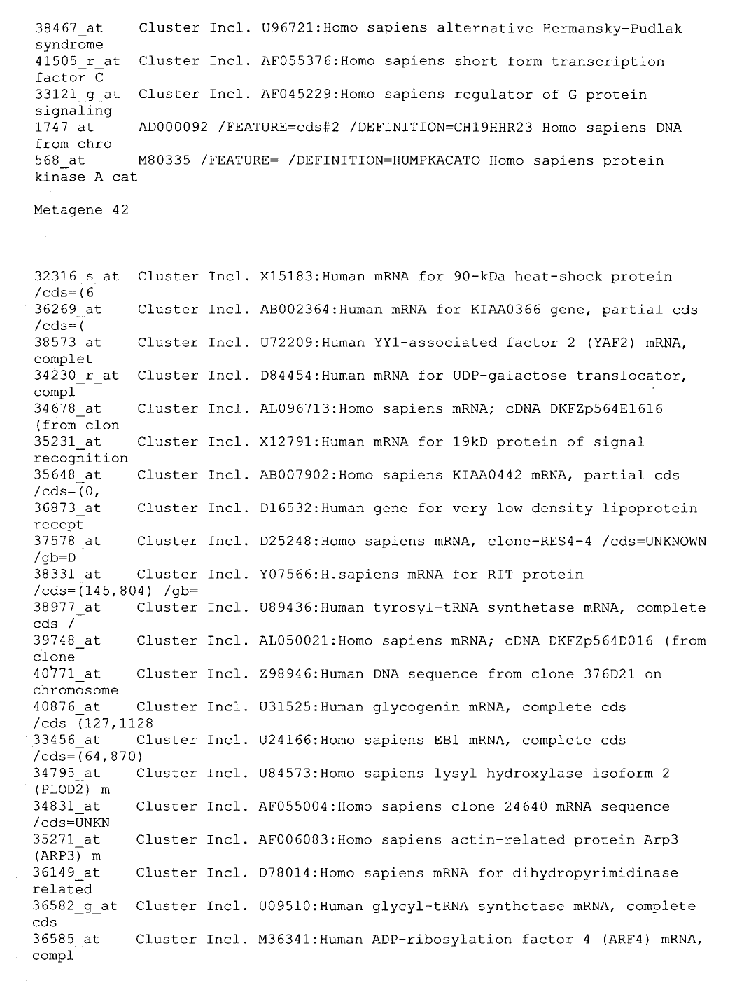

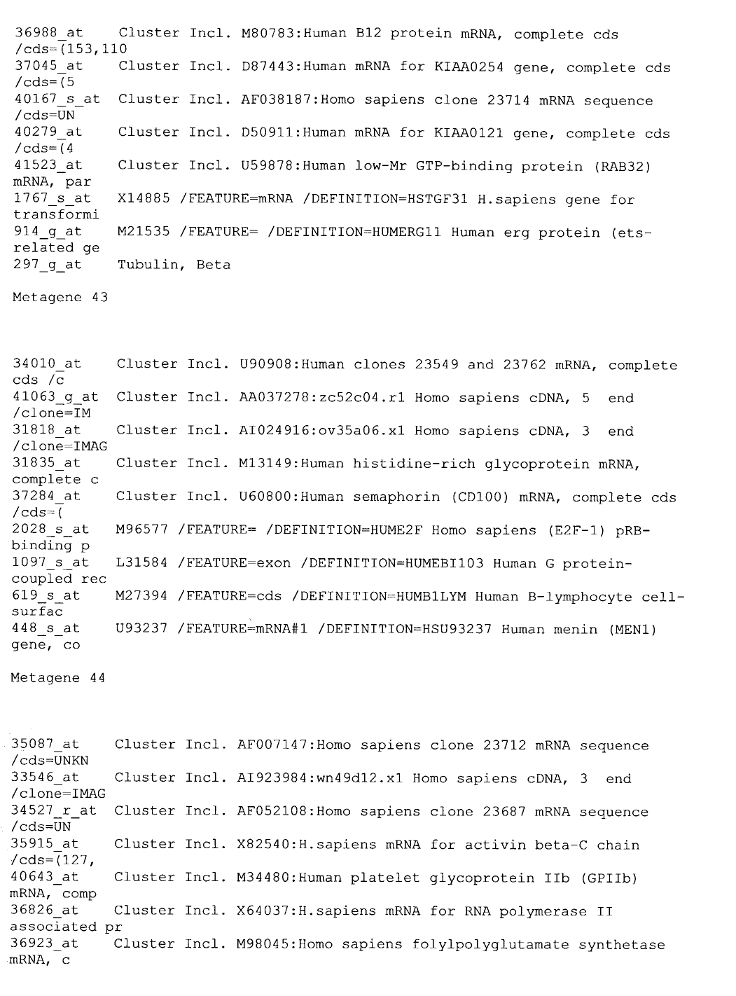

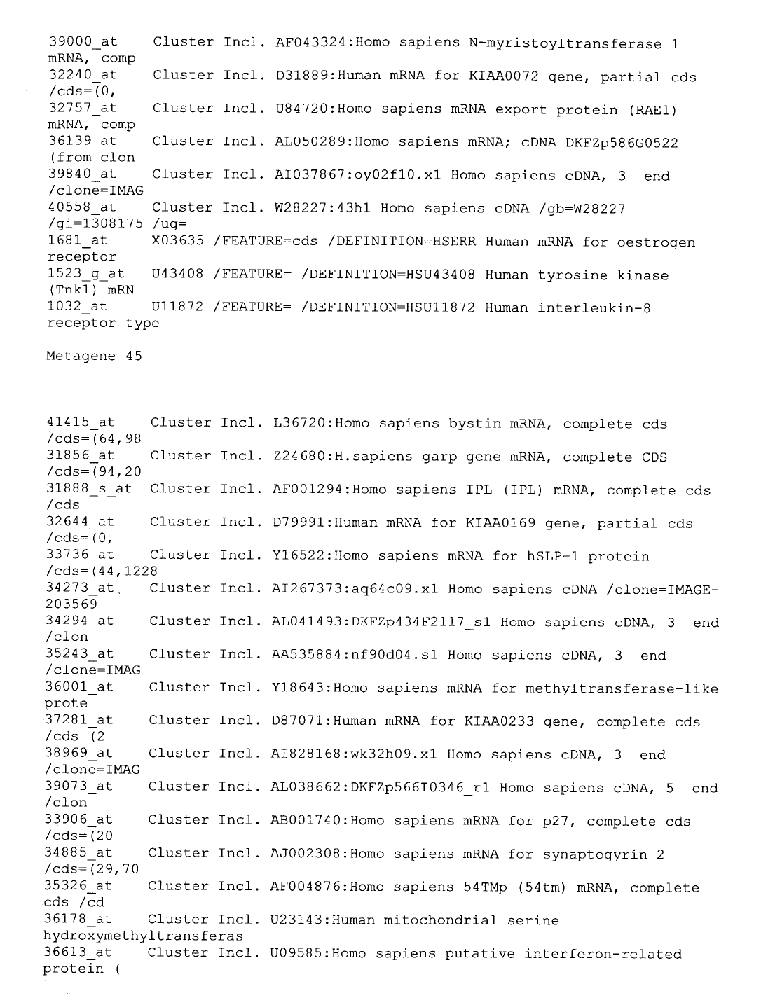

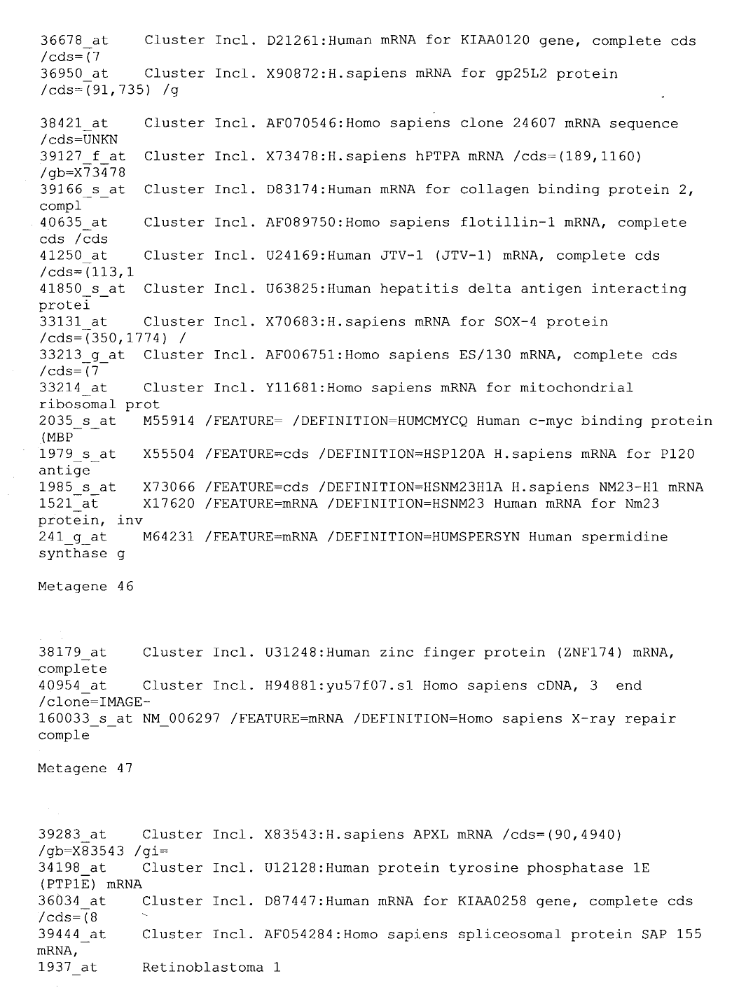

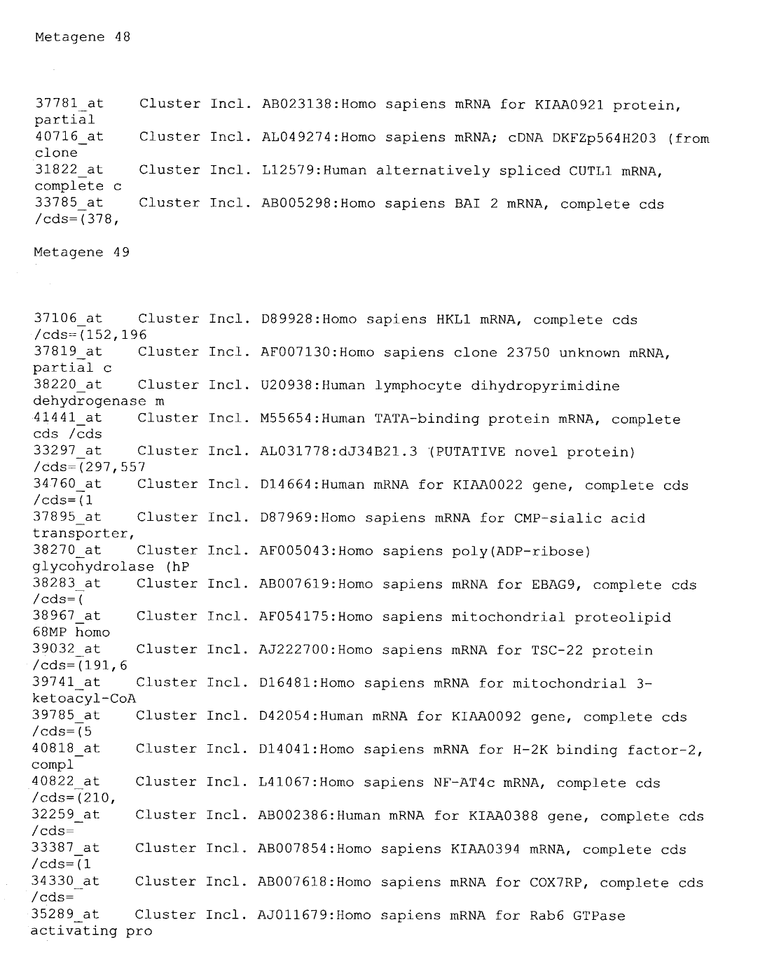

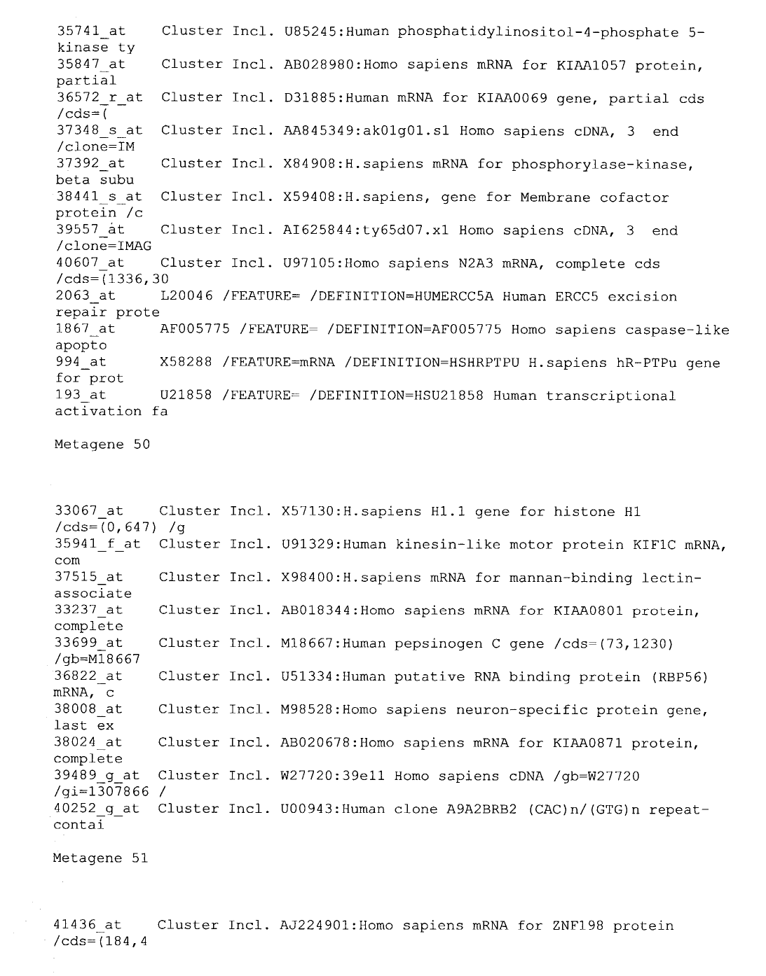

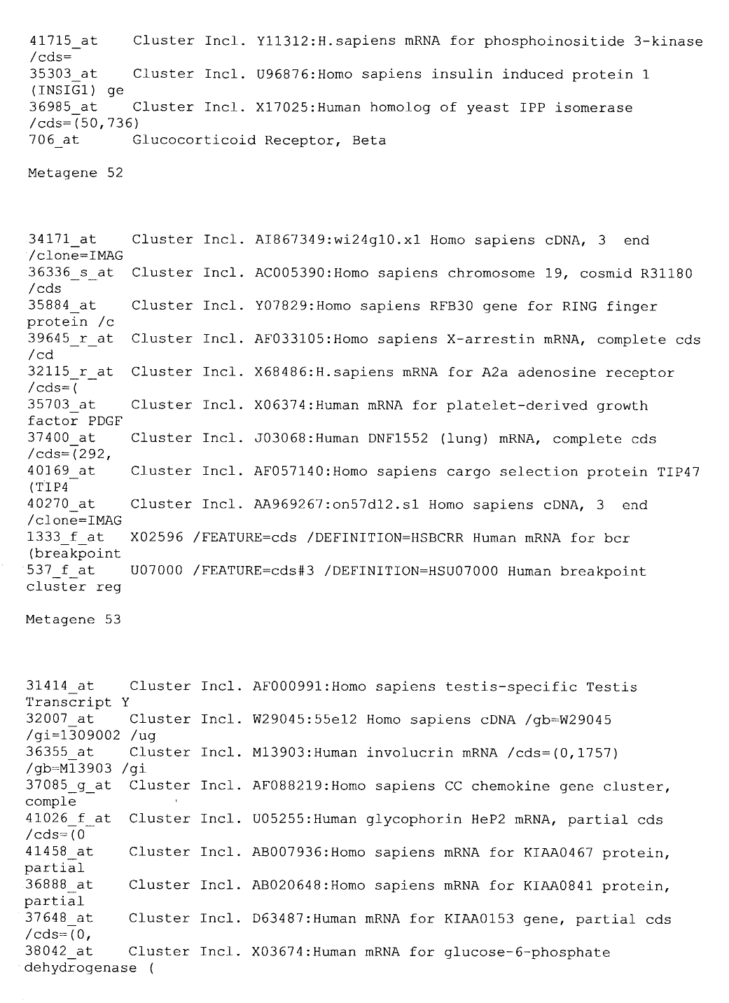

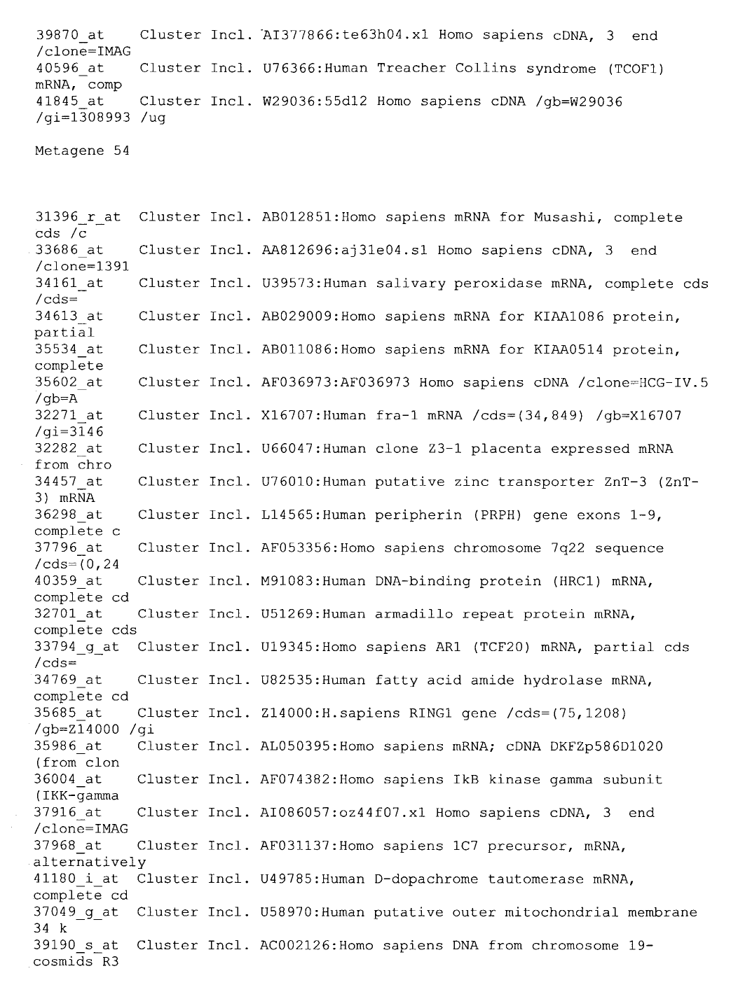

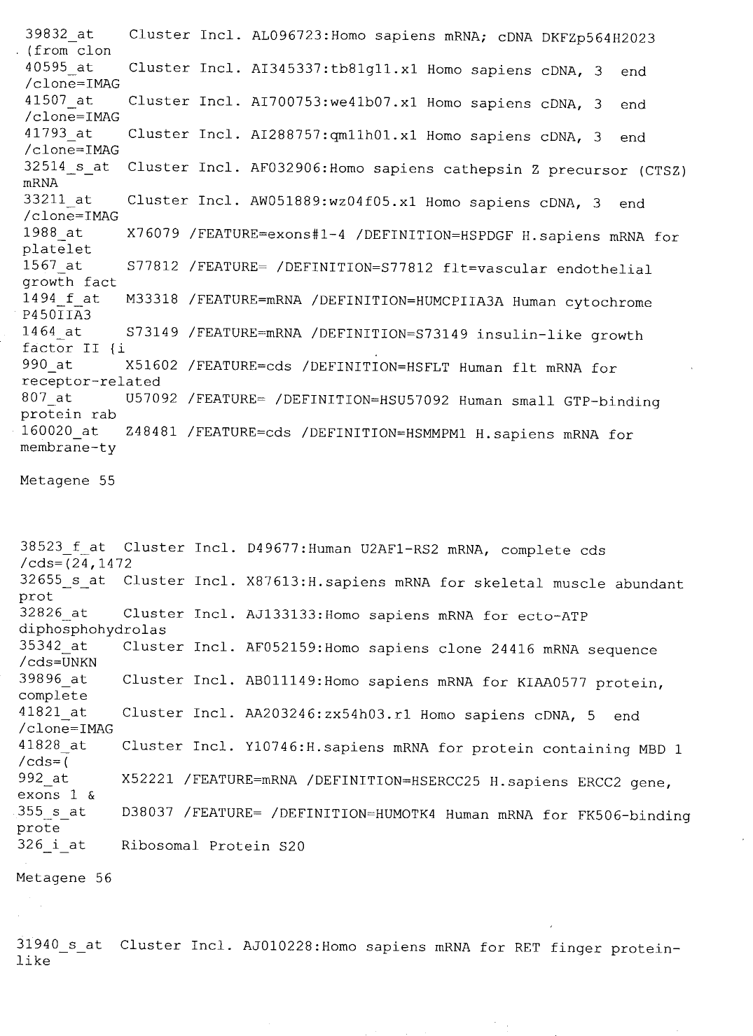

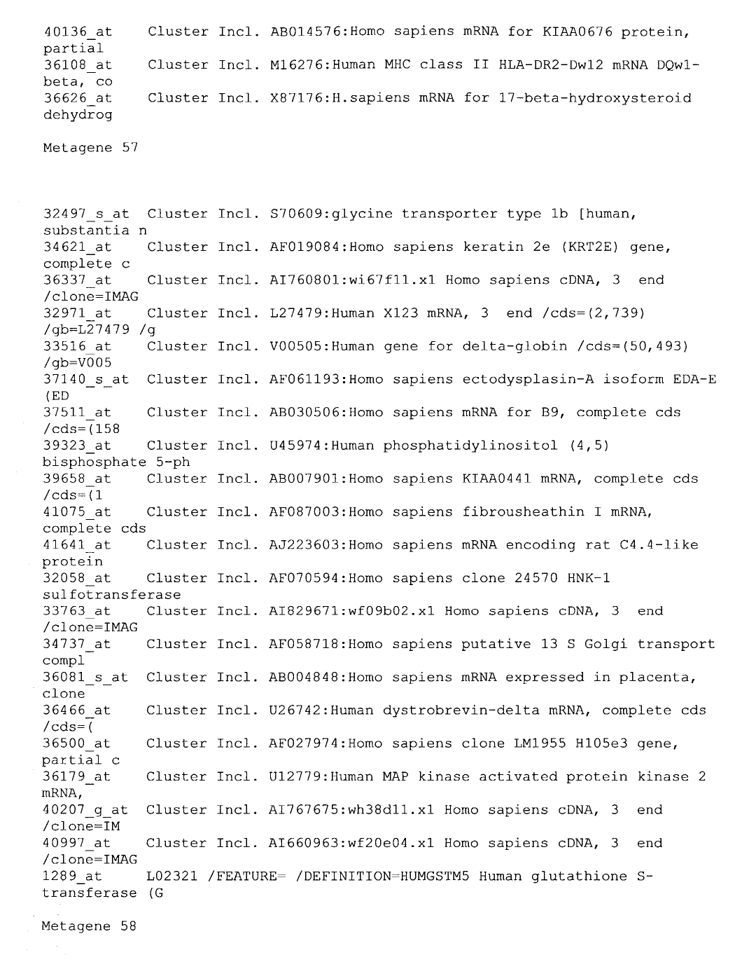

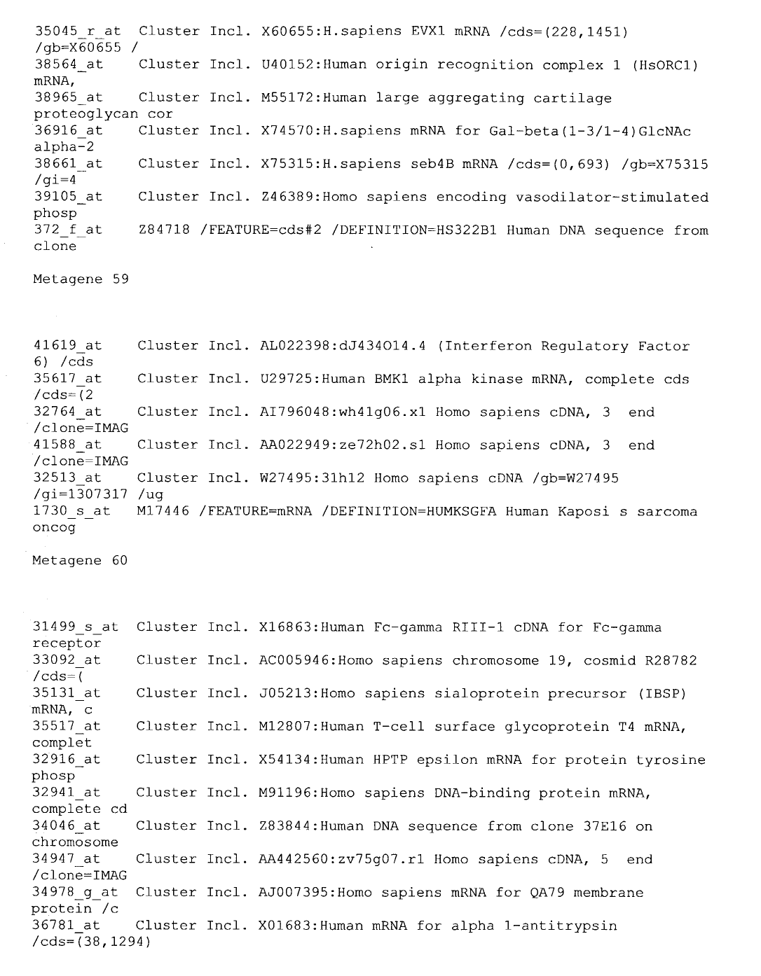

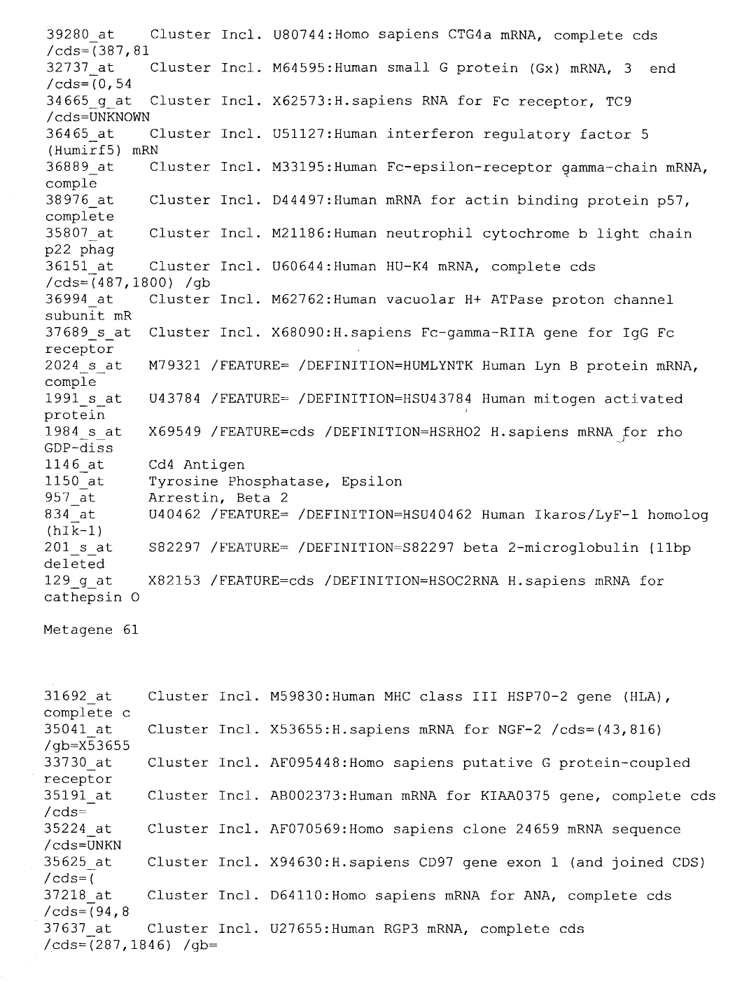

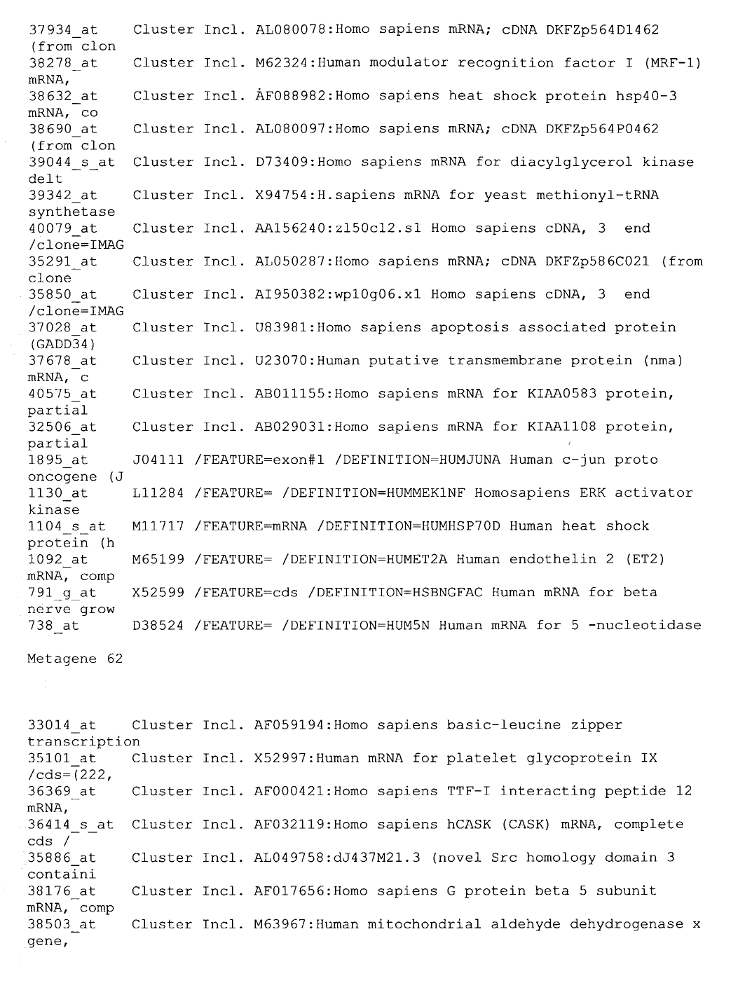

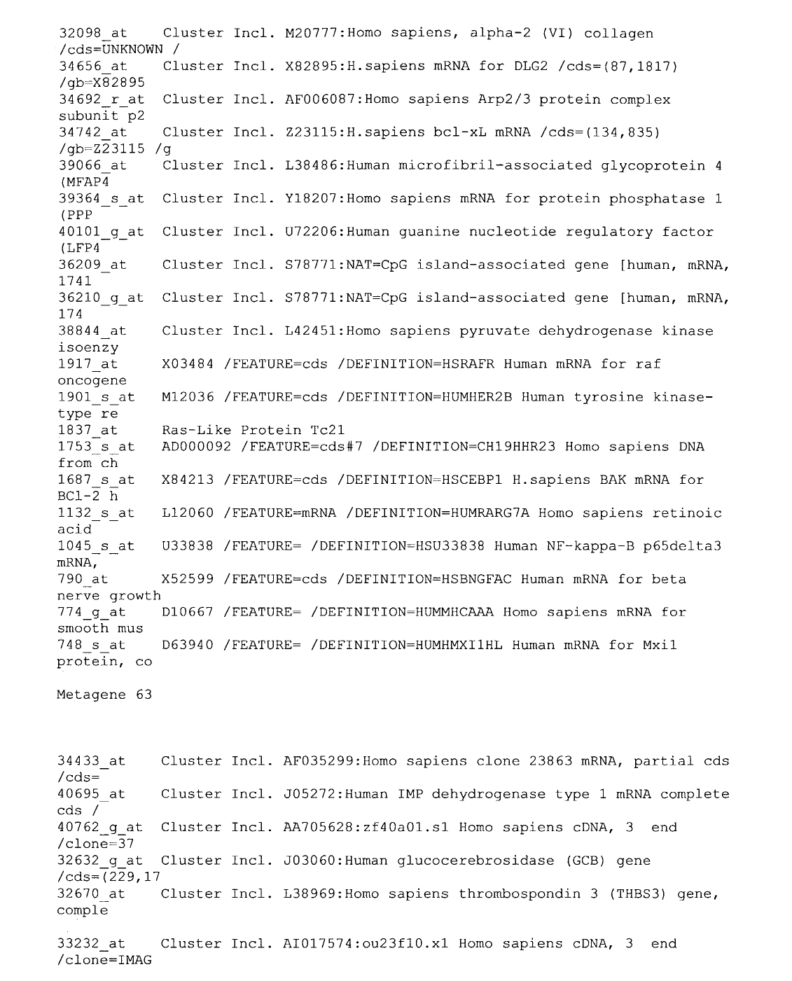

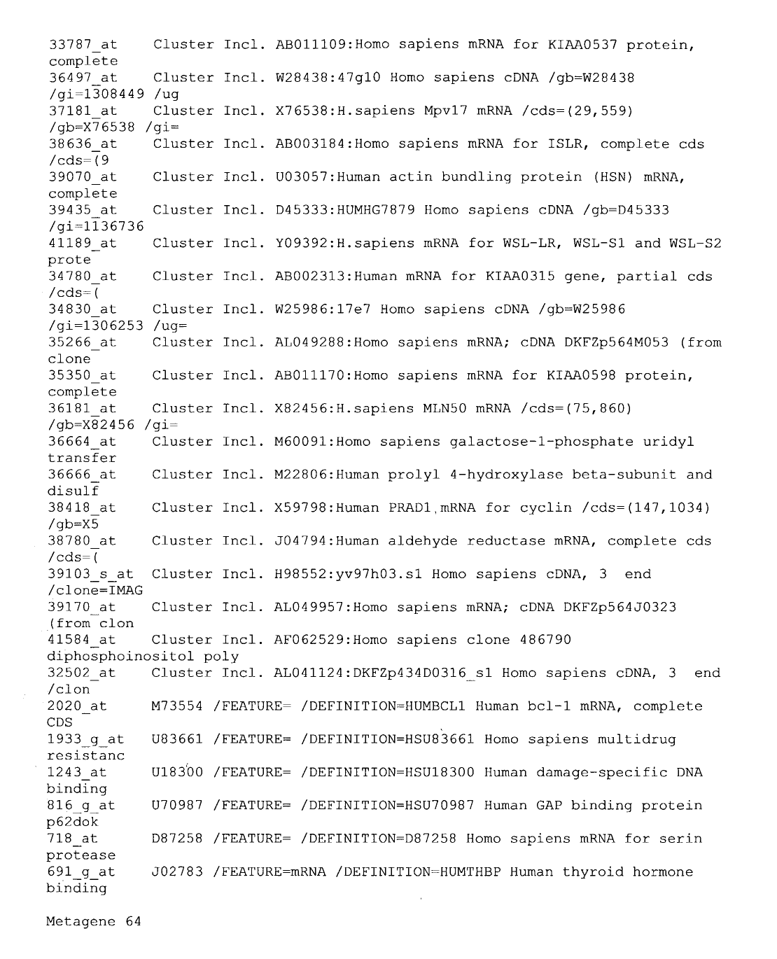

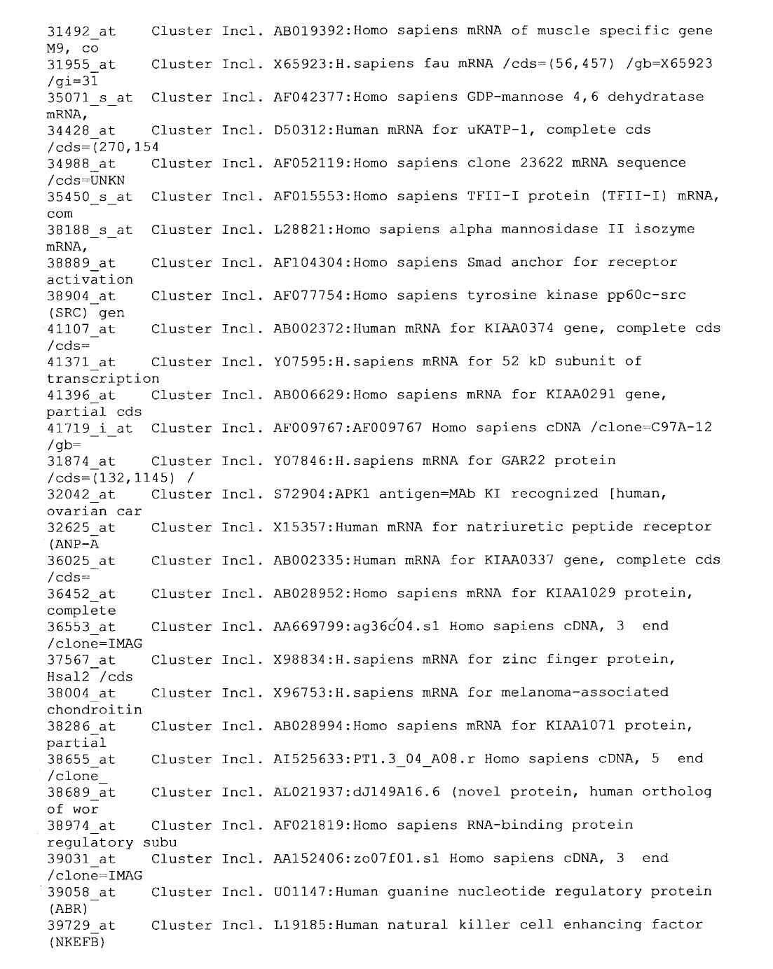

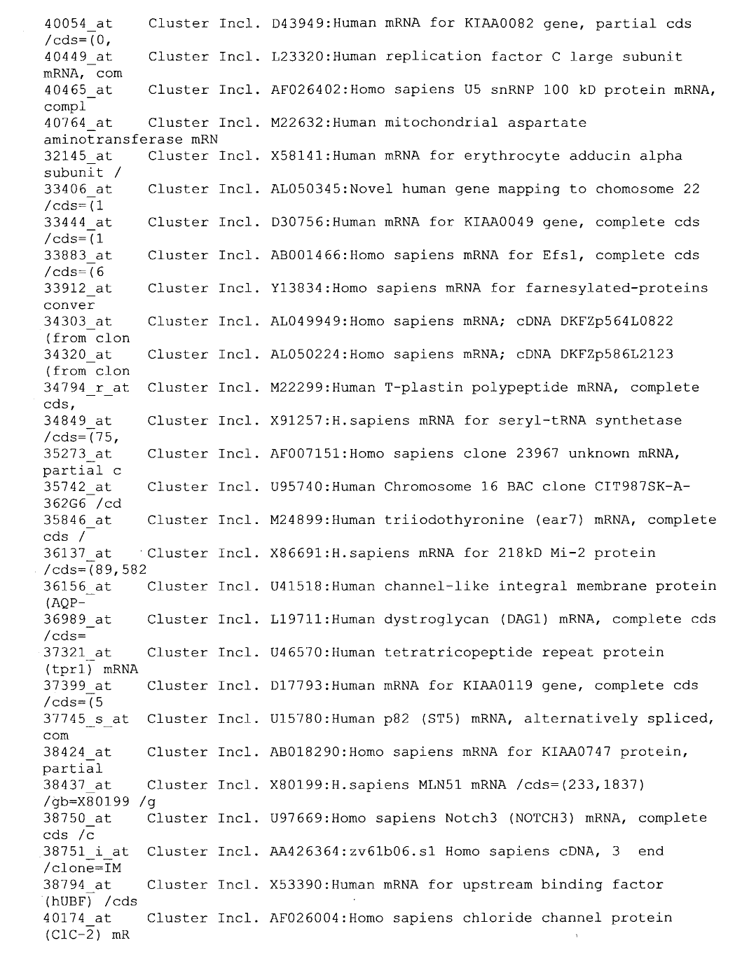

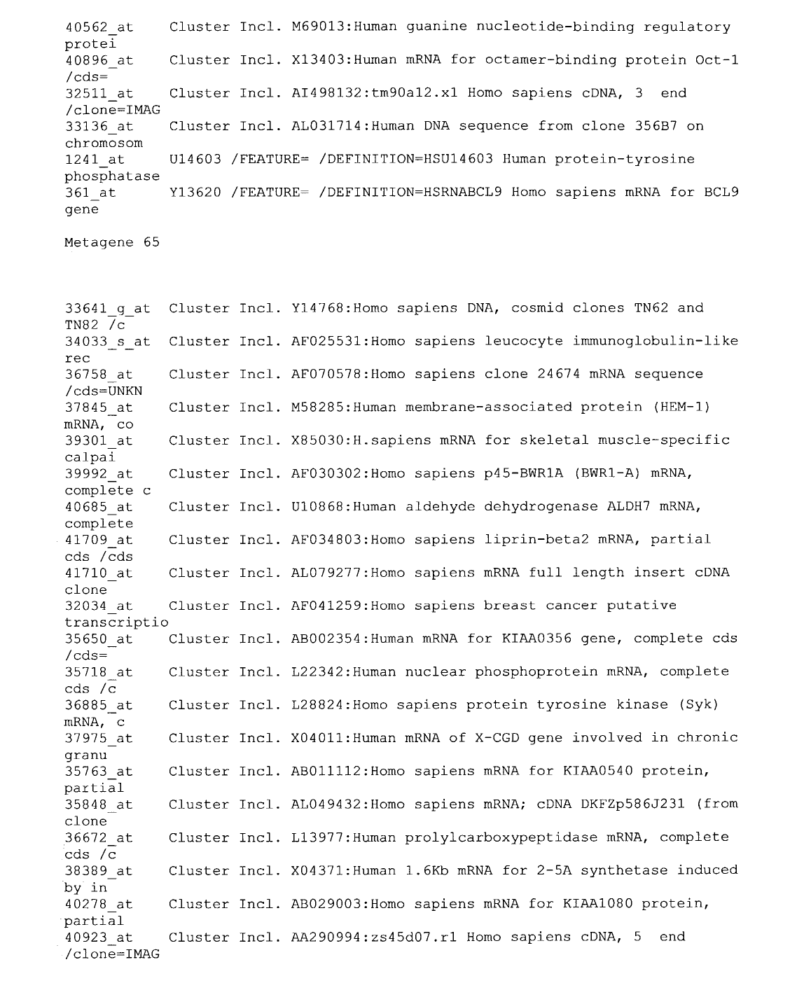

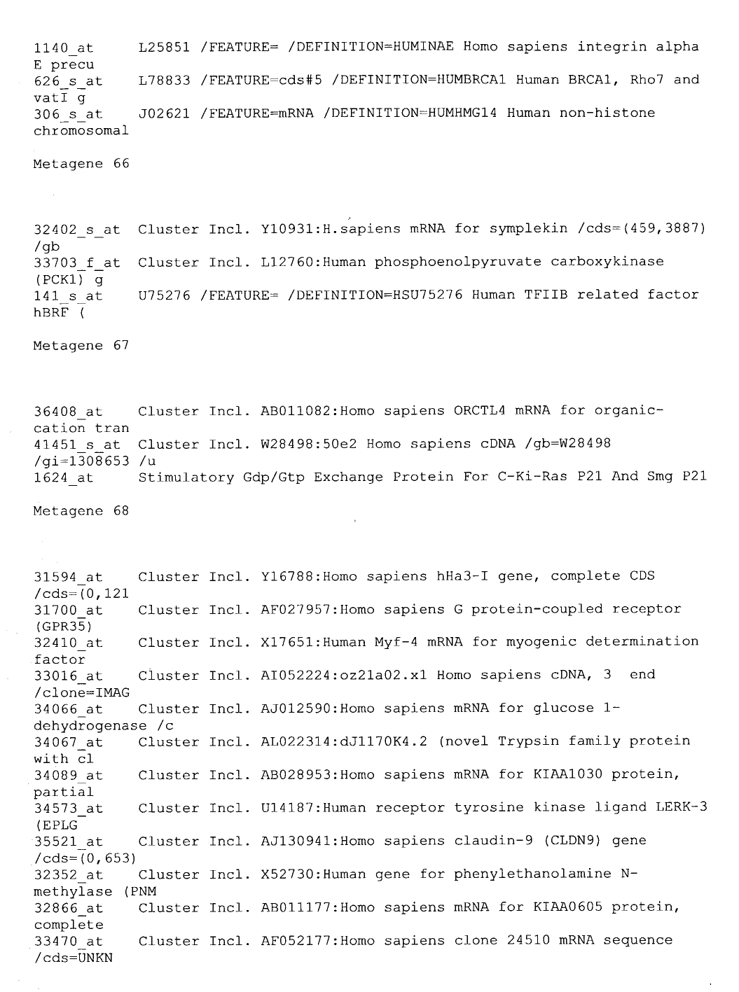

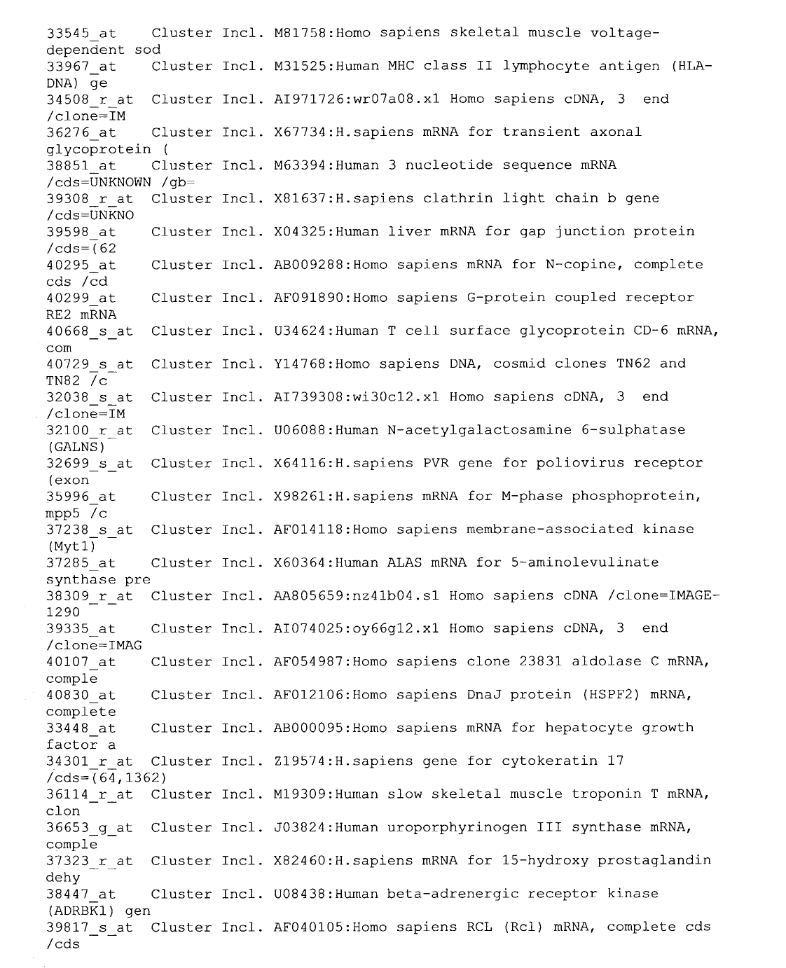

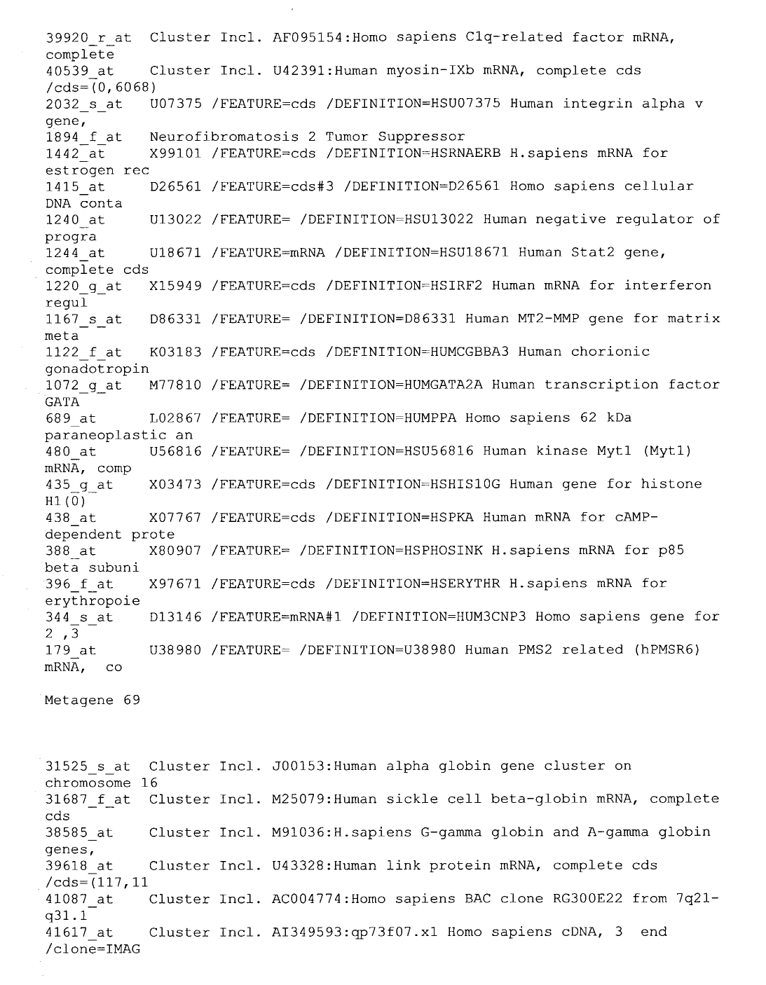

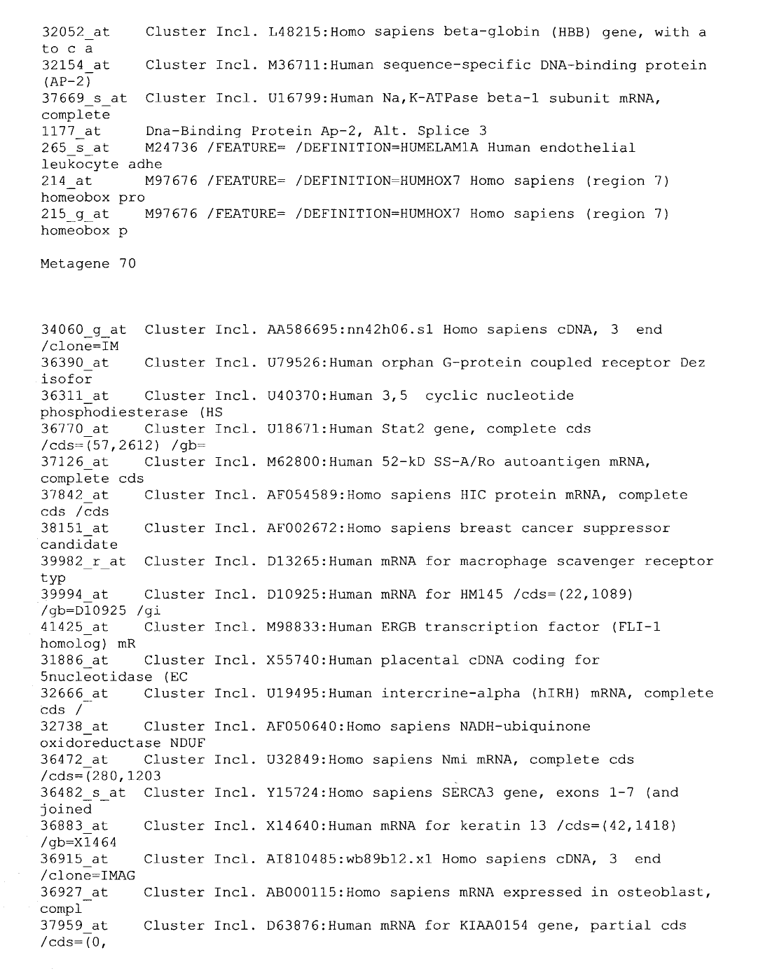

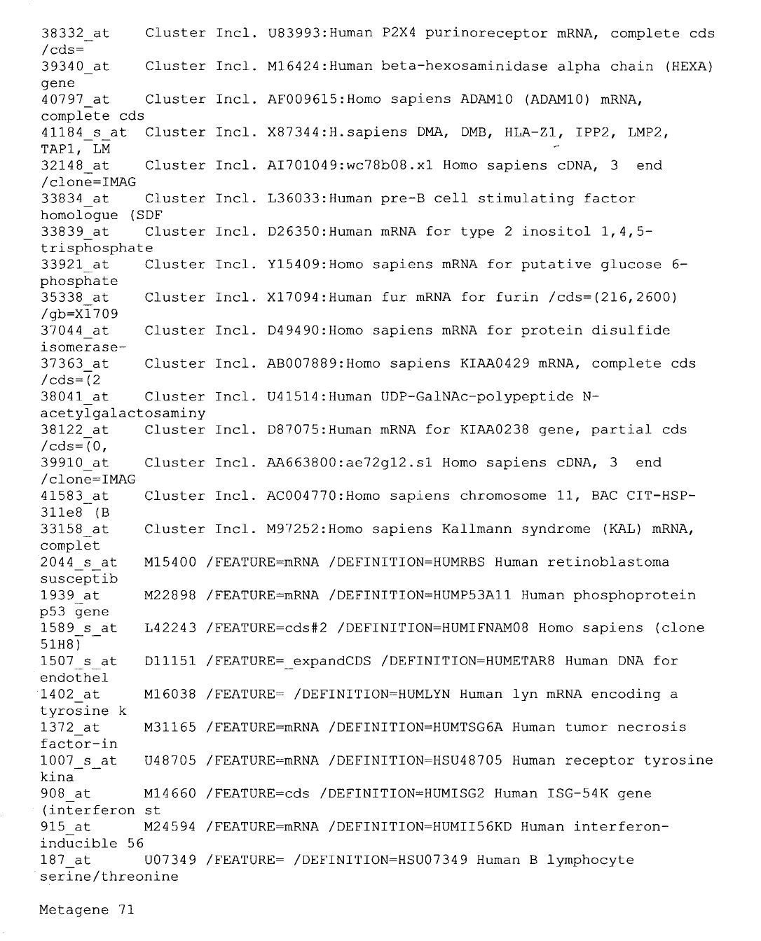

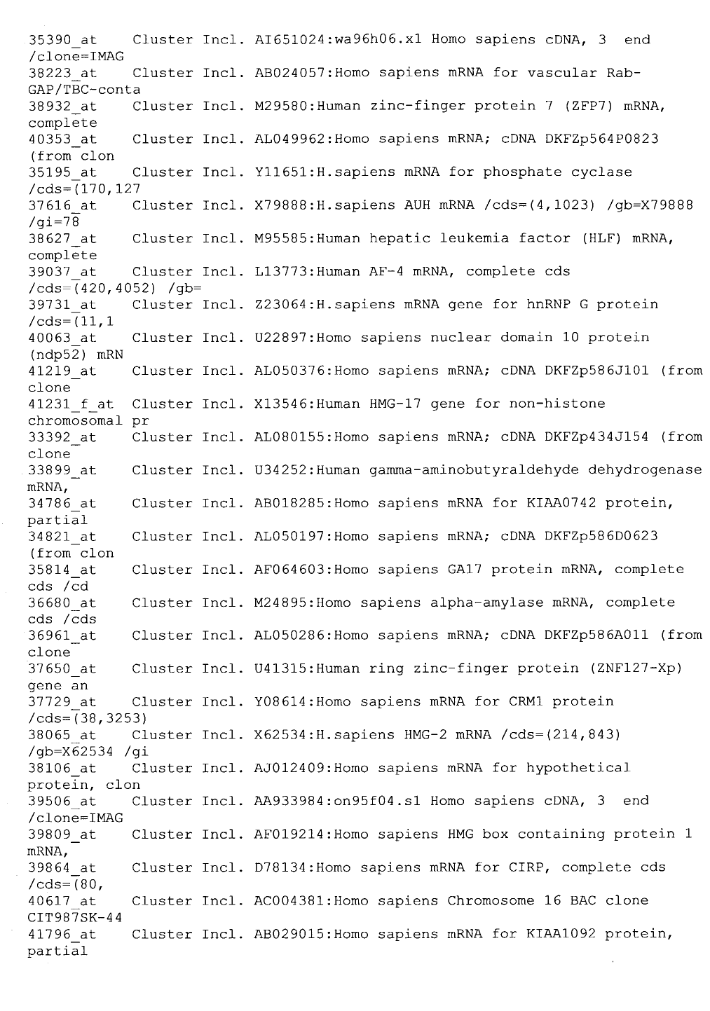

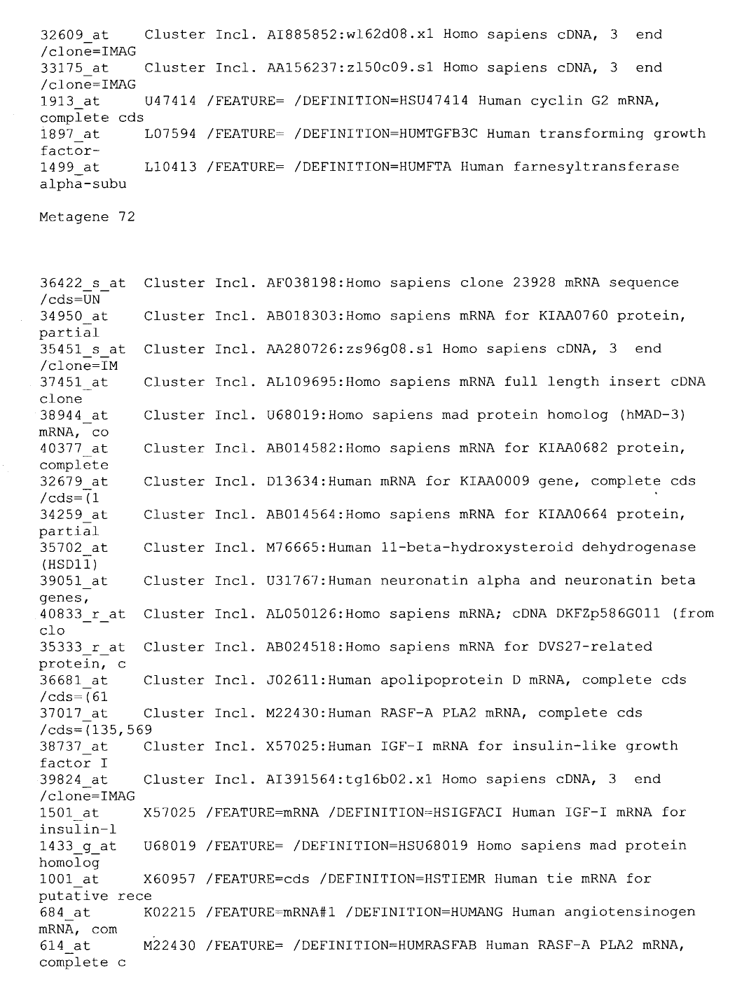

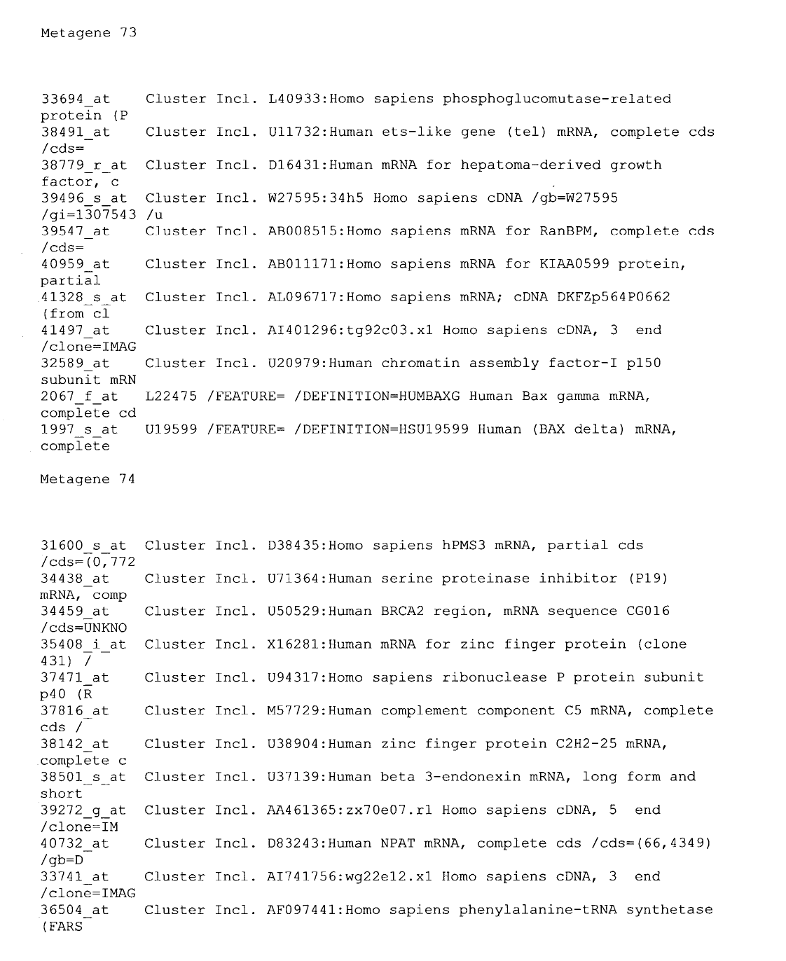

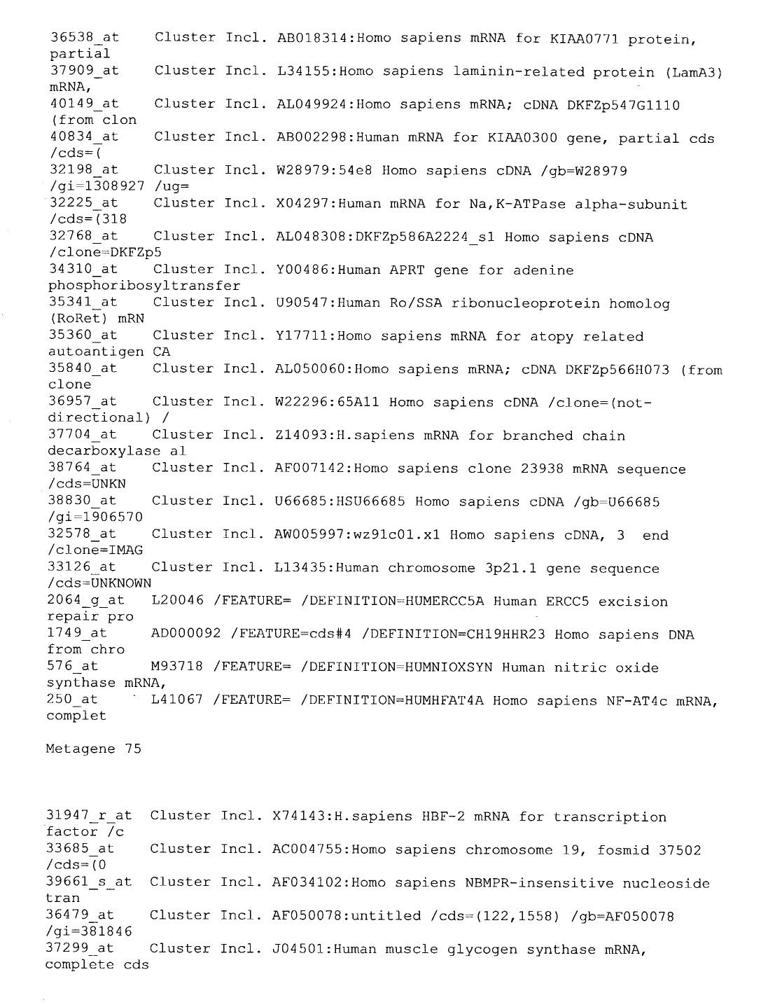

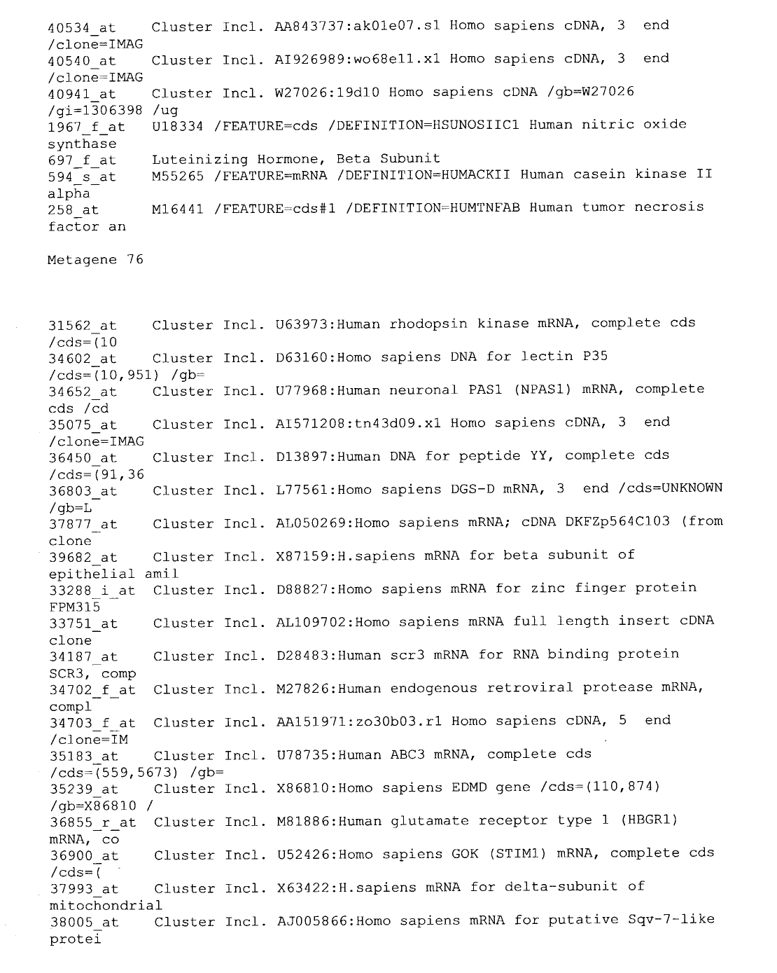

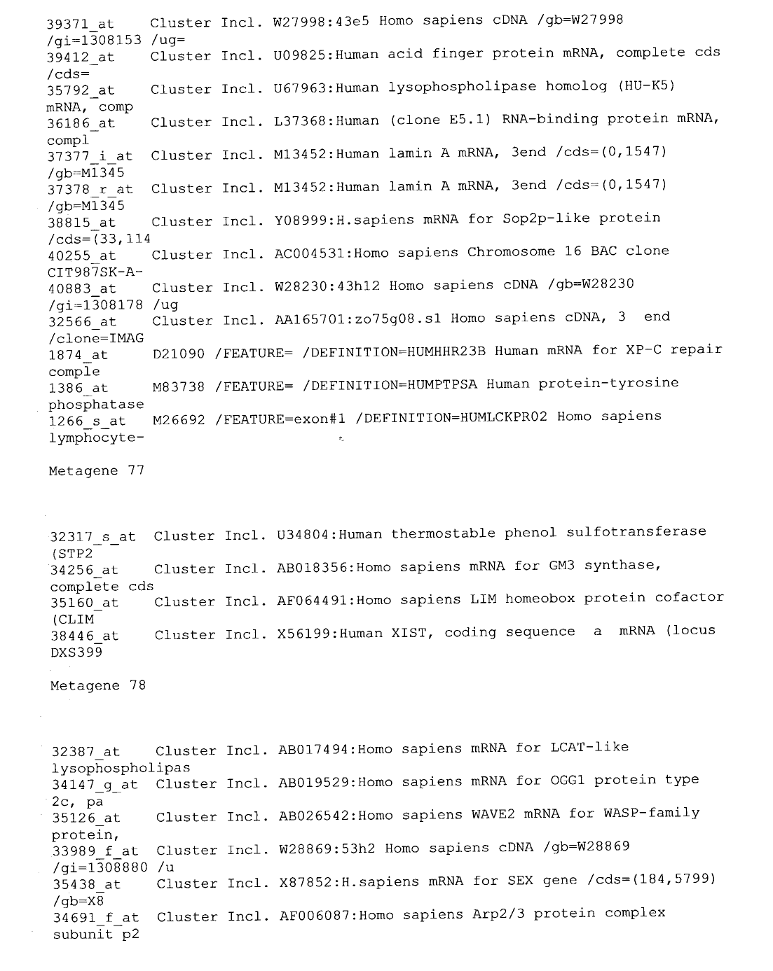

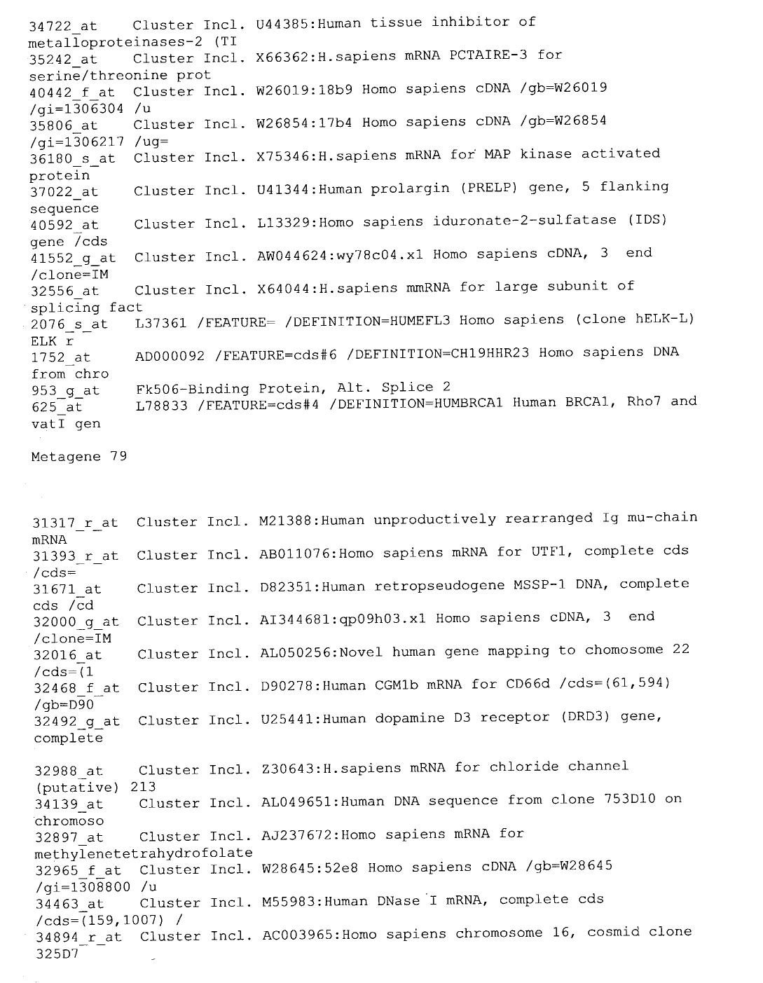

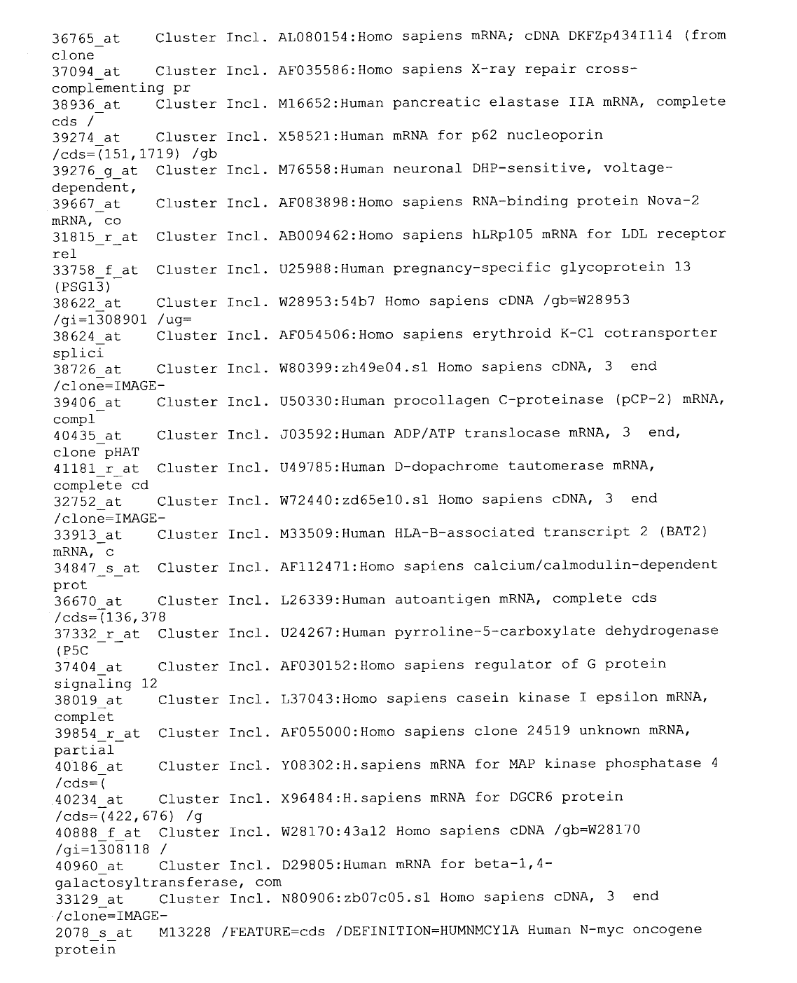

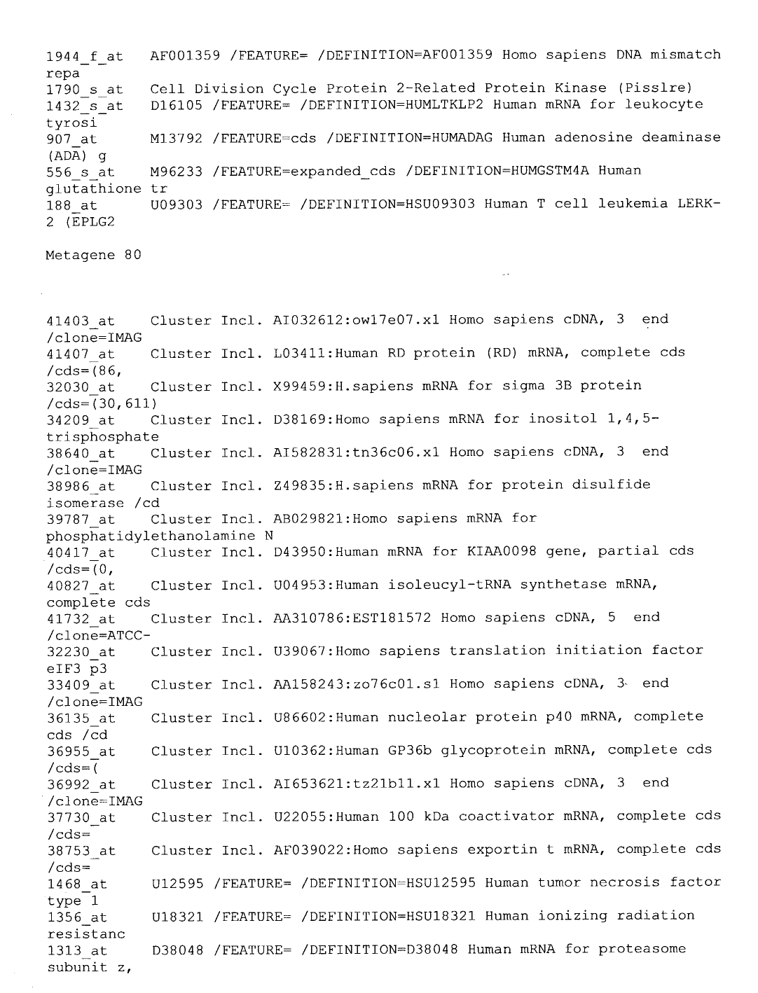

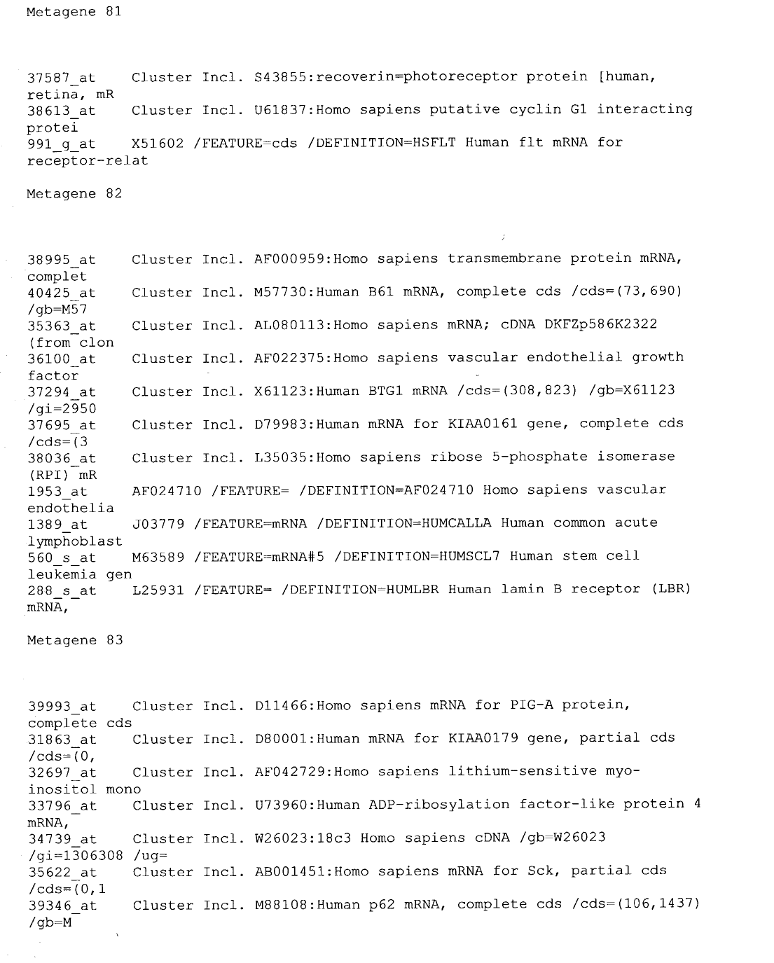

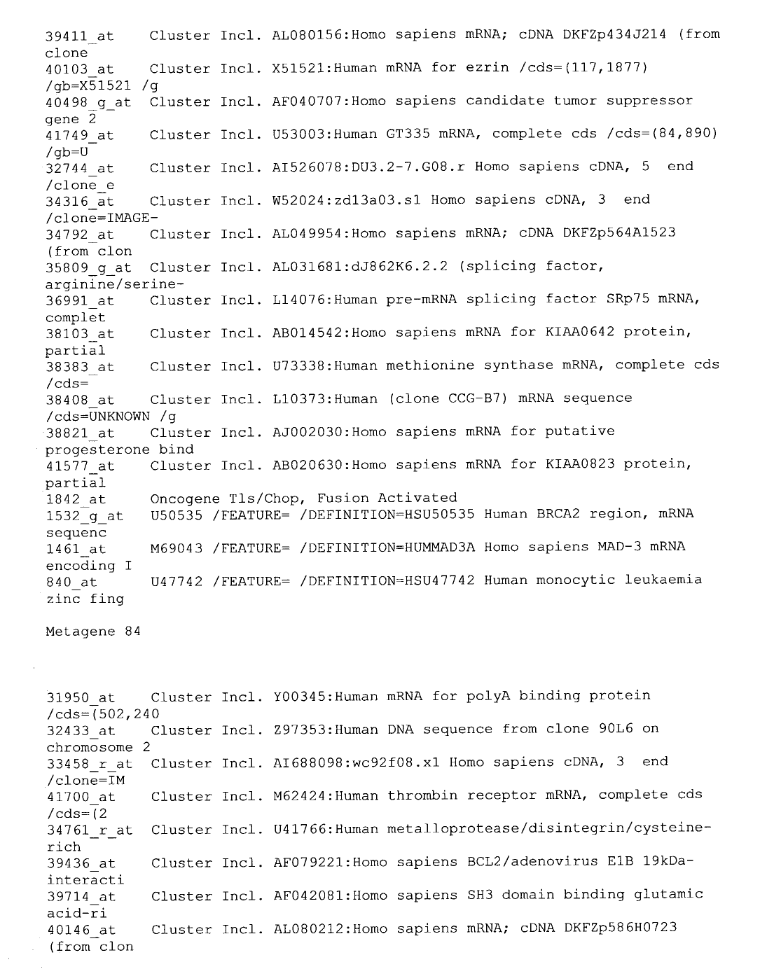

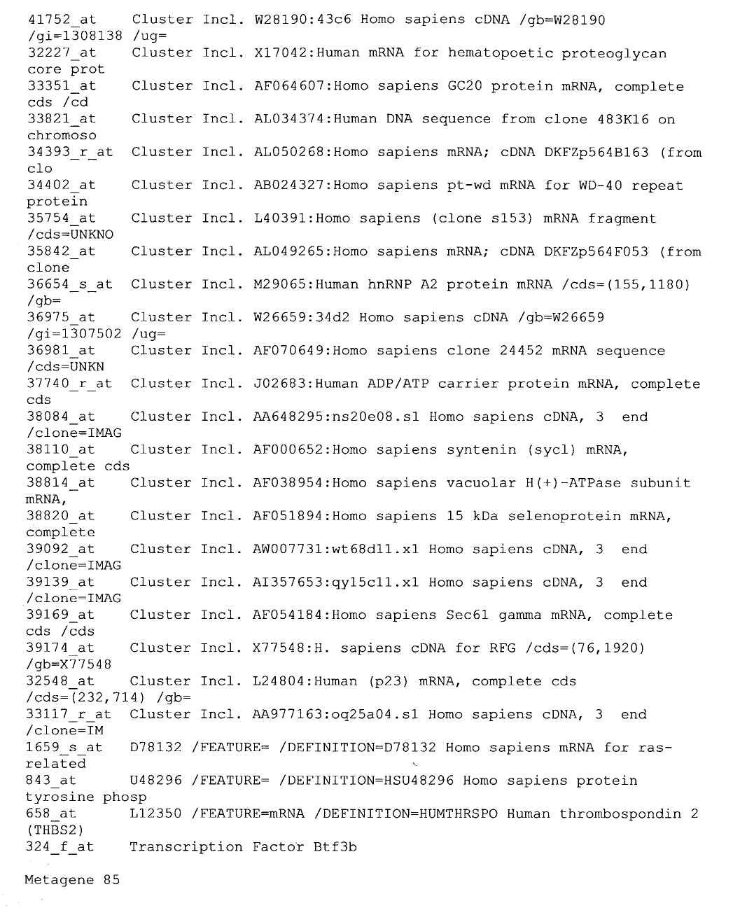

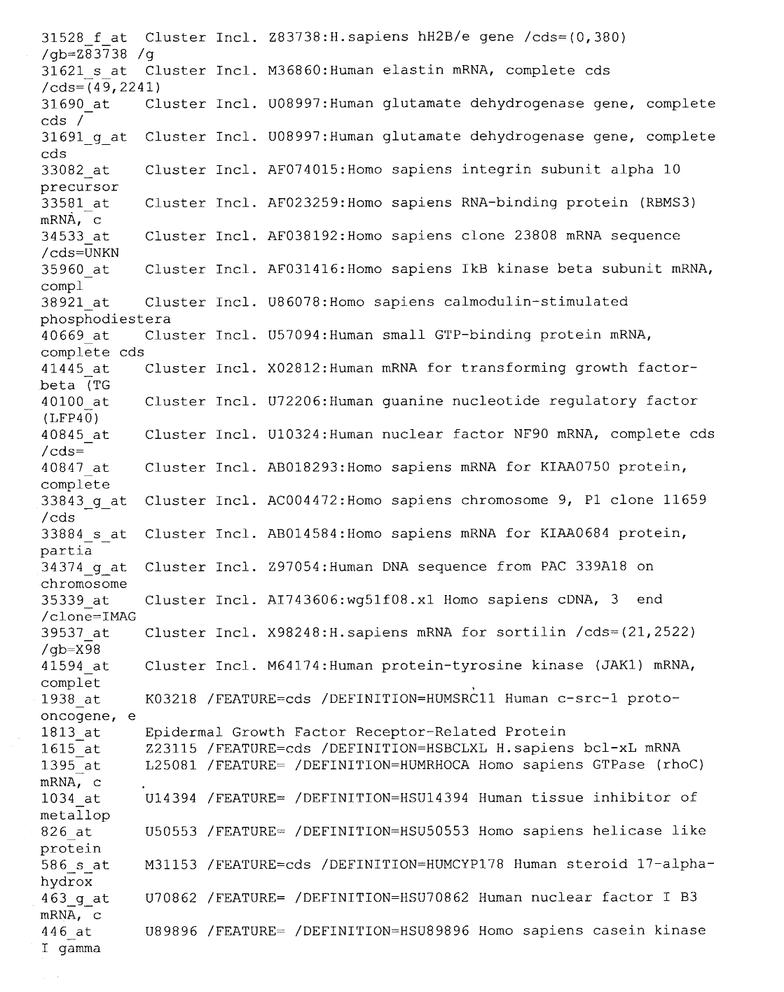

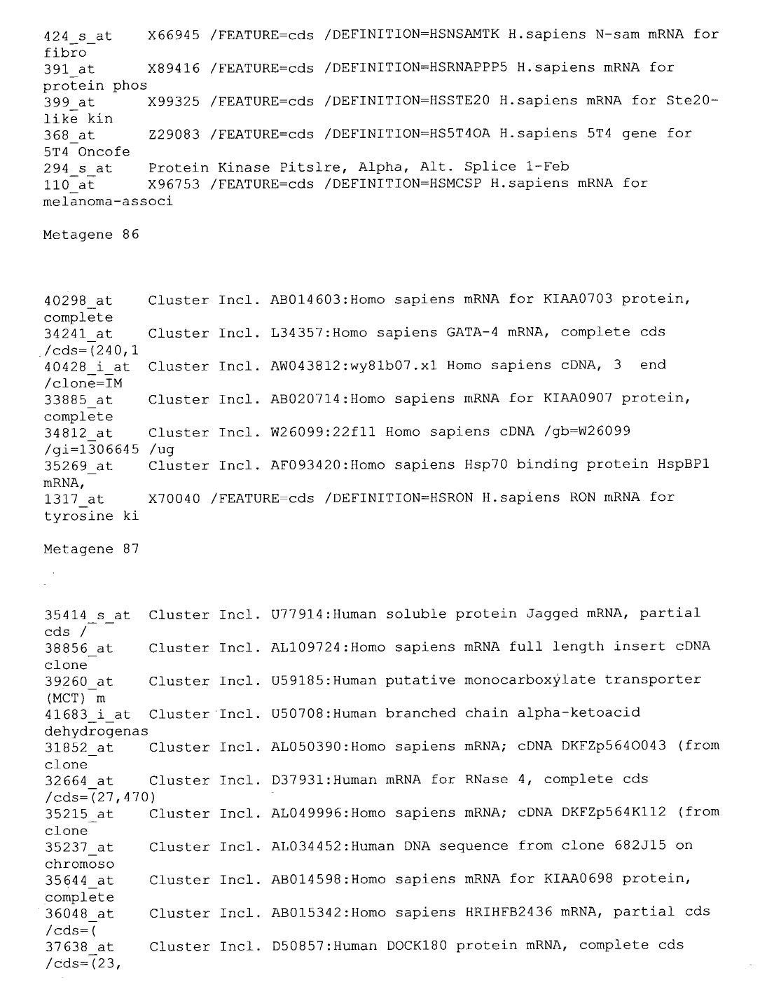

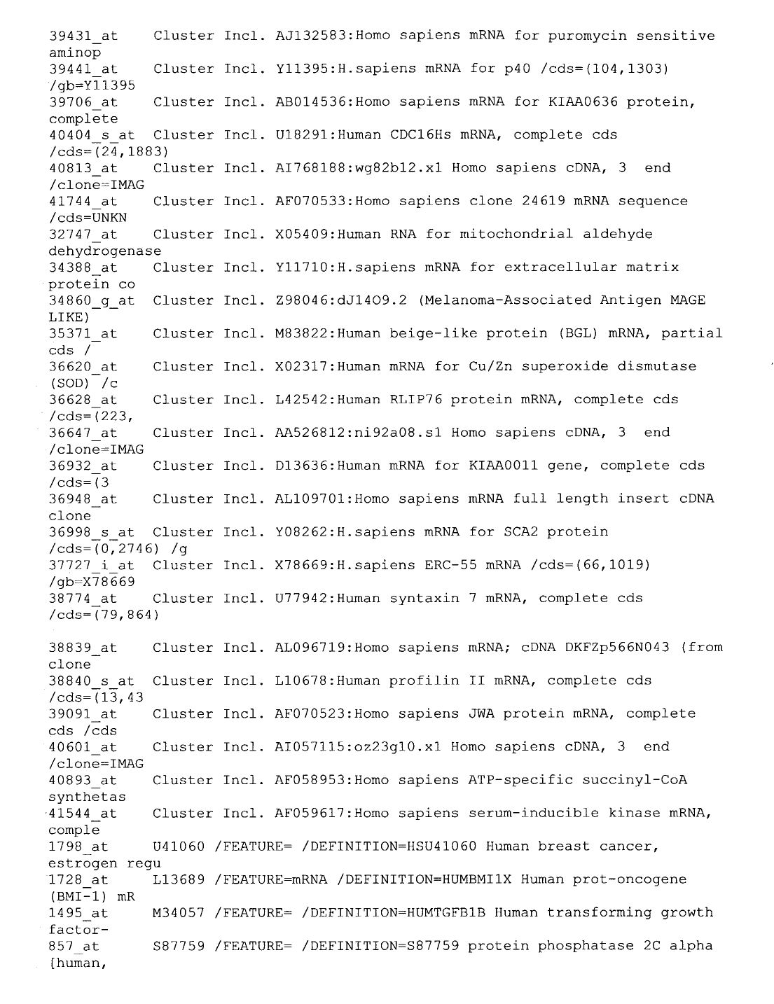

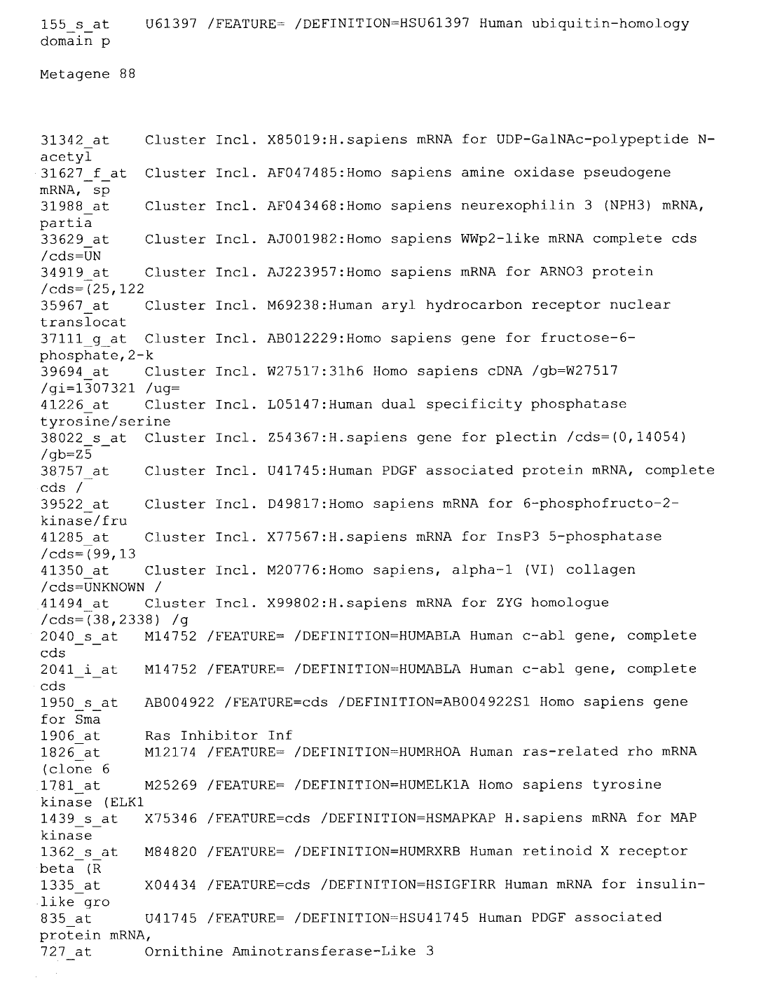

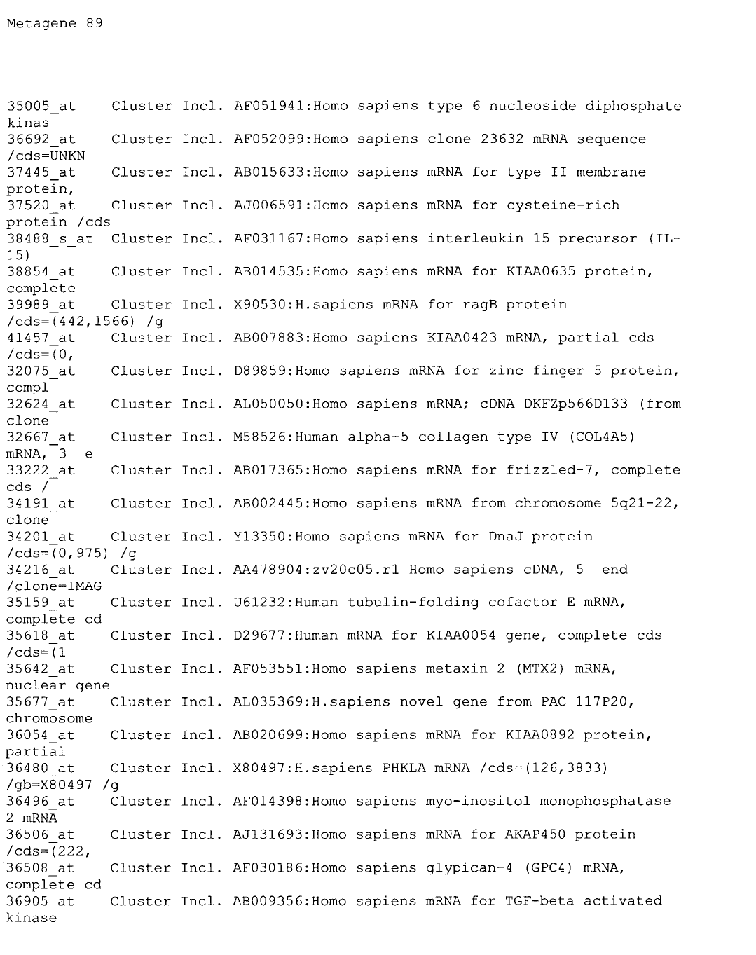

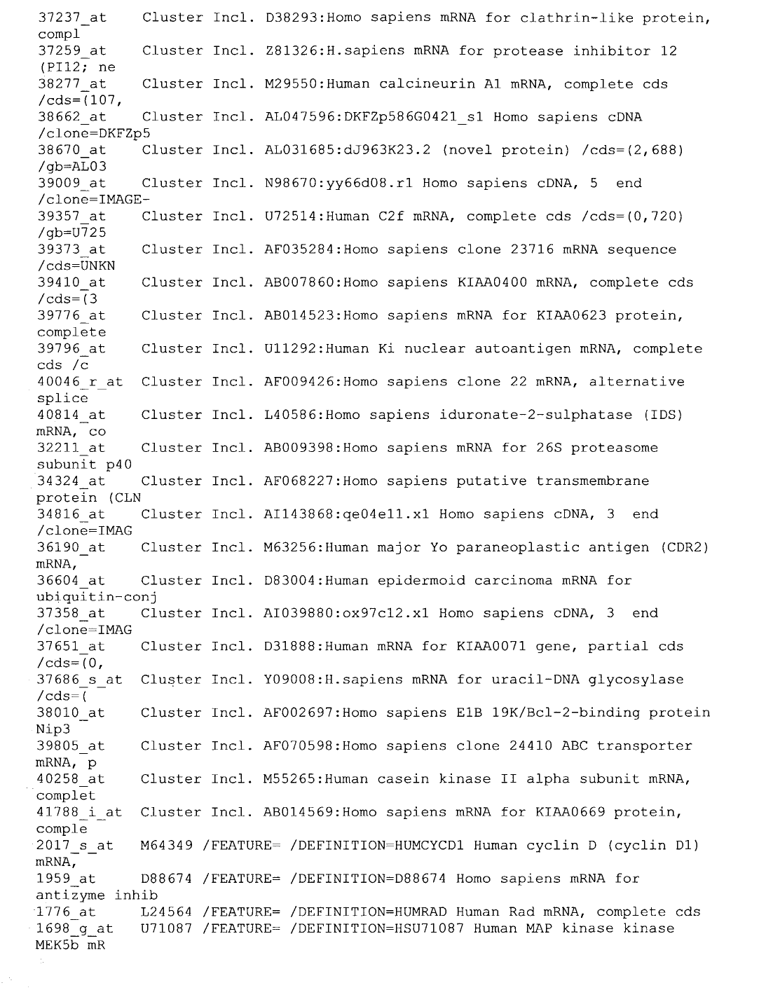

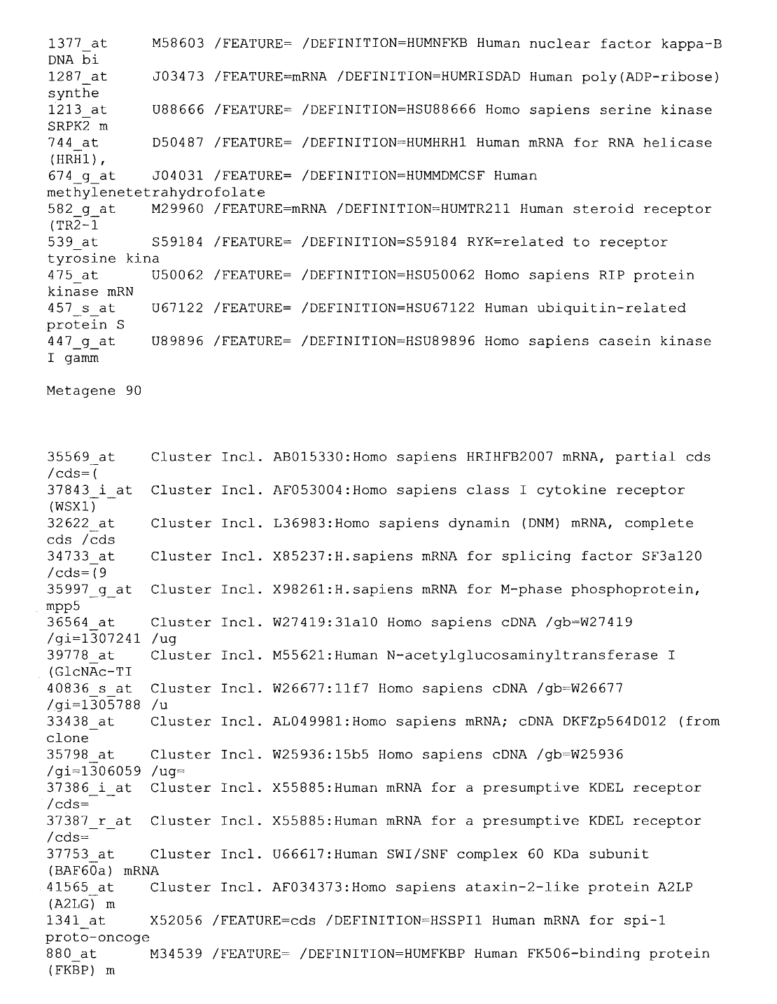

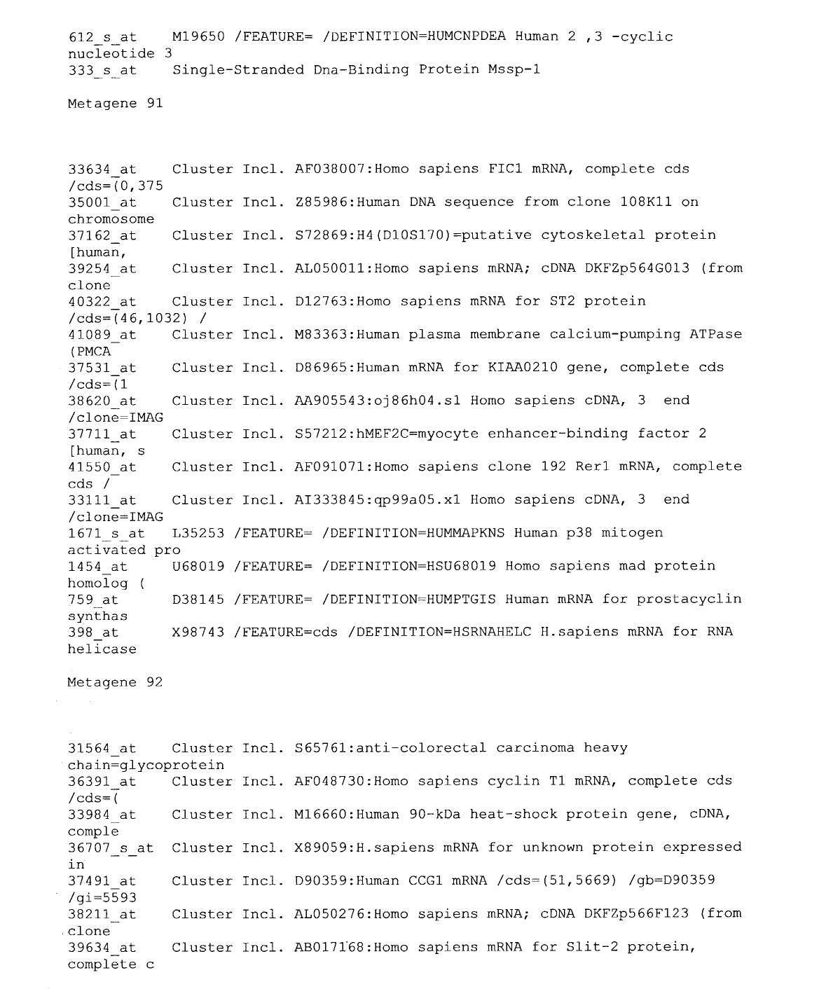

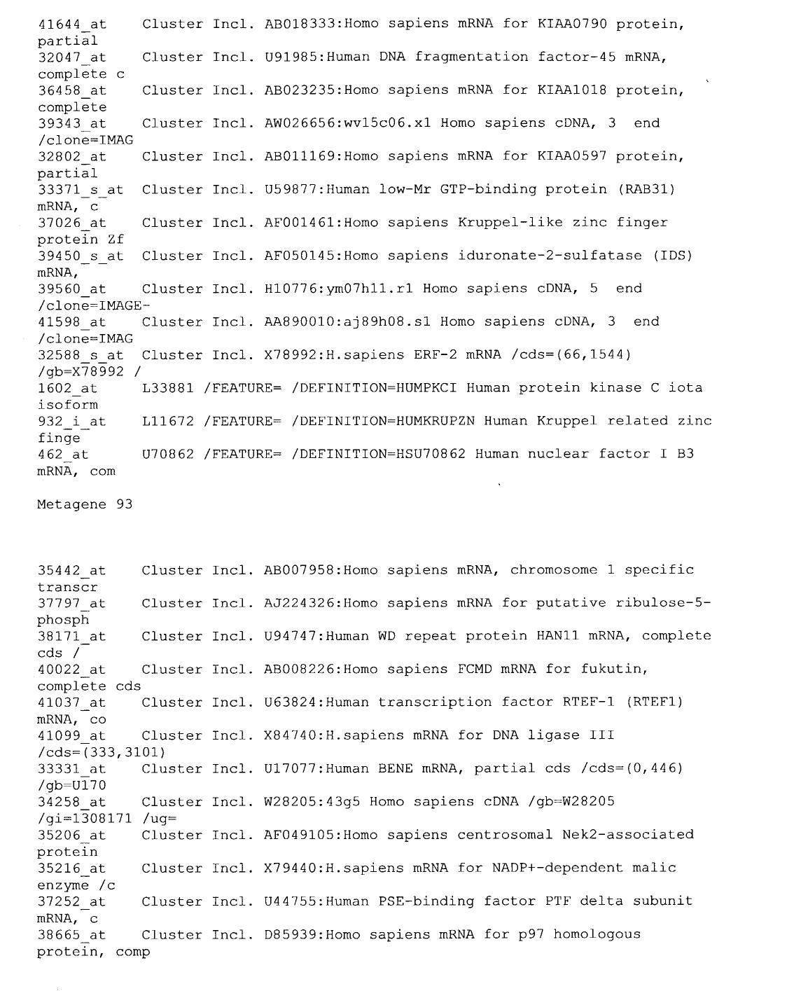

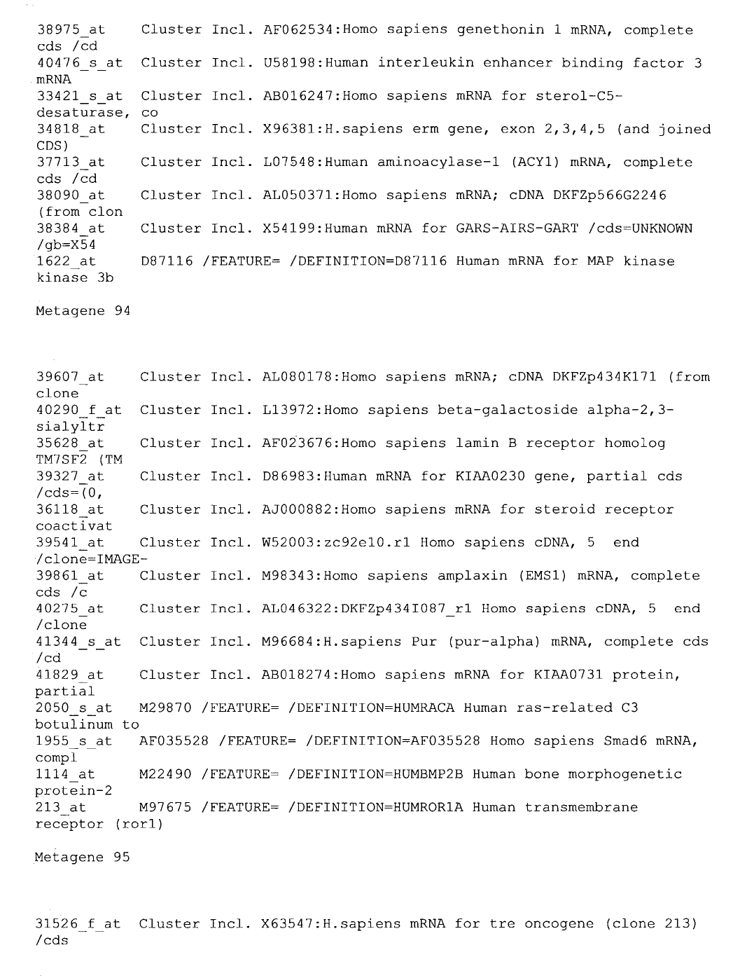

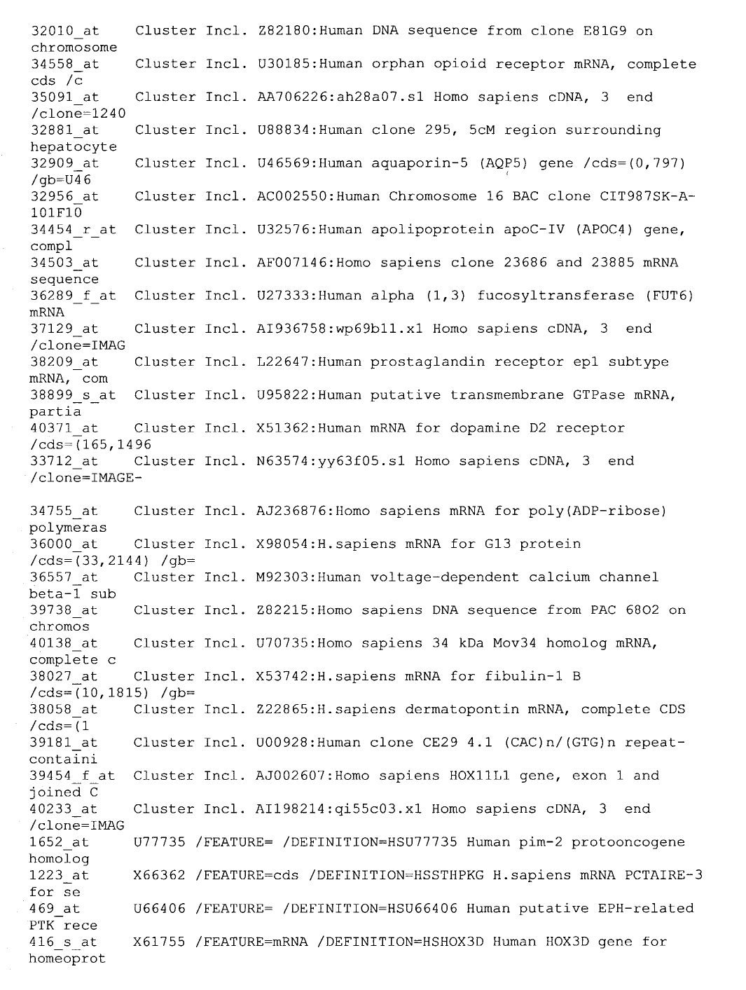

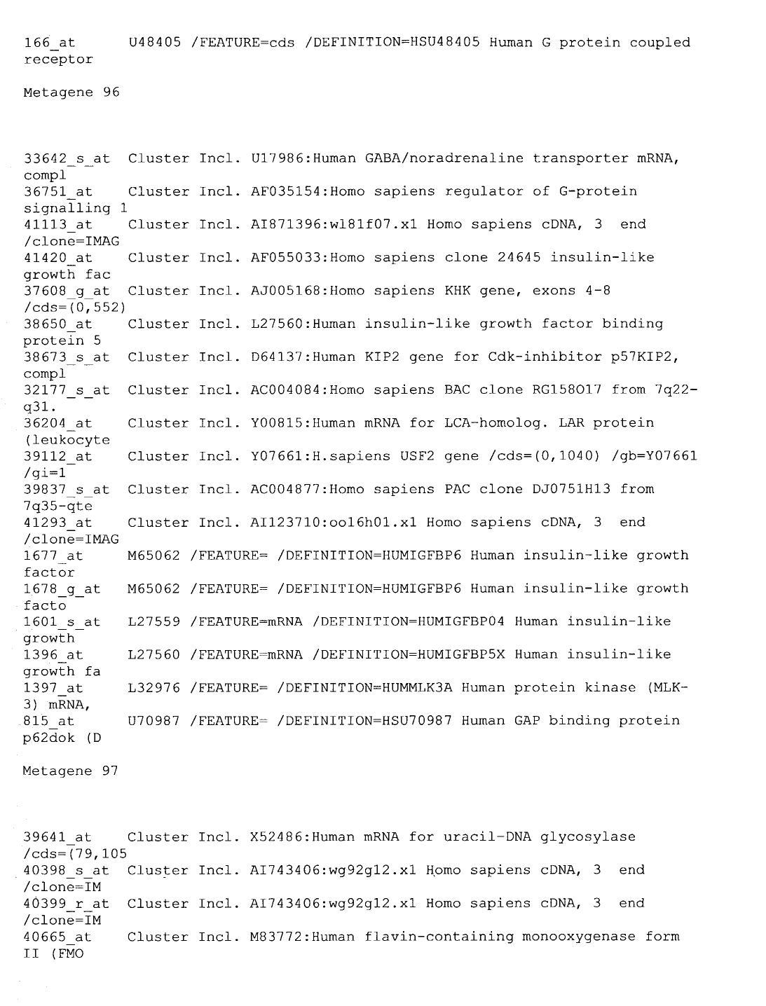

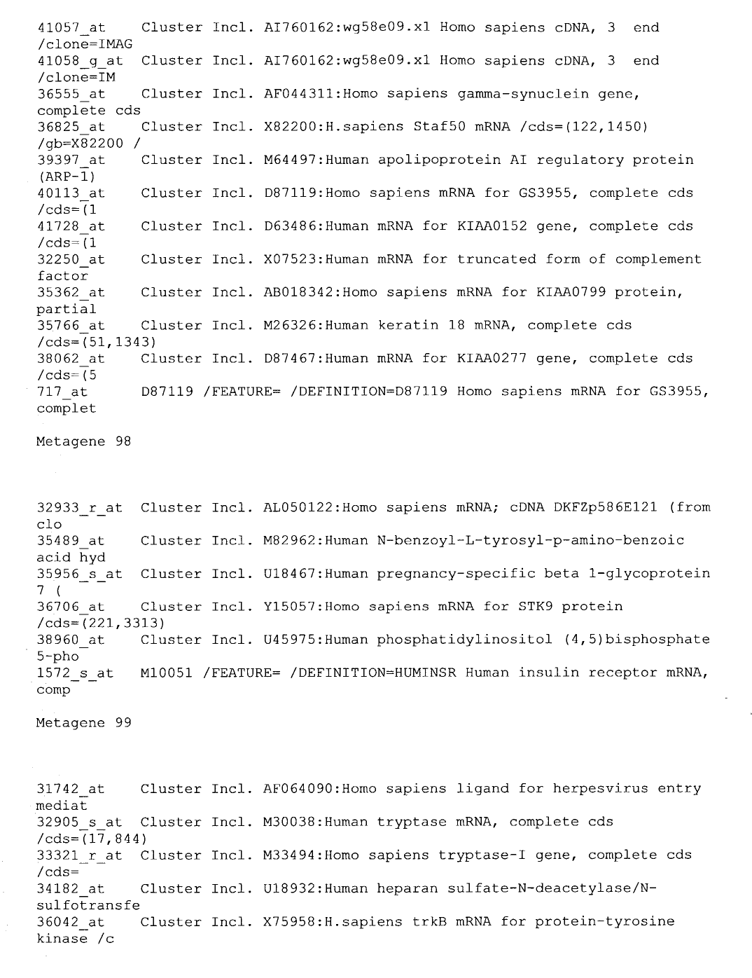

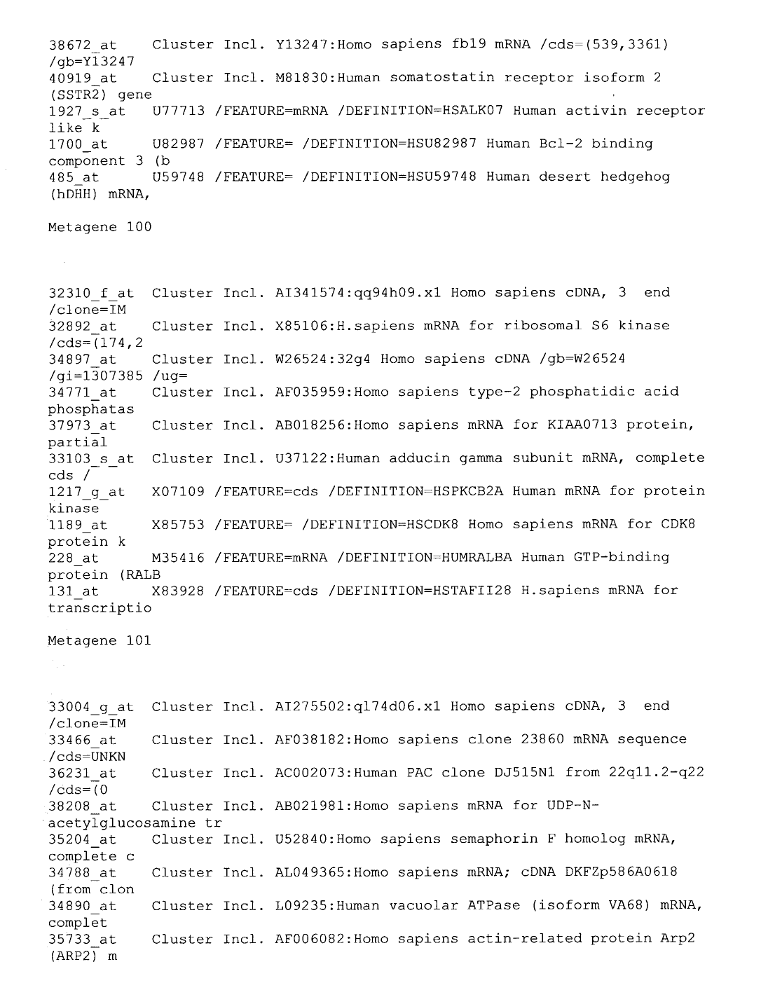

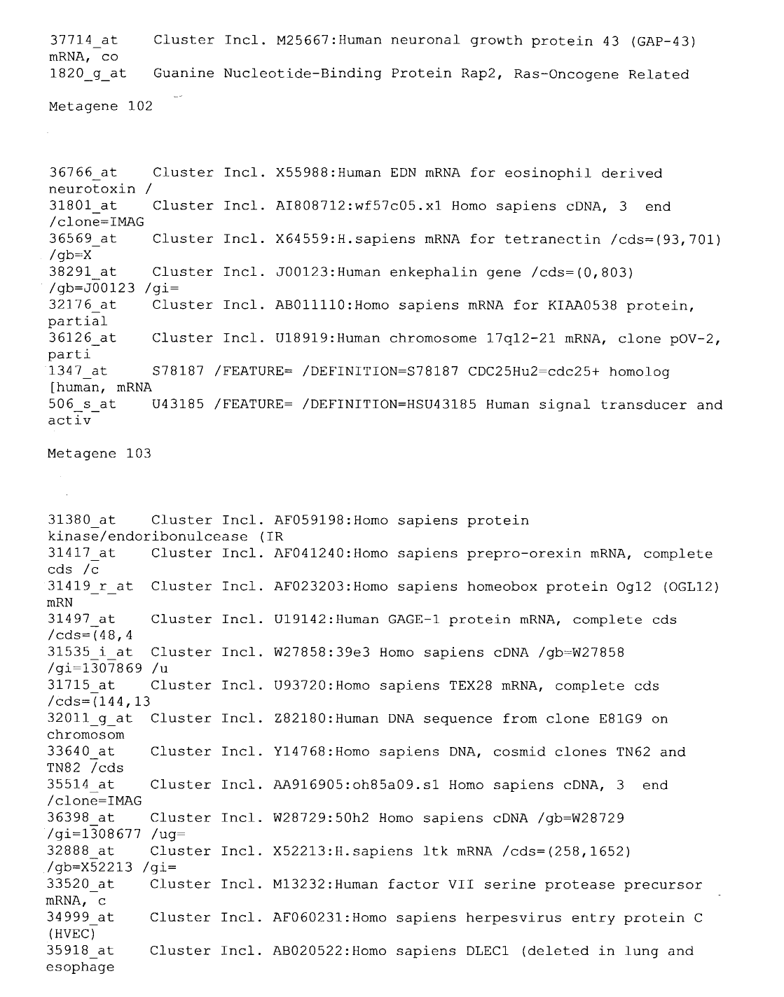

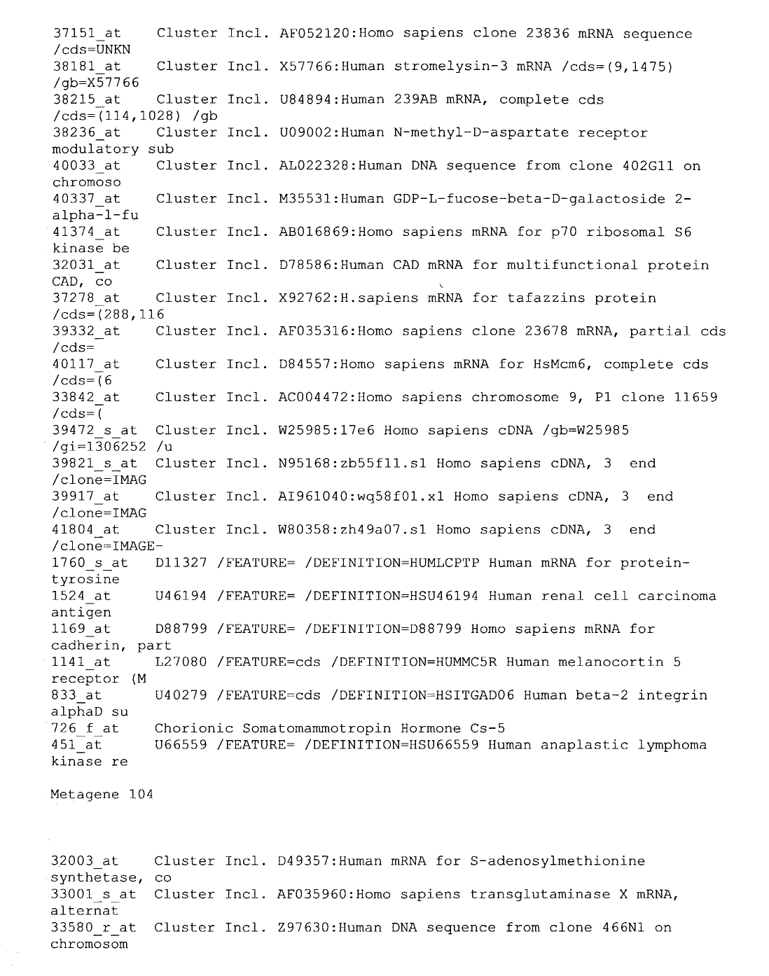

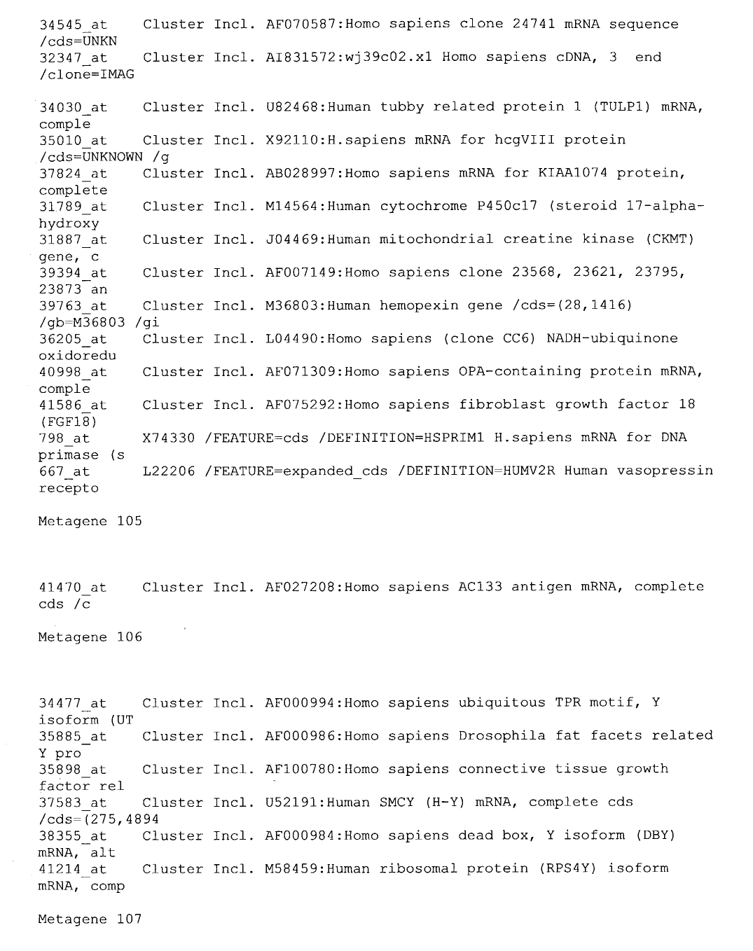

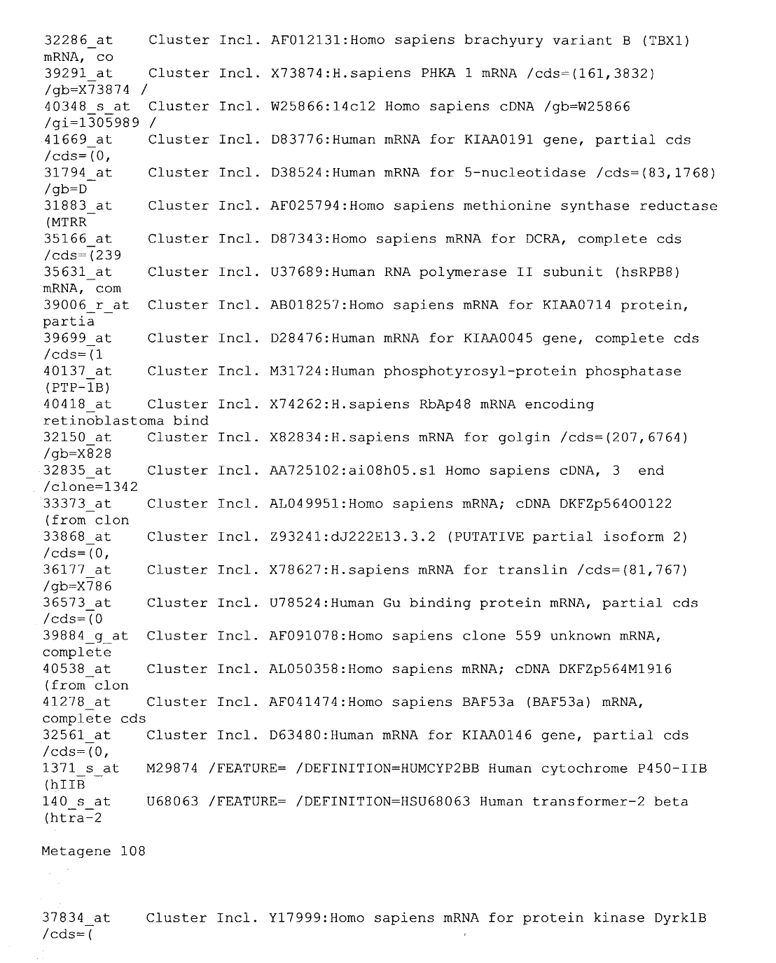

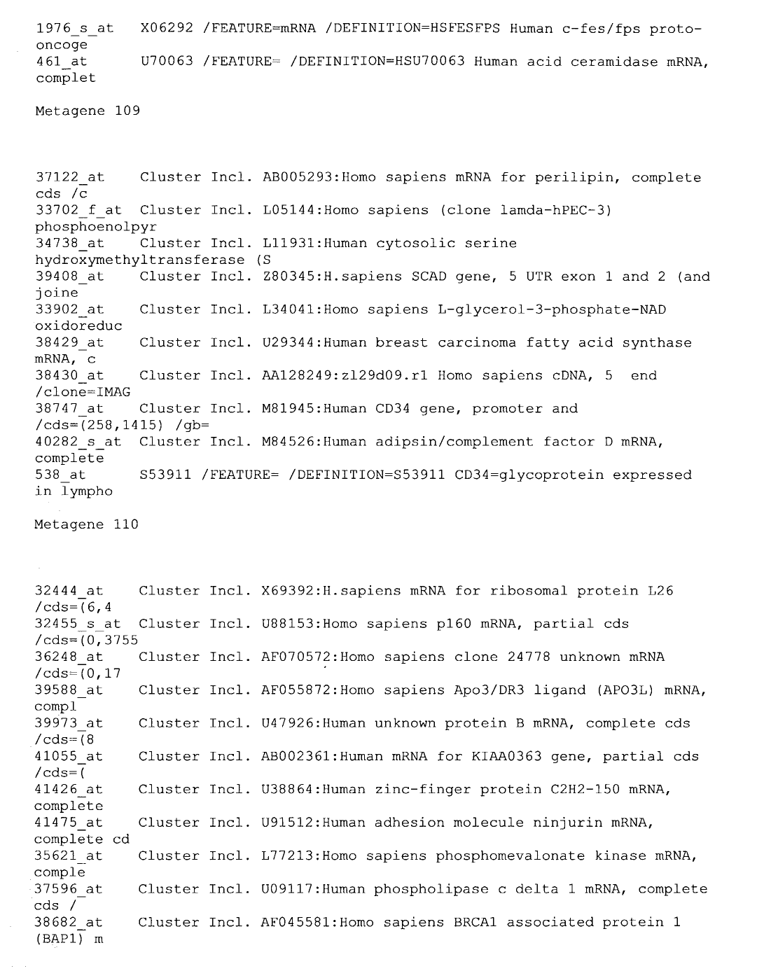

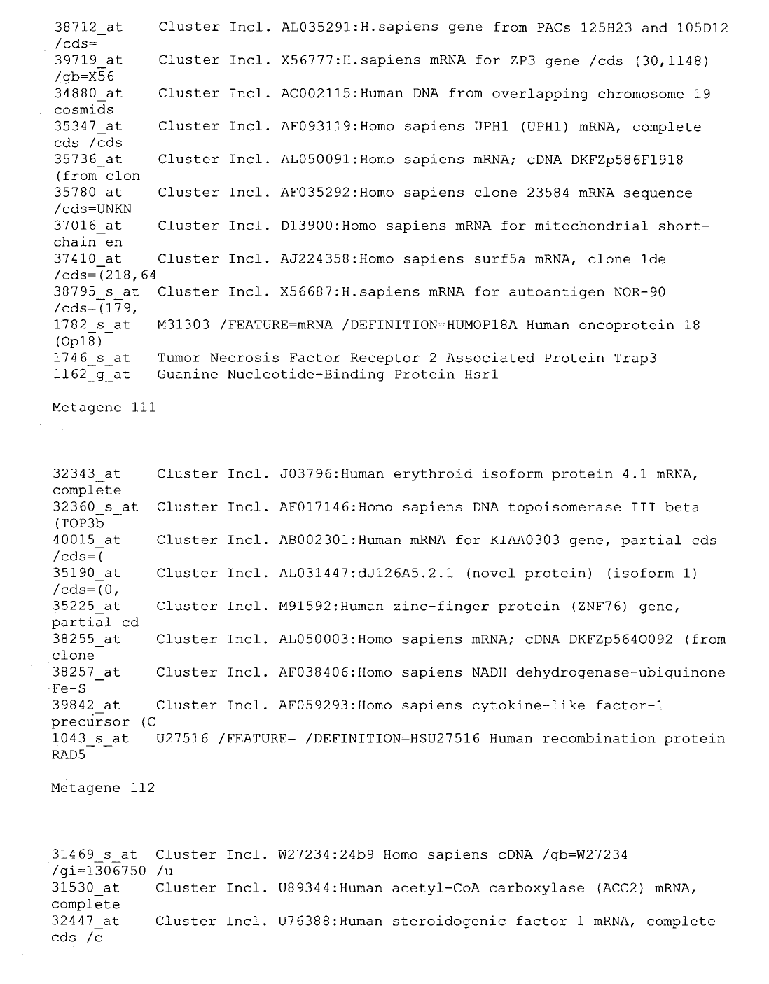

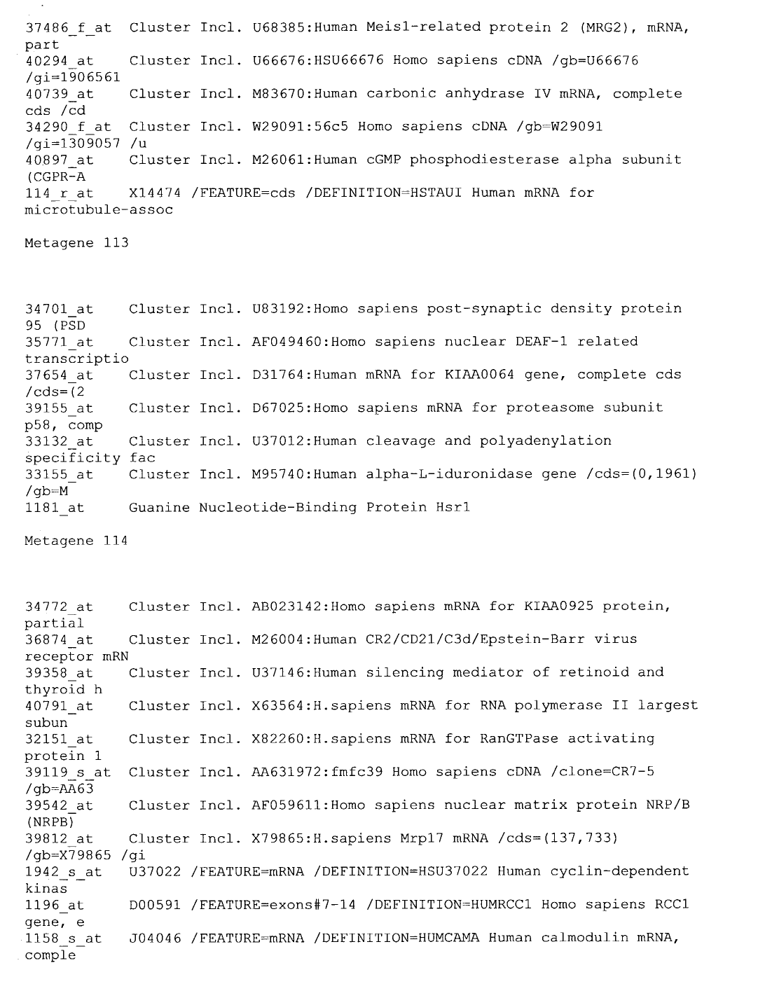

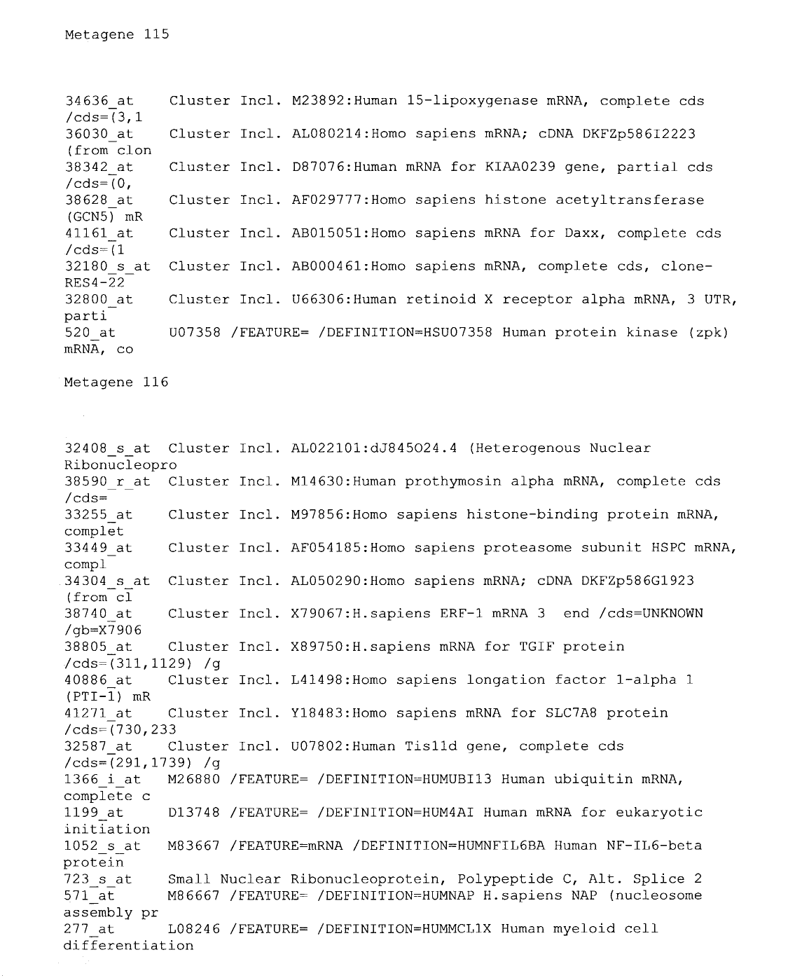

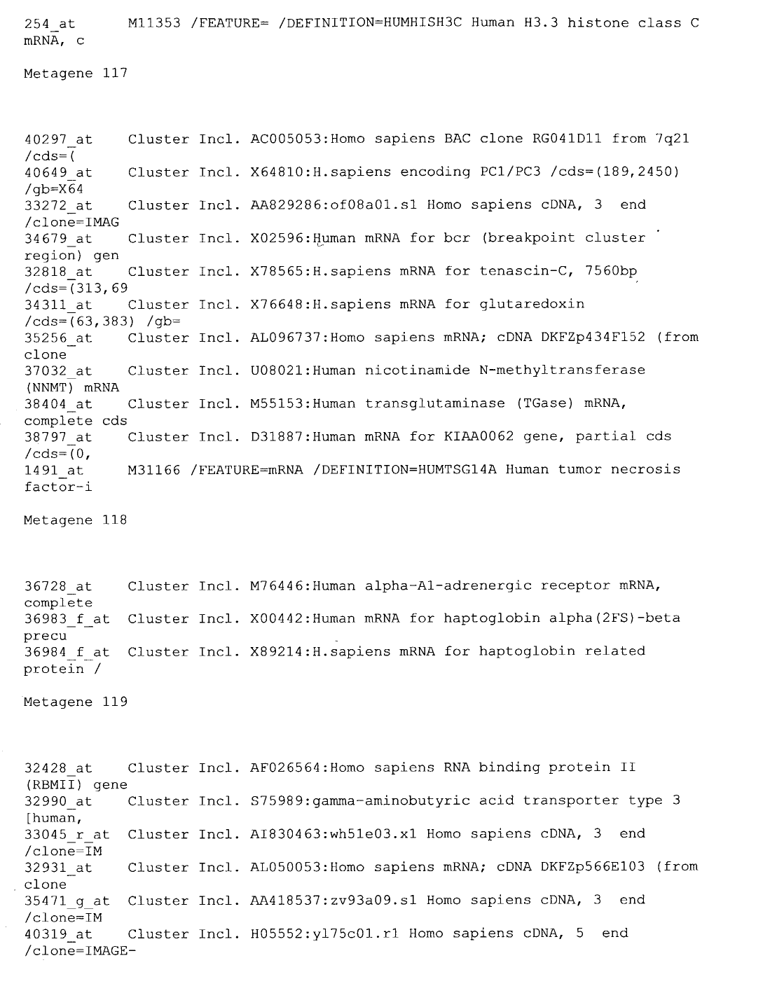

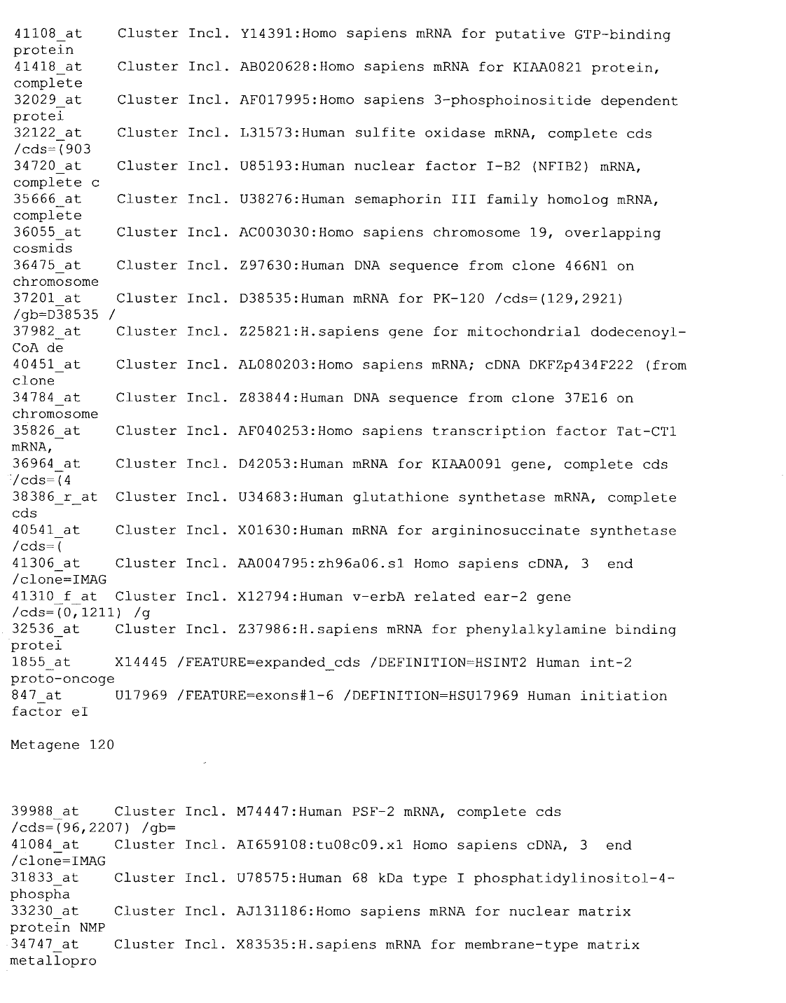

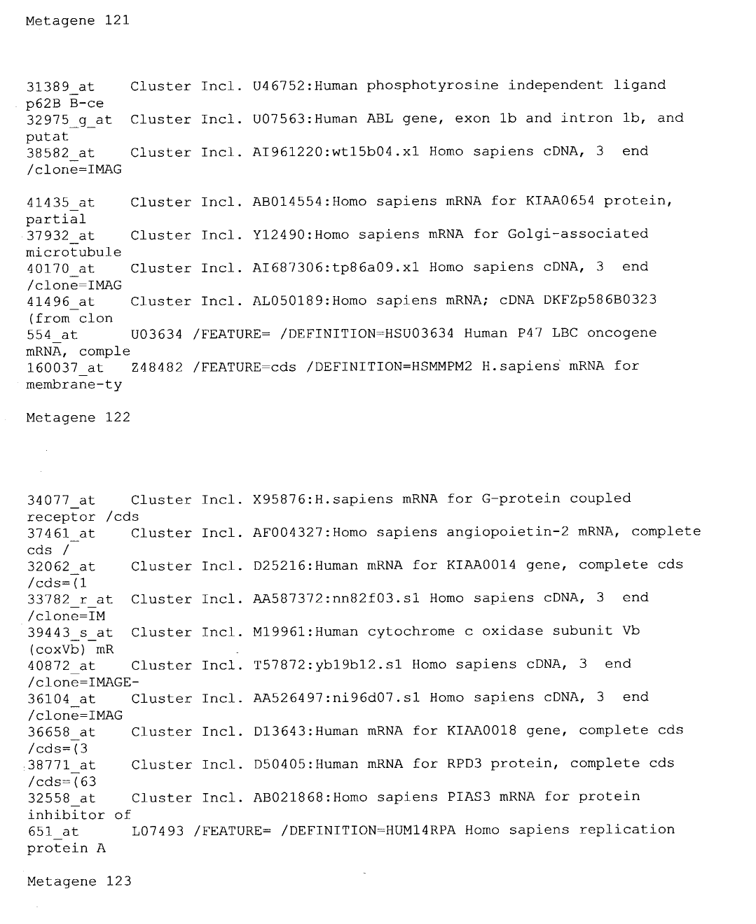

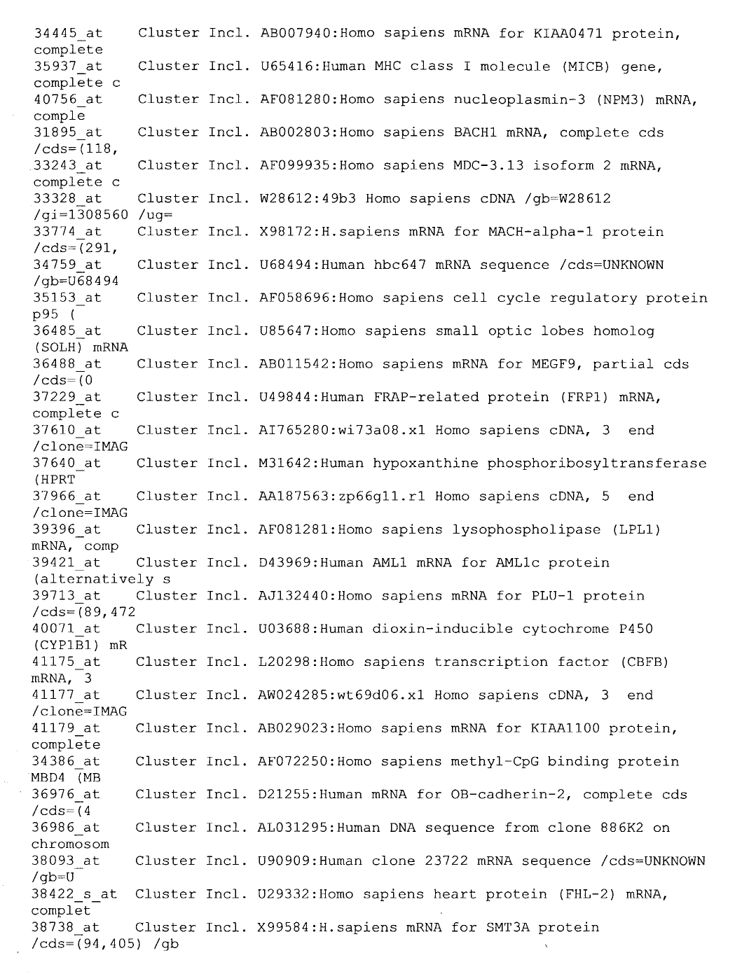

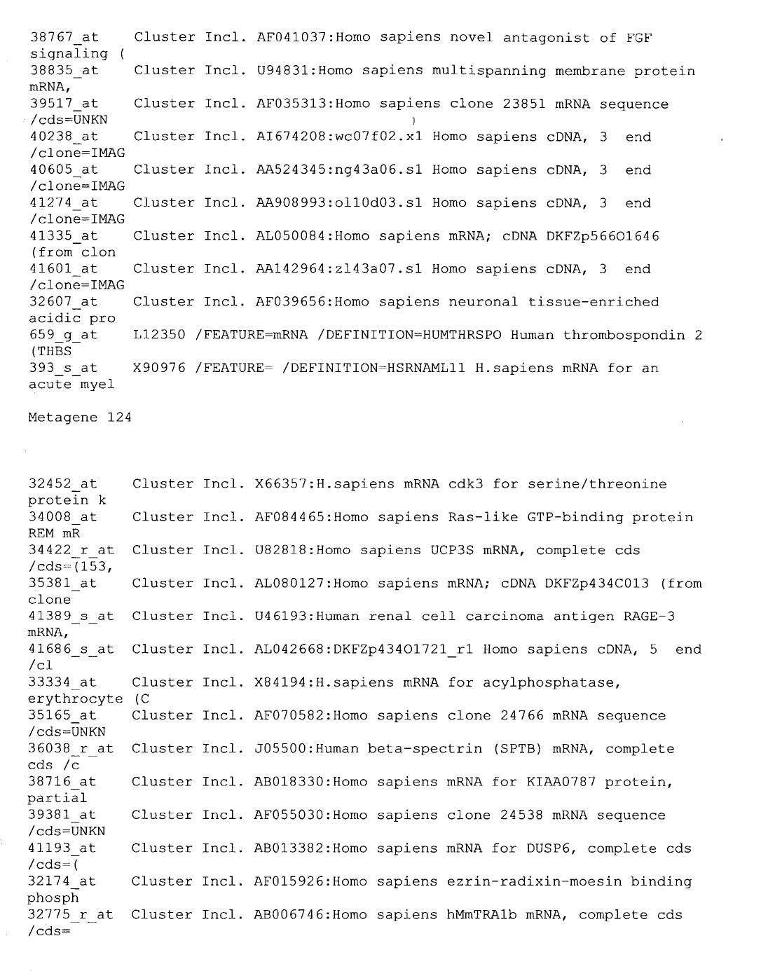

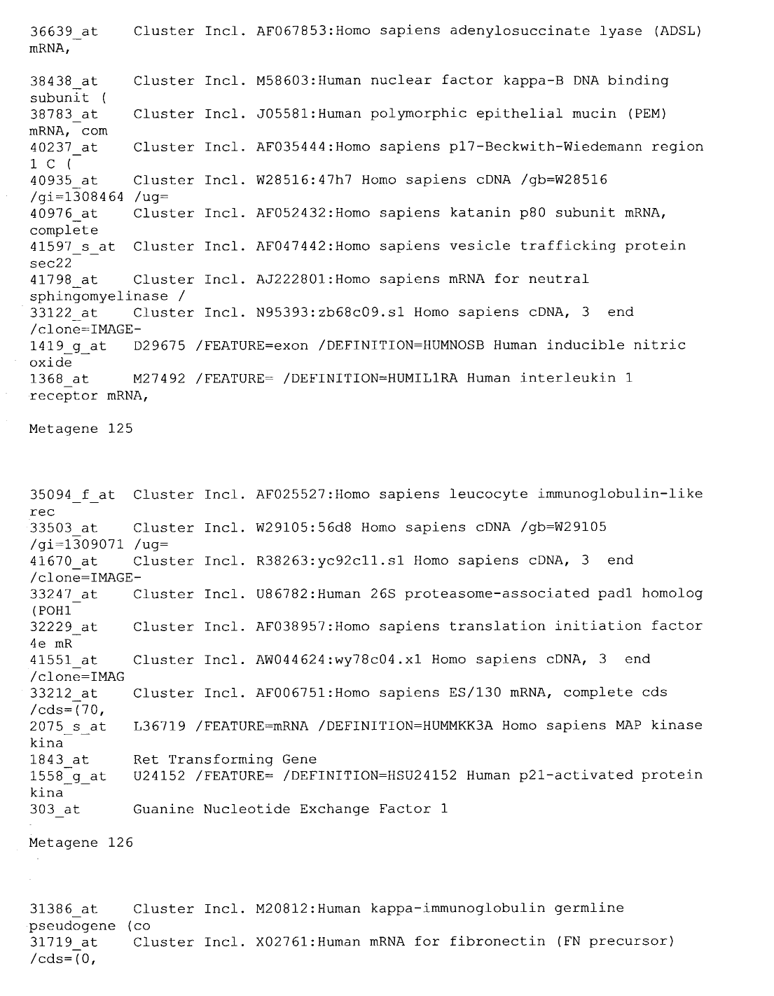

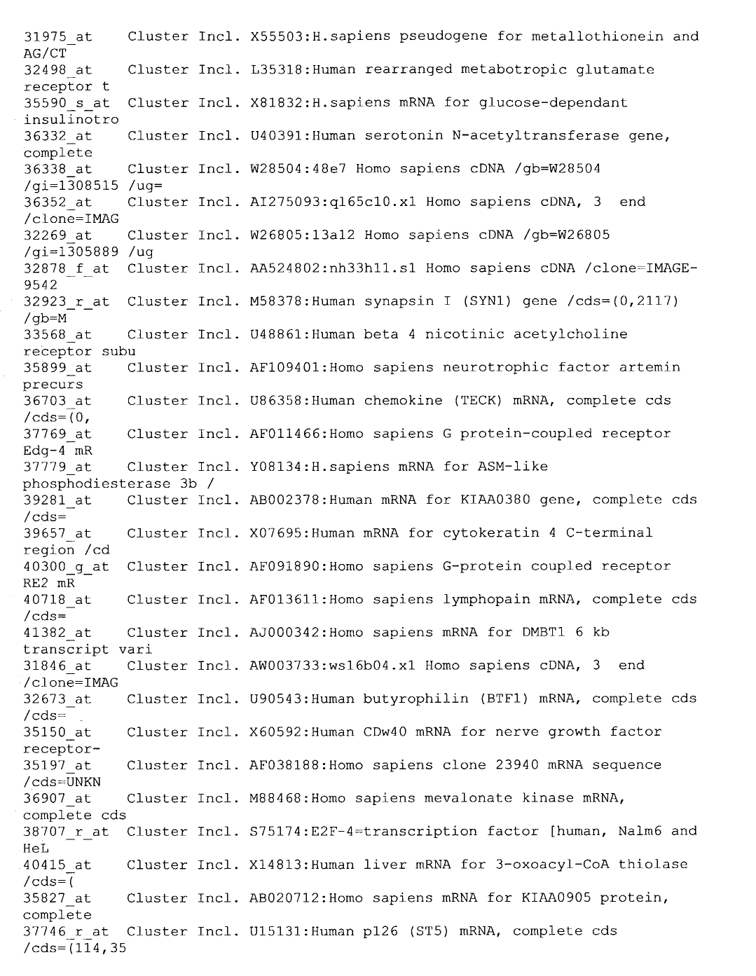

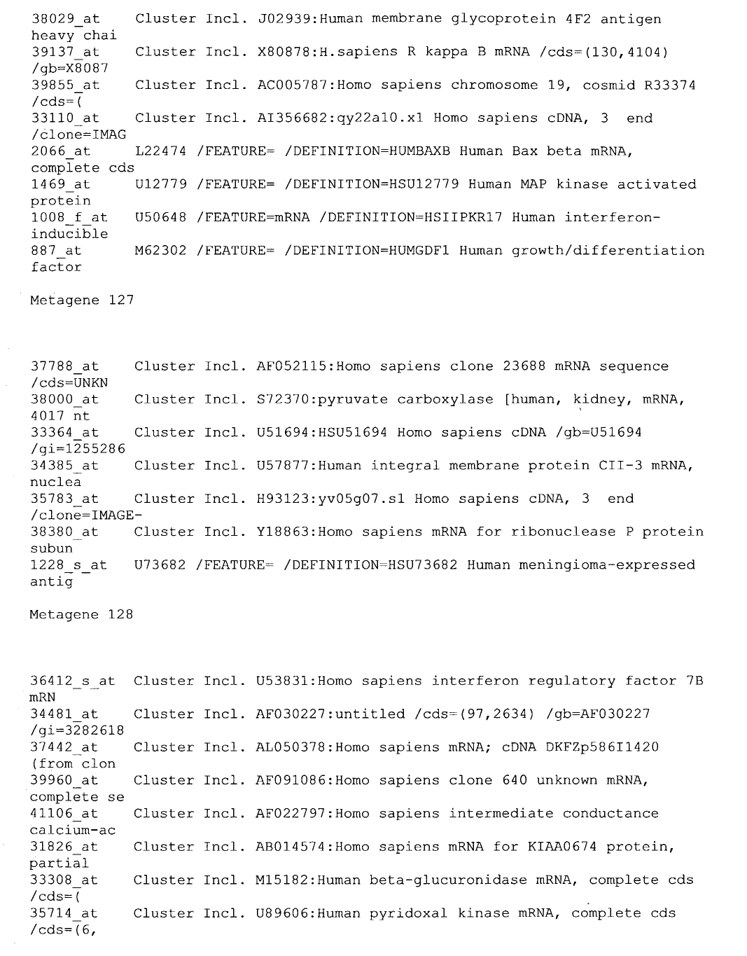

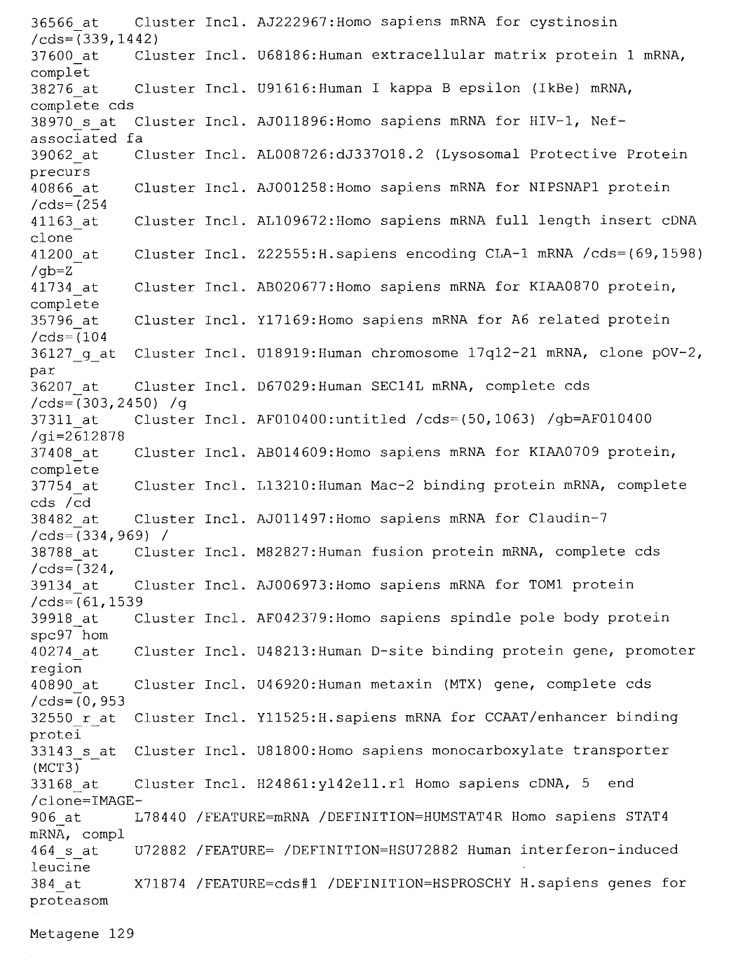

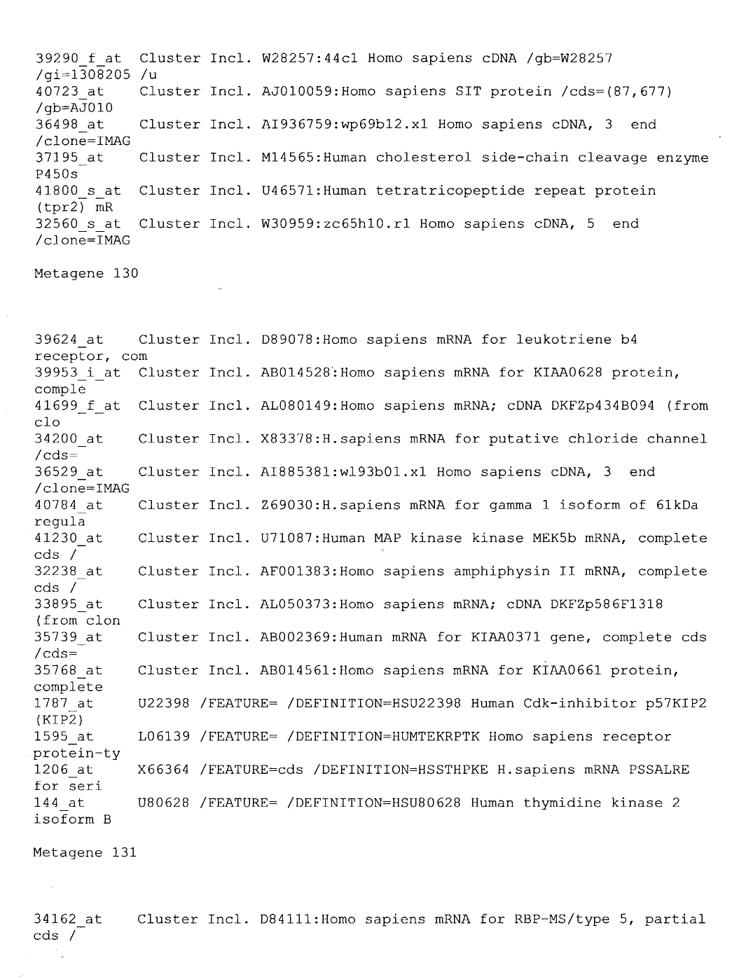

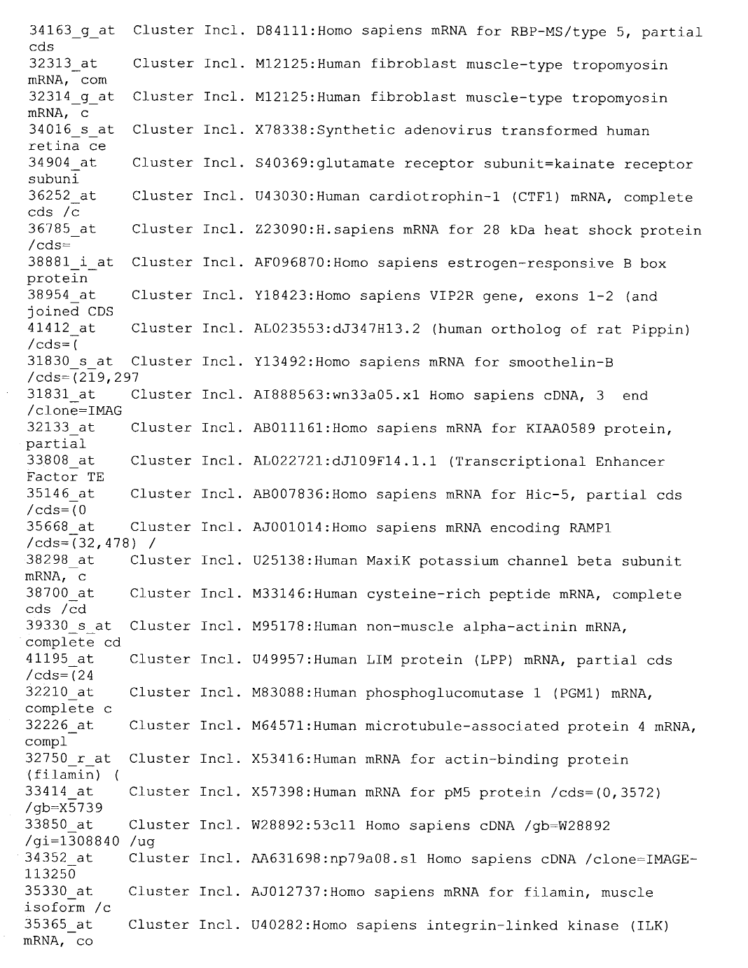

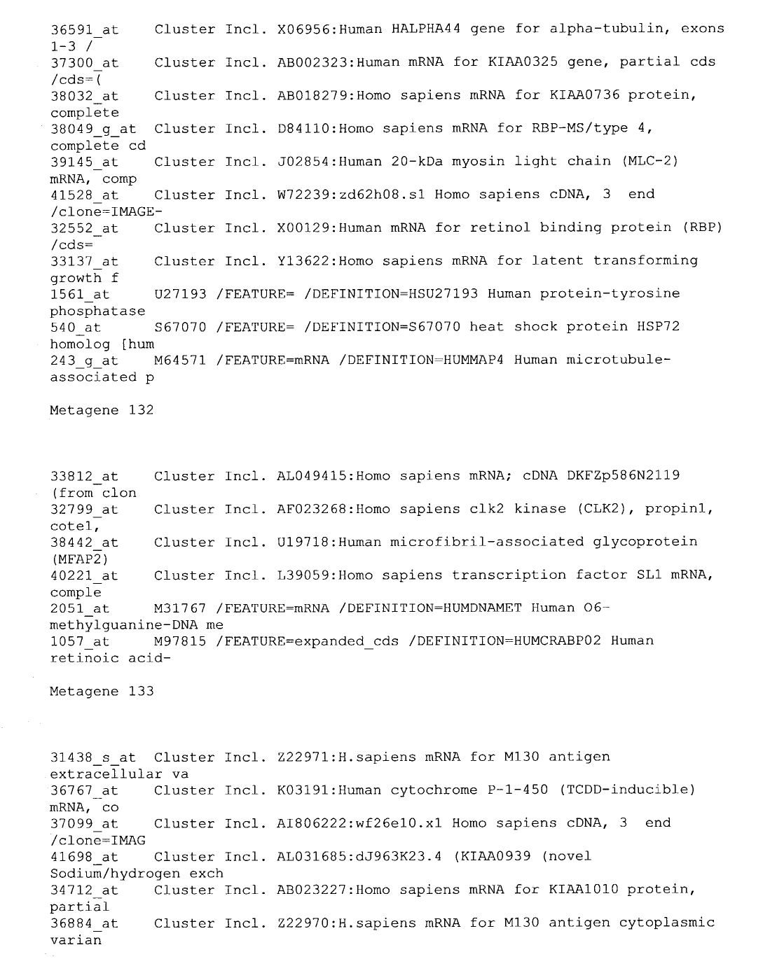

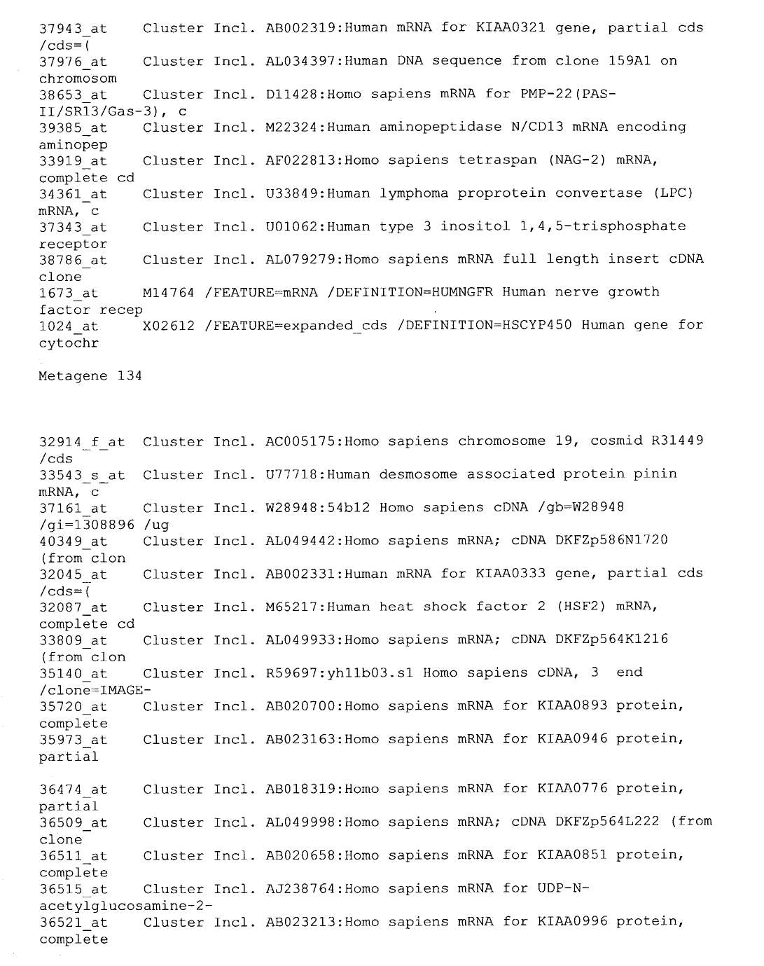

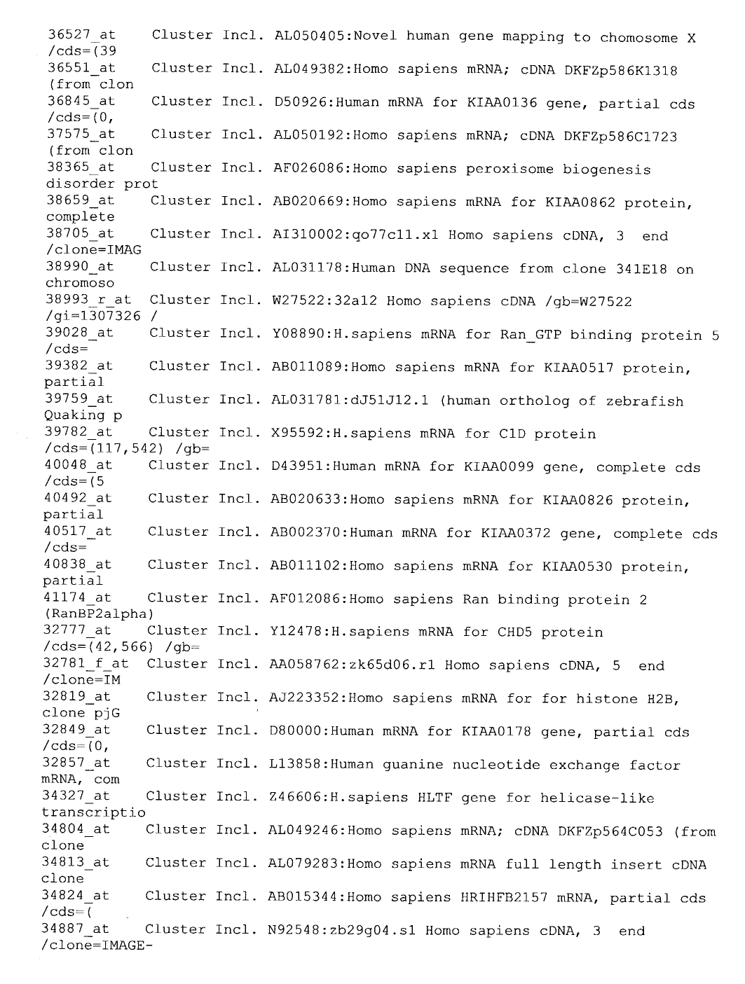

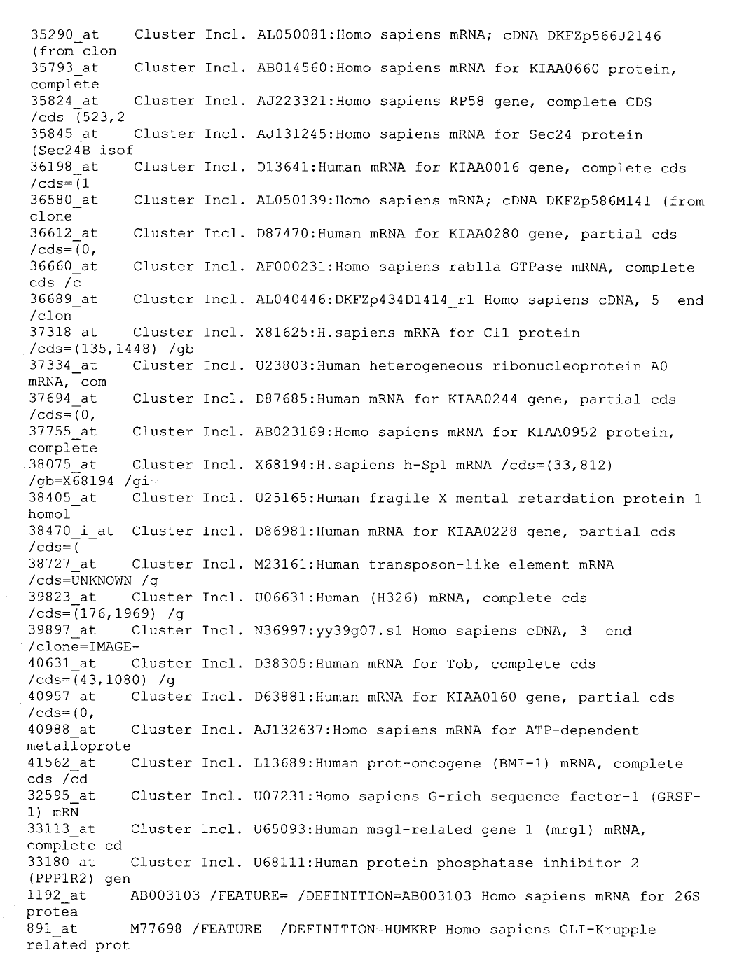

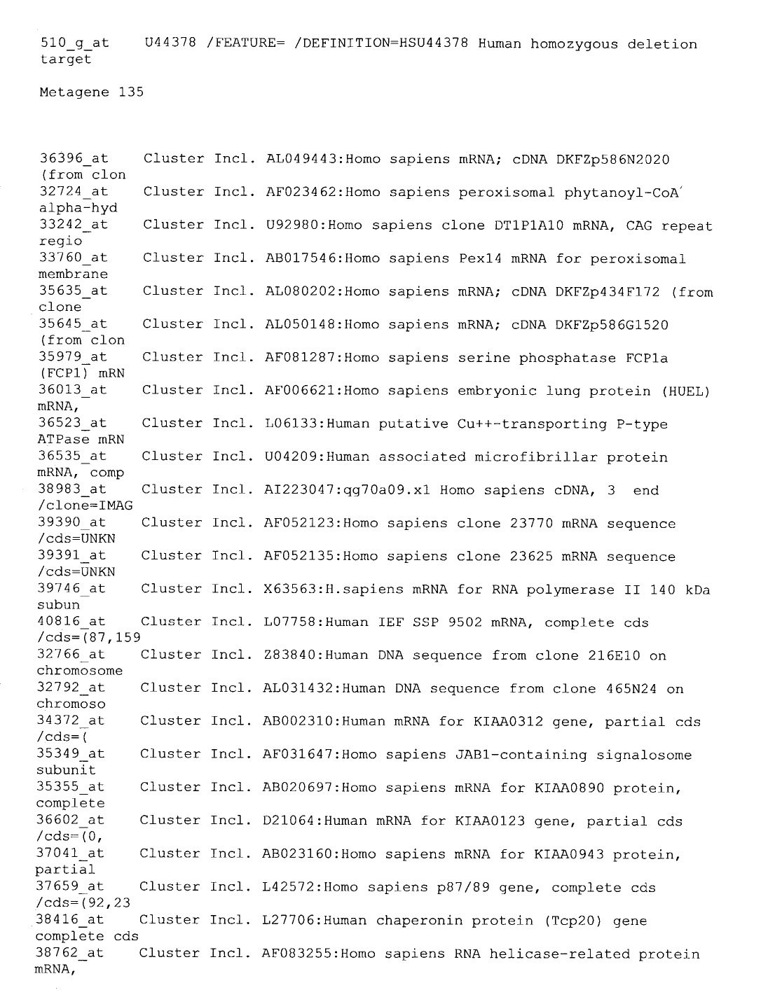

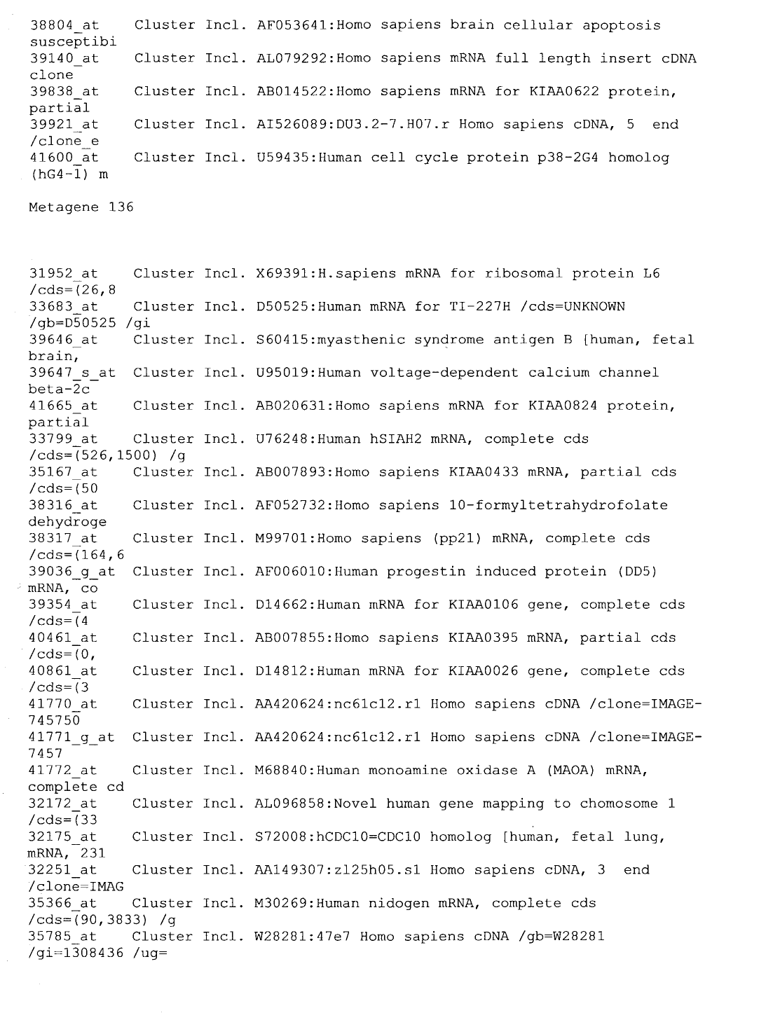

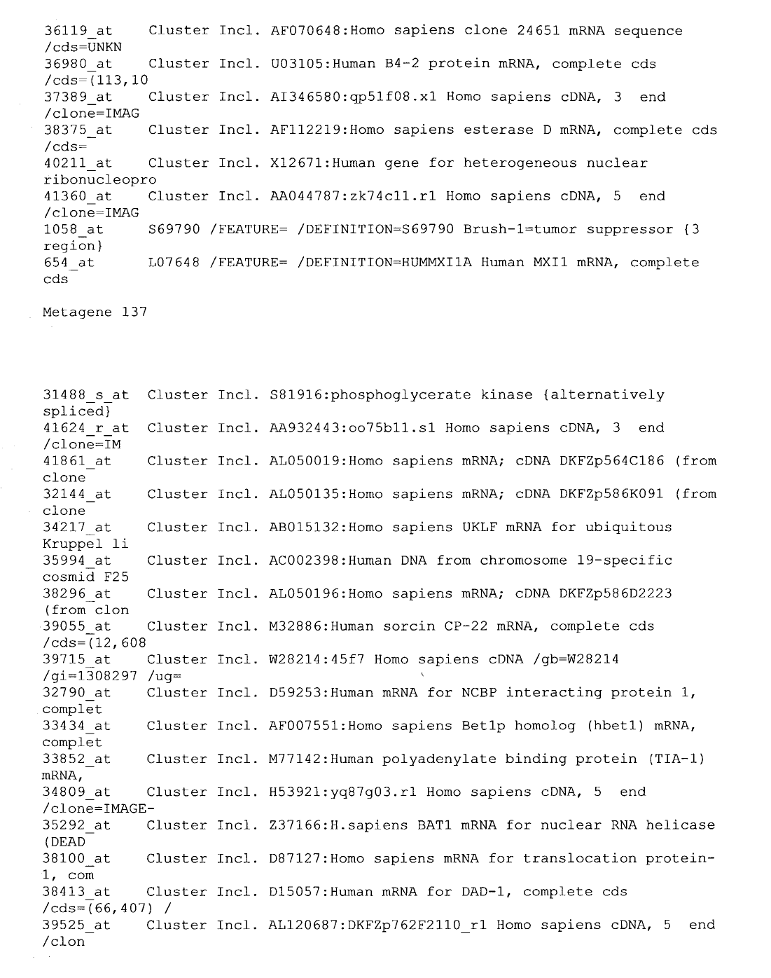

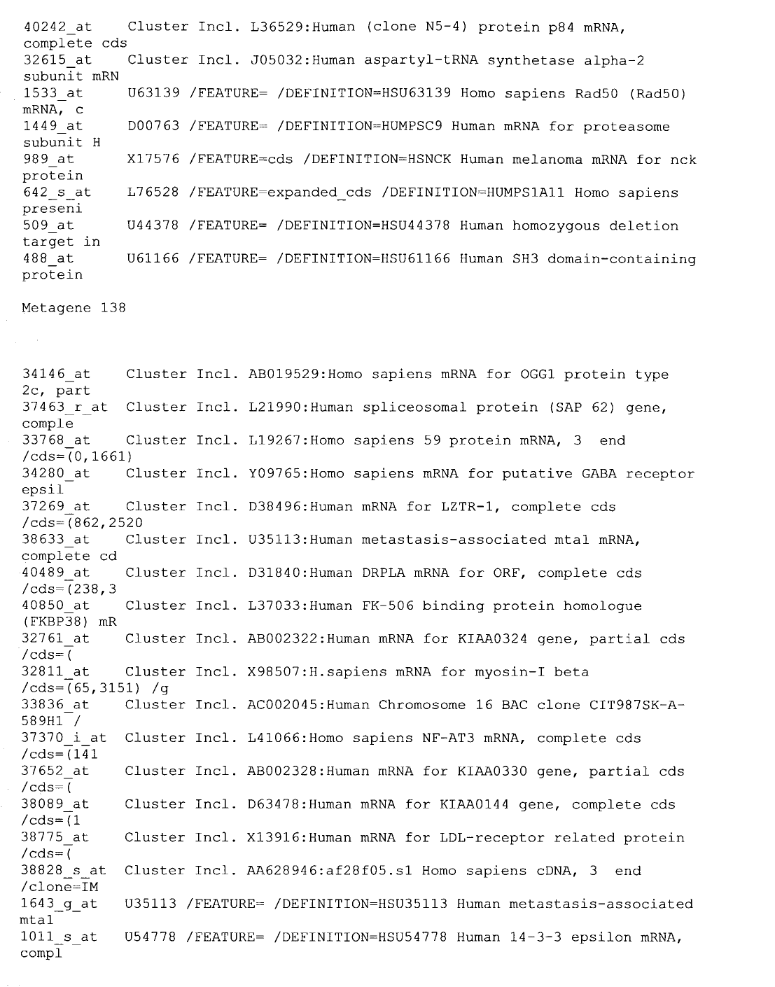

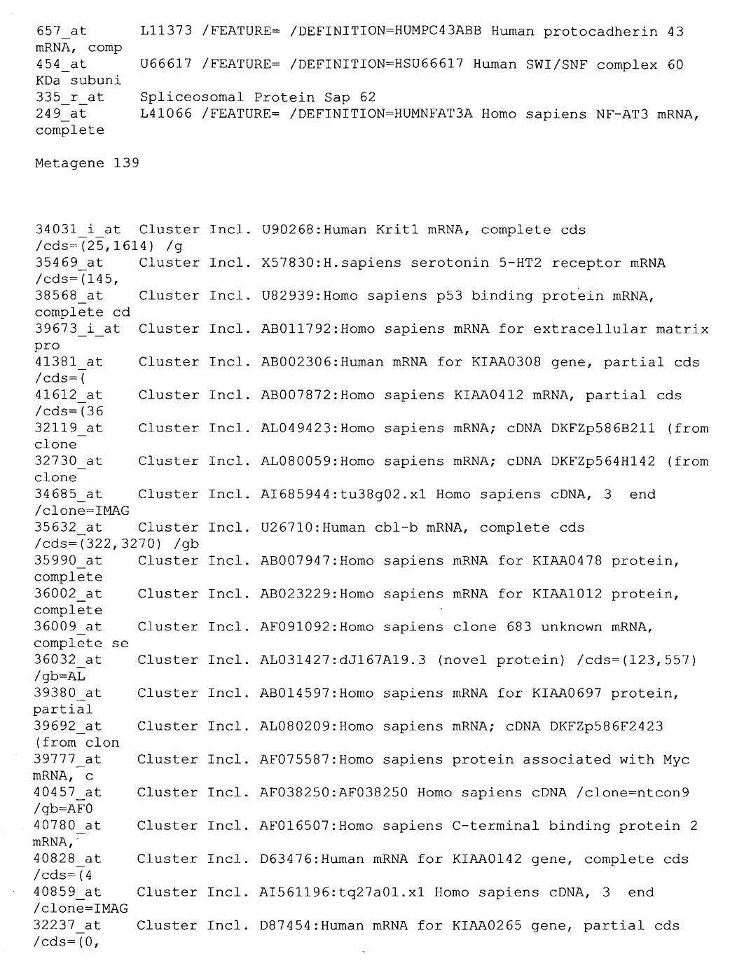

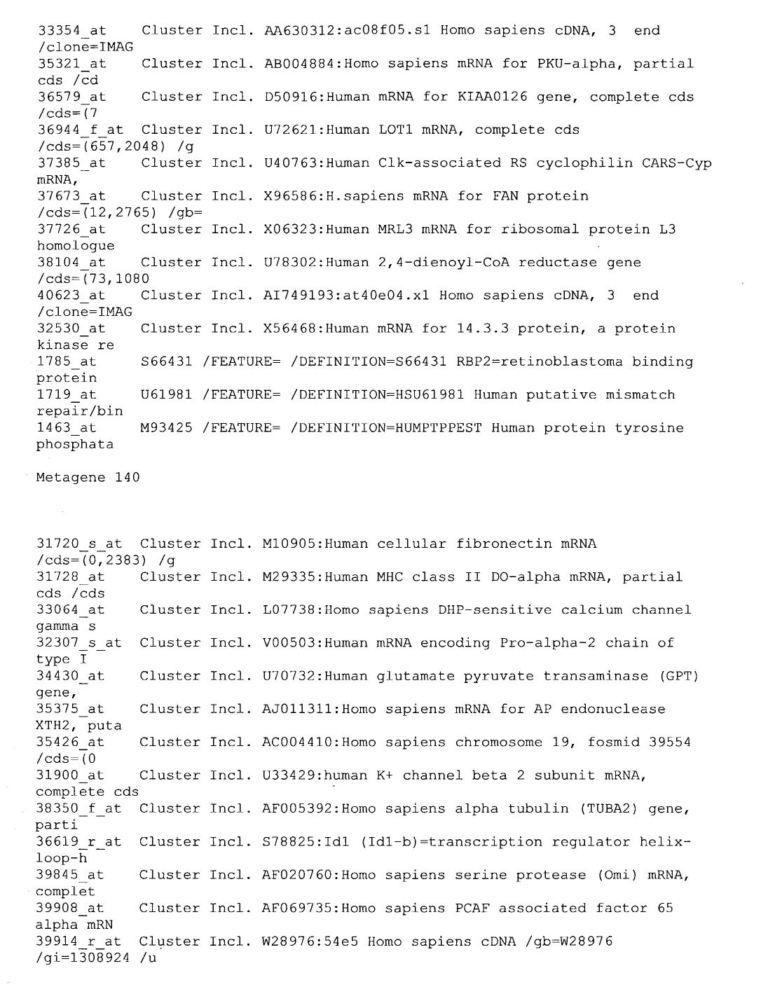

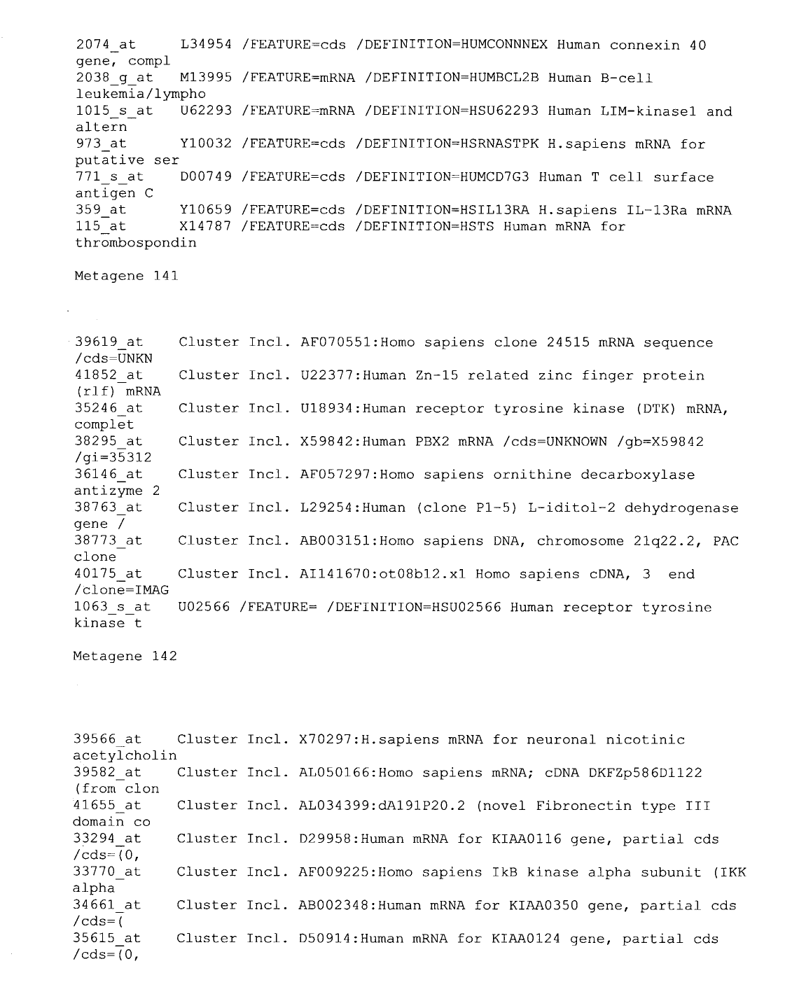

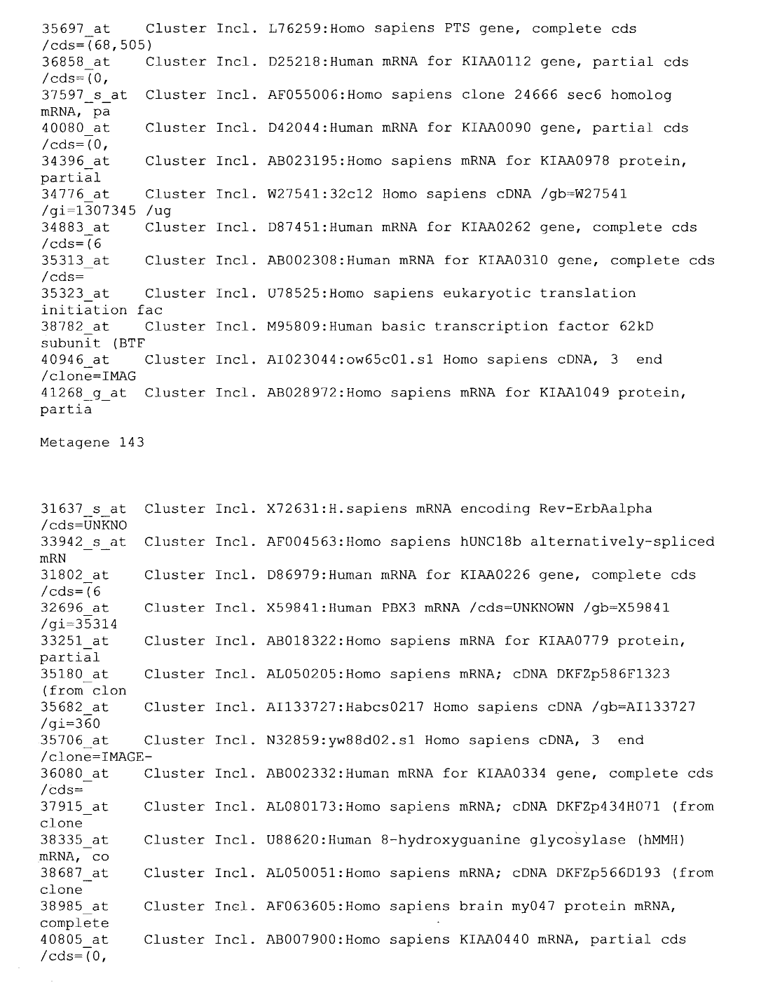

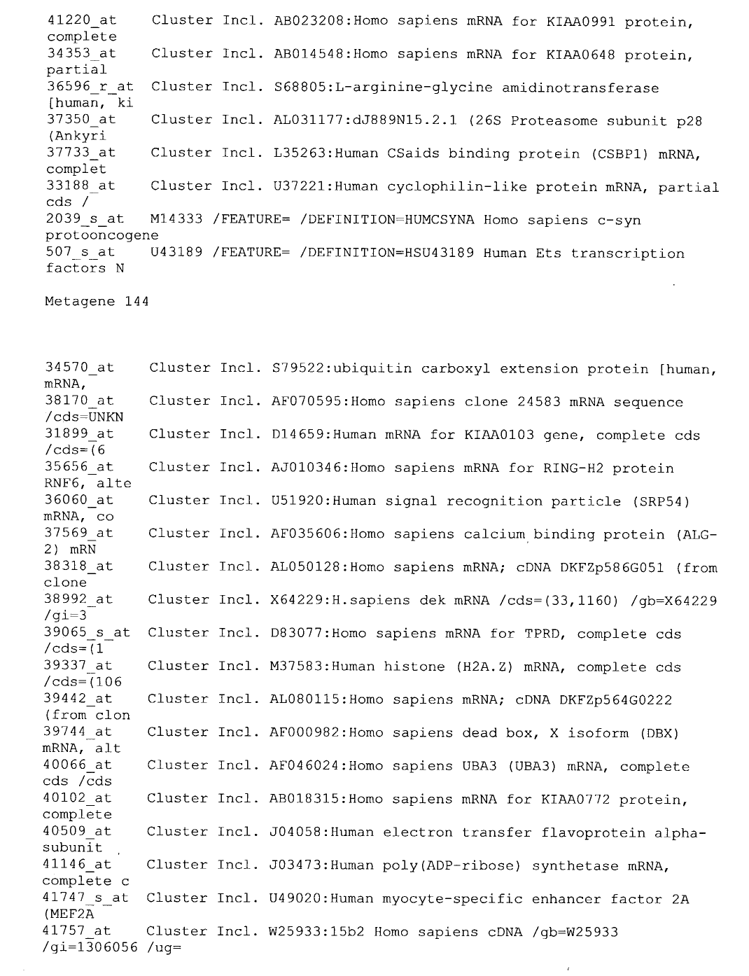

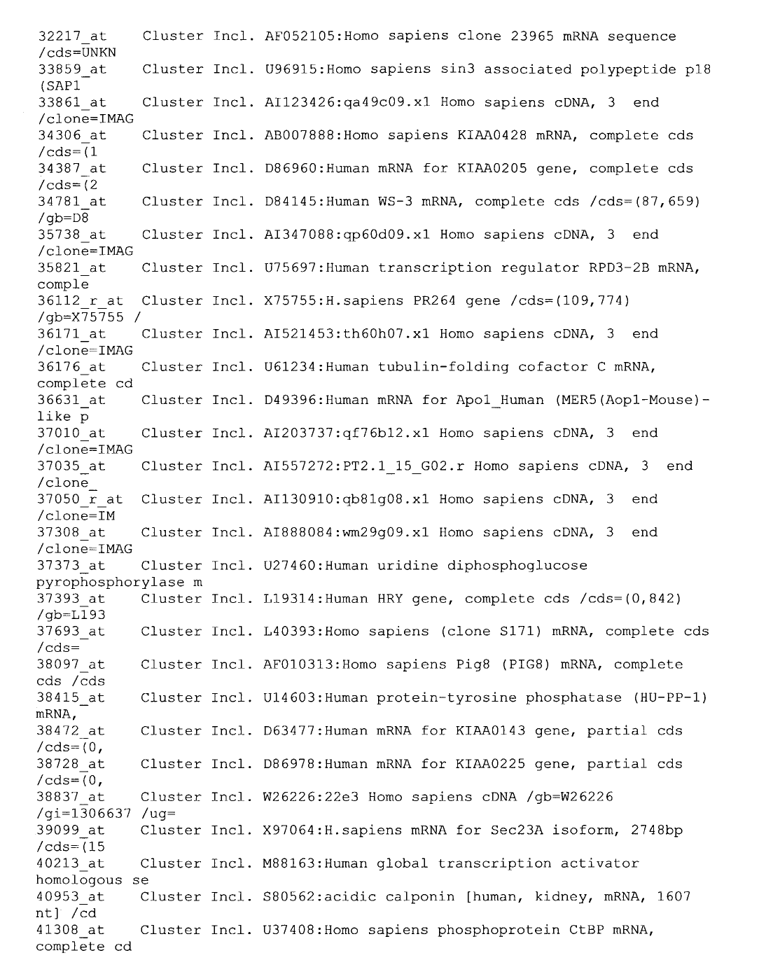

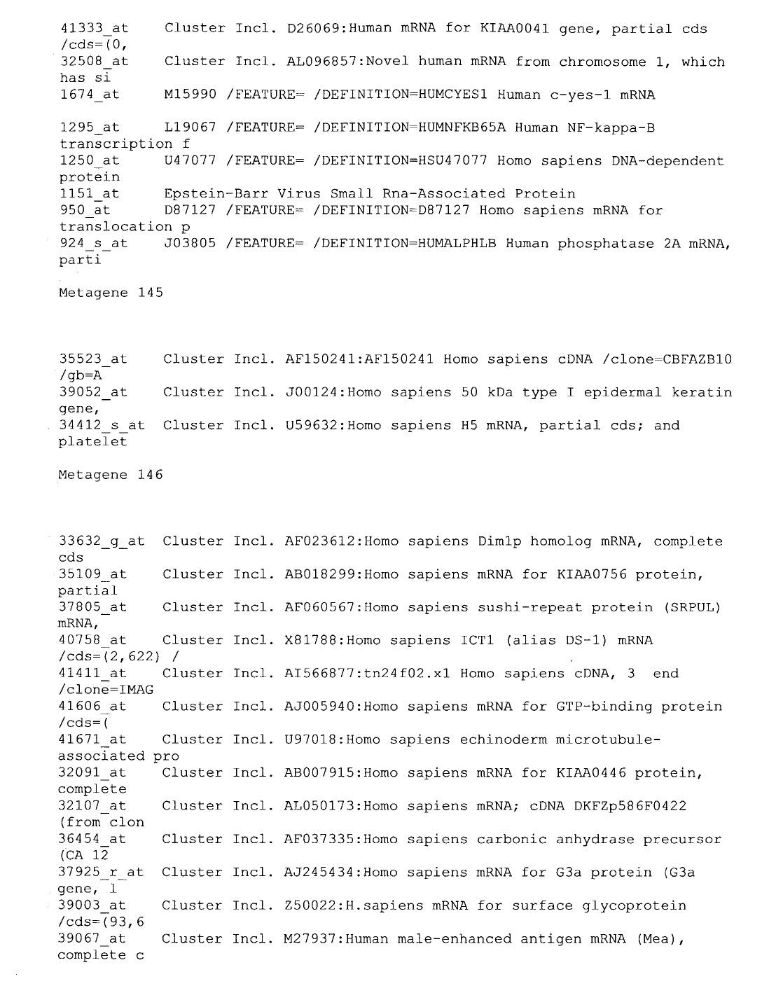

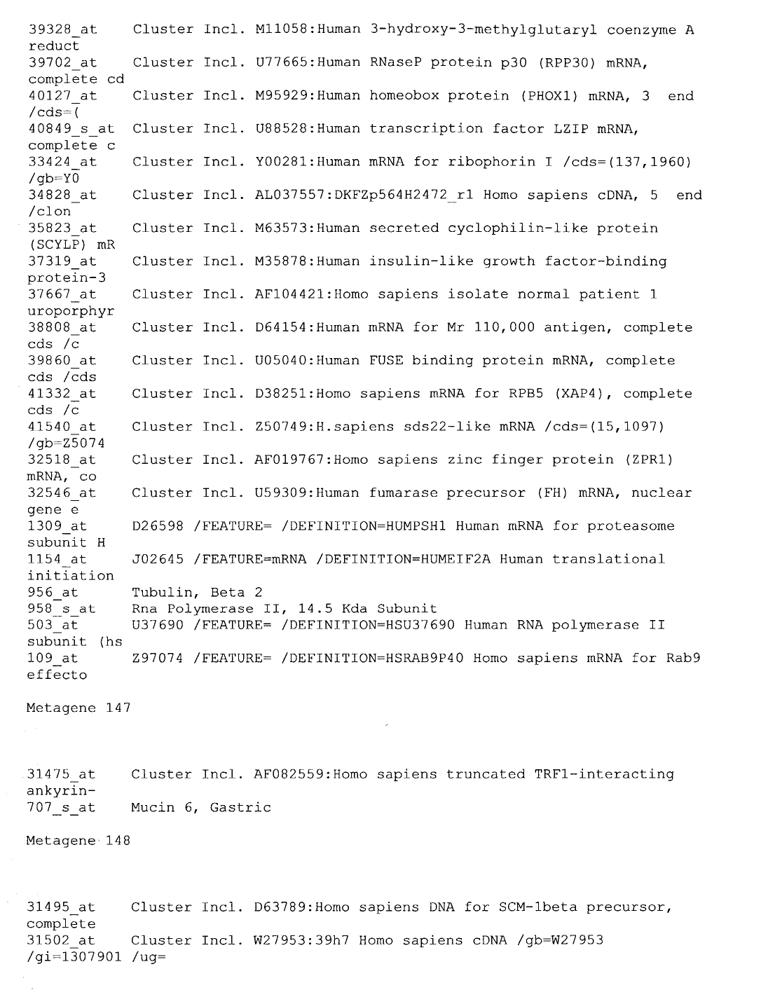

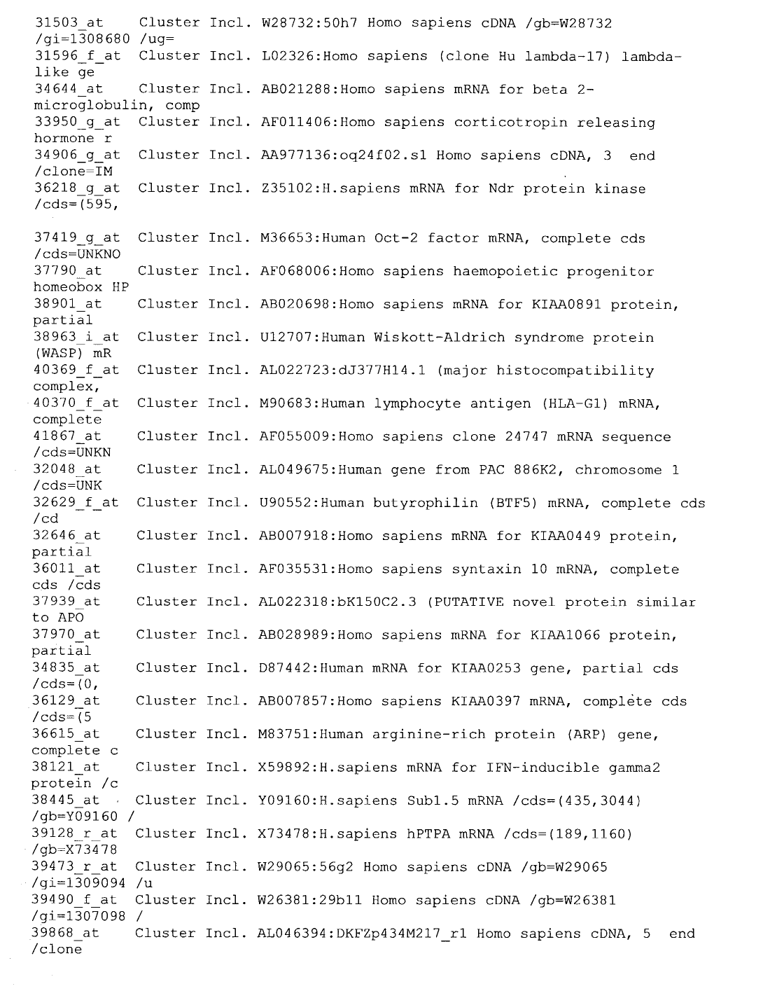

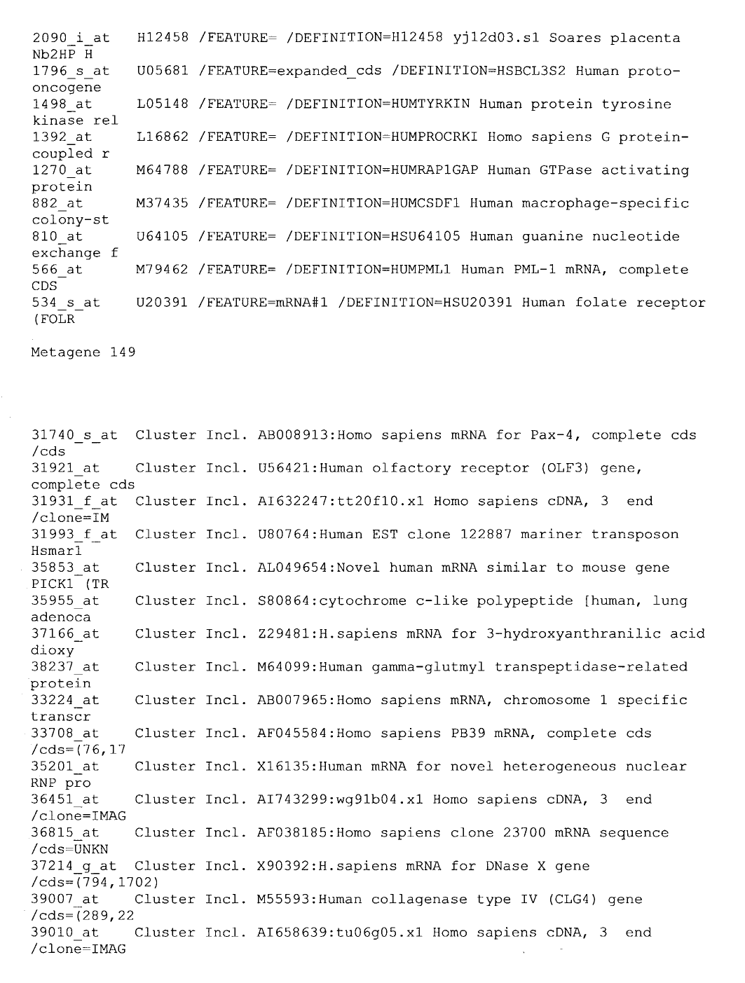

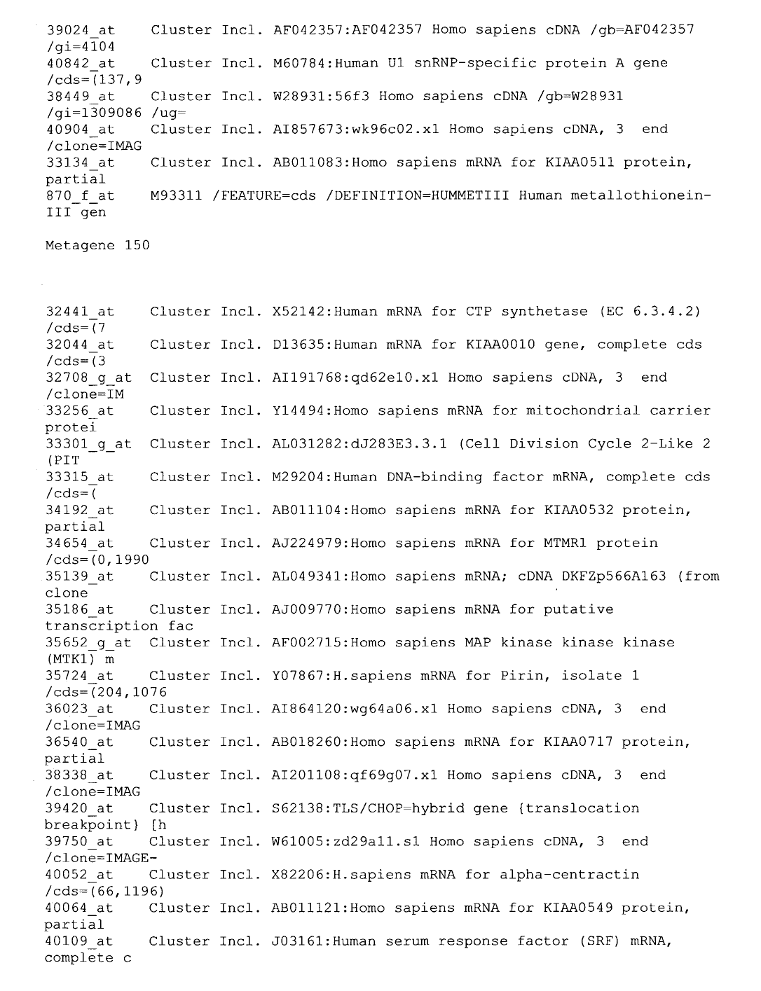

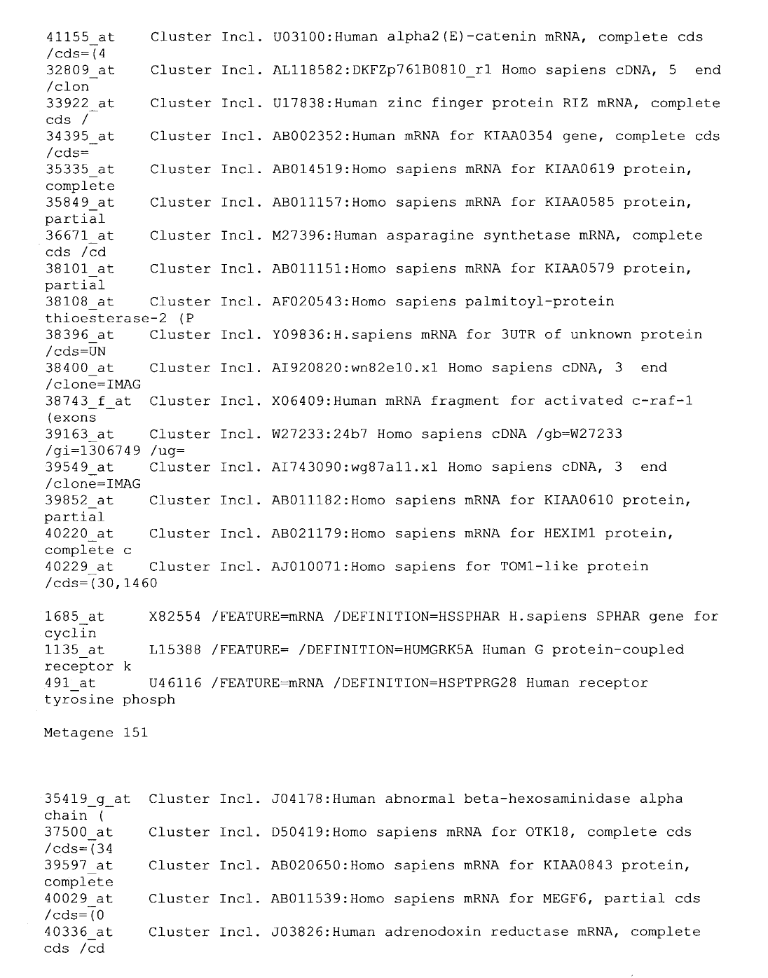

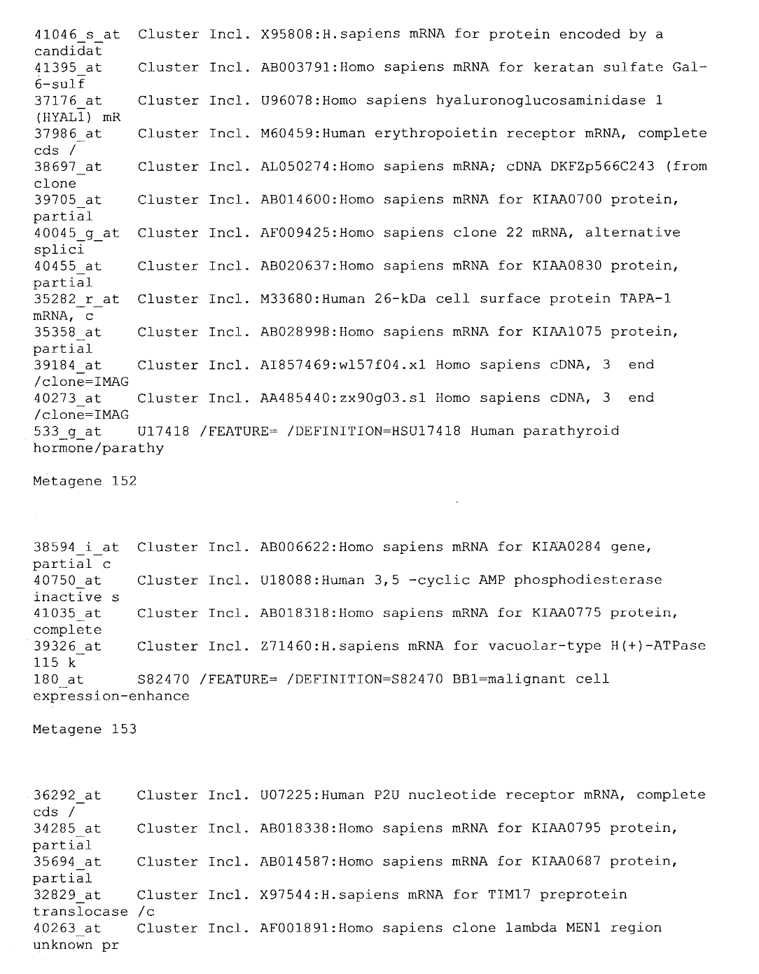

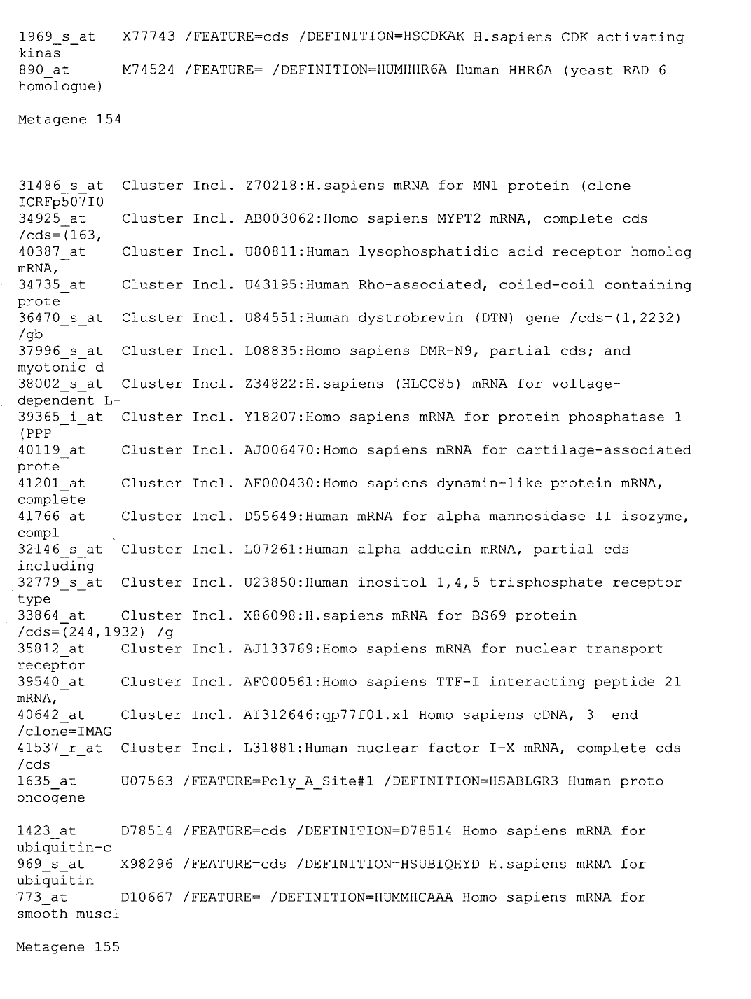

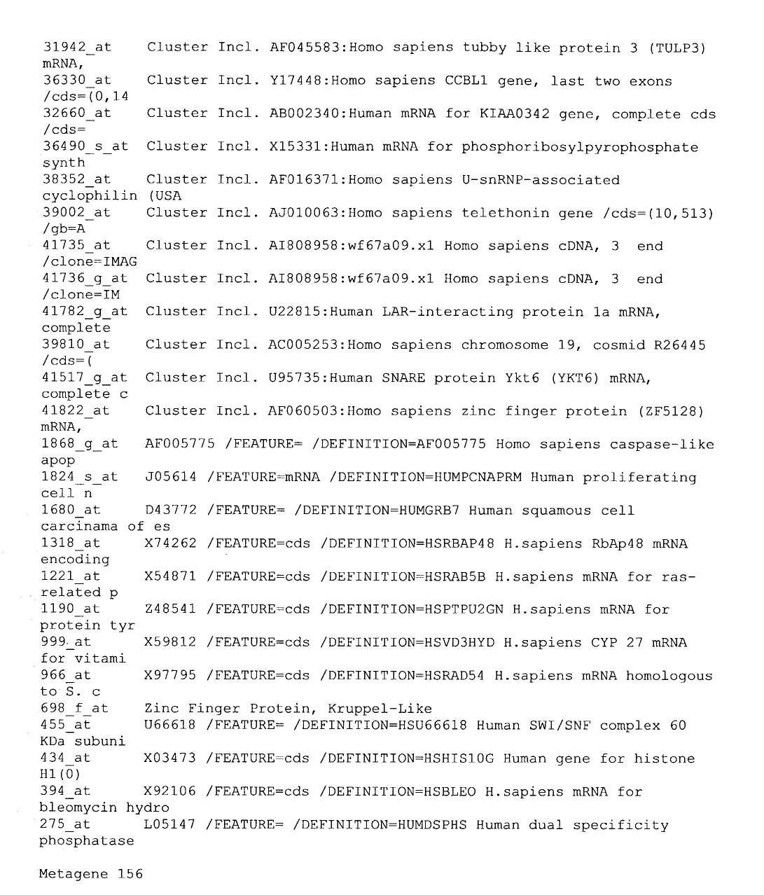

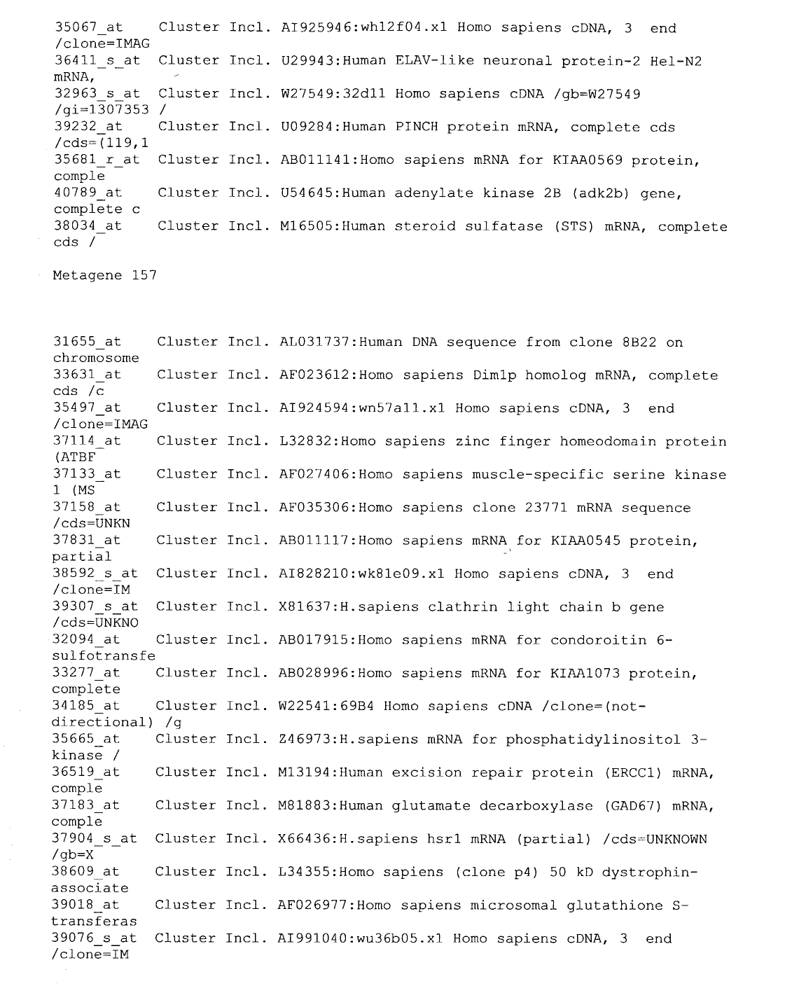

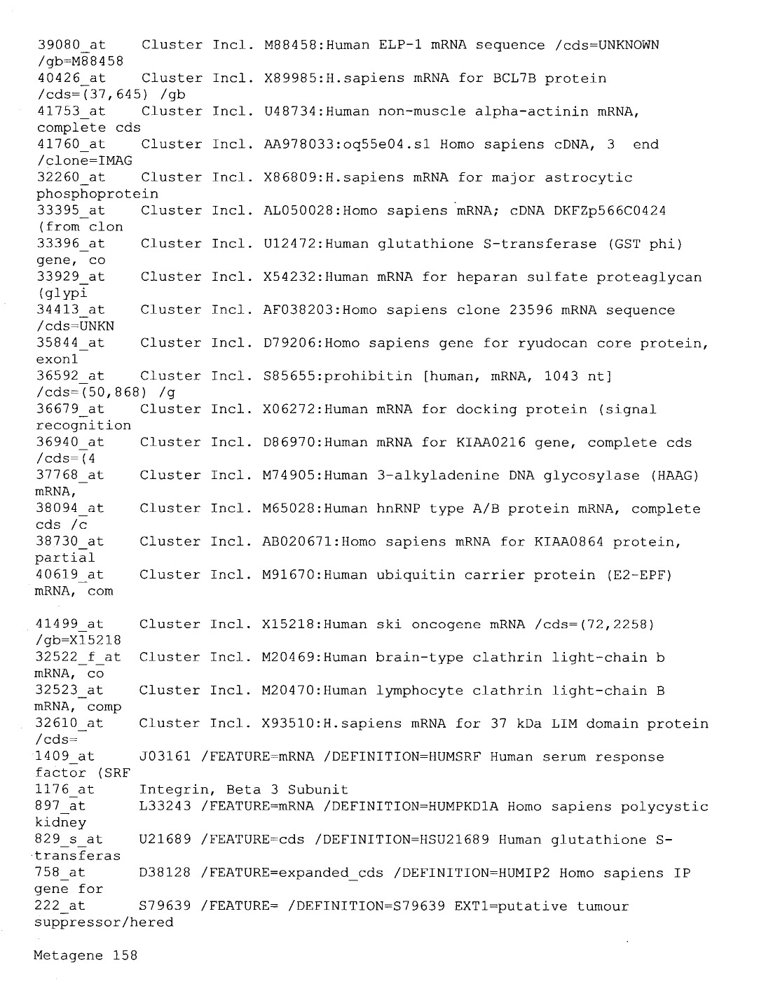

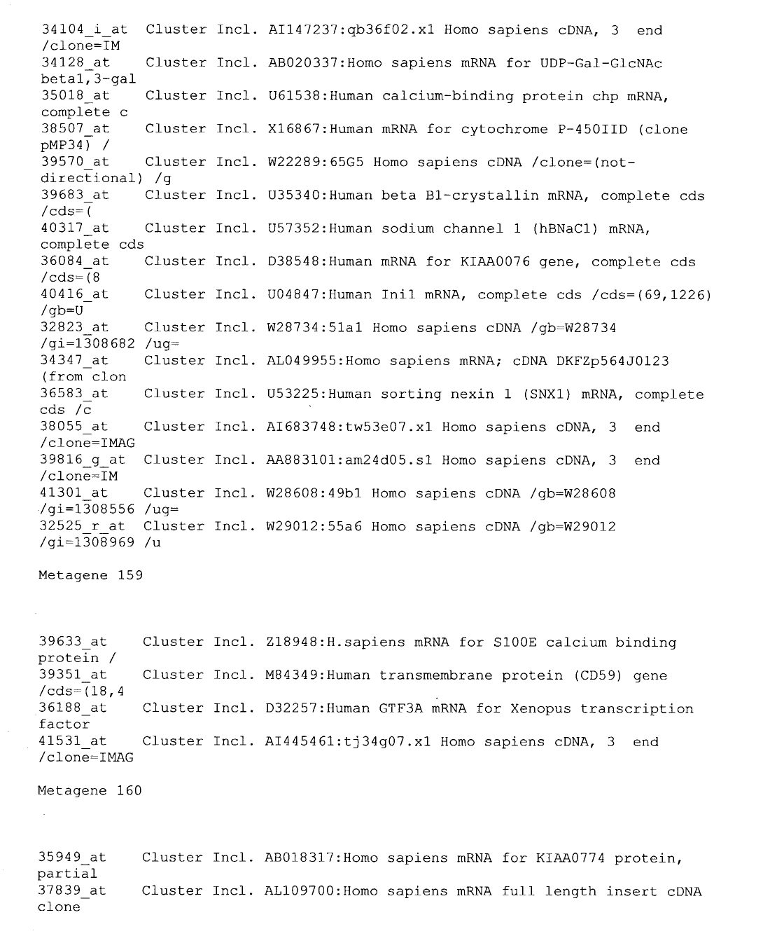

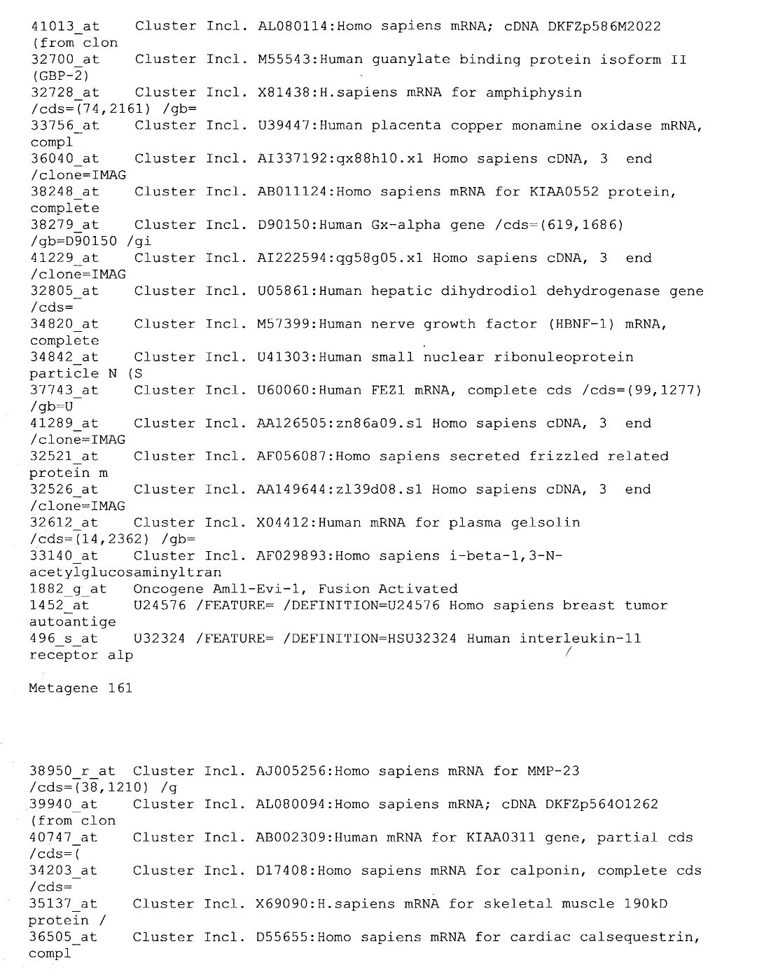

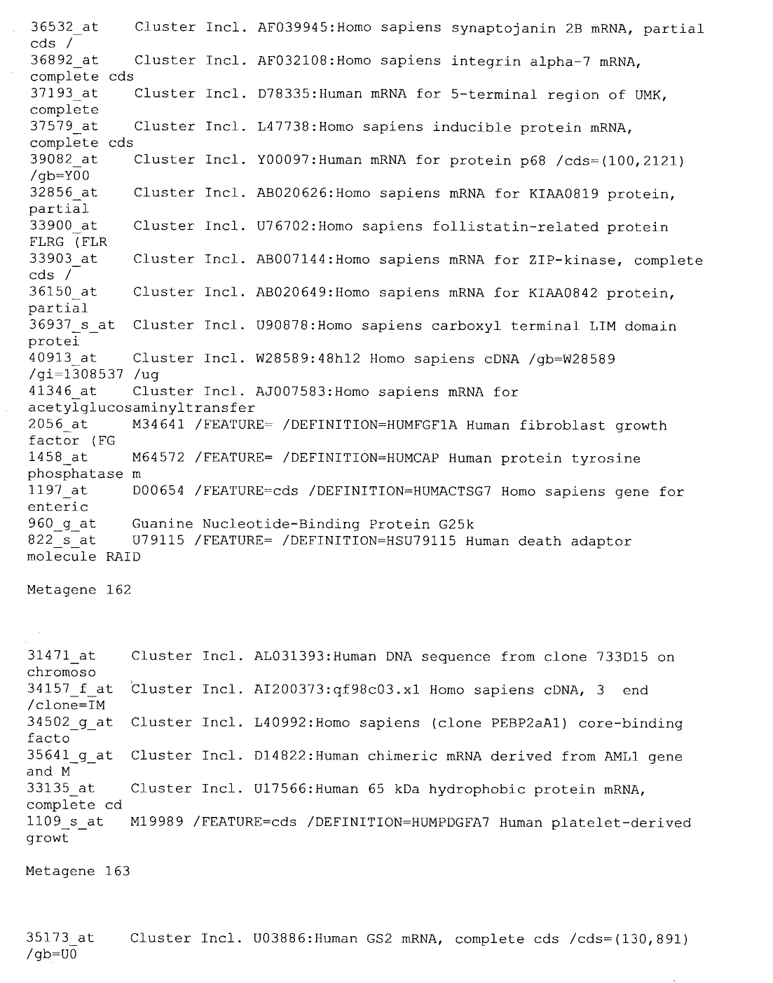

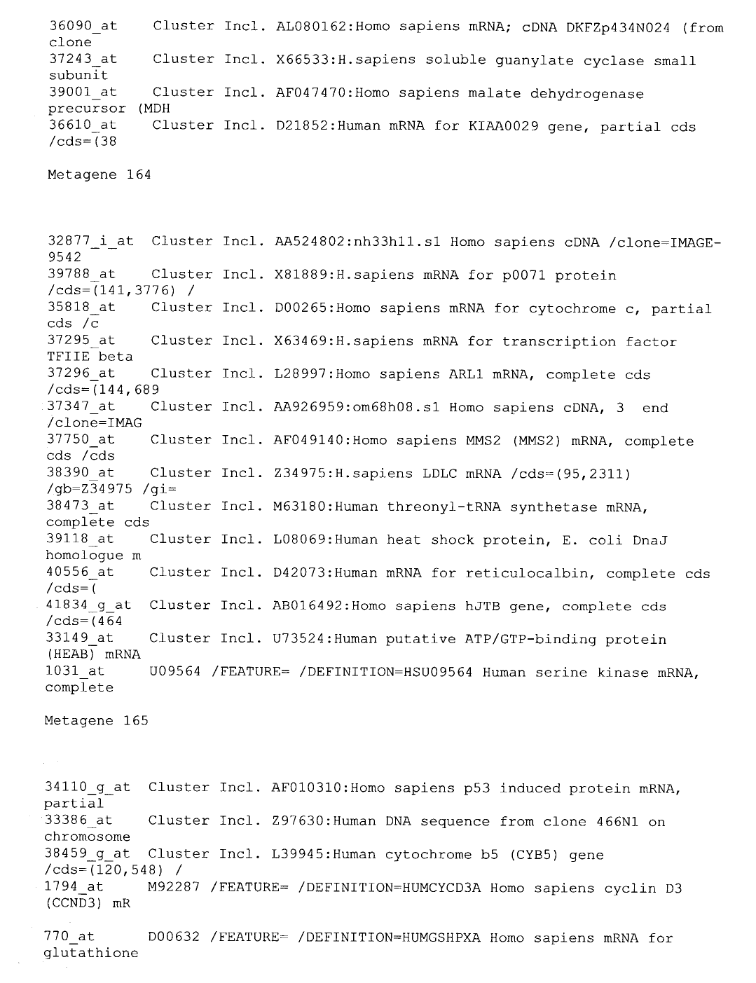

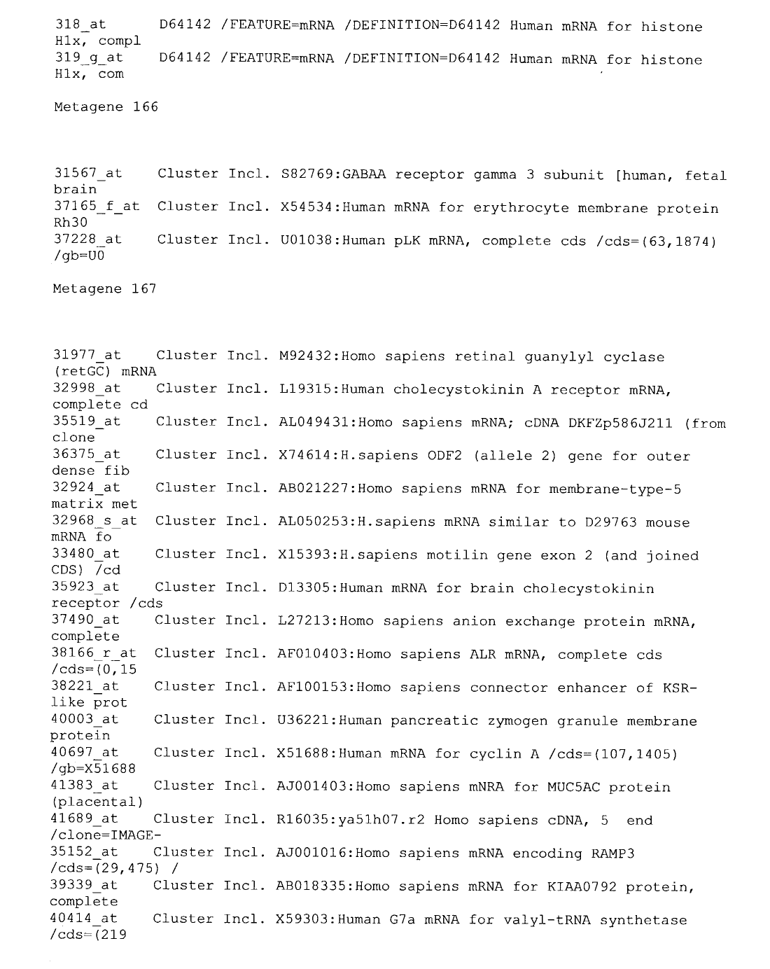

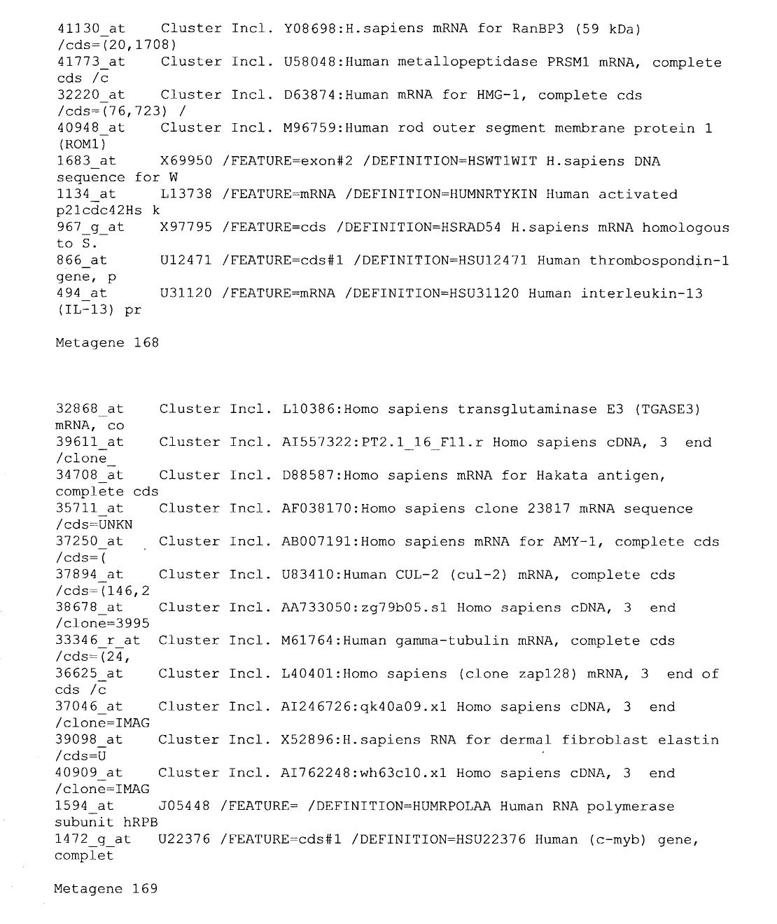

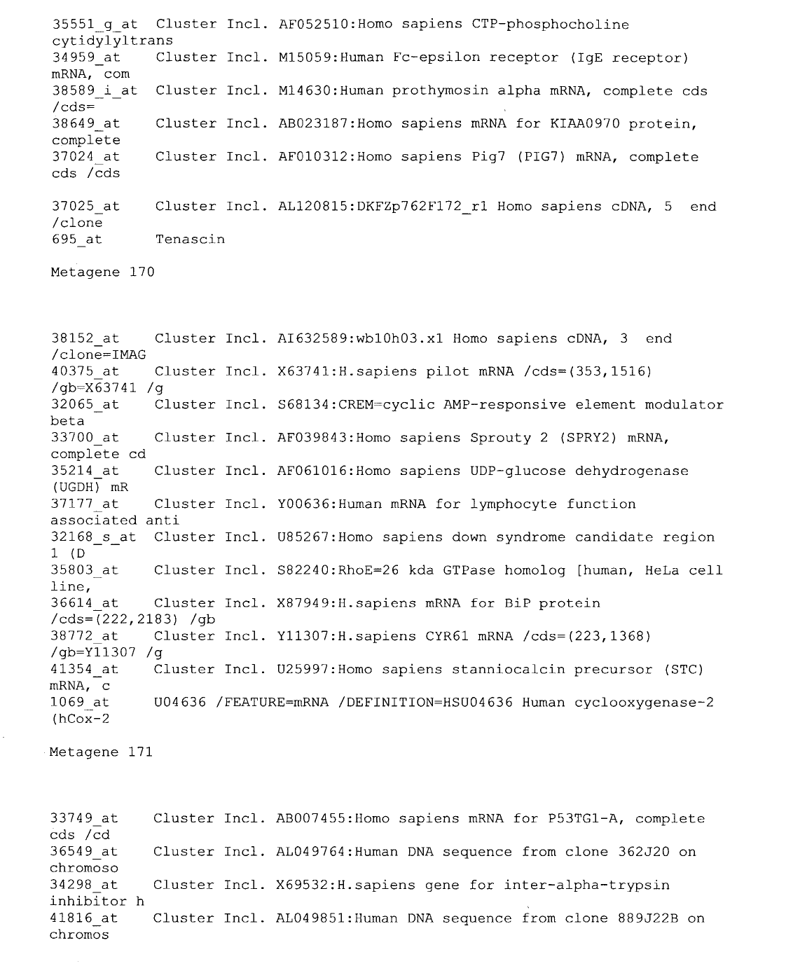

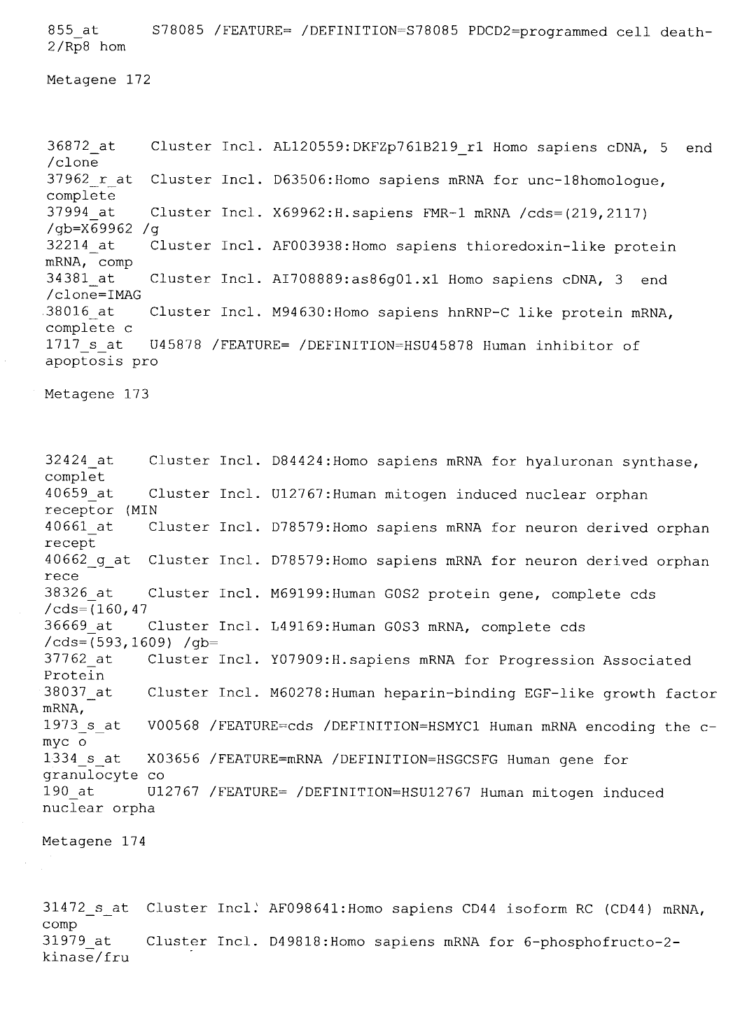

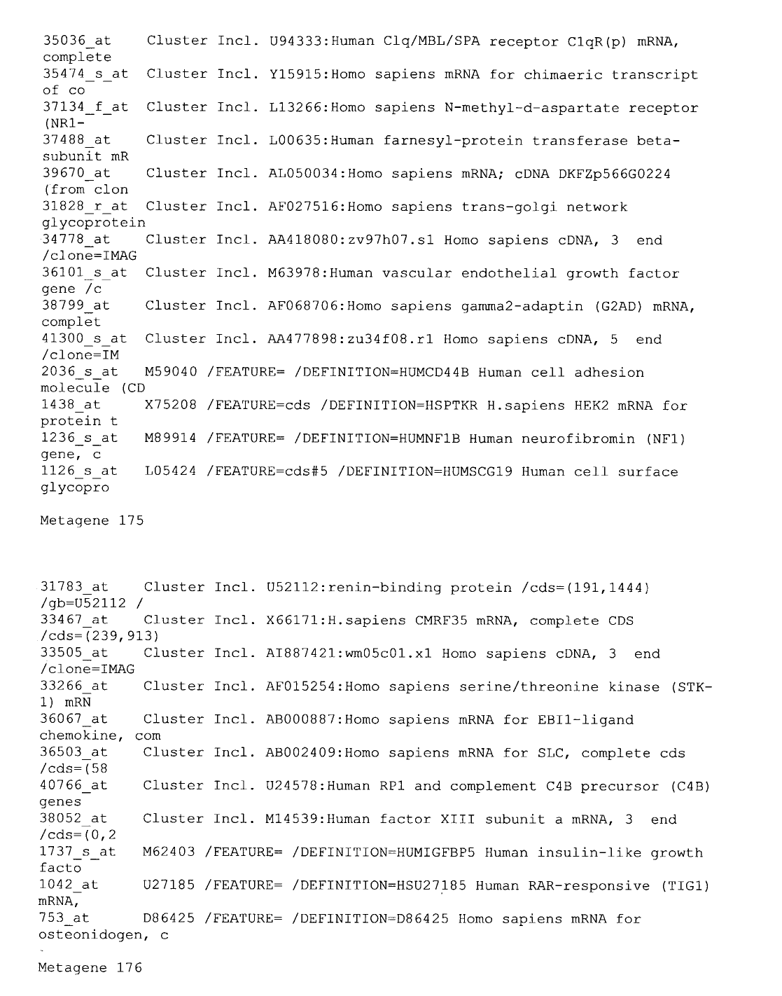

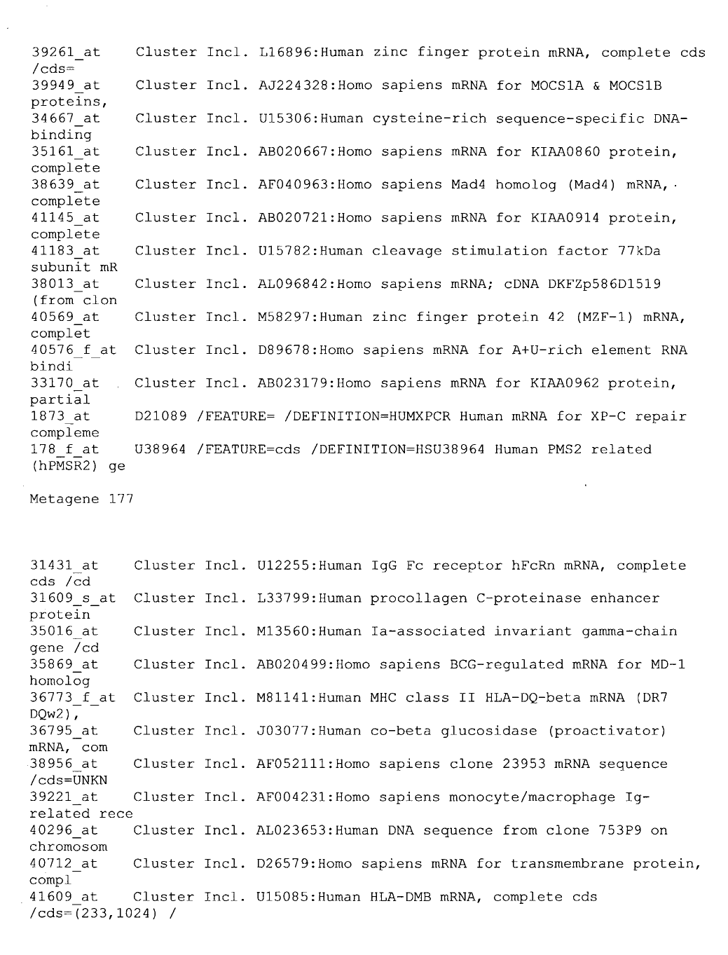

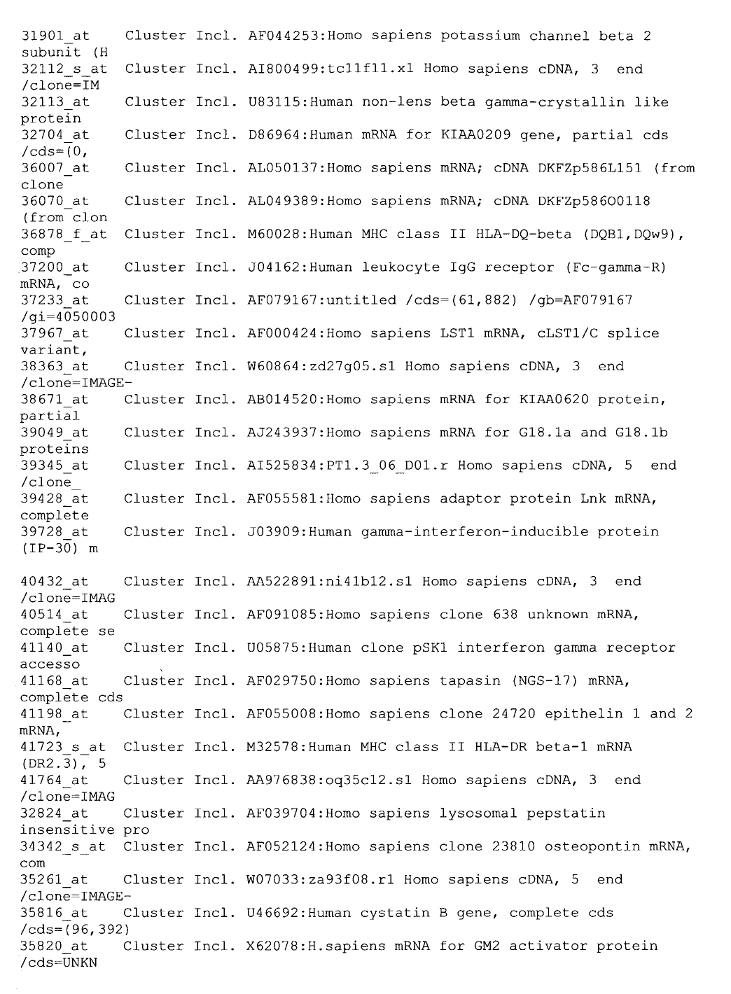

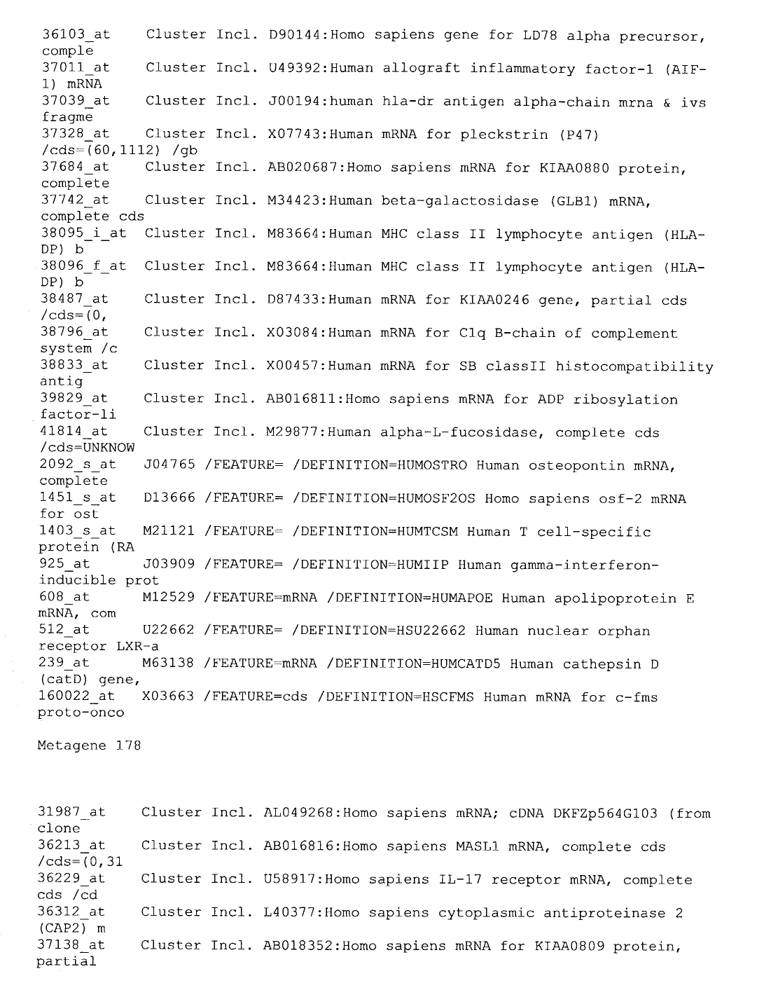

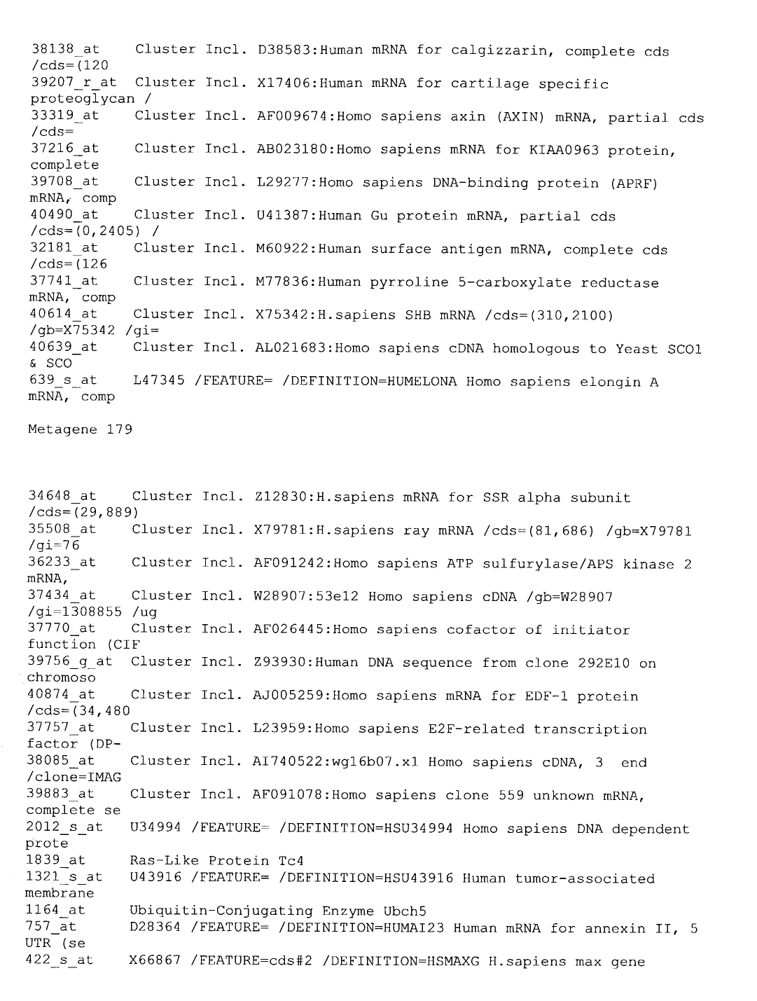

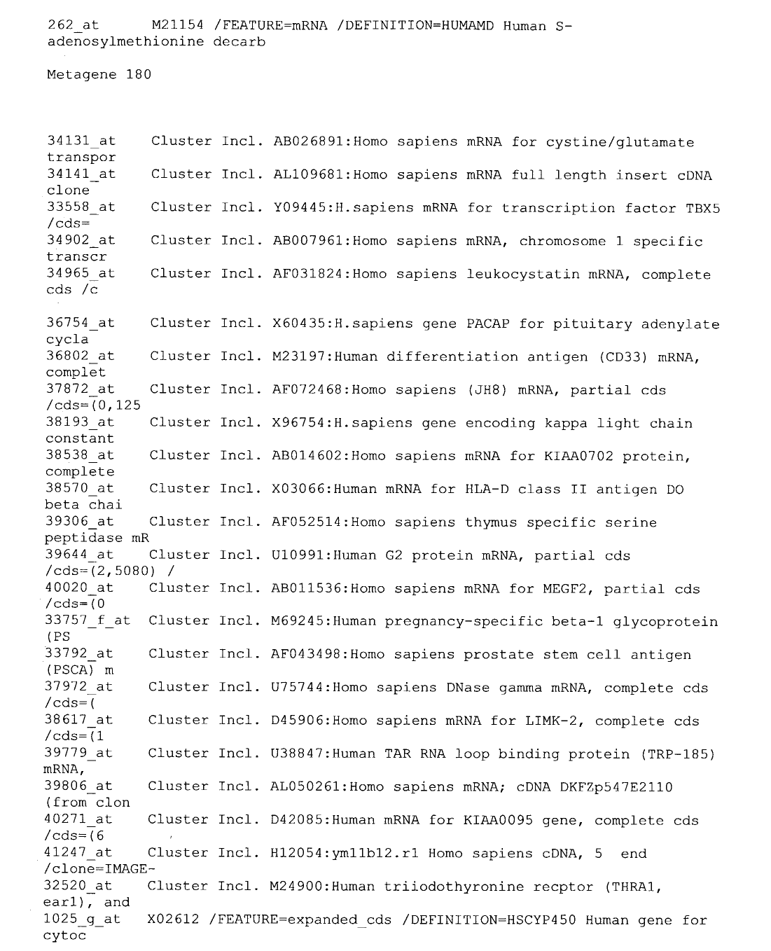

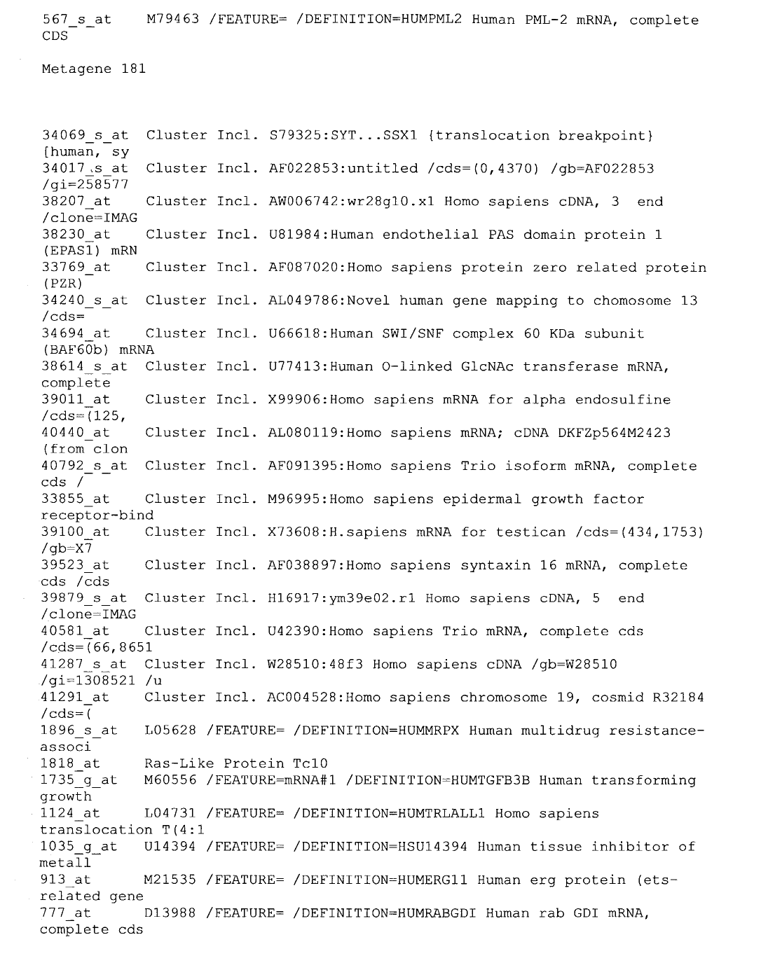

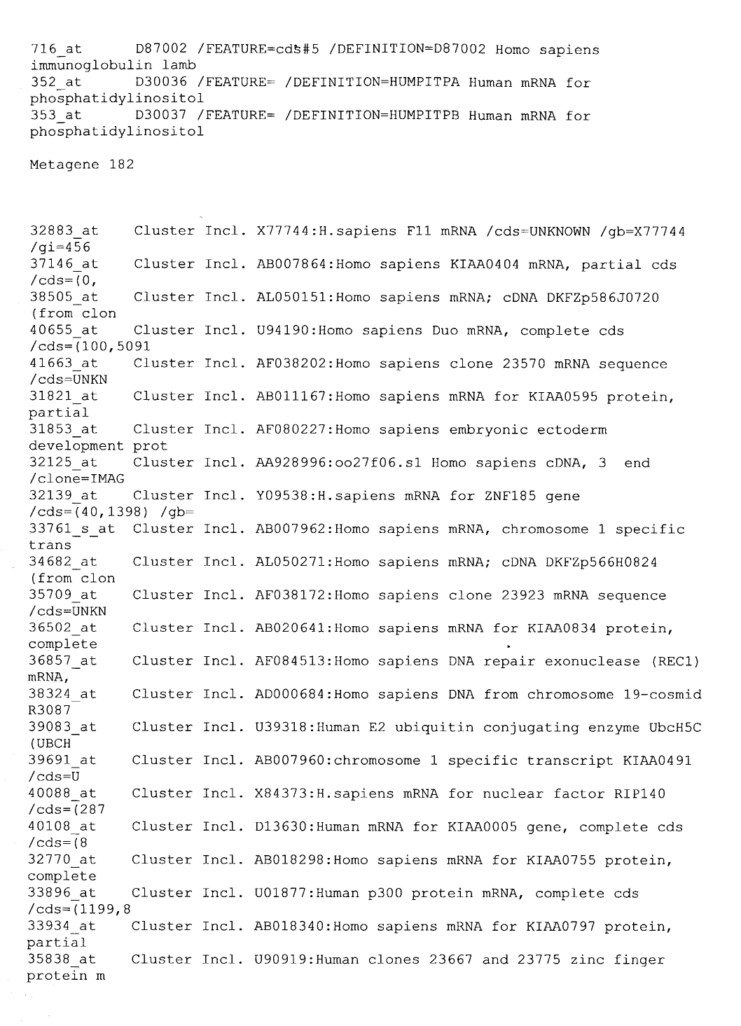

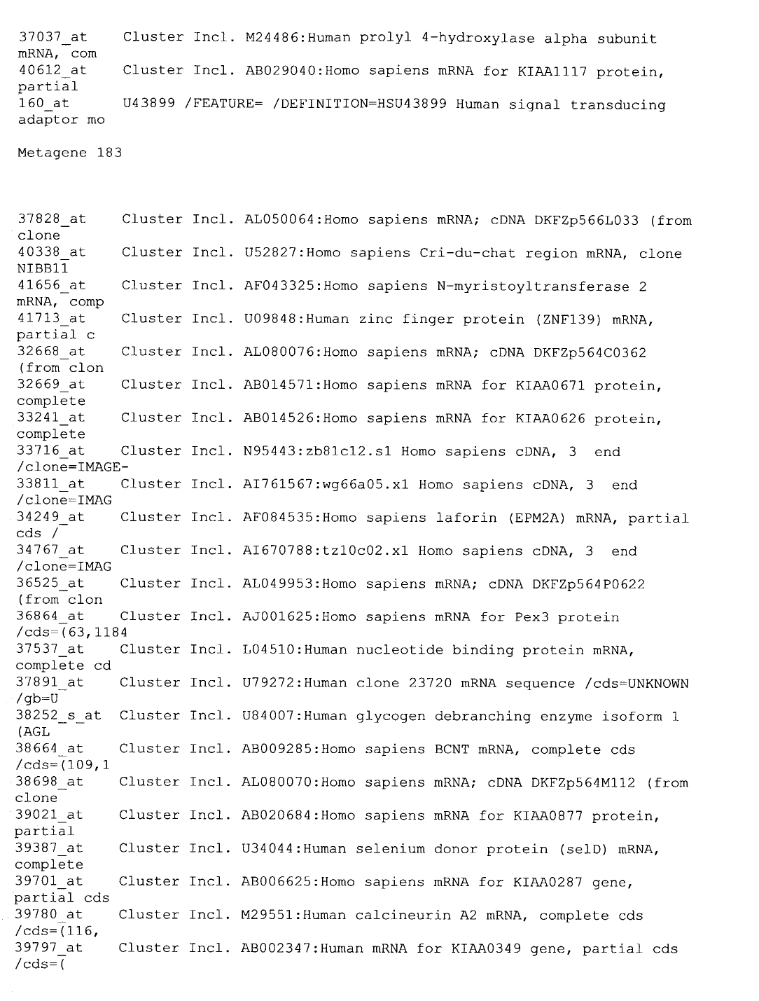

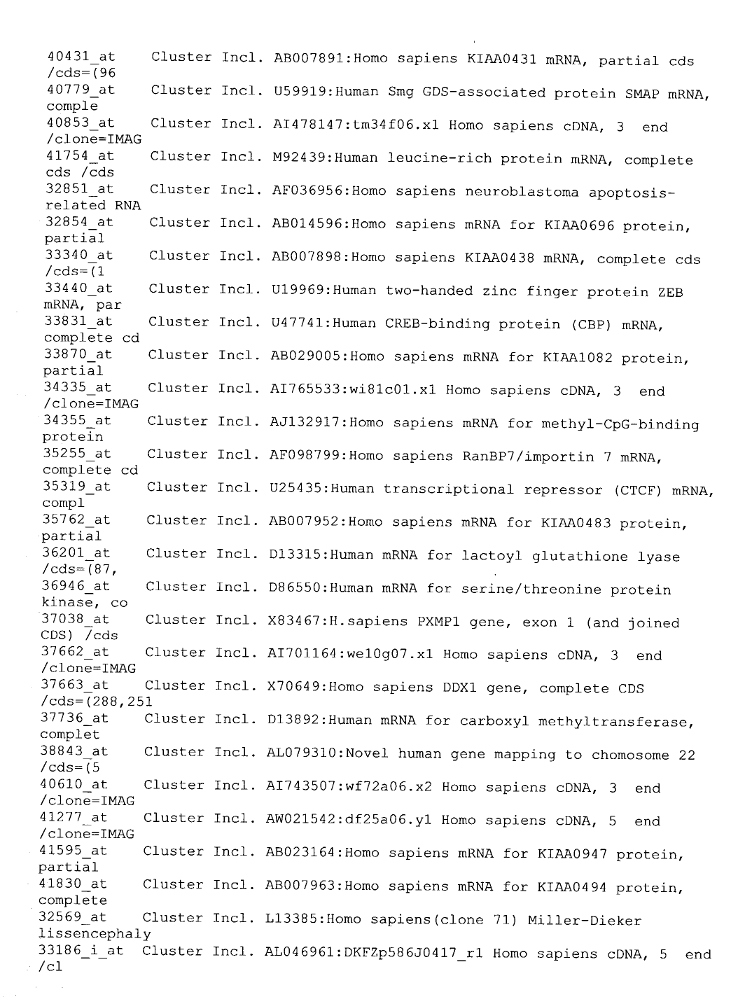

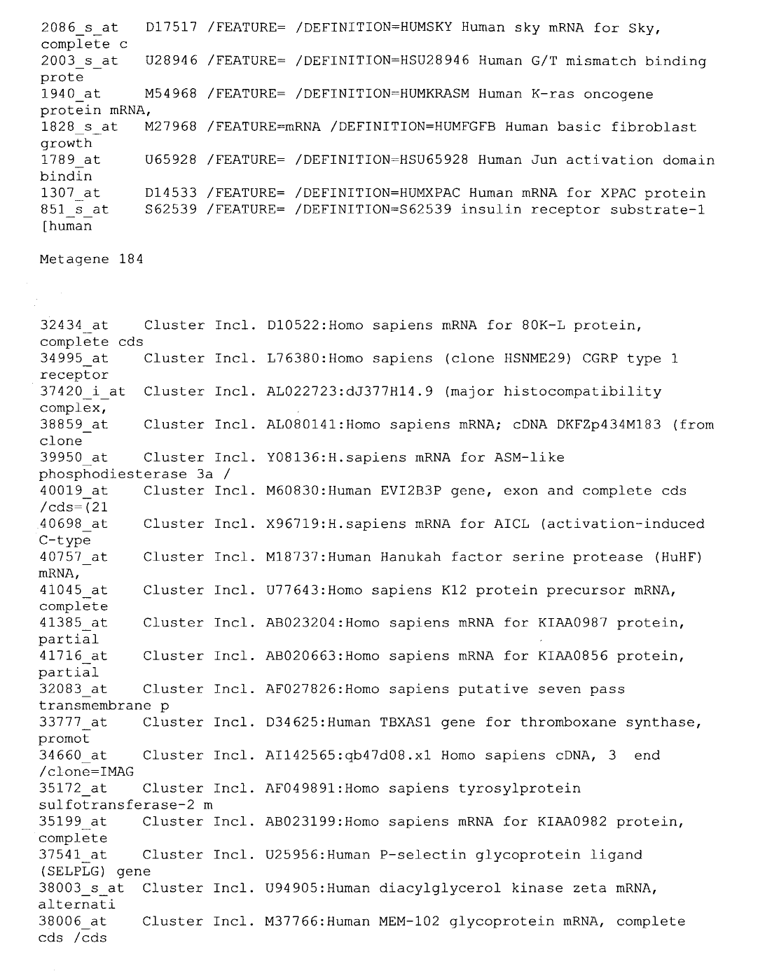

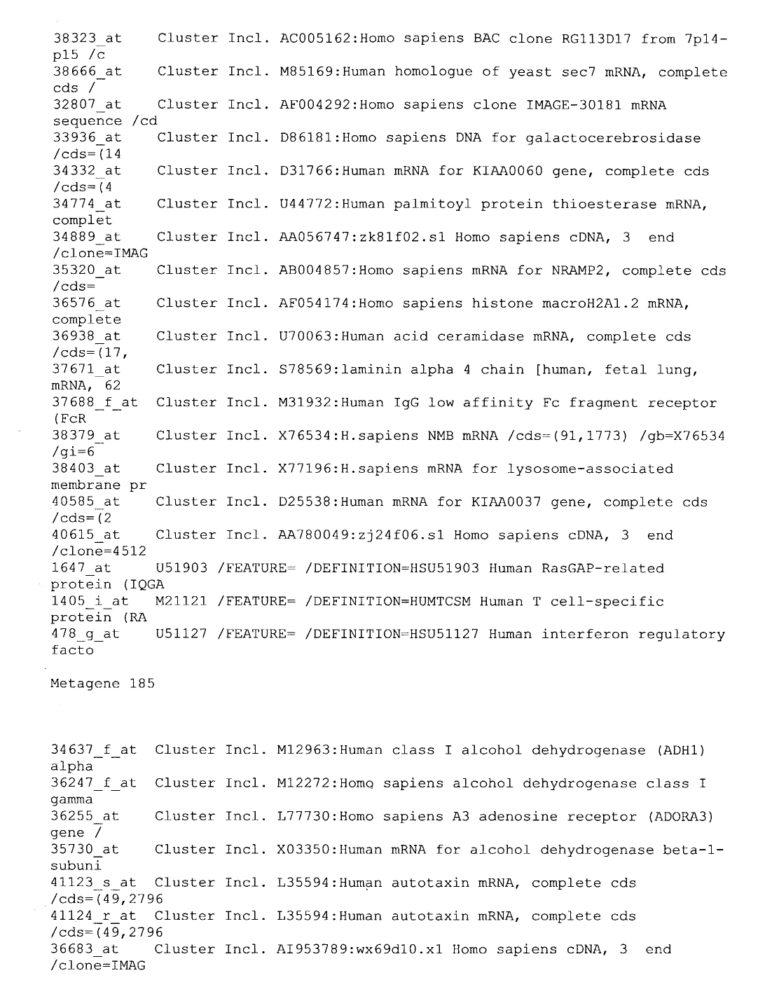

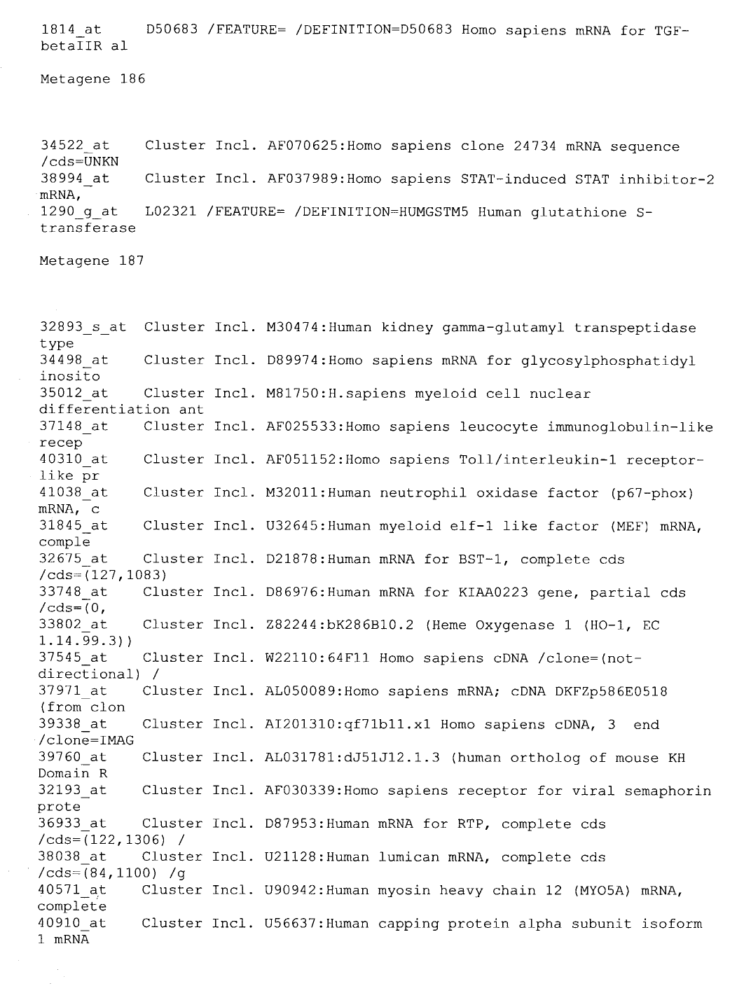

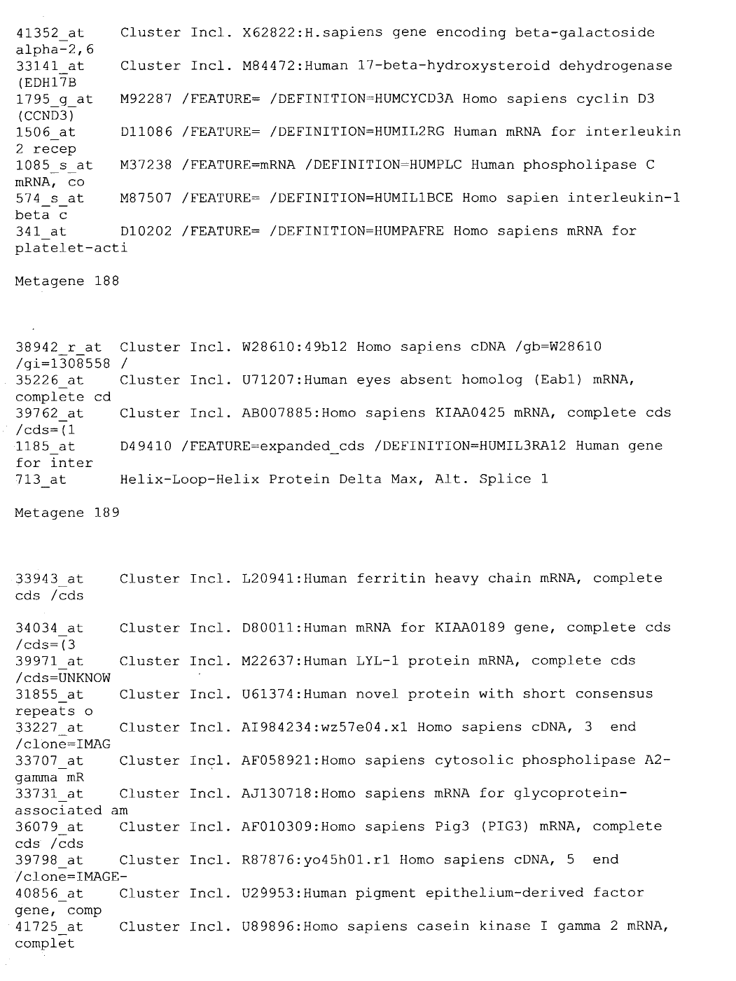

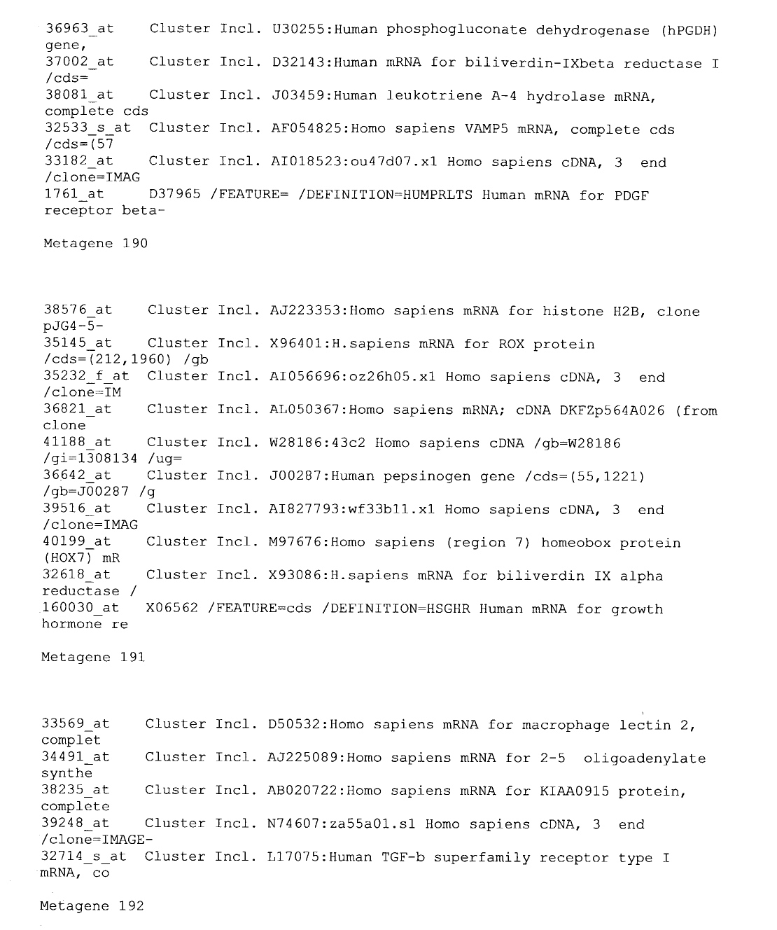

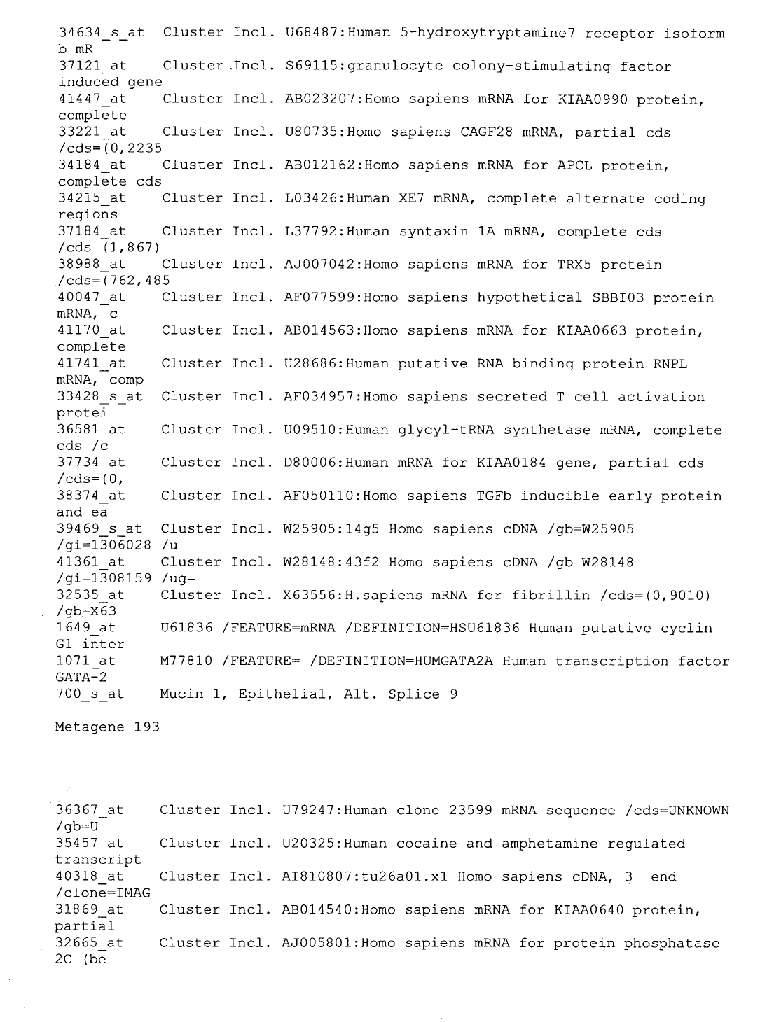

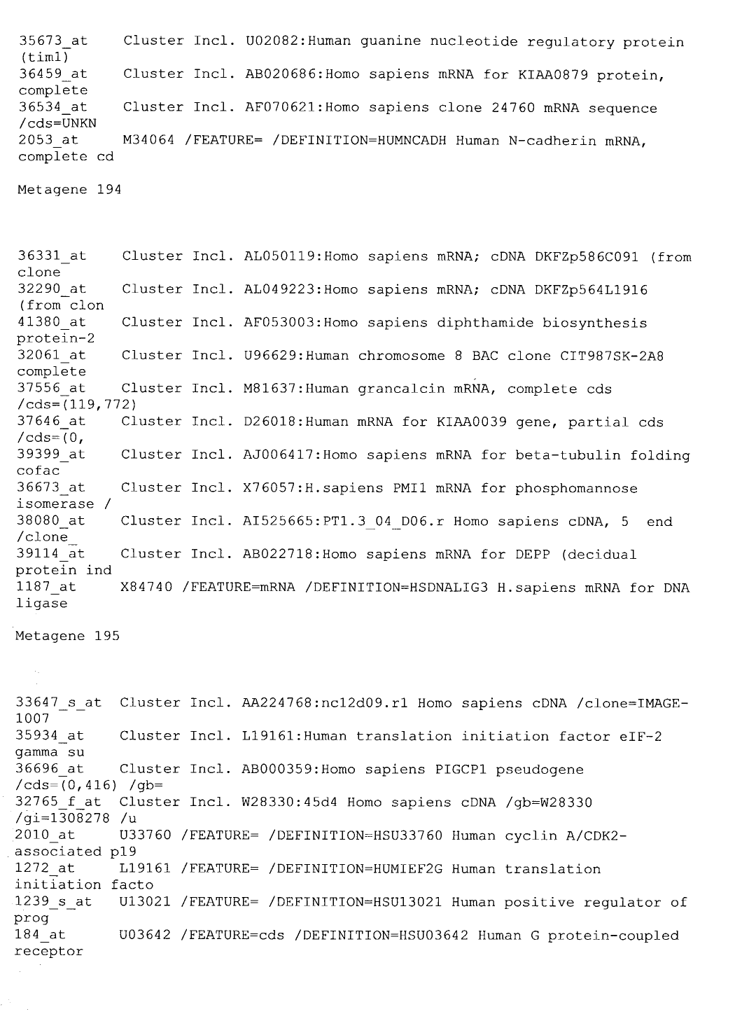

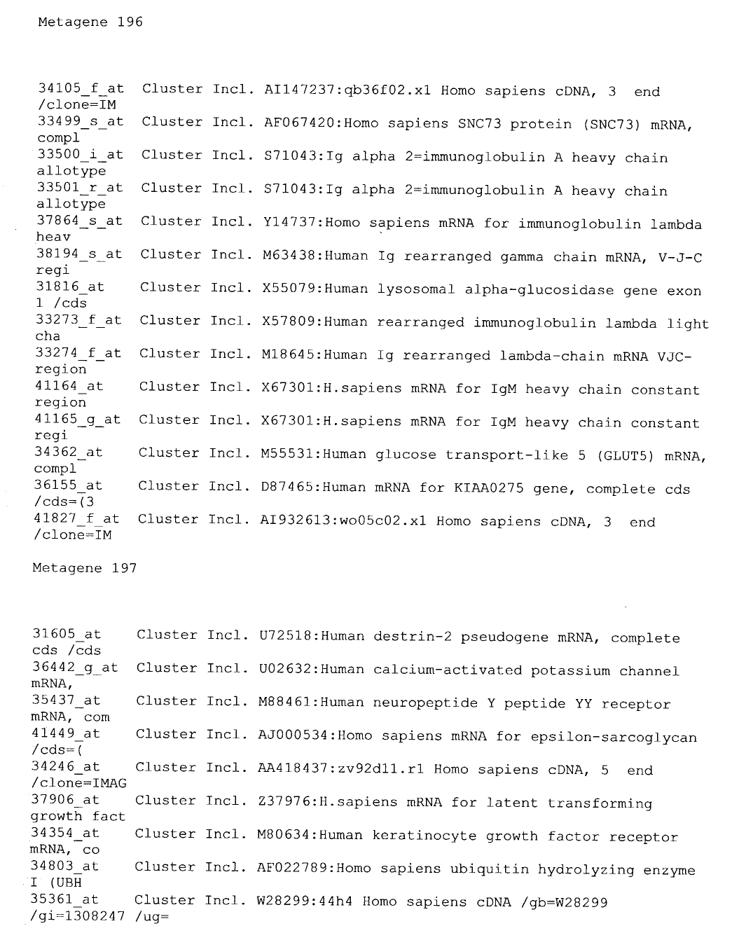

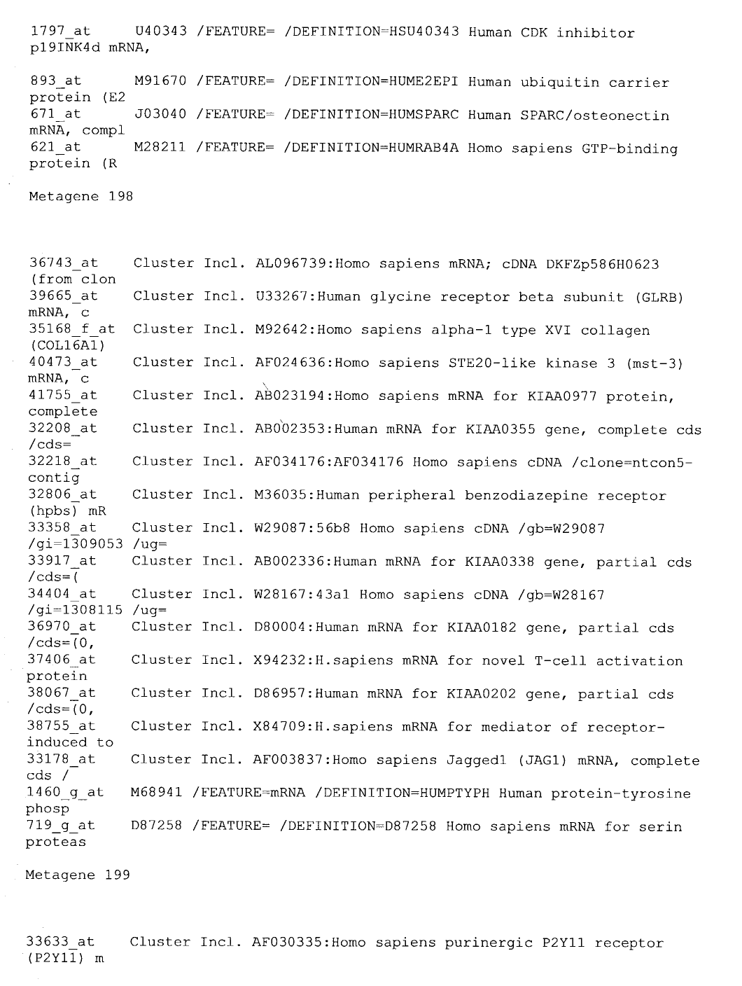

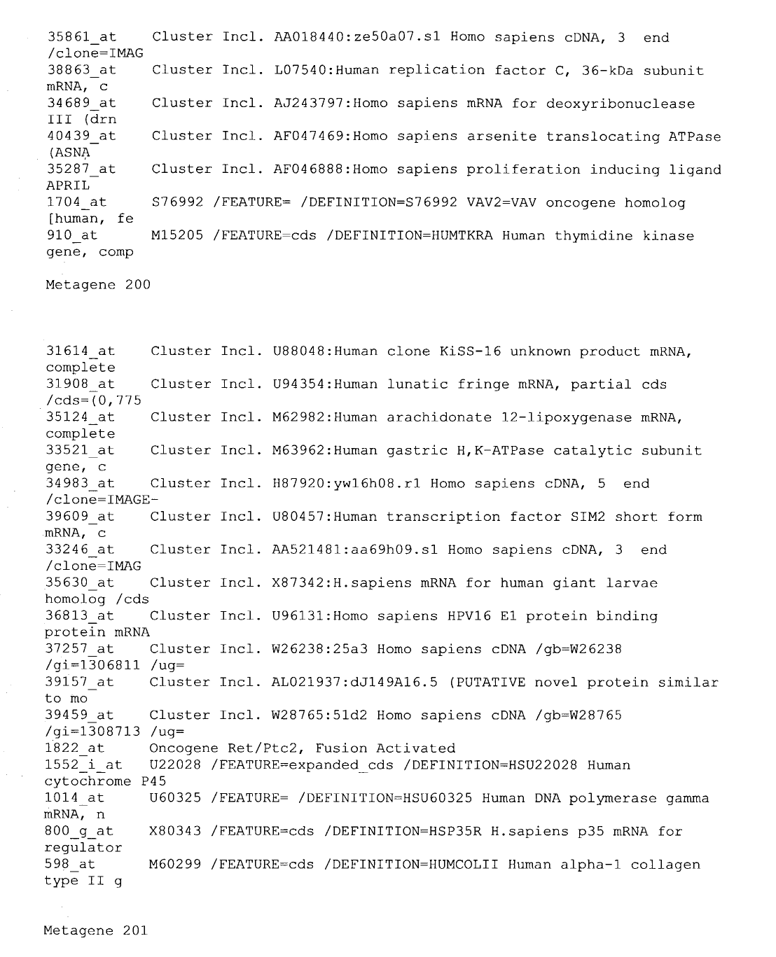

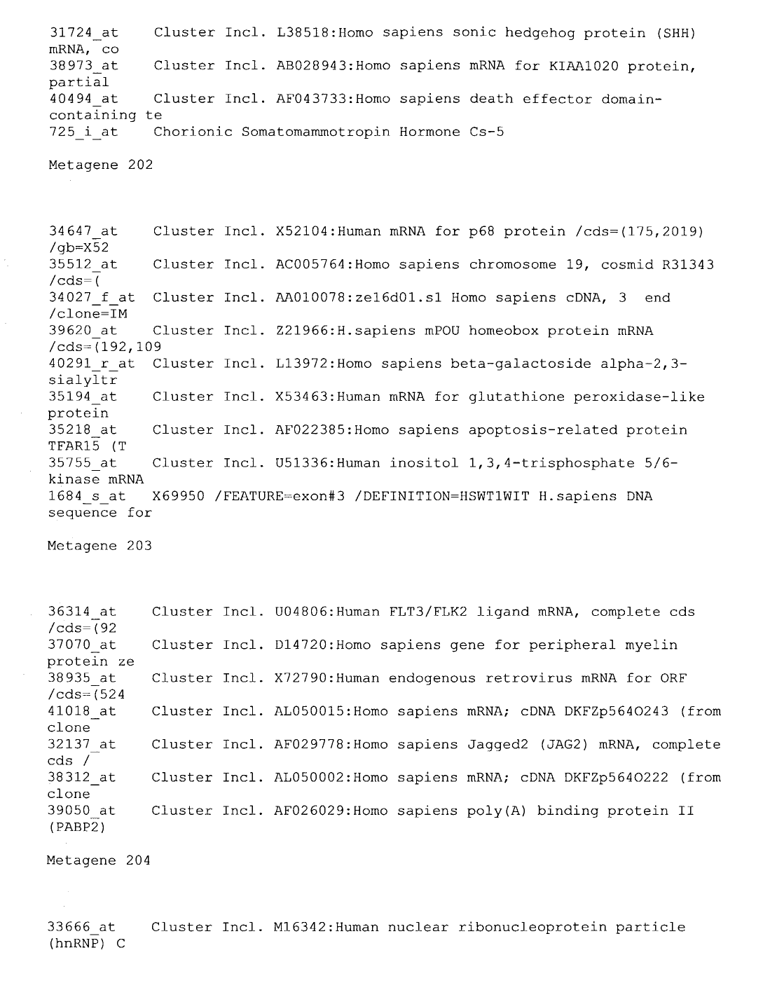

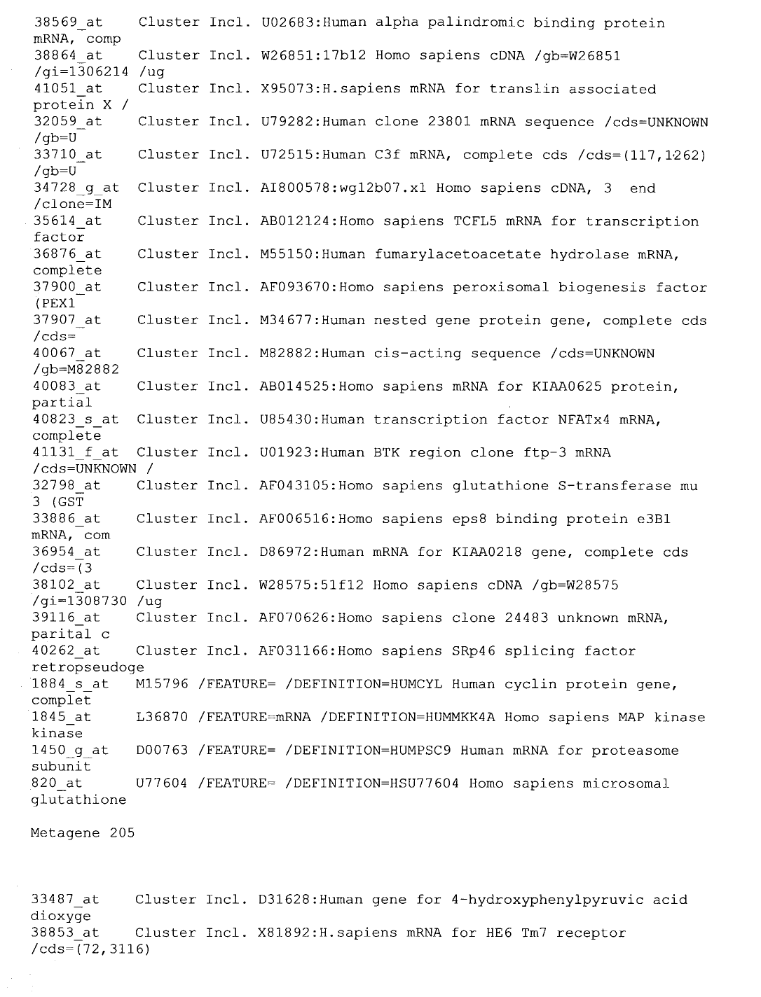

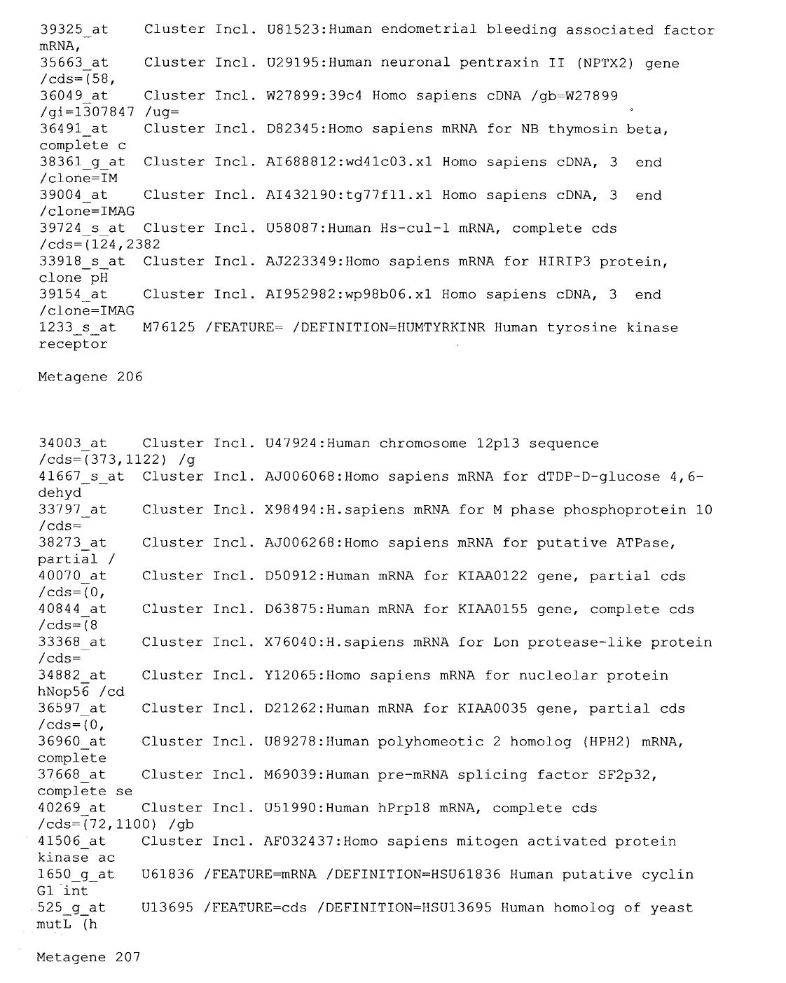

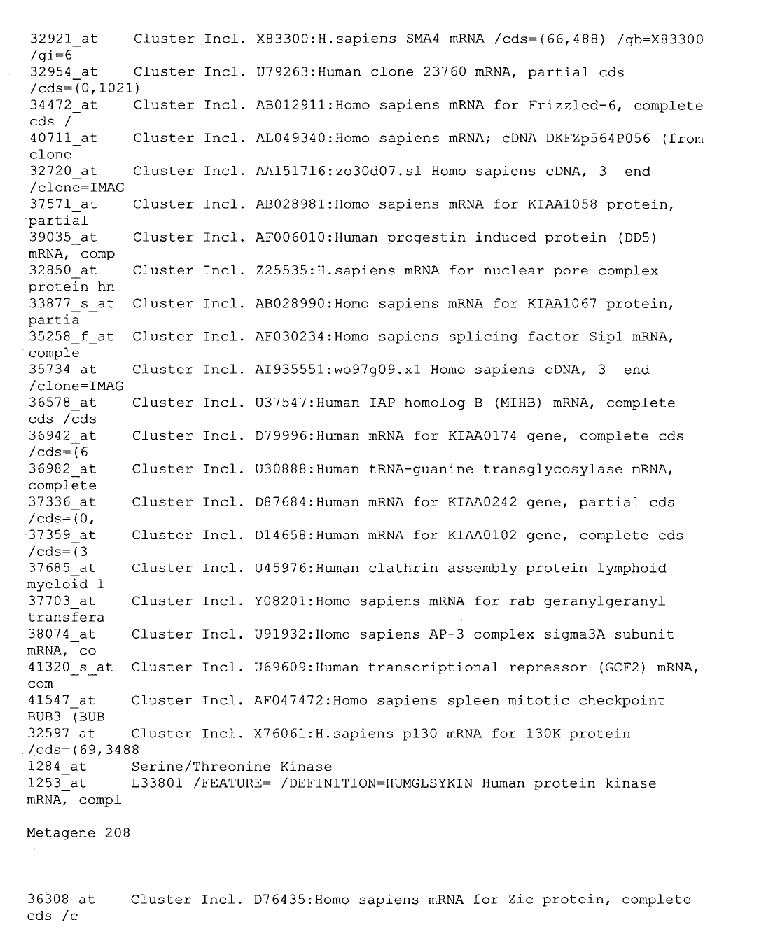

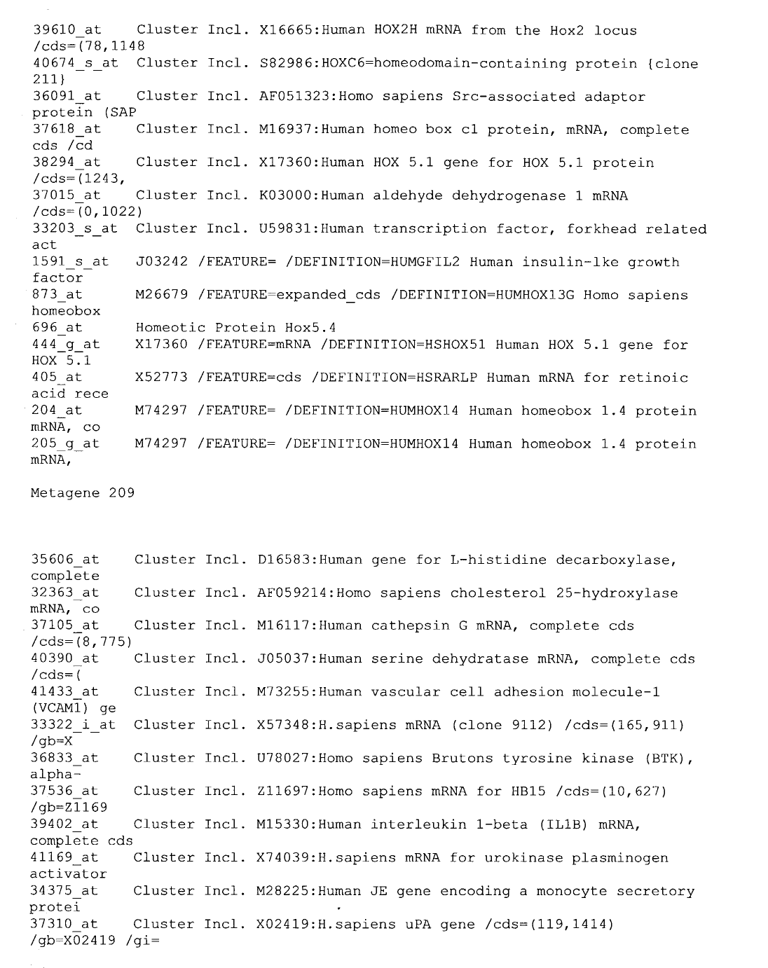

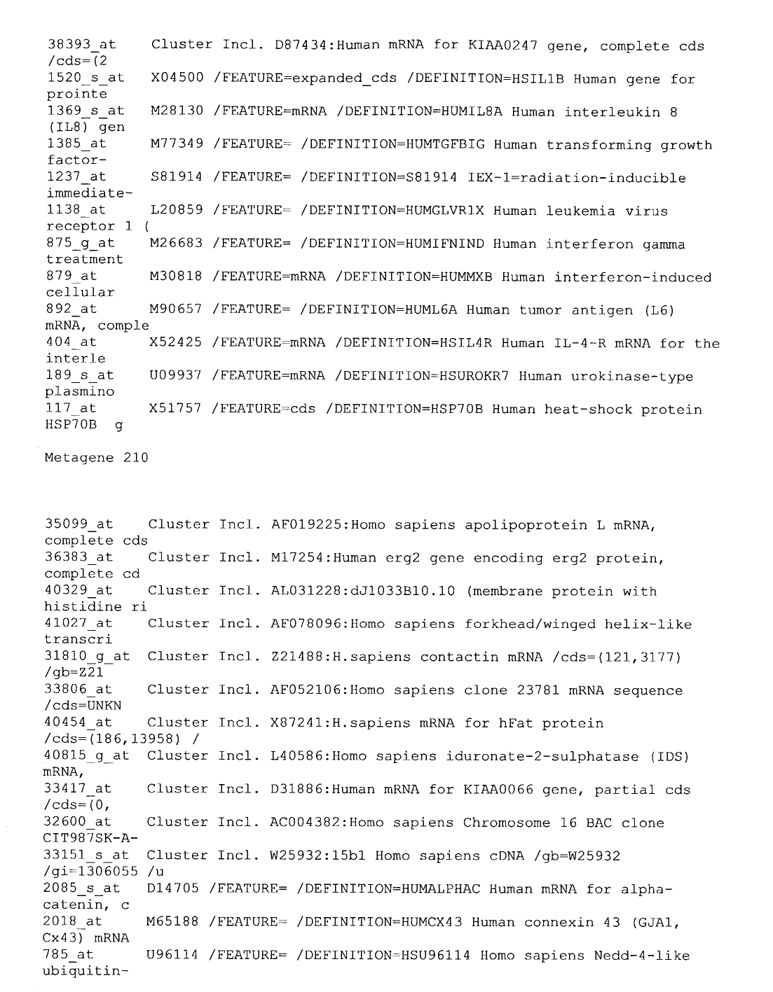

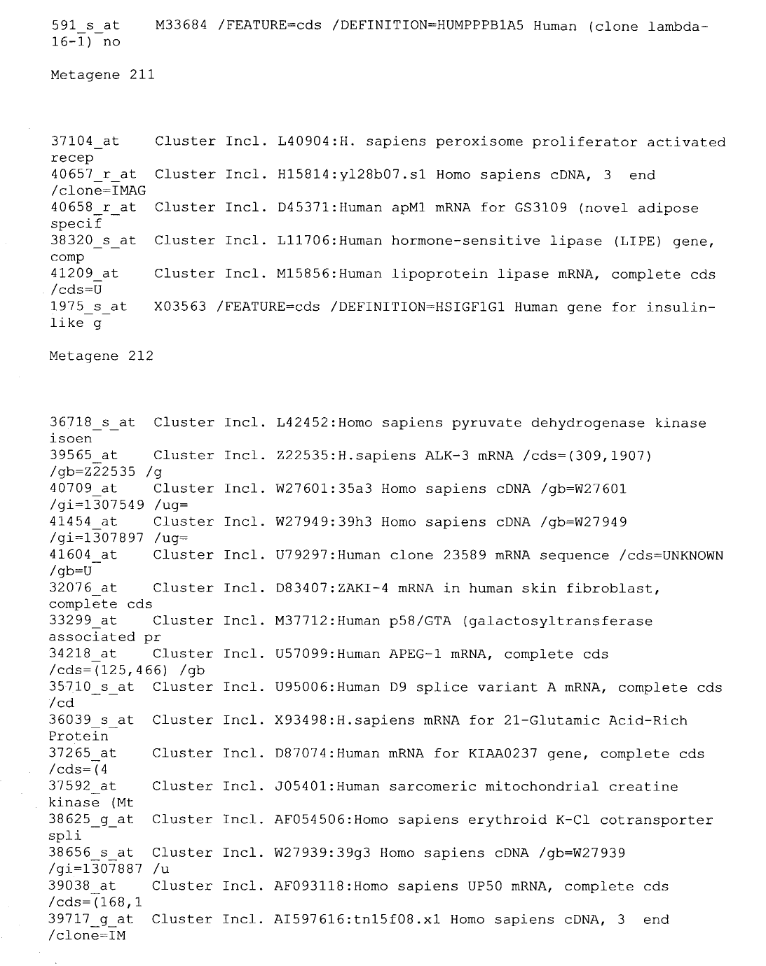

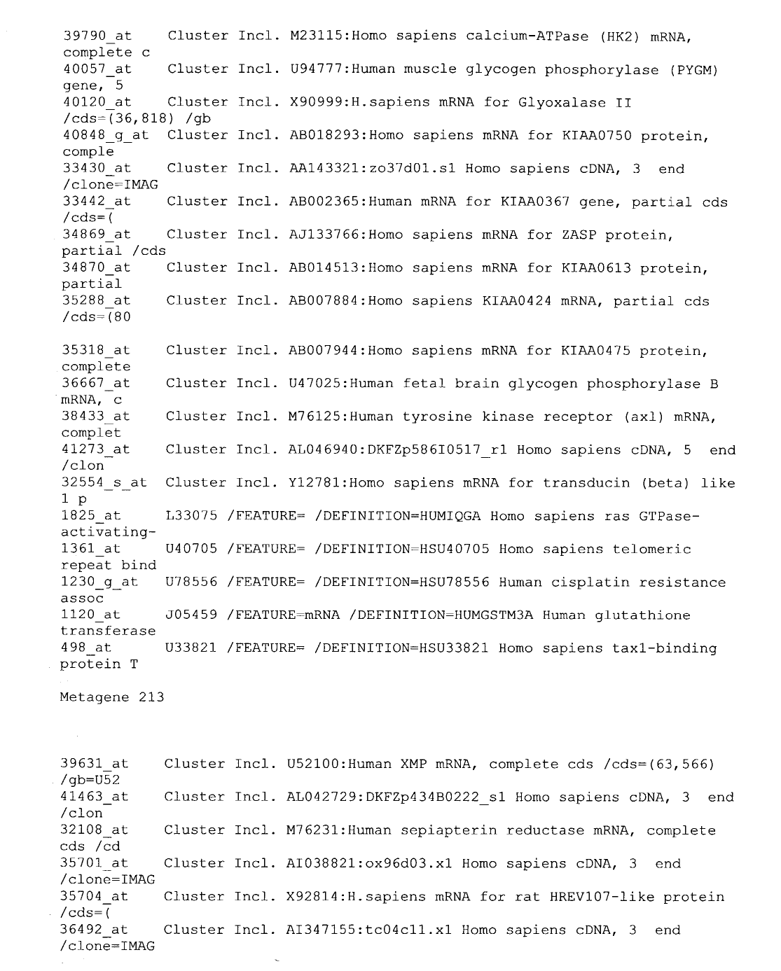

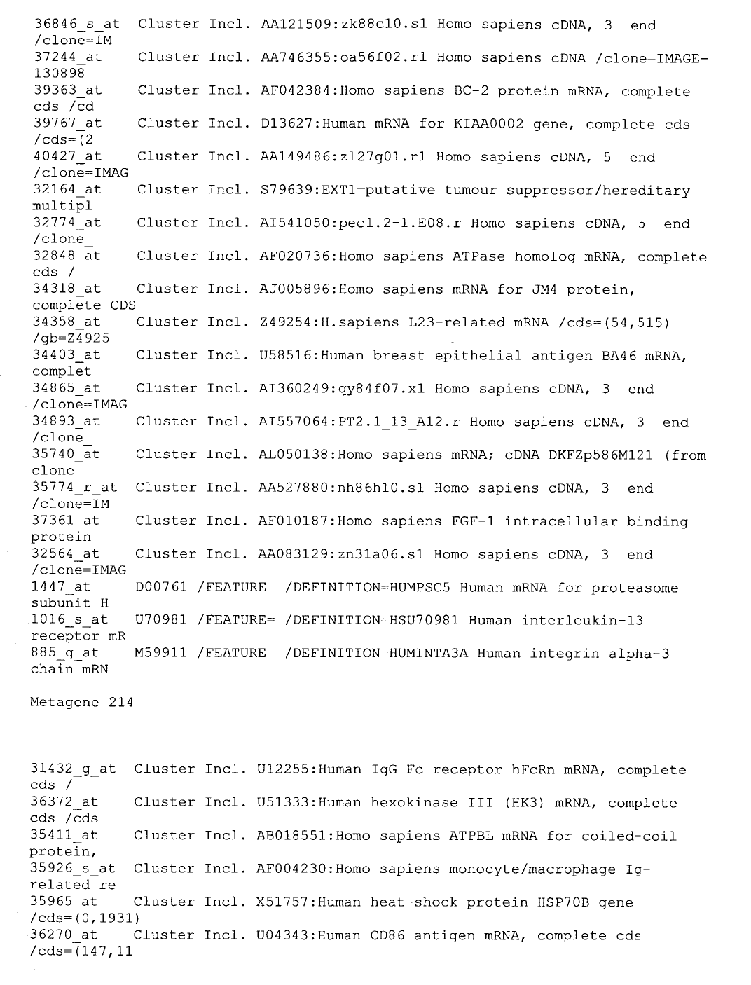

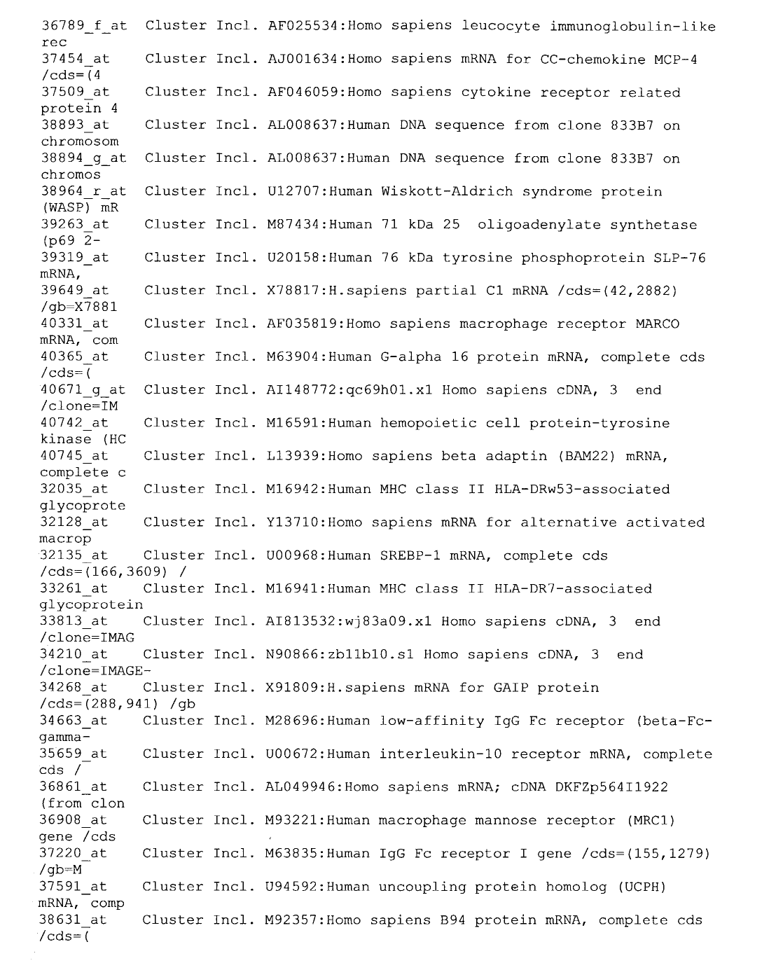

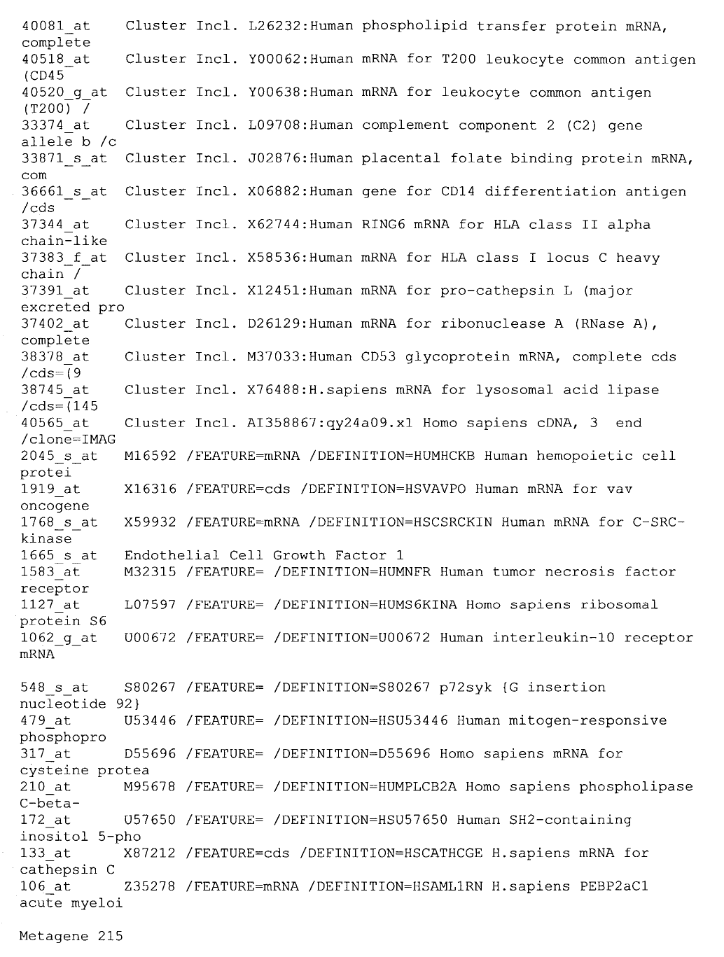

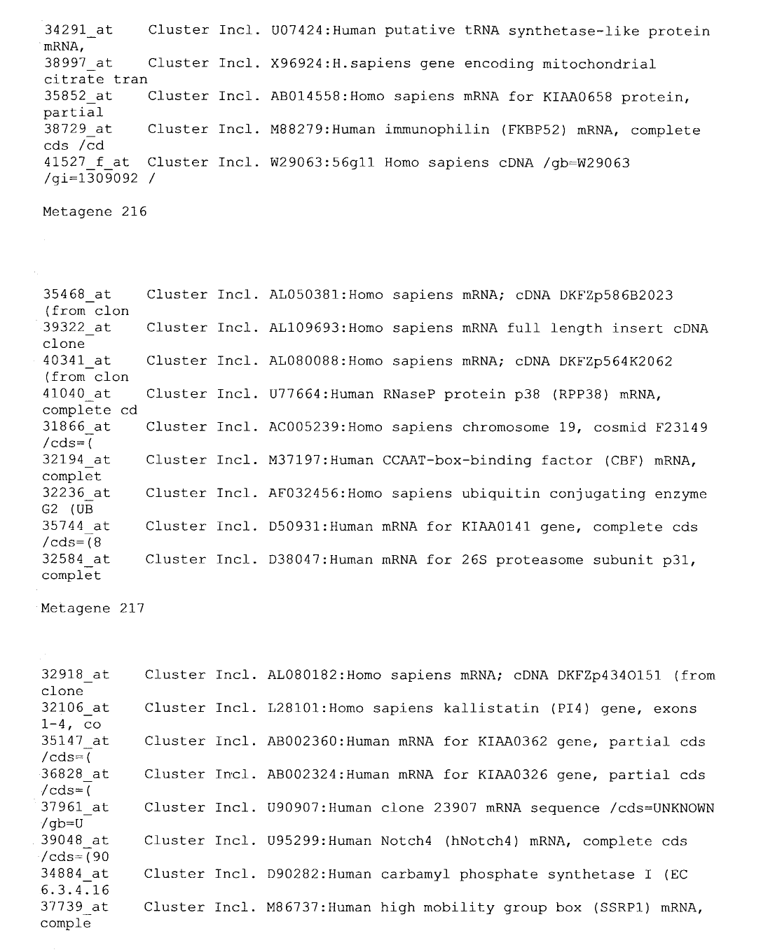

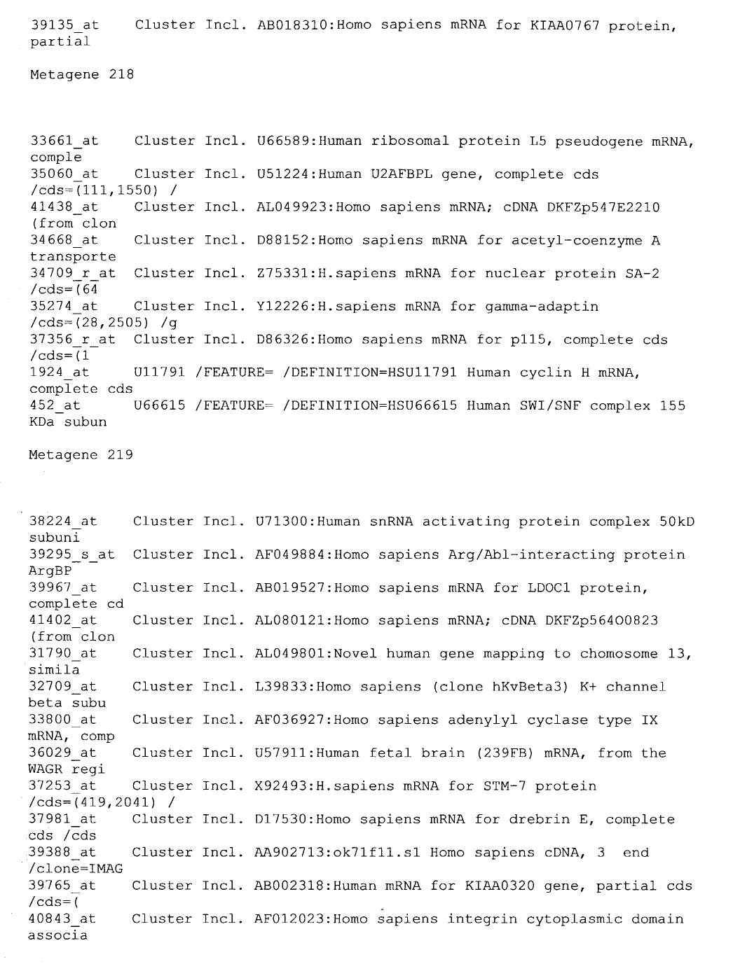

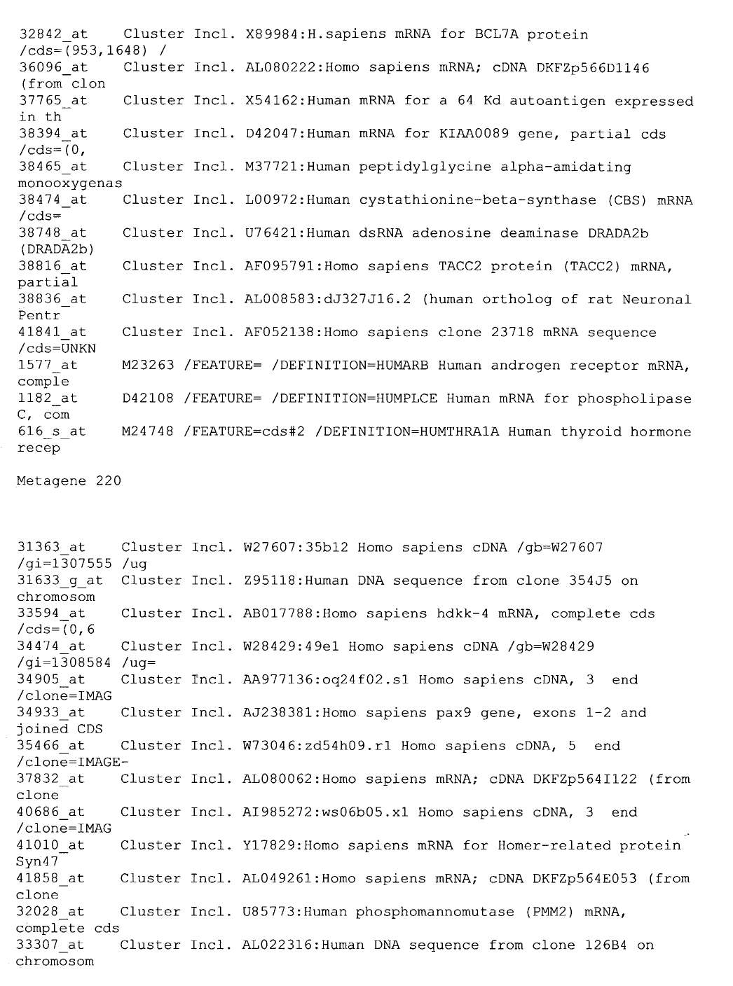

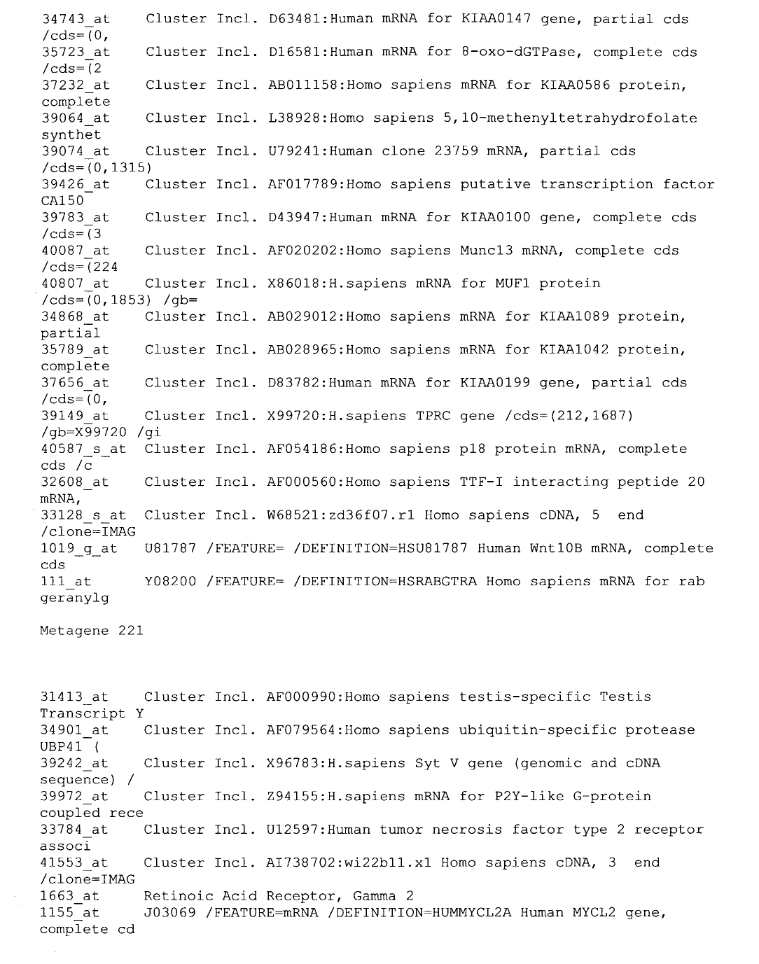

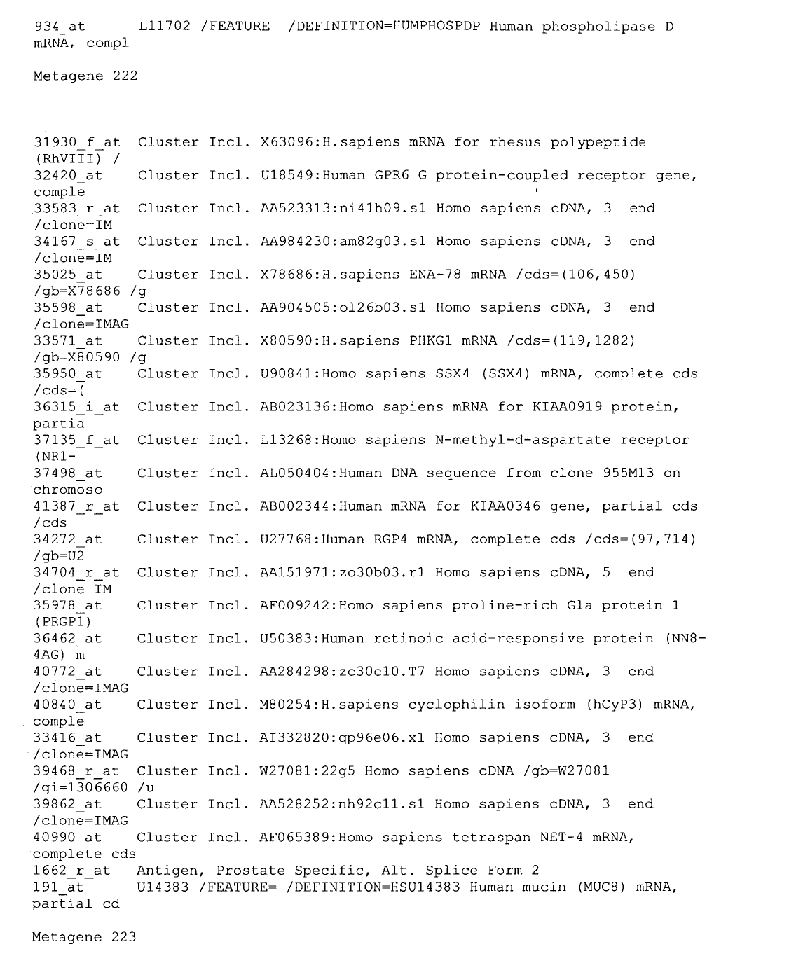

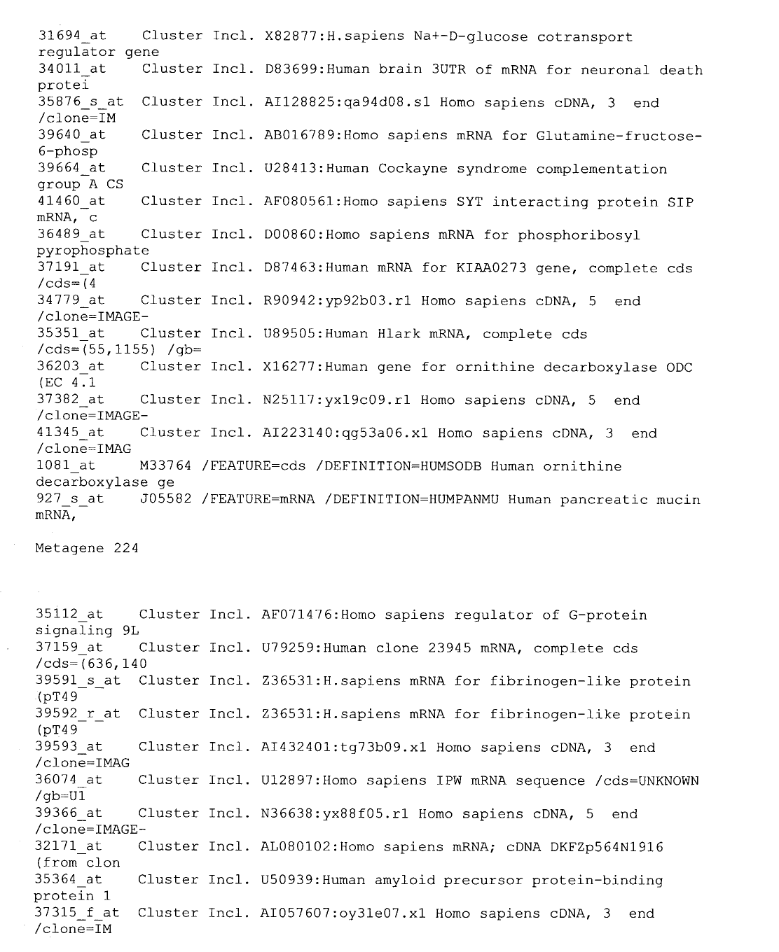

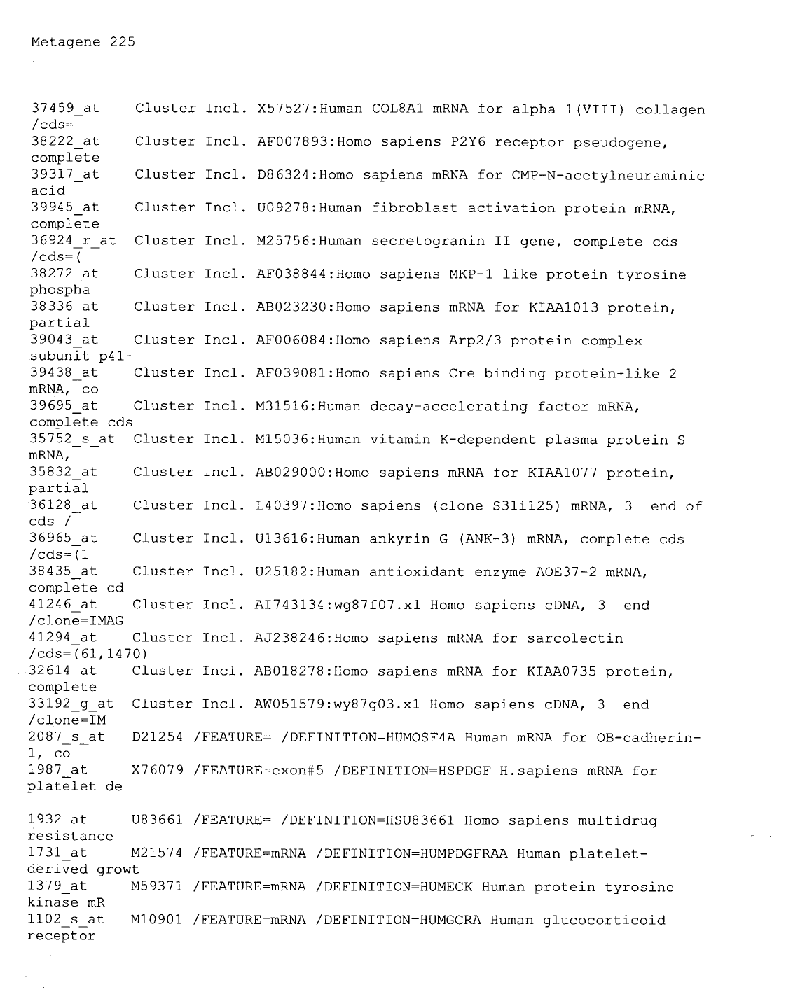

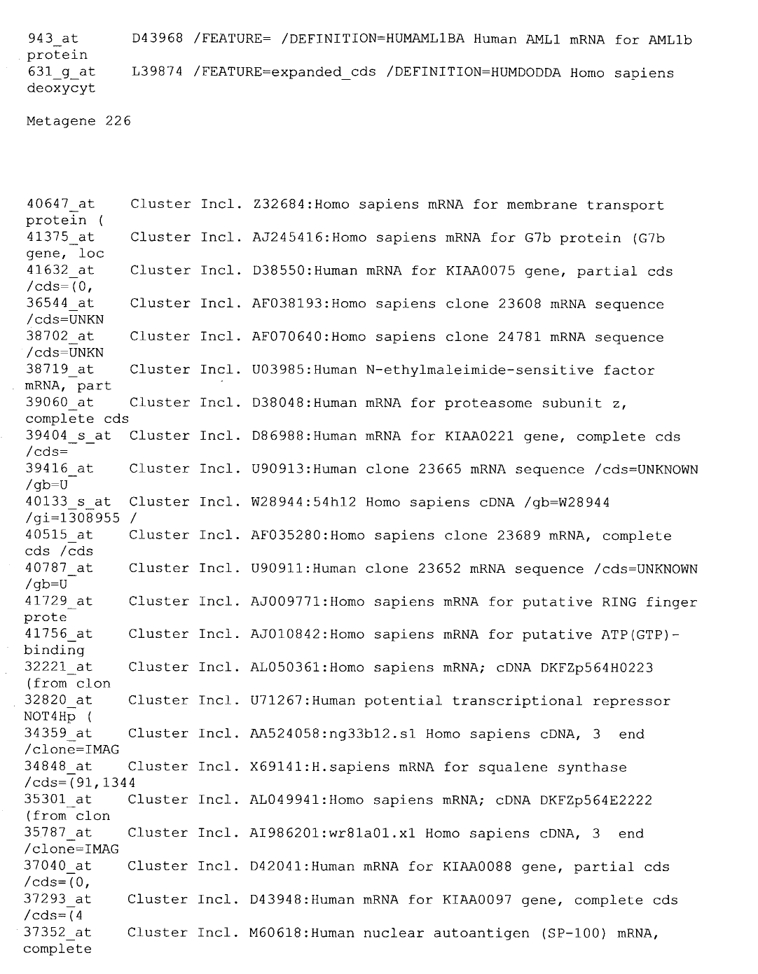

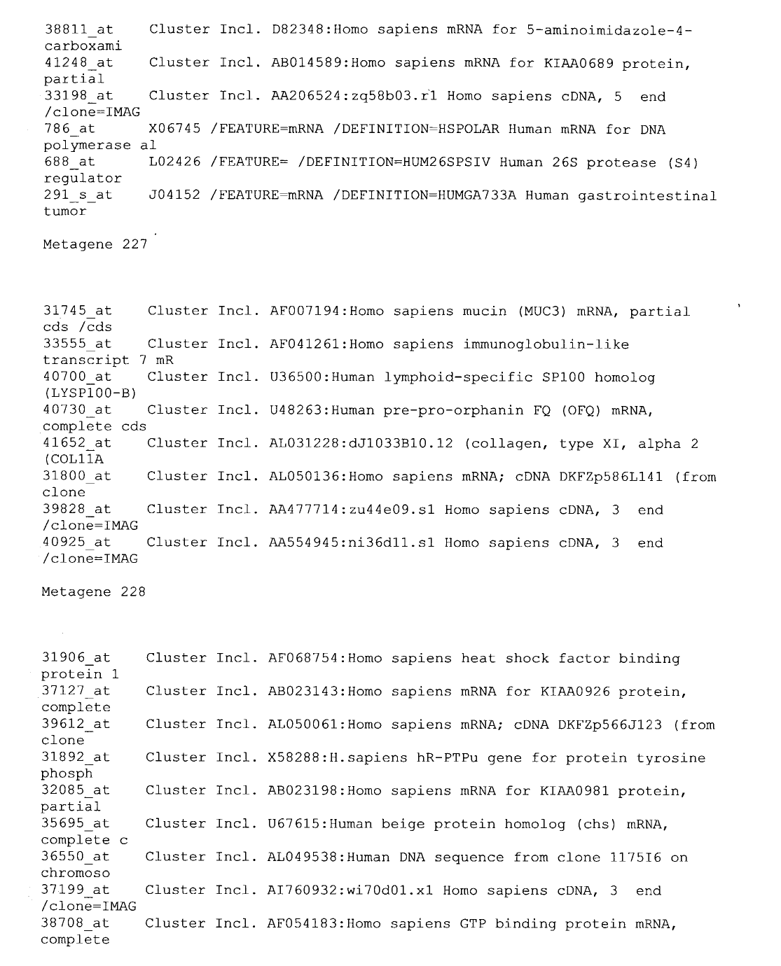

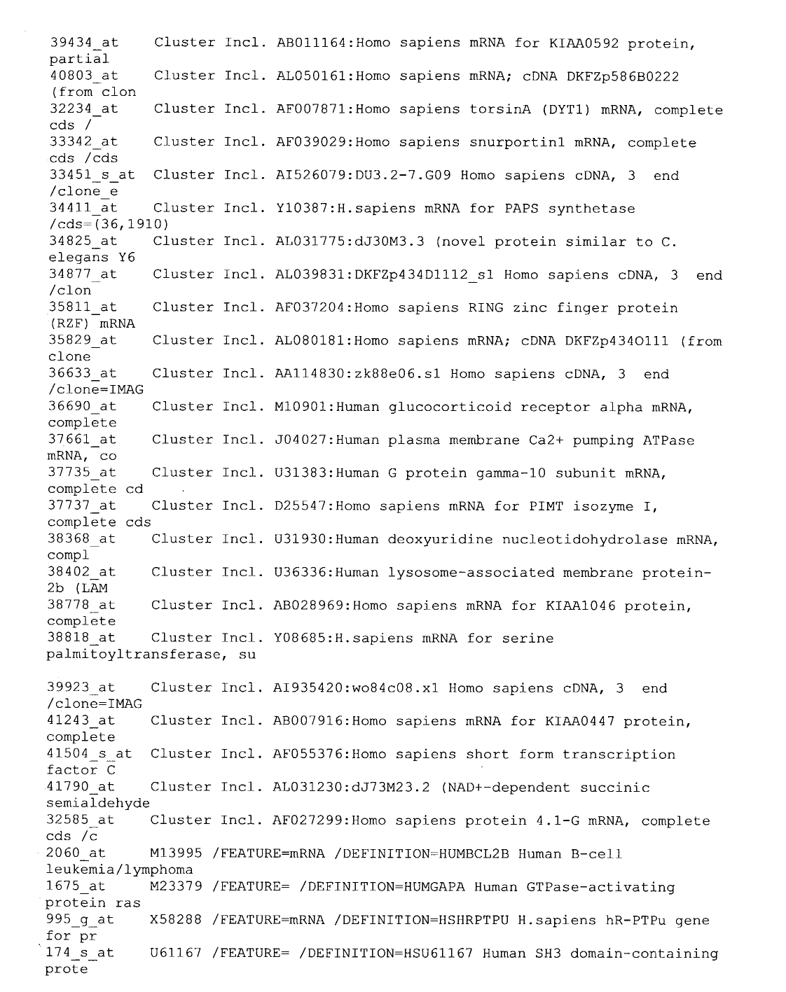

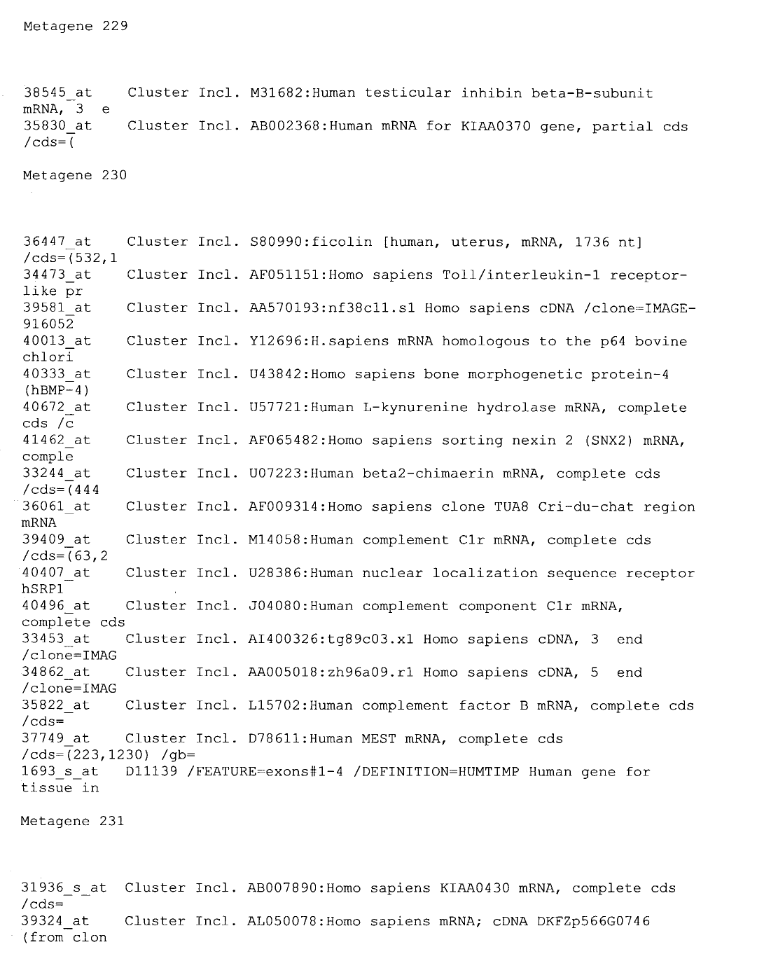

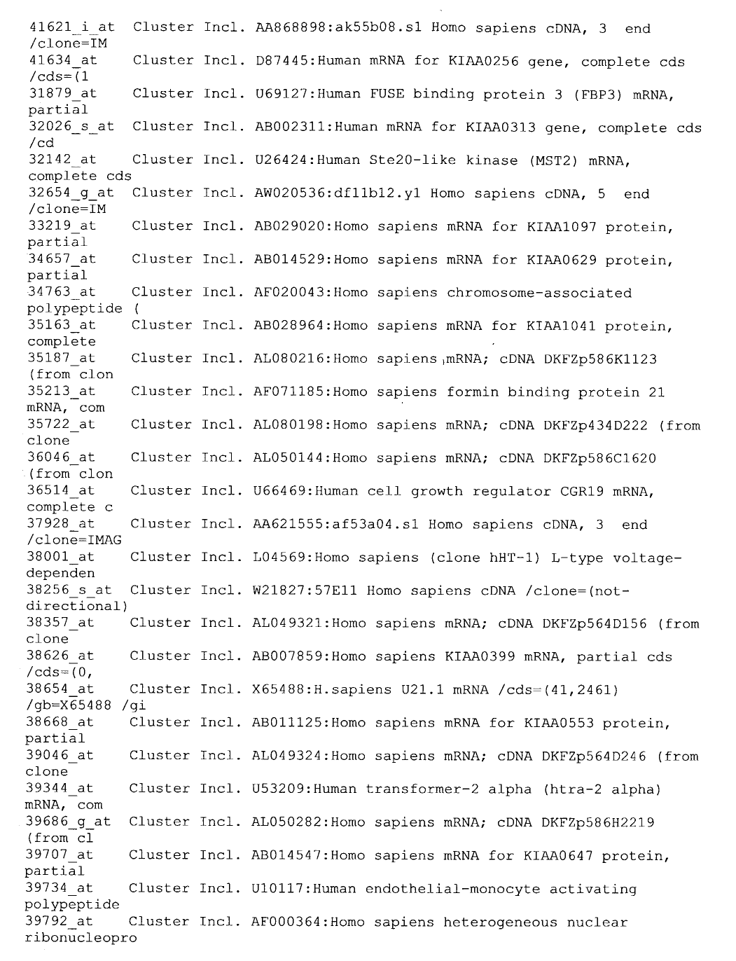

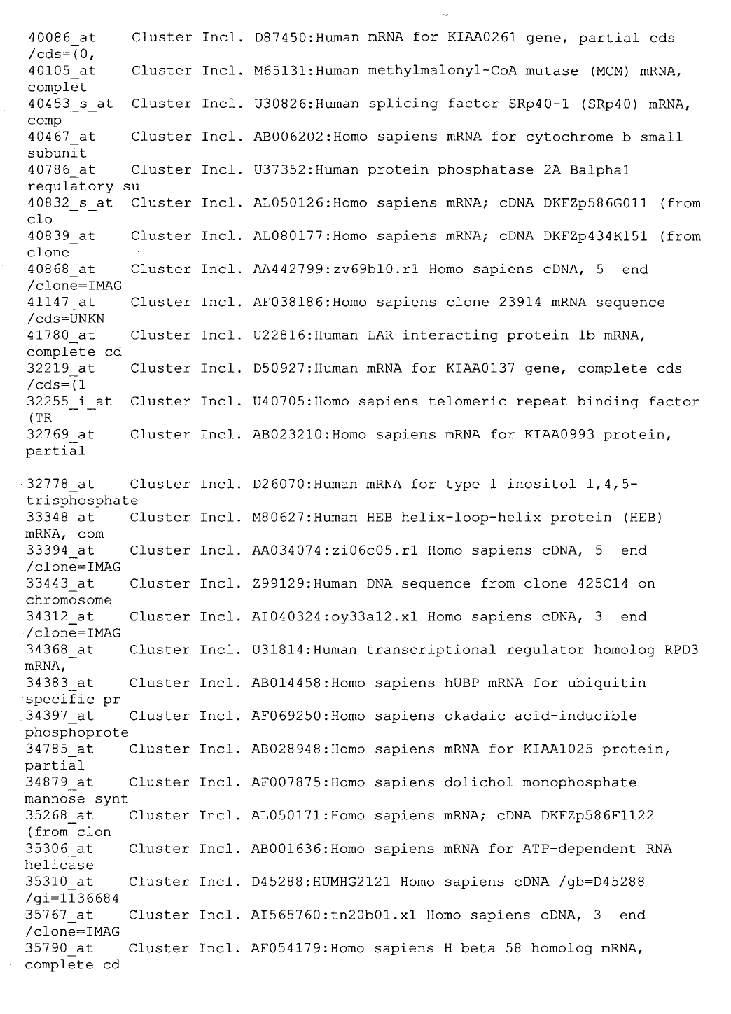

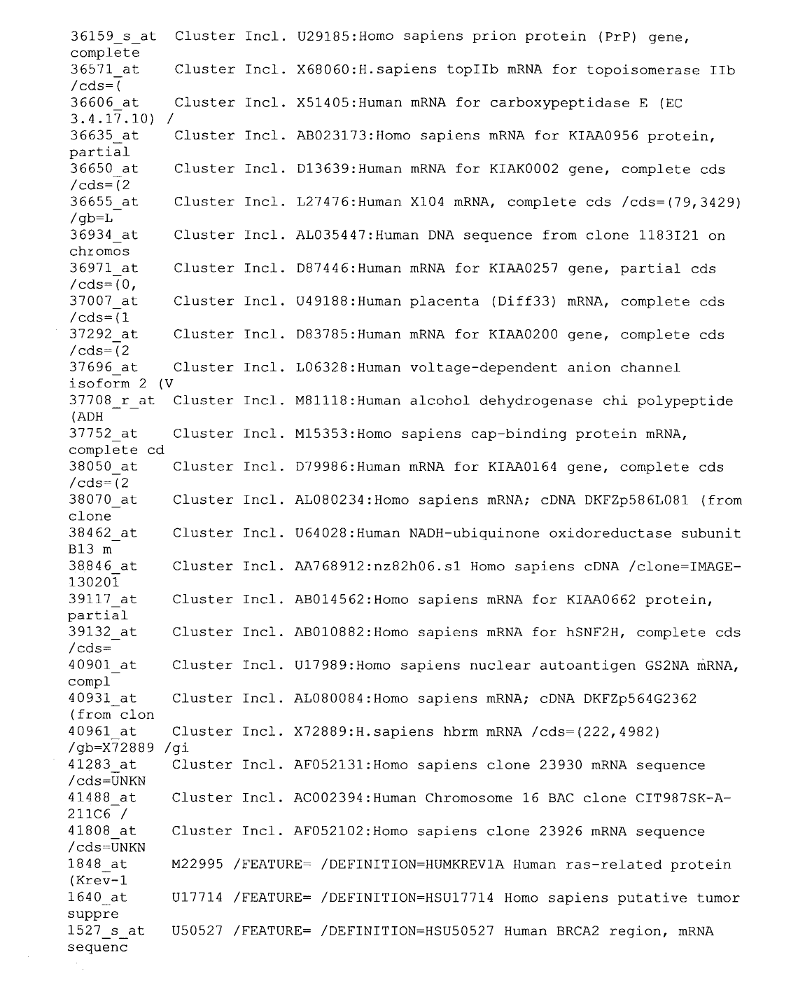

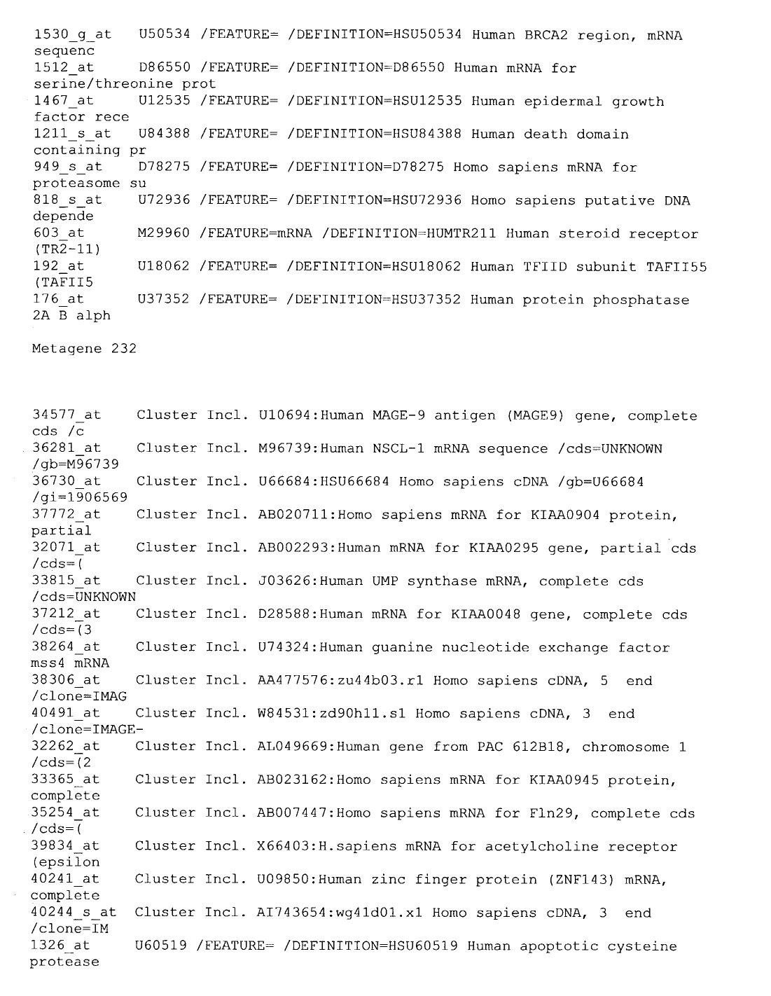

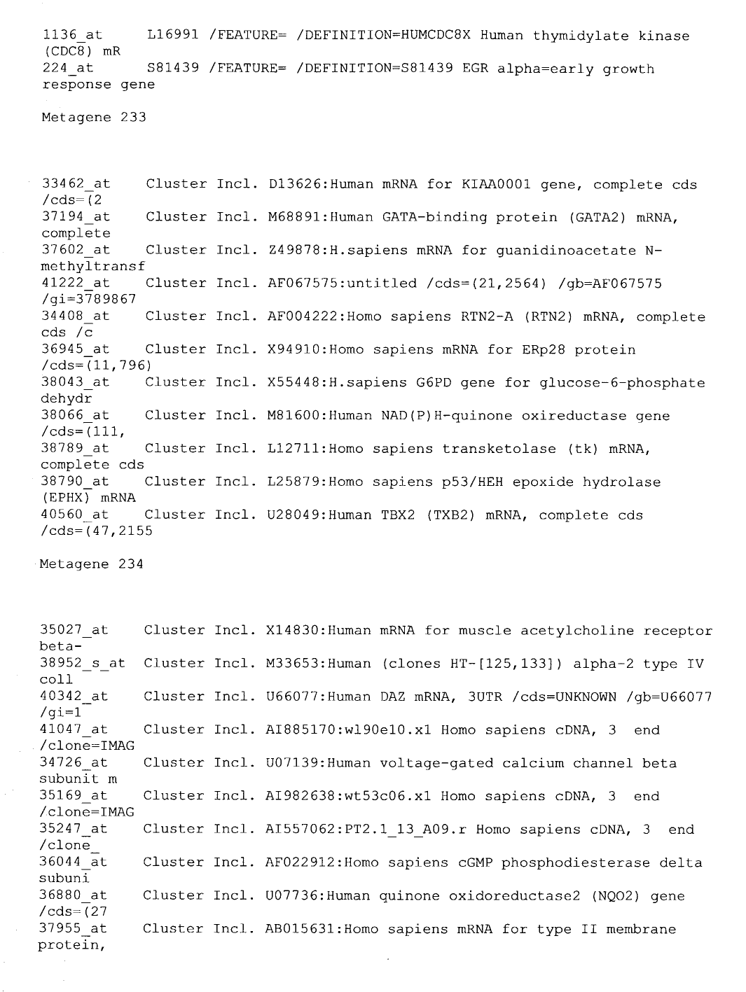

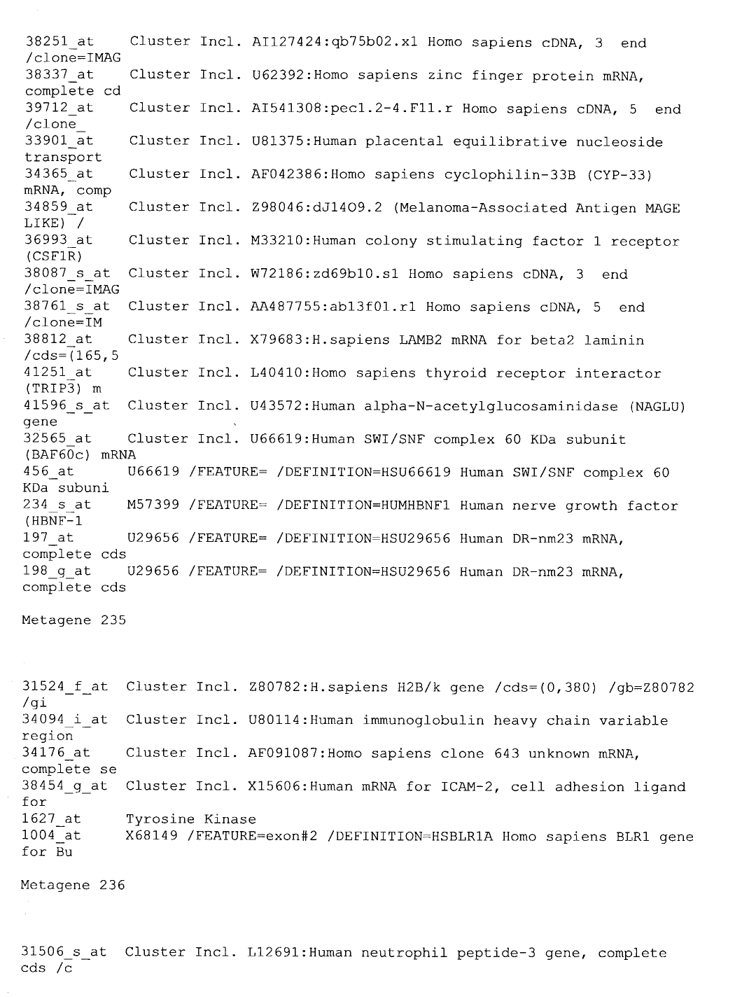

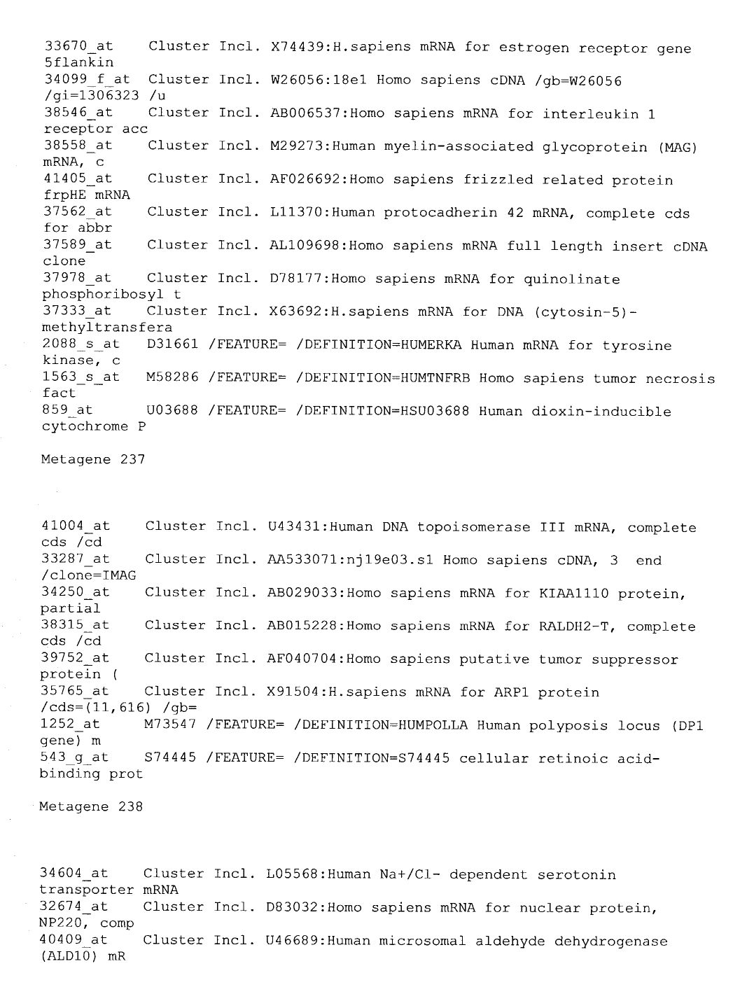

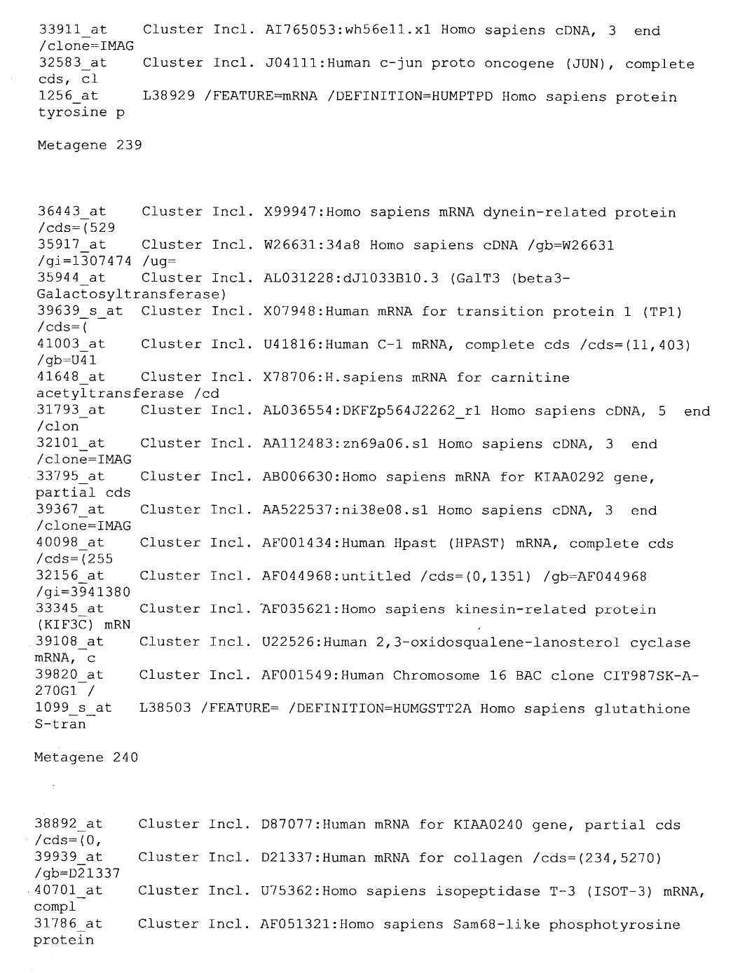

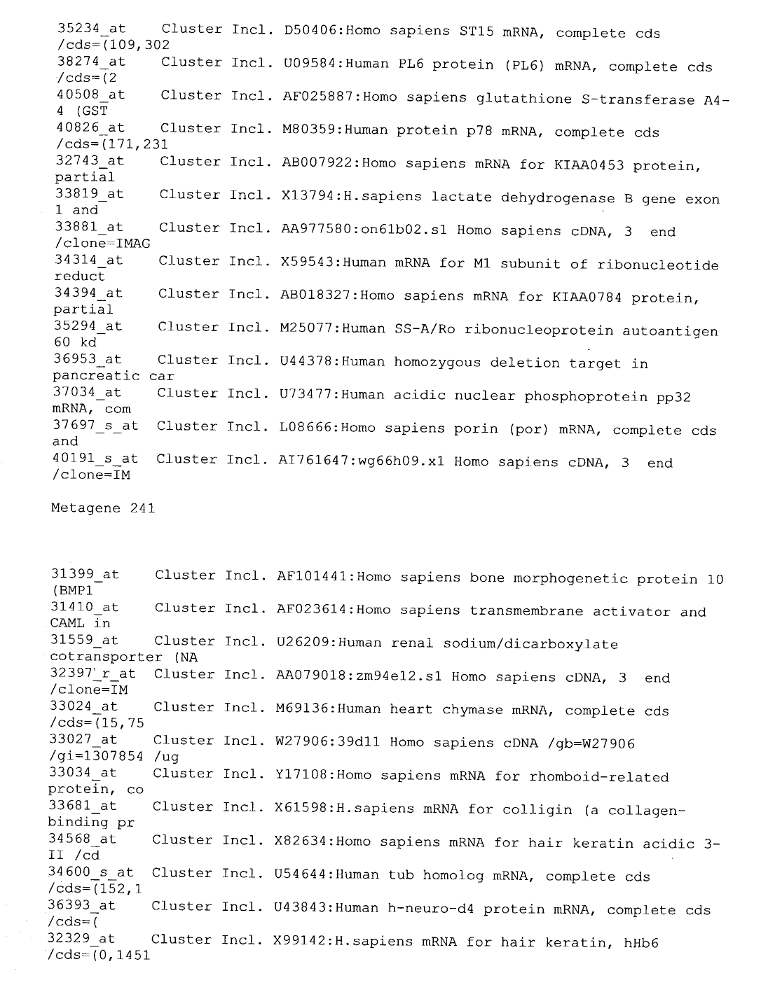

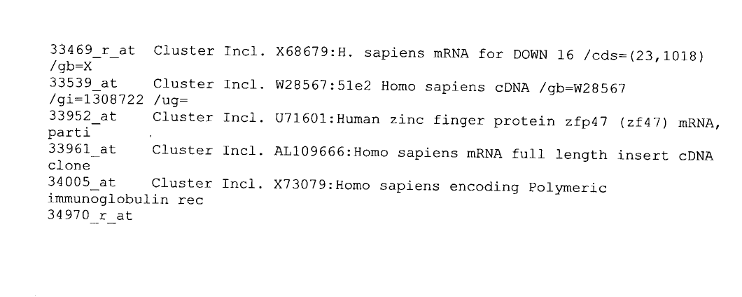

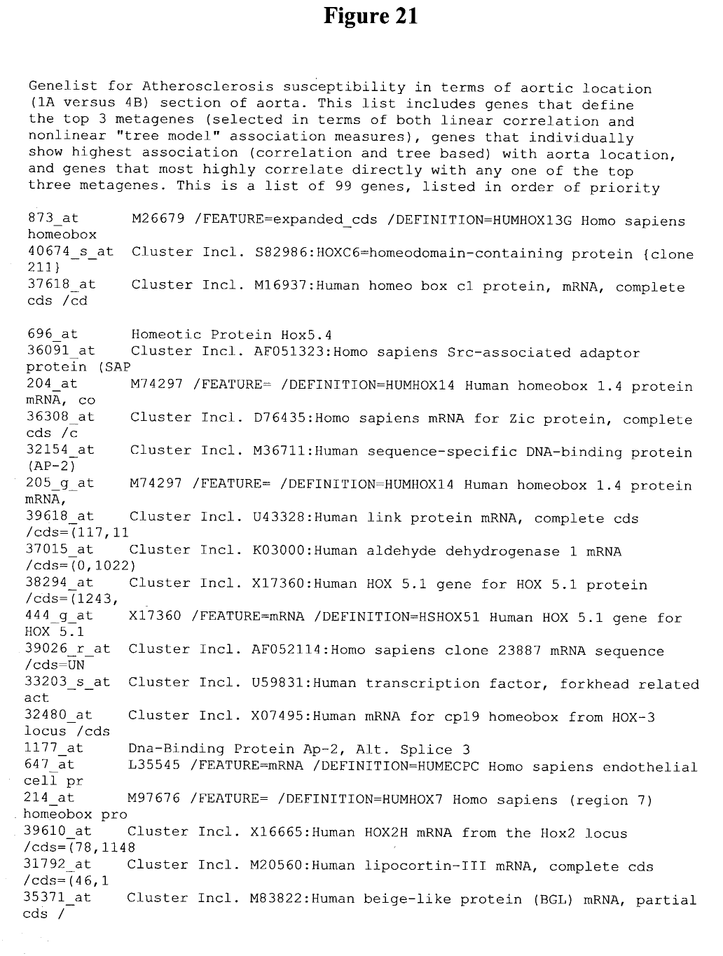

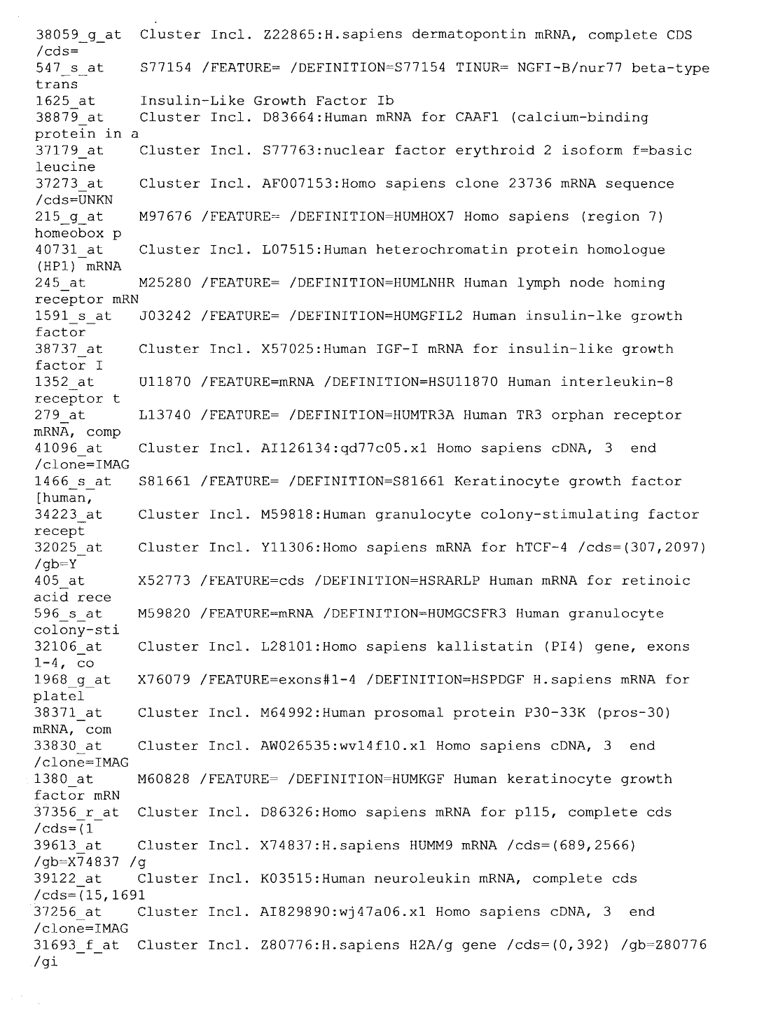

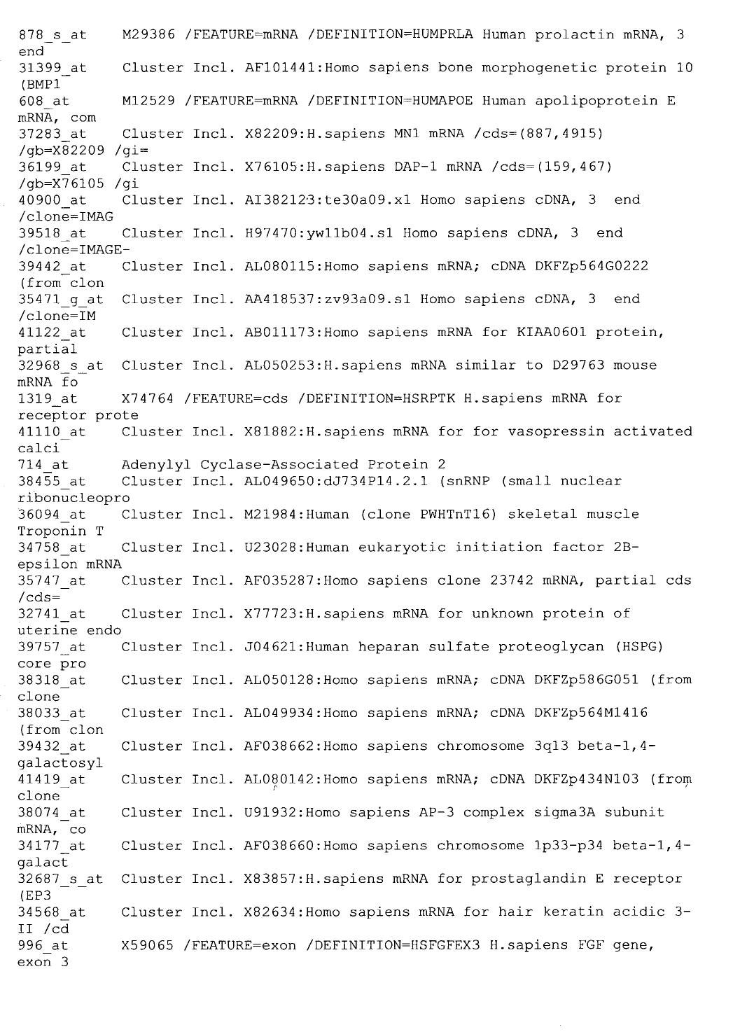

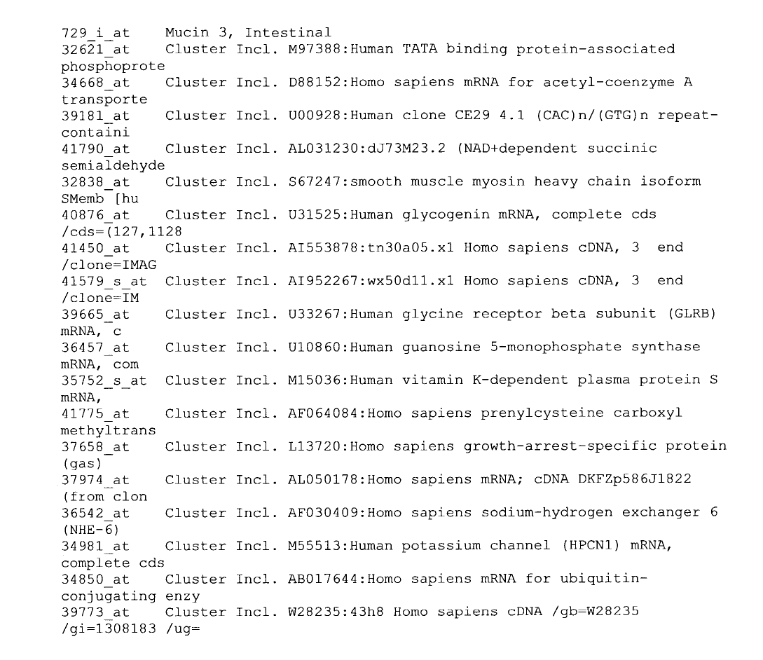

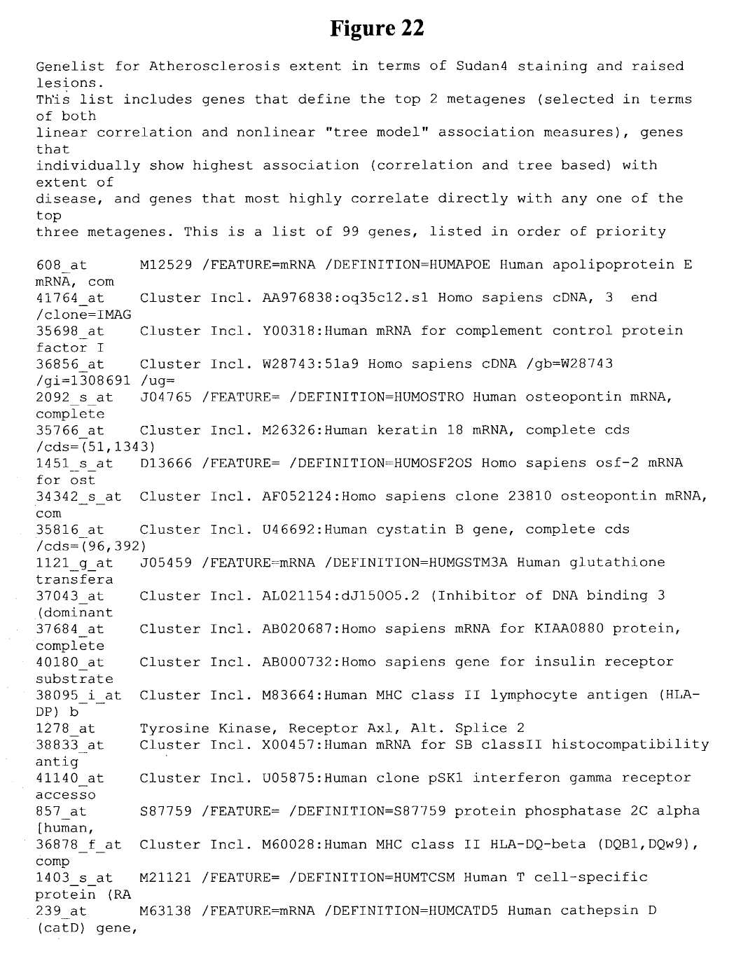

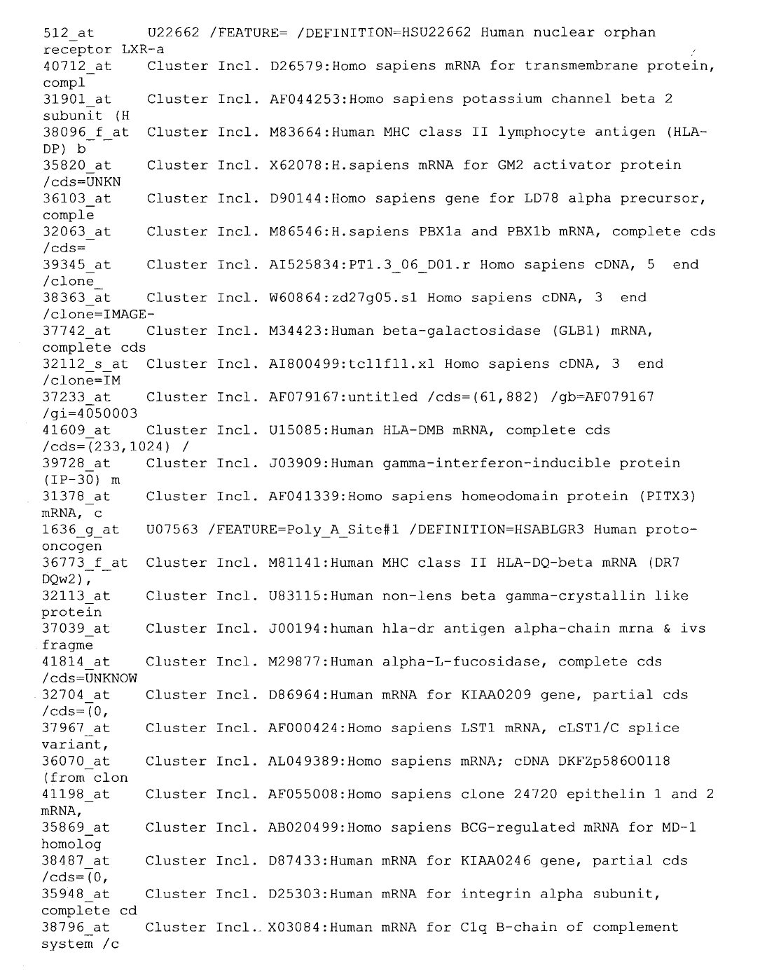

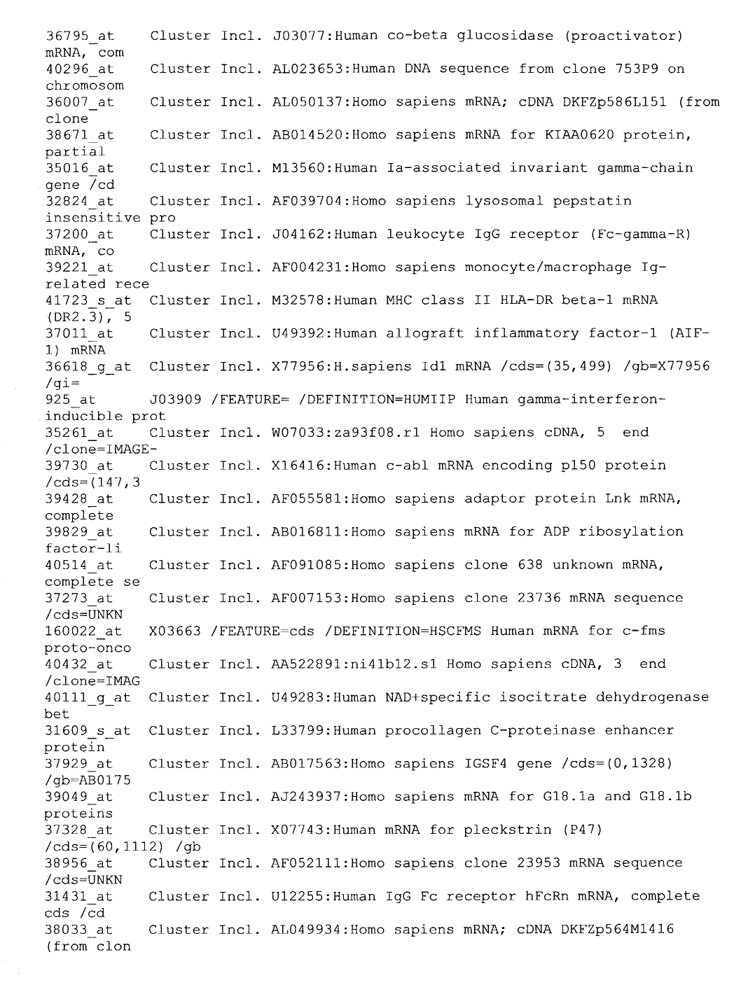

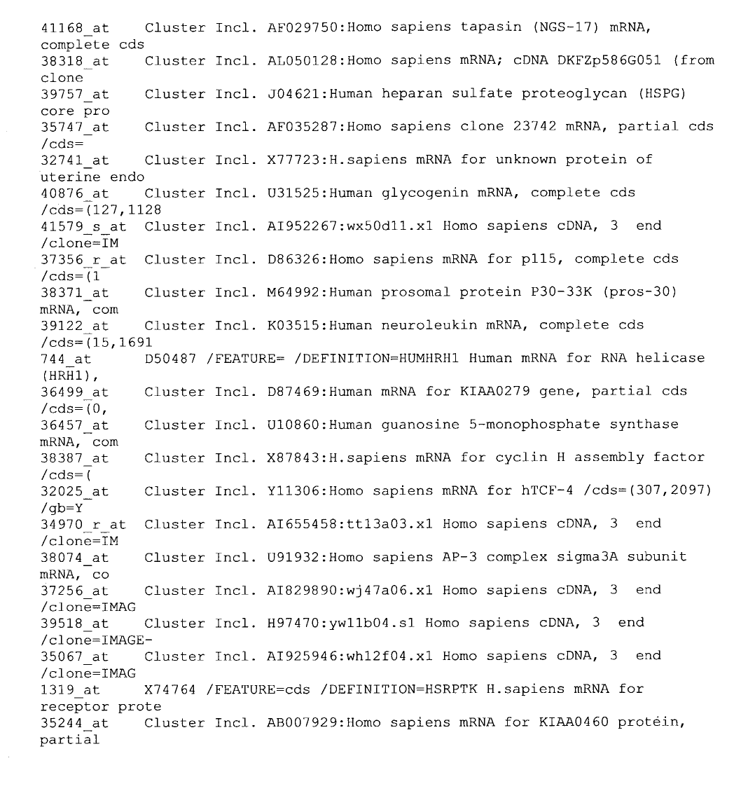

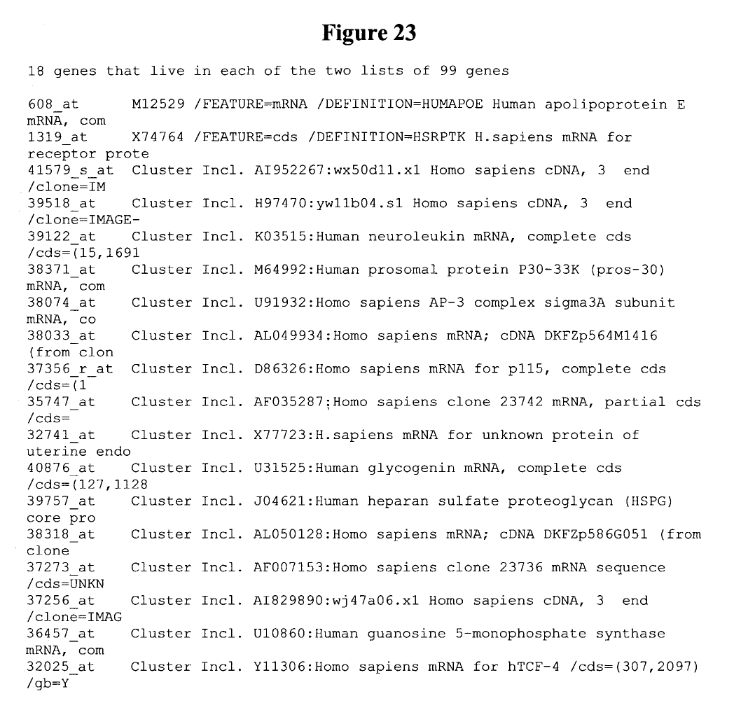

Specific atherosclerotic phenotype determinative genes of the subject invention are those listed in the Tables 1 and 2 as shown in FIGS. 7 and 8 respectively, as well as those listed in FIGS. 9, 10, 16, 17, 18, 19, 20, 21, 22, and 23. Of the list of genes, certain of the genes have functions that logically implicate them as being associated with atherosclerosis. However, the remaining genes have functions that do not readily associate them with atherosclerosis. Those genes logically implicated with atherosclerosis are listed in Table 2. However, the remaining genes have functions that do not readily associate them with atherosclerosis (all others on table 1 as shown in FIG. 7).

The subject invention provides collections of atherosclerotic phenotype determinative genes. Although the following disclosure describes subject collections in terms of the genes listed in Tables 1 and 2 (FIGS. 7 and 8 respectively), the subject collections and subsets thereof as claimed by the invention apply to all relevant genes listed in tables provided in FIGS. 9, 10, 16, 17, 18, 19, 20, 21, 22, and 23. The subject collections and subsets thereof, as well as applications directed to the use of the aforementioned subject collections only serve as an example to illustrate the invention.

The subject collections include at least 2 of the genes listed in Table 1 (See FIG. 7). In certain embodiments, the number of genes in the collection that are from Table 1 is at least 5, at least 10, at least 25, at least 50, at least 75 or more, including all of the genes listed in Table 1.

The subject collections may include only those genes that are listed in Table 1, or they may include additional genes that are not listed in Table 1. Where the subject collections include such additional genes, in certain embodiments the % number of additional genes that are present in the subject collections does not exceed about 50%, usually does not exceed about 25%. In many embodiments where additional “non-Table 1” genes are included, a great majority of genes in the collection are atherosclerotic phenotype determinative genes, where by great majority is meant at least about 75%, usually at least about 80% and sometimes at least about 85, 90, 95% or higher, including embodiments where 100% of the genes in the collection are atherosclerotic phenotype determinative genes.

In many embodiments, at least one of the genes in the collection is a gene whose function does not readily implicate it in the production of an atherosclerotic phenotype, where such genes include those genes that are listed in Table 1 but not listed in Table 2. In many embodiments, the subject collections include 2 or more genes from this group, where the number of genes that are included from in this group may be 5, 10, 20 or more, up to and including all of the genes in this group.

### Methods of Using the Subject Collections of Atherosclerotic Phenotype Determinative Genes

The subject collections find use in a number of different applications. Applications of interest include, but are not limited to: (a) diagnostic applications, in which the collections of the genes are employed to either predict the presence of, or the probability for occurrence of, an atherosclerotic phenotype; (b) pharmacogenomic applications, in which the collections of genes are employed to determine an appropriate therapeutic treatment regimen, which is then implemented; and (c) therapeutic agent screening applications, where the collection of genes is employed to identify atherosclerotic phenotype modulatory agents. Each of these different representative applications is now described in greater detail below.

Diagnostic Applications

In diagnostic applications of the subject invention, cells or collections thereof, e.g., tissues, as well as animals (subjects, hosts, etc., e.g., mammals, such as pets, livestock, and humans, etc.) that include the cells/tissues are assayed to determine the presence of and/or probability for development of, an atherosclerotic phenotype. As such, diagnostic methods include methods of determining the presence of an atherosclerotic phenotype. In certain embodiments, not only the presence but also the severity or stage of an atherosclerotic phenotype is determined. In addition, diagnostic methods also include methods of determining the propensity to develop an atherosclerotic phenotype, such that a determination is made that an atherosclerotic phenotype is not present but is likely to occur.

In practicing the subject diagnostic methods, a nucleic acid sample obtained or derived from a cell, tissue or subject that includes the same that is to be diagnosed is first assayed to generate an expression profile, where the expression profile includes expression data for at least two of the genes of Table 1, where the expression profile may include expression data for 5, 10, 20, 50, 75 or more of, including all of, the genes listed in Table 1. In many embodiments, theexpression profile also includes expression data for at least 1 of the genes listed in Table 2, wherein the expression profile may include expression data for 2, 5, 10, 20 or more, including all of the genes listed in Table 2. The number of different genes whose expression data, i.e., presence or absence of expression, as well as expression level, that are included in the expression profile that is generated may vary, but is typically at least 2, and in many embodiments ranges from 2 to about 100 or more, sometimes from 3 to about 75 or more, including from about 4 to about 70 or more.

As indicated above, the sample that is assayed to generate the expression profile employed in the diagnostic methods is one that is a nucleic acid sample. The nucleic acid sample includes a plurality or population of distinct nucleic acids that includes the expression information of the atherosclerotic phenotype determinative genes of interest of the cell or tissue being diagnosed. The nucleic acid may include RNA or DNA nucleic acids, e.g., mRNA, CRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via biopsy of tissue, using standard protocols, where cell types or tissues from which such nucleic acids may be generated include any tissue in which the expression pattern of the to be determined atherosclerotic phenotype exists, including, but not limited, to, monocytes, endothelium, and/or smooth muscle.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the atherosclerotic phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

Once the expression profile is obtained from the sample being assayed, the expression profile is compared with a reference or control profile to make a diagnosis regarding the atherosclerotic phenotype of the cell or tissue from which the sample was obtained/derived. The reference or control profile may be a profile that is obtained from a cell/tissue known to have an atherosclerotic phenotype, as well as a particular stage of atherosclerosis, and therefore may be a positive reference or control profile. In addition, the reference or control profile may be a profile from cell/tissue for which it is known that the cell/tissue utlimately developed an atherosclerotic phenotype, and therefore may be a positive prognostic control or reference profile. In addition, the reference/control profile may be from a normal cell/tissue and therefore be a negative reference/control profile.

In certain embodiments, the obtained expression profile is compared to a single reference/control profile to obtain information regarding the atherosclerotic phenotype of the cell/tissue being assayed. In yet other embodiments, the obtained expression profile is compared to two or more different reference/control profiles to obtain more in depth information regarding the atherosclerotic phenotype of the assayed cell/tissue. For example, the obtained expression profile may be compared to a positive and negative reference profile to obtain confirmed information regarding whether the cell/tissue has an atherosclerotic or normal phenotype. Furthermore, the obtained expression profile may be compared to a series of positive control/reference profiles each representing a different stage/level of atherosclerosis, so as to obtain more in depth information regarding the particular atherosclerotic phenotype of the assayed cell/tissue. The obtained expression profile may be compared to a prognostic control/reference profile, so as to obtain information about the propensity of the cell/tissue to develop an atherosclerotic phenotype.

The comparison of the obtained expression profile and the one or more reference/control profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above.

The comparison step results in information regarding how similar or dissimilar the obtained expression profile is to the control/reference profiles, which similarity/dissimilarity information is employed to determine the atherosclerotic phenotype of the cell/tissue being assayed. For example, similarity with a positive control indicates that the assayed cell/tissue has an atherosclerotic phenotype. Likewise, similarity with a negative control indicates that the assayed cell/tissue does not have an atherosclerotic phenotype.

Depending on the type and nature of the reference/control profile(s) to which the obtained expression profile is compared, the above comparison step yields a variety of different types of information regarding the cell/tissue that is assayed. As such, the above comparison step can yield a positive/negative determination of an atherosclerotic phenotype of an assayed cell/tissue. In addition, where appropriate reference profiles are employed, the above comparison step can yield information about the particular stage of an atherosclerotic phenotype of an assayed cell/tissue. Furthermore, the above comparison step can be used to obtain information regarding the propensity of the cell or tissue to develop an atherosclerotic phenotype.

In many embodiments, the above obtained information about the cell/tissue being assayed is employed to diagnose a host, subject or patient with respect to the presence of, state of or propensity to develop, atherosclerosis. For example, where the cell/tissue that is assayed is determined to have an atherosclerotic phenotype, the information may be employed to diagnose a subject from which the cell/tissue was obtained as having atherosclerosis.

Pharmaco/Surgicogenomic Applications

Another application in which the subject collections of atherosclerotic phenotype determinative genes finds use in is pharmacogenomic and/or surgicogenomic applications. In these applications, a subject/host/patient is first diagnosed for an atherosclerotic phenotype, e.g., presence or absence of atherosclerosis, propensity to develop atherosclerosis, etc., using a protocol such as the diagnostic protocol described in the preceding section.

The subject is then treated using a pharmacological and/or surgical treatment protocol, where the suitability of the protocol for a particular subject/patient is determined using the results of the diagnosis step. A variety of different pharmacological and surgical treatment protocols are known to those of skill in the art. Such protocols include, but are not limited to: surgical treatment protocols, including bypass grafting, endarterectomy, and percutaneous translumenal angioplasty (PCTA). Pharmacological protocols of interest include treatment with a variety of different types of agents, including but not limited to: thrombolytic agents, growth factors, cytokines, nucleic acids (e.g. gene therapy agents); etc.

Assessment of Therapy (Therametrics)

Another application in which the subject collections of atherosclerotic phenotype determinative genes find use is in monitoring or assessing a given treatment protocol. In such methods, a cell/tissue sample of a patient undergoing treatment for an atherosclerosis disease condition is monitored using the procedures described above in the diagnostic section, where the obtained expression profile is compared to one or more reference profiles to determine whether a given treatment protocol is having a desired impact on the disease being treated. For example, periodic expression profiles are obtained from a patient during treatment and compared to a series of reference/controls that includes expression profiles of various atherosclerotic stages and normal expression profiles. An observed change in the monitored expression profile towards a normal profile indicates that a given treatment protocol is working in a desired manner.

Therapeutic Agent Screening Applications

The present invention also encompasses methods for identification of agents having the ability to modulate an atherosclerotic phenotype, e.g., enhance or diminish an atherosclerotic phenotype, which finds use in identifying therapeutic agents for atherosclerosis.

Identification of compounds that modulate an atherosclerotic phenotype can be accomplished using any of a variety of drug screening techniques. The screening assays of the invention are generally based upon the ability of the agent to modulate an expression profile of atherosclerotic phenotype determinative genes.

The term “agent” as used herein describes any molecule, e.g., protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate agents of particular interest include, but are not limited to, antisense polynucleotides, and antibodies, soluble receptors, and the like. Antibodies and soluble receptors are of particular interest as candidate agents where the target differentially expressed gene product is secreted or accessible at the cell-surface (e.g., receptors and other molecule stablyassociated with the outer cell membrane).

Screening assays can be based upon any of a variety of techniques readily available and known to one of ordinary skill in the art. In general, the screening assays involve contacting a cell or tissue known to have an atherosclerotic phenotype with a candidate agent, and assessing the effect upon a gene expression profile made up of atherosclerotic phenotype determinative genes. The effect can be detected using any convenient protocol, where in many embodiments the diagnostic protocols described above are employed. Generally such assays are conducted in vitro, but many assays can be adapted for in vivo analyses, e.g., in an animal model of the cancer.

Screening for Drug Targets

In another embodiment, the invention contemplates identification of genes and gene products from the subject collections of atherosclerotic determinative genes as therapeutic targets. In some respects, this is the converse of the assays described above for identification of agents having activity in modulating (e.g., decreasing or increasing) an atherosclerotic phenotype, and is directed towards identifying genes that are atherosclerotic phenotype determinative genes, e.g., the genes appearing in Table 1, as therapeutic targets.

In this embodiment, therapeutic targets are identified by examining the effect(s) of an agent that can be demonstrated or has been demonstrated to modulate an atherosclerotic phenotype (e.g., inhibit or suppress an atherosclerotic phenotype). For example, the agent can be an antisense oligonucleotide that is specific for a selected gene transcript. For example, the antisense oligonucleotide may have a sequence corresponding to a sequence of a gene appearing in Table 1.

Assays for identification of therapeutic targets can be conducted in a variety of ways using methods that are well known to one of ordinary skill in the art. For example, a test cell that expresses or overexpresses a candidate gene, e.g., a gene found in Table 1, is contacted with the known atherosclerotic agent, the effect upon a atherosclerotic phenotype and a biological activity of the candidate gene product assessed. The biological activity of the candidate gene product can be assayed be examining, for example, modulation of expression of a gene encoding the candidate gene product (e.g., as detected by, for example, an increase or decrease in transcript levels or polypeptide levels), or modulation of an enzymatic or other activity of the gene product.

Inhibition or suppression of the atherosclerotic phenotype indicates that the candidate gene product is a suitable target for atherosclerotic therapy. Assays described herein and/or known in the art can be readily adapted in for assays for identification of therapeutic targets. Generally such assays are conducted in vitro, but many assays can be adapted for in vivo analyses, e.g., in an appropriate, art-accepted animal model of atherosclerosis.

### Reagents and Kits

Also provided are reagents and kits thereof for practicing one or more of the above described methods. The subject reagents and kits thereof may vary greatly. Reagents of interest include reagents specifically designed for use in production of the above described expression profiles of atherosclerotic phenotype determinative genes.

One type of such reagent is an array probe nucleic acids in which the atherosclerotic phenotype determinative genes of interest are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In many embodiments, the arrays include probes for at least 2 of the genes listed in Table 1, above. In certain embodiments, the number of genes that are from Table 1 that is represented on the array is at least 5, at least 10, at least 25, at least 50, at least 75 or more, including all of the genes listed in Table 1. The subject arrays may include only those genes that are listed in Table 1, or they may include additional genes that are not listed in Table 1. Where the subject arrays include probes for such additional genes, in certain embodiments the number % of additional genes that are represented does not exceed about 50%, usually does not exceed about 25%. In many embodiments where additional “non-Table 1” genes are included, a great majority of genes in the collection are atherosclerotic phenotype determinative genes, where by great majority is meant at least about 75%, usually at least about 80% and sometimes at least about 85, 90, 95% or higher, including embodiments where 100% of the genes in the collection are atherosclerotic phenotype determinative genes. In many embodiments, at least one of the genes represented on the array is a gene whose function does not readily implicate it in the production of an atherosclerotic phenotype, where such genes include those genes listed in Table 2. In many embodiments, the subject arrays include 2 or more genes from Table 2, where the number of genes that are included from Table 2 may be 5, 10, 20 or more, up to an including all of the genes listed in Table 2.

Another type of reagent that is specifically tailored for generating expression profiles of atherosclerotic phenotype determinative genes is a collection of gene specific primers that is designed to selectively amplify such genes. Gene specific primers and methods for using the same are described in U.S. Pat. No. 5,994,076, the disclosure of which is herein incorporated by reference. Of particular interest are collections of gene specific primers that have primers for at least 2 of the genes listed in Table 1, above. In certain embodiments, the number of genes that are from Table 1 that have primers in the collection is at least 5, at least 10, at least 25, at least 50, at least 75 or more, including all of the genes listed in Table 1. The subject gene specific primer collections may include only those genes that are listed in Table 1, or they may include primers for additional genes that are not listed in Table 1. Where the subject gene specific primer collections include primers for such additional genes, in certain embodiments the number % of additional genes that are represented does not exceed about 50%, usually does not exceed about 25%. In many embodiments where additional “non-Table 1” genes are included, a great majority of genes in the collection are atherosclerotic phenotype determinative genes, where by great majority is meant at least about 75%, usually at least about 80% and sometimes at least about 85, 90, 95% or higher, including embodiments where 100% of the genes in the collection are atherosclerotic phenotype determinative genes. In many embodiments, at least one of the genes represented on collection of gene specific primers is a gene whose function does not readily implicate it in the production of an atherosclerotic phenotype, where such genes include those genes listed in Table 2. In many embodiments, the subject gene specific primer collections include 2 or more genes from Table 2, where the number of genes that are included from Table 2 may be 5, 10, 20 or more, up to an including all of the genes listed in Table 2.

The kits of the subject invention may include the above described arrays and/or gene specific primer collections. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Compounds and Methods for Treatment of Cardiovascular Disease

Also provided are methods and compositions whereby cardiovascular disease symptoms may be ameliorated. The subject invention provides methods of ameliorating, e.g., treating, an atherosclerotic disease conditions, by modulating the expression of one or more target genes or the activity of one or more products thereof, where the target genes are one or more of the atherosclerotic phenotype determinative genes of Table 1.

Certain cardiovascular diseases are brought about, at least in part, by an excessive level of gene product, or by the presence of a gene product exhibiting an abnormal or excessive activity. As such, the reduction in the level and/or activity of such gene products would bring about the amelioration of cardiovascular disease symptoms. Techniques for the reduction of target gene expression levels or target gene product activity levels are discussed below.

Alternatively, certain other cardiovascular diseases are brought about, at least in part, by the absence or reduction of the level of gene expression, or a reduction in the level of a gene product's activity. As such, an increase in the level of gene expression and/or the activity of such gene products would bring about the amelioration of cardiovascular disease symptoms. Techniques for increasing target gene expression levels or target gene product activity levels are discussed below.

### Compounds that Inhibit Expression, Synthesis or Activity of Mutant Target Gene Activity

As discussed above, target genes involved in cardiovascular disease disorders can cause such disorders via an increased level of target gene activity. As summarized in Table 1, above, a number of genes are now known to be up-regulated in cells/tissues under disease conditions. A variety of techniques may be utilized to inhibit the expression, synthesis, or activity of such target genes and/or proteins. For example, compounds such as those identified through assays described which exhibit inhibitory activity, may be used in accordance with the invention to ameliorate cardiovascular disease symptoms. As discussed, above, such molecules may include, but are not limited to small organic molecules, peptides, antibodies, and the like. Inhibitory antibody techniques are described, below.

For example, compounds can be administered that compete with an endogenous ligand for the target gene product, where the target gene product binds to an endogenous ligand. The resulting reduction in the amount of ligand-bound gene target will modulate endothelial cell physiology. Compounds that can be particularly useful for this purpose include, for example, soluble proteins or peptides, such as peptides comprising one or more of the extracellular domains, or portions and/or analogs thereof, of the target gene product, including, for example, soluble fusion proteins such as Ig-tailed fusion proteins. (For a discussion of the production of Ig-tailed fusion proteins, see, for example, U.S. Pat. No. 5,116,964.). Alternatively, compounds, such as ligand analogs or antibodies, that bind to the target gene product receptor site, but do not activate the protein, (e.g., receptor-ligand antagonists) can be effective in inhibiting target gene product activity. Furthermore, antisense and ribozyme molecules which inhibit expression of the target gene may also be used in accordance with the invention to inhibit the aberrant target gene activity. Such techniques are described, below. Still further, also as described, below, triple helix molecules may be utilized in inhibiting the aberrant target gene activity.

### Inhibitory Antisense Ribozyme and Triple Helix Approaches

Among the compounds which may exhibit the ability to ameliorate cardiovascular disease symptoms are antisense, ribozyme, and triple helix molecules. Such molecules may be designed to reduce or inhibit mutant target gene activity. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the 10 and +10 regions of the target gene nucleotide sequence of interest, are preferred. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays. Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex. It is possible that the antisense, ribozyme, and/or triple helix molecules described herein may reduce or inhibit the transcription (triple helix) and/or translation (antisense, ribozyme) of mRNA produced by both normal and mutant target gene alleles. In order to ensure that substantially normal levels of target gene activity are maintained, nucleic acid molecules that encode and express target gene polypeptides exhibiting normal activity may be introduced into cells via gene therapy methods such as those described, below, that do not contain sequences susceptible to whatever antisense, ribozyme, or triple helix treatments are being utilized. Alternatively, it may be preferable to co-administer normal target gene protein into the cell or tissue in order to maintain the requisite level of cellular or tissue target gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### Antibodies for Target Gene Products

Antibodies that are both specific for target gene protein and interfere with its activity may be used to inhibit target gene function. Such antibodies may be generated using standard techniques known in the art against the proteins themselves or against peptides corresponding to portions of the proteins. Such antibodies include but are not limited to polyclonal, monoclonal, Fab fragments, single chain antibodies, chimeric antibodies, etc.

In instances where the target gene protein is intracellular and whole antibodies are used, internalizing antibodies may be preferred. However, lipofectin liposomes may be used to deliver the antibody or a fragment of the Fab region which binds to the target gene epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (e.g., see Creighton, 1983, supra; and Sambrook et al., 1989, supra). Alternatively, single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (Marasco, W. et al., 1993, Proc. Natl. Acad. Sci. USA 90:7889-7893).

In some instances, the target gene protein is extracellular, or is a transmembrane protein. Antibodies that are specific for one or more extracellular domains of the gene product, for example, and that interfere with its activity, are particularly useful in treating cardiovascular disease. Such antibodies are especially efficient because they can access the target domains directly from the bloodstream. Any of the administration techniques described, below which are appropriate for peptide administration may be utilized to effectively administer inhibitory target gene antibodies to their site of action.

### Methods for Restoring Target Gene Activity

Target genes that cause cardiovascular disease may be underexpressed within cardiovascular disease situations. As summarized in Table 1, above, several genes are now known to be down-regulated under disease conditions. Alternatively, the activity of target gene products may be diminished, leading to the development of cardiovascular disease symptoms. Described in this Section are methods whereby the level of target gene activity may be increased to levels wherein cardiovascular disease symptoms are ameliorated. The level of gene activity may be increased, for example, by either increasing the level of target gene product present or by increasing the level of active target gene product which is present.

For example, a target gene protein, at a level sufficient to ameliorate cardiovascular disease symptoms may be administered to a patient exhibiting such symptoms. Any of the techniques discussed, below, may be utilized for such administration. One of skill in the art will readily know how to determine the concentration of effective, non-toxic doses of the normal target gene protein, utilizing techniques known to those of ordinary skill in the art.

Additionally, RNA sequences encoding target gene protein may be directly administered to a patient exhibiting cardiovascular disease symptoms, at a concentration sufficient to produce a level of target gene protein such that cardiovascular disease symptoms are ameliorated. Any of the techniques discussed, below, which achieve intracellular administration of compounds, such as, for example, liposome administration, may be utilized for the administration of such RNA molecules. The RNA molecules may be produced, for example, by recombinant techniques as is known in the art.

Further, patients may be treated by gene replacement therapy. One or more copies of a normal target gene, or a portion of the gene that directs the production of a normal target gene protein with target gene function, may be inserted into cells using vectors which include, but are not limited to adenovirus, adeno-associated virus, and retrovirus vectors, in addition to other particles that introduce DNA into cells, such as liposomes. Additionally, techniques such as those described above may be utilized for the introduction of normal target gene sequences into human cells.

Cells, preferably, autologous cells, containing normal target gene expressing gene sequences may then be introduced or reintroduced into the patient at positions which allow for the amelioration of cardiovascular disease symptoms. Such cell replacement techniques may be preferred, for example, when the target gene product is a secreted, extracellular gene product.

### Pharmaceutical Preparations and Methods of Administration

The identified compounds that inhibit target gene expression, synthesis and/or activity can be administered to a patient at therapeutically effective doses to treat or ameliorate cardiovascular disease. A therapeutically effective dose refers to that amount of the compound sufficient to result in amelioration of symptoms of cardiovascular disease. Effective Dose Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Methods of Identifying Atherosclerotic Phenotype Determinative Genes

Also provided are methods of identifying atherosclerotic phenotype determinative genes, i.e., genes whose expression is associated with a disease phenotype. In these methods, an expression profile for a nucleic acid sample obtained from a source having the atherosclerotic phenotype is prepared using the gene expression profile generation techniques described above, with the only difference being that the genes that are assayed are candidate genes and not genes necessarily known to be atherosclerotic phenotype determinative genes. Next, the obtained expression profile is compared to a control profile, e.g., obtained from a source that does not have an atherosclerotic phenotype.

Following this comparison step, genes whose expression correlates with said the atherosclerotic phenotype are identified. A feature of the subject invention is that the correlation is based on at least one parameter that is other than expression level. As such, a parameter other than whether a gene is up or down regulated is employed to find a correlation of the gene with the atherosclerotic phenotype. In many embodiments, the correlation is determined using a Bayesian analysis.

More specifically, of interest is a correlation analysis that uses standard binary regression models combined with singular value decompositions (SVDs), also referred to as singular factor decompositions, and with stochastic regularization using Bayesian analysis. See e.g., Gelman, A., Carlin, J. B., Stem, H. S. & Rubin, D. B. (1996) Bayesian Data Analysis (Chapman & Hall, London). In this method, the classification probability for each of the two possible outcomes for each sample is structured as a probit regression model in which the expression levels of genes are scored by regression parameters in a regression vector b. Analysis estimates this regression vector and the resulting classification probabilities for both training and validation samples. The estimated regression vector itself is used not only to define the predictive classification but also in scoring genes as to their contribution to the classification. This screening strategy computes sample correlation coefficients between gene expression and disease vs. normal binary outcomes and selects those genes giving the largest absolute values of this correlation. More detail regarding this particular analysis, which one of skill in the art may employed to readily practice this method without practicing undue experimentation, is found in West et al., Proc. Natl. Acad. Sci. USA, Vol. 98, Issue 20, 11462-11467, Sep. 25, 2001, and the information available on the corresponding website: http://www.pnas.org/cgi/content/abstract/201162998v1.

The above gene expression analysis approach to the identification of atherosclerotic phenotype determinative genes may be combined with one or more additional selection protocols in a “multi-prong” gene selection approach for identifying genes associated with an atherosclerotic phenotype. Additional selection protocols that can be employed in conjunction with the subject selection protocol include: (1) selection protocols that identify all currently known genes that are associated with atherosclerosis (e.g., as determined by using existing biological and clinical databases, e.g., by performing a thorough review of the published literature concerning biological research on atherosclerosis mechanism and clinical research related to drugs that have shown a beneficial, or detrimental, effect on patients with atherosclerotic clinical manifestations); (2) genes that have been identified as associated with atherosclerosis using human genetic studies, e.g., genetic linkage analysis (for example, one analyzes the genome of individuals who have presented with premature coronary heart disease (CAD, hard manifestations of CAD before 45 for men and before 50 for women, such as myocardial infarction or bypass surgery), and their siblings and studies markers within the genome of these individuals that co-segregate with the disease process. The localition of such markers across the entire genome allows for identification of “hot spots” that contain 10-300 genes. These genes become candidates for further analysis); (3) genes that have been identified as associated with atherosclerosis using mouse genetic studies, e.g., using mouse models of human disease (Using established mouse models of atherosclerosis, such as ApoE knock-out mice, one searches for “modifiers” that alter the development of the disease process, either increase or reduce, that come into play upon changing the genetic background of the mice. The modifiers thus identified, or their human equivalents, in turn, become candidate genes for further studies on human atherosclerosis); (4) genes that have been identified as associated with atherosclerosis using epigenetic and methylation studies (It is know that with aging, gene expression can be altered, yet the mechanism(s) for such altered expression remains an enigma. Changes in methylation of CpG islands within the promoter region of a multitude of genes can result in altered transcription of such genes, and we have shown that such changes can occur in cardiovascular tissues with aging. Typically, methylation of the CpG island within the promoter of a gene results in silencing of this gene. Such changes in DNA methylation have been called “epigenetic” as they do not represent necessarily inherited changes. We have been surveying the genome of human aortas for the presence of genes whose methylation is altered within atherosclerotic regions compared to normal aorta tissue. The technique that we have used for this survey is called restriction landmark genome scanning, or RLGS. We have already identified two genes, nucleolin and monocarboxylate transporter 3 (MCT3) that are differently methylated between normal and diseased aorta tissues. These genes have become members of our pool of “candidates”). Where the above expression analysis approach is combined with one or more additional approaches to identify genes that are atherosclerotic phenotype determinative genes, the initial genes identified using each disparate selection protocol may be combined into a single set for further use, as described below, using a number of different combination protocols. For example, each of the initially identified subsets may be additively combined to produce a master set of genes for further use. Alternatively, only the common genes of one or more subsets may be placed in the final set of genes for further use. For example, where one develops five initial subsets of genes using five different selection criteria, such as the specific criteria listed above, only those genes common to at least two or more, three or more, or four or more of the initial subsets, including all of the initial subsets, may be chosen for inclusion in the final set.

The resultant final or master set of genes may be used as a collection of atherosclerotic phenotype determinative genes as described above. In addition, such a set may be used as an initial set or “library” of candidate genes for further study to identify SNPs that cause or are otherwise associated with an atherosclerotic phenotype.

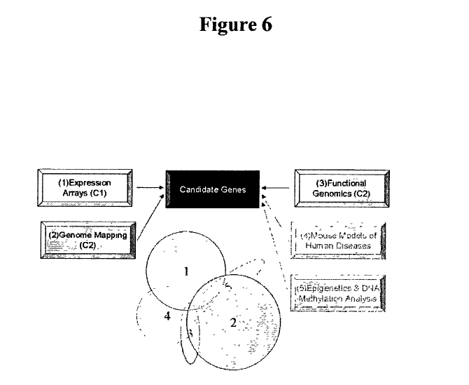

FIG. 6 provides a flow diagram showing a selection procedure as described above as it would be used to identify atherosclerotic phenotype determinative gene variants, e.g., SNPs, which are then used, either singly or in combination, in a variety of different applications, including the applications described above in connection with the specific atherosclerotic phenotype determinative genes identified herein.

While the above selection approach of the subject invention is described above in terms of the identification of atherosclerotic phenotype determinative genes, included within the scope of the invention is the use of the above approach to identify genes that are determinative of other phenotypes, including other disease phenotypes, such as cancer, etc. For example, the above gene identification approaches have been successfully used to predict the status of breast cancer, as described in West et al., Proc. Natl. Acad. Sci. USA, Vol. 98, Issue 20, 11462-11467, Sep. 25, 2001, which is available at http://www.pnas.org/cgi/content/abstract/201162998v1.

The following examples are offered by way of illustration and not by way of limitation.

Experimental

I. Tissue/Sample Procurement for Gene Expression Analysis

A serious challenge at the inception of this study was to find human arterial material that would be suitable for study of various stages of atherosclerosis and concurrent gene expression profiling. Although the most straightforward approach to the analysis of disease tissue would be the collection of material from individuals who have either succumbed to heart disease or those who are undergoing a heart transplant, this has the significant disadvantage of utilizing tissue at the end-stage of disease. Many previous studies have demonstrated that atherosclerosis is a long-term process associated with aging, with development of disease preceding the development of overt signs of disease. Hence, it is likely that end-stage tissue would not reflect events associated with initiation and progression of disease, but instead molecular events that reflect response to injury and associated repair processes.

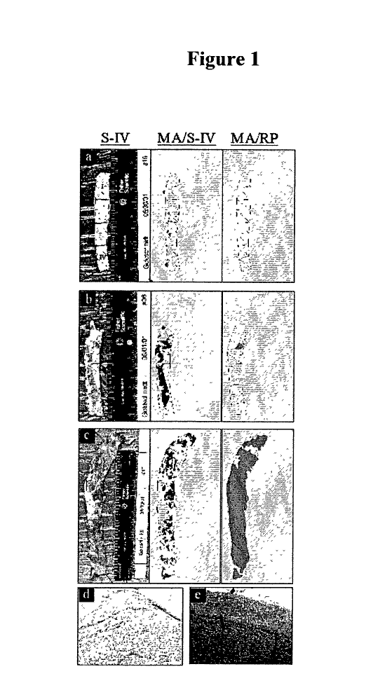

As an alternative approach, we have collected the thoracic aorta of heart donors at the time of cardiac harvest, to minimize post-mortem changes in gene expression, and to provide sufficient mass of tissue for multifaceted analysis. Considering the inherent sagittal symmetry of the human aortic tissue in terms of distribution of atherosclerotic lesions, as demonstrated by the Pathological Determinants of Atherosclerosis in the Youth (PDAY) study, this model provides the unique opportunity to investigate both the atherosclerotic burden and expression profile on matched segments of the aorta. Furthermore, aorta samples, although collected from clinically unaffected heart donors, did present various degrees of atherosclerosis burden, from absent or mild to severe lesions (FIG. 1).

We observed that the atherosclerotic lesions were not distributed uniformly across aorta samples, and indeed formed a mosaic, where a gradient in lesion intensity was observed from proximal to distal segments, with more severe lesions found distally. Hence, for this analysis, the aorta samples were divided into four equivalent segments, and the distal quarter (segment IV, with most advanced pathology) was compared to the proximal one, which was virtually free of atherosclerotic lesions (segment I). We measured the atherosclerotic burden of the segments using well established techniques, applied to study atherosclerosis burden in the PDAY investigation of more than 3,000 human aorta samples. Both early (Soudan IV positive) and more complex (raised plaques) lesions of atherosclerosis were measured, and data were expressed as percent of affected area versus total surface of the segment under study. As expected, the extent of atherosclerotic lesions, both fatty streaks (Soudan IV positive plaques) and raised plaques, was significantly greater in segment IV relative to segment I (p<0.01). Thus, the binary classification of proximal vs. distal was relevant to comparing two sets of samples with significantly different atherosclerotic burden.

While relative to a long axis equidistant to the intercostal arteries, the right half of the segments was used to measure the atherosclerosis score, the matching left half was extracted for RNA studies.

II. Gene Expression Analysis

Aorta tissue was homogenized to yield RNA, and the extracted RNA was further analyzed for quality prior to Affymetrix GENECHIP analysis, first by checking the 28S: 18S ribosomal RNA ratio using an Agilent Bioanalyzer. The targets for Affymetrix DNA microarray analysis were prepared according to the manufacturer's instructions.

All assays used the human HuGeneFL GENECHIP microarray. Arrays were hybridized with the targets at 45° C. for 16 h and then washed and stained by using the GENECHIP Fluidics. DNA chips were scanned with the GENECHIP scanner, and signals obtained by the scanning were processed by GENECHIP Expression Analysis algorithm (version 3.2) (Affymetrix, Santa Clara, Calif.).

III. Statistical Analysis and Screening via Binary Regression

Initial analysis of our human aorta sample (n=27, 15 segment I, “healthy”, aortic tissue and 12 segment IV, “diseased”, tissue) explored the discriminatory ability of a screened set of genes selected according to raw correlations with the binary classification into aortic site (I vs. IV). The study ignored pairing of samples within aorta, as examination of the initial data set suggested that the patterns of differential expression of a number of genes identified was not obscured by between-individual variation, and thus a direct comparison of sites without regard to pairing appeared valid. Our analysis utilized a modification of the Affymetrix average difference (AD) expression index, using a log2 scale after truncation of the raw measure at 1 unit on normalized AD scale, according to our previous work (West et al. PNAS, http://www.pnas.org/cgi/content/abstract/201162998v1). Next, binary regression models combined with singular value decompositions (SVDs) and with stochastic regularization were developed using Bayesian analysis. The classification probability for each of the two possible outcomes for each sample was structured as a probit regression model in which the expression levels of genes are scored by regression parameters in a regression vector b. Analysis estimates this regression vector and the resulting classification probabilities for both training and validation samples. The estimated regression vector itself is used not only to define the predictive classification but also in scoring genes as to their contribution to the classification. Our screening strategy computed sample correlation coefficients between gene expression and distal vs. proximal binary outcomes and selected those genes giving the largest absolute values of this correlation. Thus, binary regression models combined with singular value decompositions (SVDs) and with stochastic regularization were developed using Bayesian analysis. Our screening highlighted 83 genes {provided in Table 1}, following model re-analysis with varying numbers of genes. The classification probability for each of the two possible outcomes (I vs. IV) for each sample was structured as a probit regression model in which the expression level of genes was scored by regression parameters in a regression vector b. Analysis estimated this regression vector and the resulting classification probabilities, and the estimated regression vector itself was used not only to define the predictive classification but also in scoring genes as to their contribution to the classification.

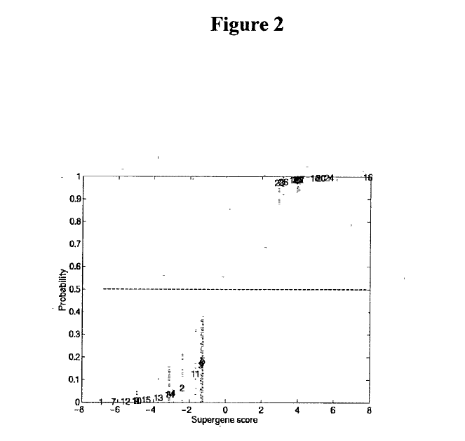

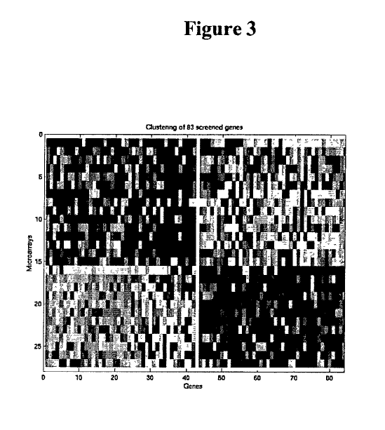

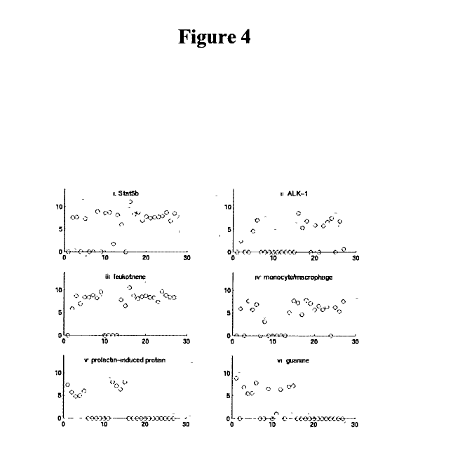

Genes listed in Table 1 were ordered according to estimated regression coefficients. In this context, a positive coefficient is associated with genes for whom increased expression levels favored the segment IV, “diseased”, aortic region, and vice-versa. A display of the probabilistic within-sample discrimination based on this analysis, giving estimated classification probabilities that identify tissues as likely “diseased” (segment IV) versus “healthy” (segment I) based on the expression profile of the 83 genes and summarized in terms of the implied “supergene” regression predictor, is shown in FIG. 2. Approximate 95% prediction intervals accompany the point predictions, and the data indicated that all cases are accurately classified, as confirmed by simple cluster analysis of this subset of genes. A display of the expression levels following clustering of the genes into two groups is shown in FIG. 3, showing the natural grouping according to samples; thus, samples 1-15 were from segments I, whereas samples 16-27 were from the diseased segments IV. Based on this sample ordering, the patterns of expression of genes by sample did clearly indicate the correct classification.

Specific genes identified in screening and discrimination analysis As is to be expected, the most highly scored genes represent those for which the differences in average levels of expression between the two groups of tissues are greatest. Among the highly scoring genes, the patterns of difference are structured in a very informative manner, and are worth. Display of expression levels (log2 scale) of six selected genes whose patterns are representative of a larger group is shown in FIG. 4. In cases i-iv, expression levels were elevated (modest to high levels) in virtually all segments IV, whereas the distribution among segments I was mixed. Thus, for some segments I, gene expression was at levels comparable to the segments IV, whereas the gene was unexpressed in other segments I. There were additional genes in the discriminatory set of 83 with these general features. Assuming this behavior represented a population characteristic, one could infer that expression of such genes is necessary characteristic of the segment IV condition. Cases v-vi were two examples of genes showing a reverse characteristic: genes that were apparently unexpressed in all segments IV, but that may or may not be expressed in segments I. Again, lack of expression of these genes, and others sharing this pattern, is another necessary characteristic of the segment IV group.

The unique patterns observed in the expression of a number of the discriminatory genes suggested that a simple partition as expressed versus unexpressed will provide accurate classification based on a small number of genes, and hence focus the gene selection process on genes that have predictive capacity based on the notion of a gene being “necessary” in one of the tissue sites. The potential for this approach lies in the recognition of the predictive utility of these kinds of patterns by relating them across subsets of genes. Thus, we are able to achieve perfect classification of all 27 in a one-at-atime cross-validation analysis that uses only 4 genes, and is based on a nonlinear conjunction rule that capitalized on the above notion of necessary genes. By removing each sample and classifying it based on the remainder, we can perfectly classify all cases using these and other discriminatory genes. Hence, our analysis strongly points at these genes for their potentially unique biological contribution to atherogenesis. A limitation of our study resided in the fact that cross-validating predictions from the binary regression model—in which we predicted the binary categorization of each sample based on analysis of the rest—likely would be inaccurate. The reason was that the distribution of expression of most discriminatory genes is essentially bimodal in the case of the segments I. Hence, tissues with appreciable levels of expression of genes such as in i-iv (FIG. 4) will be difficult to classify out-of-sample. The form of the binary regression model does not easily accommodate this instructive general pattern of variation within one of the two binary categories, and so must be modified or extended to utilize other statistical concepts. One set of concepts raised in the proposal related to the notion of tree-structured models and methods of partitioning, in which the relevant information in selected genes is based on which “partition” the expression levels lie in.

We note that this exploratory analysis relates closely to a form of classification tree, or partitioning models; the development of statistical methods that combine predictive regression modeling with classification trees and partitioning models was explicitly discussed in the proposal, and this analysis, though quite preliminary, gives additional support to that proposed line of development. A formal statistical analysis of such rule-based classification allows for extrapolation to the population of aortic tissues and will be developed once validated in larger samples.

The genes identified as having discriminatory expression coded either for known proteins, or were genes whose annotation was not yet established. Those coding for know proteins, belonged to categories that one would expect to identify in a survey of atherosclerosis tissue. Thus, many of these genes coded for proteins belonging to growth signaling pathways, pathways involved in cellcell communication, cell migration, and metabolic functions. Some of these coded proteins, such as endothelin, had been associated with atherogenesis. Interestingly, many of the discriminatory genes would not have been linked to atherosclerosis, if the absence of our nonbiased approach. Though this pilot study is very preliminary and based on a rather small sample, it does suggest that the proposed approach to gene identification will be very valuable once much larger samples are available.

An examination of the genes identified as having discriminatory expression properties include several that could be seen logically to play a role in the development of atherosclerosis (Table 1). For instance, the Alk-1 gene encodes a member of the TGF-1 Family of receptors specific for endothelial cells and known to play a role in endothelial cell proliferation. Additionally, genes such as endothelin have been previously associated with atherogenesis. Many of the other genes coded for proteins belonging to growth signaling pathways, pathways involved in cell-cell communication, cell migration, and metabolic functions. Based on these selected examples of genes identified as discriminatory that are logically linked proliferative processes in the vascular tissue, we conclude that the expression analysis does indeed have the capacity to identify genes whose expression is related to the process of atherosclerosis.

V. Relating Soudan IV Staining to Gene Expression

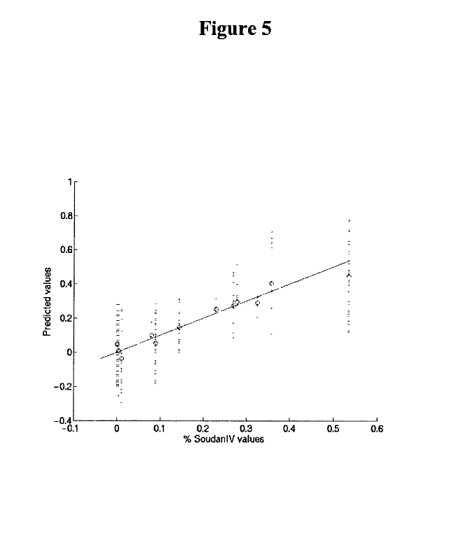

Although the analysis of gene expression patterns in segments I versus IV provides a reasonable initial approach to classifying gene expression that relates to development of atherosclerosis, it is important to relate the gene expression patterns to quantitative aspects of disease development. In particular, the associations between patterns of gene expression and extent of atherosclerotic burden as measured by Soudan IV staining. In spite of a relatively small sample size, present in the invention applies a similar basic conceptual approach to gene screening using a statistical regression model for prioritizing selected genes. The Bayesian binary regression analysis and its use of singular value decomposition methods to allow regression on many genes with very limited data has a counterpart in the more standard linear regression framework. Using similar methods of stochastic regularization, linear regressions were fit in which the % area affected by atherosclerotic scaling (as measured by Soudan IV staining) is predicted by an optimized linear function of expression levels of selected genes. Gene subset selection in this pilot analysis follows that of the binary analysis in selecting genes most highly correlated with the Soudan IV measure, and choosing the number selected by refitting the model repeatedly to different numbers of genes. FIG. 5 provides one summary of such an analysis using 55 genes selected this way. The 55 genes are listed in Table 3 as shown in FIG. 9. Each of the 12 segment IV tissue samples is represented by its measured % scaling, on the horizontal axis, and by the corresponding predicted value from the linear regression model using these 55 genes, on the vertical axis. The line of equality is drawn; a “perfect” model fit to the data would have all 12 circles sitting on this line. The vertical dashed lines represent approximate 95% probability intervals that represent uncertainty in the predictions. The point predictions alone represent a fit to the data with a traditional regression R2 measure of fit of about 94%, consistent with a statistically significant model. Although this is an analysis that is obviously limited by the size of the dataset, it is certainly very encouraging that gene expression patterns can be identified that predict the extent of atherosclerosis development within the tissue.

IV. Statistical Analysis and Screening Using a Predictive Tree Model with Bayesian Analysis

The statistical analysis described and claimed is a predictive statistical tree model that overcomes several problems observed in prior statistical models and regression analyses, while ensuring greater accuracy and predictive capabilities. Although the claimed use of the predictive statistical tree model described herein is directed to the prediction of atherosclerosis in individuals, the claimed model can be used for a variety of applications including the prediction of disease states, susceptibility of disease states or any other biological state of interest, as well as other applicable non-biological states of interest.

This model first screens genes to reduce noise, applies k-means correlation-based clustering targeting a large number of clusters, and then uses singular value decompositions (SVD) to extract the single dominant factor (principal component) from each cluster. This generates a statistically significant number of cluster-derived singular factors, that we refer to as metagenes, that characterize multiple patterns of expression of the genes across samples. The strategy aims to extract multiple such patterns while reducing dimension and smoothing out gene-specific noise through the aggregation within clusters. Formal predictive analysis then uses these metagenes in a Bayesian classification tree analysis. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. We perform iterative out-of-sample, cross-validation predictions: leaving each biological sample out of the data set one at a time, refitting the model from the remaining biological samples and using it to predict the hold-out case. This rigorously tests the predictive value of a model and mirrors the real-world prognostic context where prediction of new cases as they arise is the major goal.

Combined use of multiple metagenes, in the context of the tree selection model building process, ultimately yields a pattern that has the capacity to accurately predict the clinical outcome. A critical element of this approach is the acid test of out-of-sample predictive assessment via cross-validation. Note that any selection of gene, metagene or clinical variables must be part of each cross-validation analysis. The results of such “feature selection” will vary each time a biological sample is analyzed, and can dramatically impact on predictive accuracy. Analyses that select a set of predictors based on the entire dataset, including the individual to be predicted, in advance of predictive evaluation are inappropriate, and lead to misleadingly overoptimistic conclusions about predictive value.

The data subject to this true model analysis was collected and screened as follows:

Tissue Collection and Characterization

The thoracic aortas of heart donors were collected at the time of organ harvest, kept on ice in University of Wisconsin solution to minimize post-mortem changes and provide sufficient tissue for multifaceted analysis. Each aorta was sectioned prior to further processing (FIG. 1a and 1b). Segments A and B were snap frozen in liquid nitrogen for RNA extraction and microarray analysis. Strip C was preserved in formalin and used for atherosclerosis characterization. We exploited the inherent sagittal symmetry of atherosclerotic development and the gradient in lesion intensity from proximal to distal segments as originally described in the Pathobiological Determinants of Atherosclerosis in the Youth (PDAY) study (FIG. 1b). See Cornhill J F, Herderick E E, Stary H C. Topography of human aortic sudanophilic lesions. Monogr Atheroscler 1990; 15:13-9. To assess fatty streaks (early atherosclerotic plaques), the area of Sudan IV staining for each aorta sample was obtained by automated image processing software. See Cornhill J F, Barrett W A, Herderick E E, Mahley R W, Fry D L, Topographic study of sudanophilic lesions in cholesterol-fed minipigs by image analysis, Arteriosclerosis 1985; 5:415-26. We also applied PDAY methodologies to evaluate raised atherosclerotic lesions (advanced plaques). See Cornhill J F, Barrett W A, Herderick E E, Mahley R W, Fry D L. Topographic study of sudanophilic lesions in cholesterol-fed minipigs by image analysis. Arteriosclerosis 1985; 5:415-26. The data were expressed as a ratio of affected area over total surface of the studied segment.

RNA Preparation, Microarray Processing, and Statistical Analysis

All techniques for microarray analysis have been reported previously as described in West M, Blanchette C, Dressman H, et al, Predicting the clinical status of human breast cancer by using gene expression profiles, Proc Natl Acad Sci USA; 98:11462-7 (2001). Briefly, 35 aorta samples were used in the study; 19 low susceptibility samples and 16 high susceptibility samples. Aortic tissue was ground to powder in liquid nitrogen and further processed with a tissue homogenizer. The RNA was extracted using a standard Gibco Trizol extraction protocol and purified with the Qiagen RNeasy Mini kit. The RNA was analyzed for quality by assessing the 28S:18S ribosomal RNA profile and ratio with Agilent Bioanalyzer. A further quality check evaluated the scaling factors of housekeeping genes with Affymetrix Test chips.

The targets for DNA microarray analysis were prepared and hybridized to U95Av2 Affymetrix microarrays surveying about 12,600 genes. The chips were scanned and processed with the GENECHIP system and average difference (AD) measures of expression were obtained. The average difference gene expression index was converted to a log2 scale following thresholding at an absolute level of 64, and then transformed using quantile normalization to remove minor non-linear distortions induced by the Affymetrix scanning. See West M, Blanchette C, Dressman H, et al. Predicting the clinical status of human breast cancer by using gene expression profiles. Proc Natl Acad Sci USA; 98:11462-7 (2001).

Data Analysis

This analysis prioritized genes by their ability to predict two clinical phenotypes: (a) susceptibility for the development of atherosclerosis and (b) severity of atherosclerotic burden.

The basis for the analysis of atherosclerotic susceptibility is the conclusive results from the PDAY study that showed the progression of disease form the distal to proximal areas of the aorta. These data indicate that the distal sections were clearly more susceptible to disease development relative to the proximal sections. Hence, for this analysis, the proximal or 1A sections were compared to the distal or 4B sections to detect gene expression patterns that reflect the susceptibility for atherosclerosis development. A total of 22-1 A and 23-4B sections were used.

For the analysis of genes associated with the severity of atherosclerotic burden, Sudan staining and raised lesion analysis was used to define minimally and severely diseased groups. The minimally diseased group represented the first quartile of Sudan IV staining with little to no Sudan staining and also contained no raised lesions. The severely diseased group represented samples with both significant amounts of Sudan IV staining and raised lesions. There were a total of 14 minimally diseased and 12 severely diseased samples.

For the statistical analysis, we first identified subsets of genes were identified using kmeans clustering followed by the application of singular factor (principal components) analysis to each of the gene clusters to produce a single factor that represents each cluster. These factor or metagenes are used to determine non-linear associations that exist between the binary outcomes of low vs high susceptibility to atherosclerosis and minimal vs severe atherosclerotic burden. The association measure takes each metagene and determines the threshold at which each metagene best partitions the aorta samples relative to the respective clinical classification being considered. Statistical trees were then developed where each node represents the metagene that optimally partitions the aorta samples into the correct classification. Once the metagenes used for the tree models were identified, the genes that lend the highest weight to each metegene then became the genes that were important for the clinical phenotype in question. Out of sample cross-validation analysis was then applied to test the model.

Gene Annotation

The list of candidate genes generated by the statistical model was annotated using the GenBank, Unigene and LocusLink databases. Further information was obtained from PubMed and MEDLINE.

Results

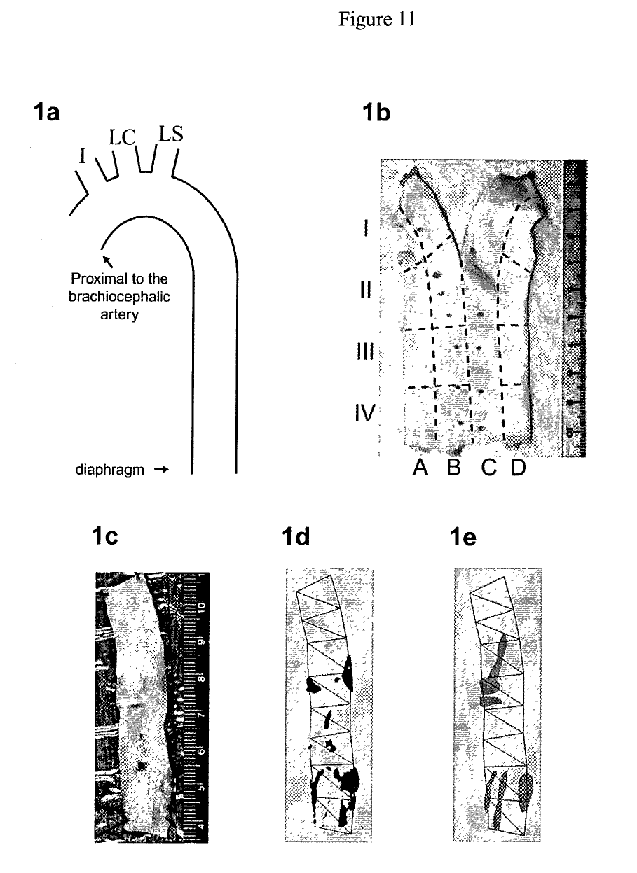

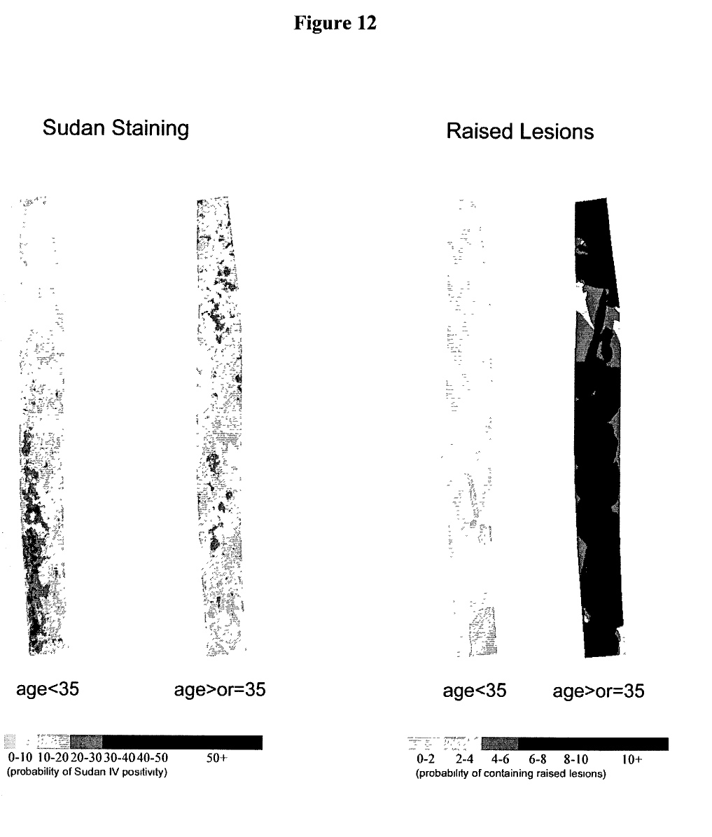

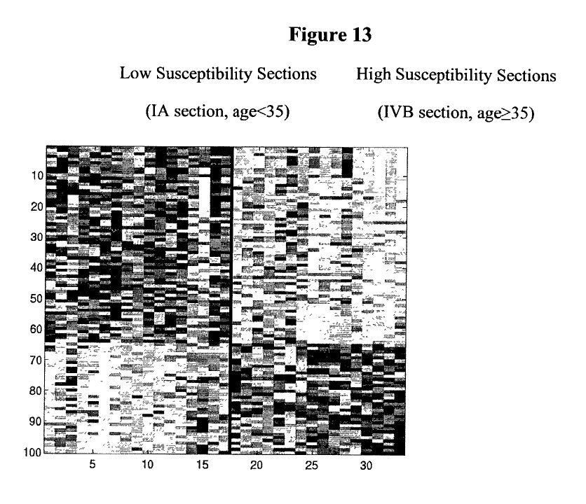

Assessment of atherosclerosis burden in aorta samples. With techniques and software programs developed by the PDAY investigators, two binary images from each sample were produced, one for Sudan IV positive areas (fatty streaks) and one for raised lesions (FIGS. 11c, 11d and 11e). Two analyses were performed using the aortas available for the study—comparison of low vs. high susceptibility to atherosclerosis and minimally vs. severely diseased sections.

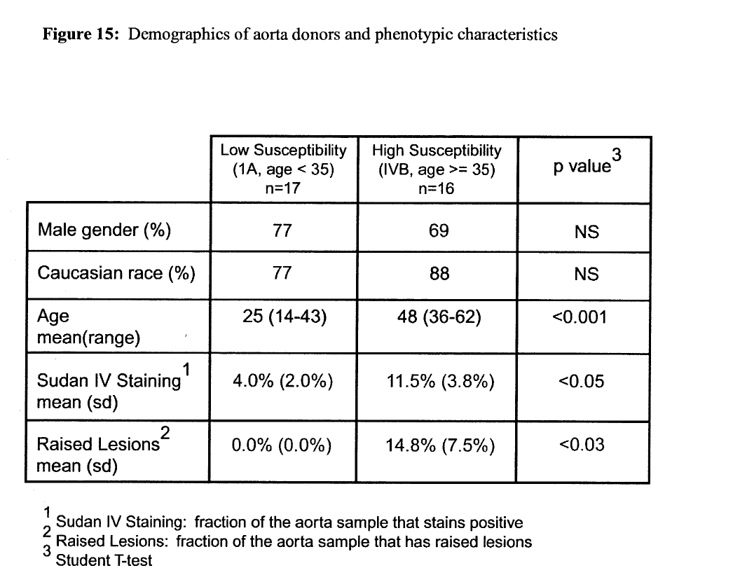

In the first analysis, genes associated with susceptibility for atherosclerosis by comparing the gene expression profiles on the 1A and 4B sections (low susceptibility vs. high susceptibility) were determined. This analysis is based on the observation that detectable lesions are found earlier in 4B than in 1A sections, thus suggesting a greater susceptibility of the 4B section. There was no difference between the two sections in either the extent of Sudan IV staining (11%±11% vs 16%3≡16%, p<0.26) or raised lesions (9%±21% vs 10%±26%, p<0.86) (See Table in FIG. 15).

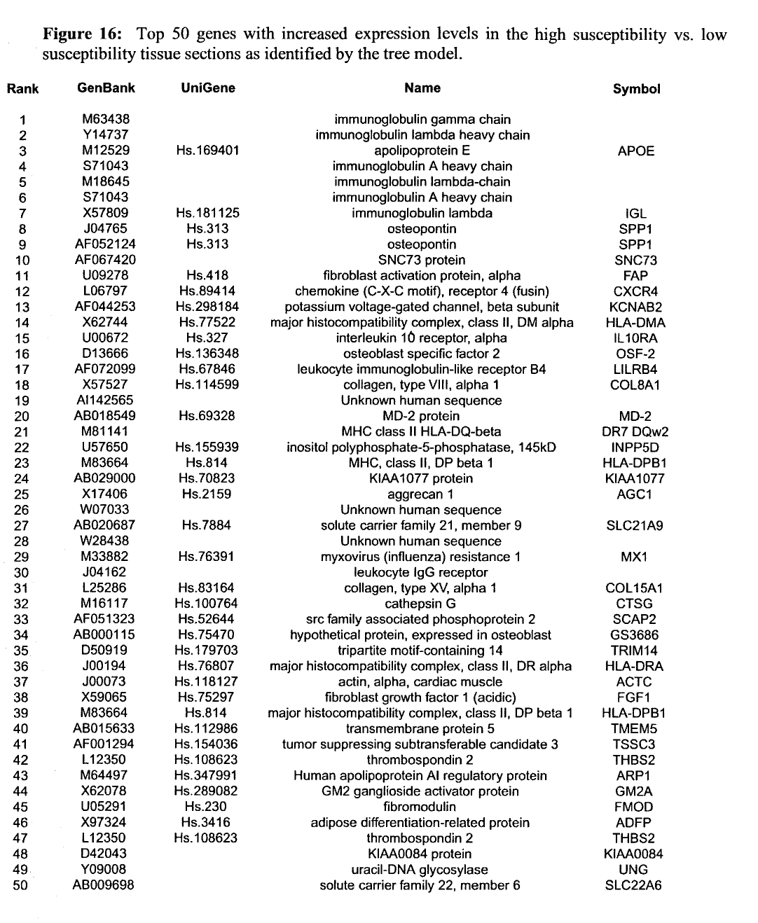

In the second analysis, genes associated with severity of atherosclerotic burden as assessed by percent of total area affected by Sudan IV and raised lesions were determined. There was a significant difference in measures of atherosclerosis burden between the minimally diseased and severely diseased groups. There was significantly less Sudan IV staining in the minimally diseased samples (0.20%±0.40% vs. 19.3%±17.2%, p<0.0003). There was significantly less raised lesions in the minimally diseased samples (0.00%±0.00% vs. 42.4%±26.1%, p<0.0000002) (See Table in FIG. 16).

Tree analysis models. The extracted RNA displayed satisfactory quality and was used for target probe synthesis and hybridization to Affymetrix GeneChip microarrays to produce our expansion database. Two analyses were performed, one compared the low vs. high susceptibility areas for atherosclerosis and the other compared minimally vs. severely diseased samples.

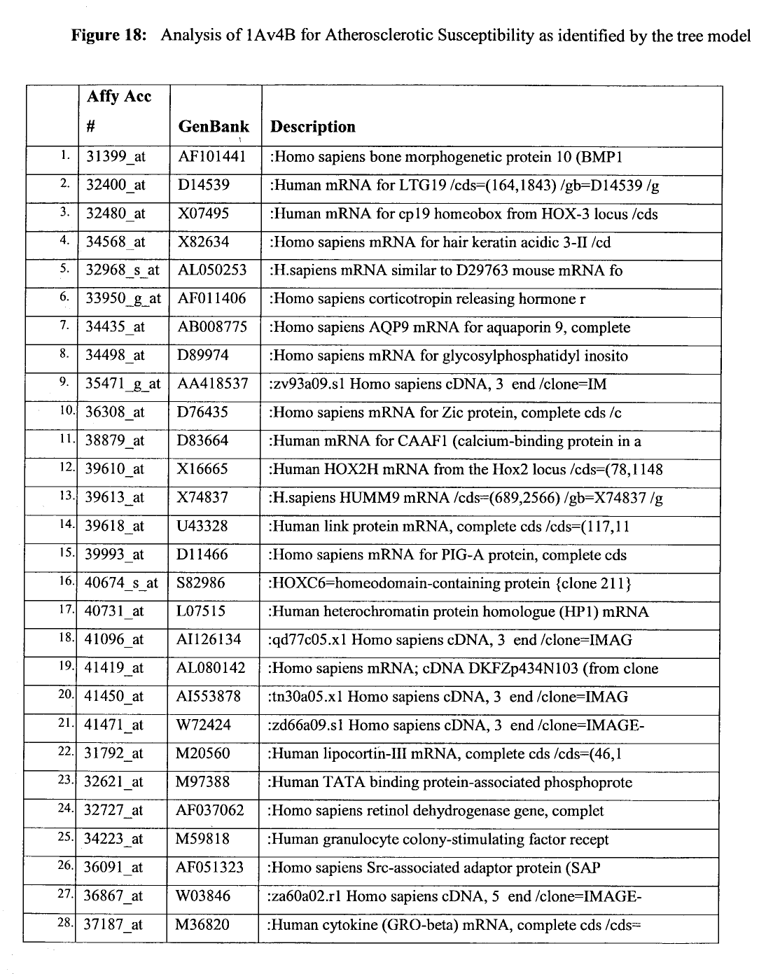

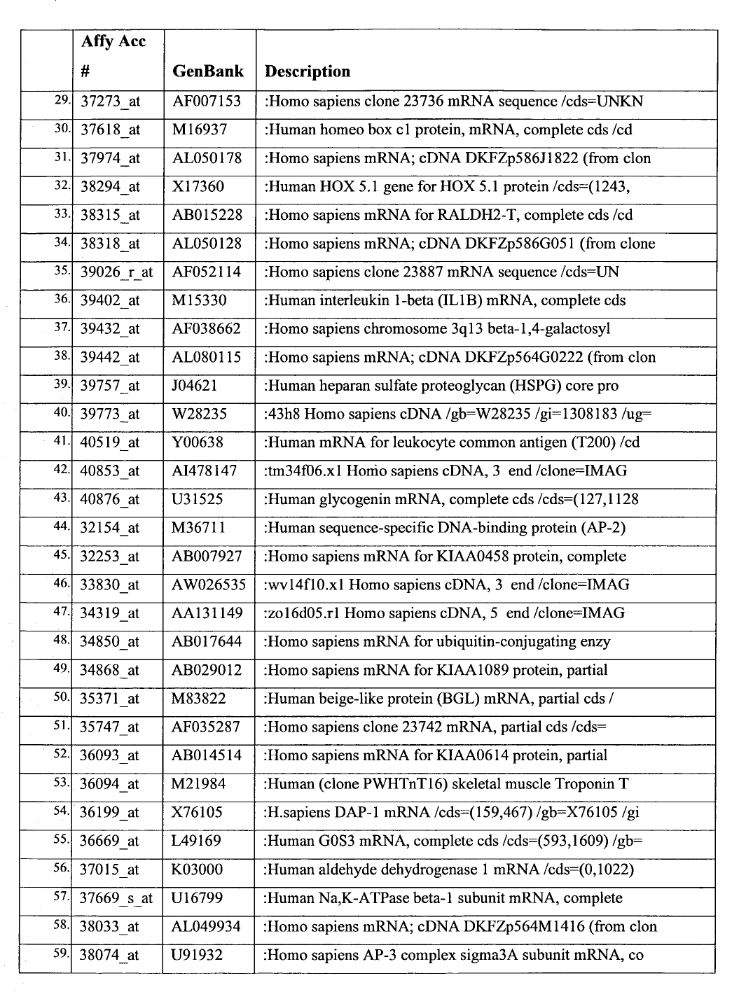

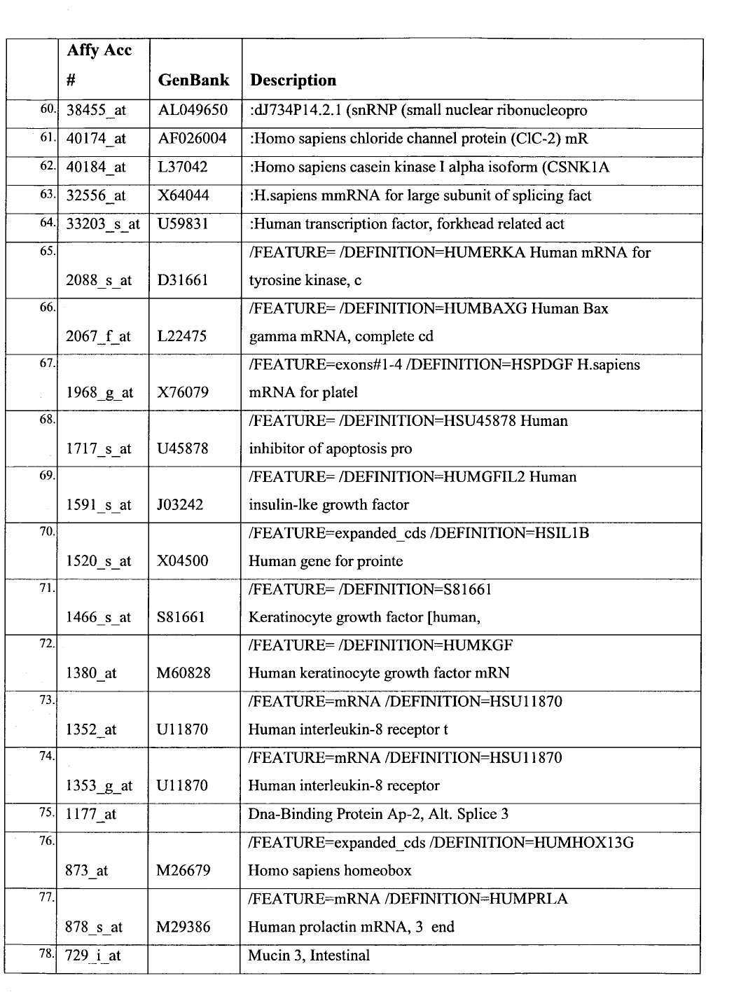

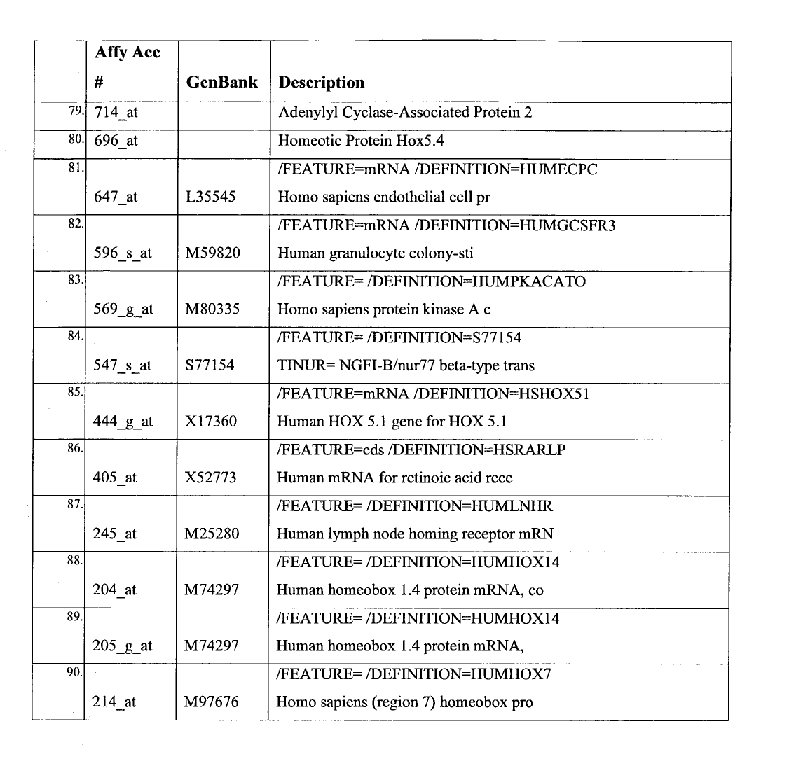

Comparison of low vs. high susceptibility regions. In the first analysis, genes associated with susceptibility for development of atherosclerosis (low threshold segments) were identified by comparing the gene expression profiles of the 1A (low susceptibility) and 4B (high susceptibility) sections. 95 genes that discriminate between sections with low susceptibility and high susceptibility for atherosclerosis were identified (See Table in FIG. 18). FIG. 3 show standardized expression levels of the 95 genes for the 45 samples with a qualitative display that clearly shows differential expression patterns between low susceptibility and high susceptibility tissue samples.

An out-of-sample cross validation test was performed to test the predictive capability of the tree model to classify an unknown sample. Of the 45 samples available for the analysis, the tree model was calculated using 44 samples and was then used to classify the 45^{th} sample. This was repeated for each of the 45 samples (FIG. 4). Of the 45 samples, 39 samples were correctly classified as being 1A vs. 4B for an overall accuracy of 87%.

The genes identified by this analysis code for both known proteins and others whose annotation has not been established. Some of the genes, such as interleukin 1 (IL-1), interleukin 8 (IL-8) and insulin-like growth factor 2 (IGF-2), have been previously associated with atherosclerosis in the literature. Another group of genes code for proteins that belong to categories that one would expect from a survey of atherosclerotic tissue but have not been directly linked to atherosclerosis. Genes in thid category belong to inflammatory, growth signaling, and cell-cell communications pathways. Interesting and unexpected candidates included genes such as fibroblast growth factor 7 (FGF-7) and platelet derived growth factor receptor (PDGF-R).

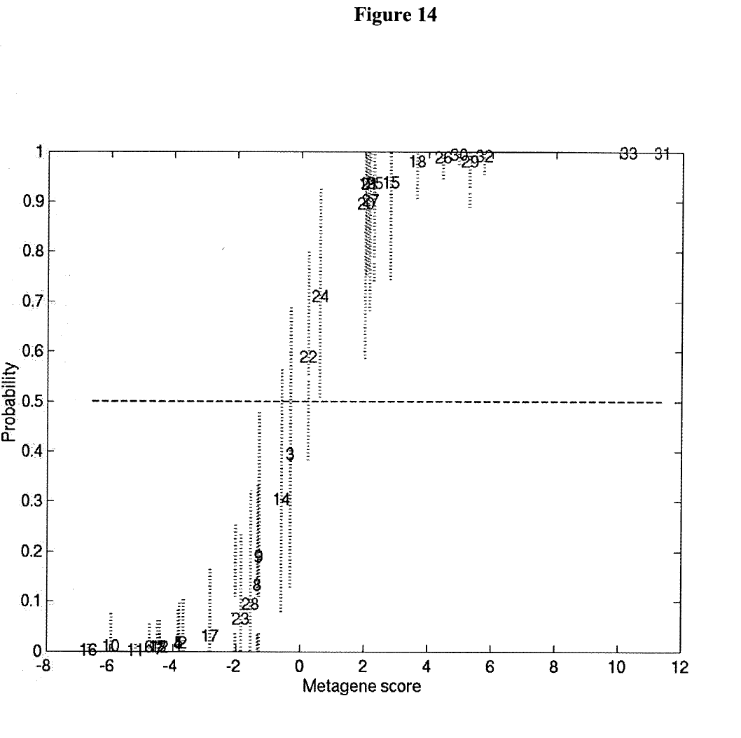

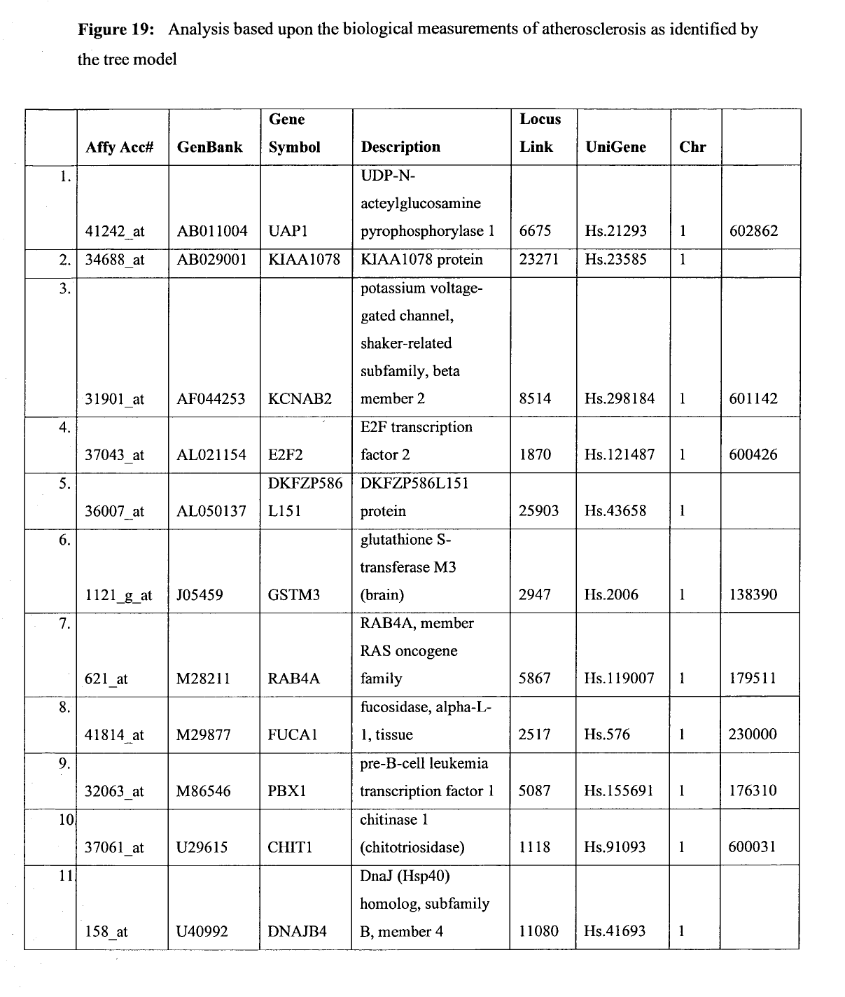

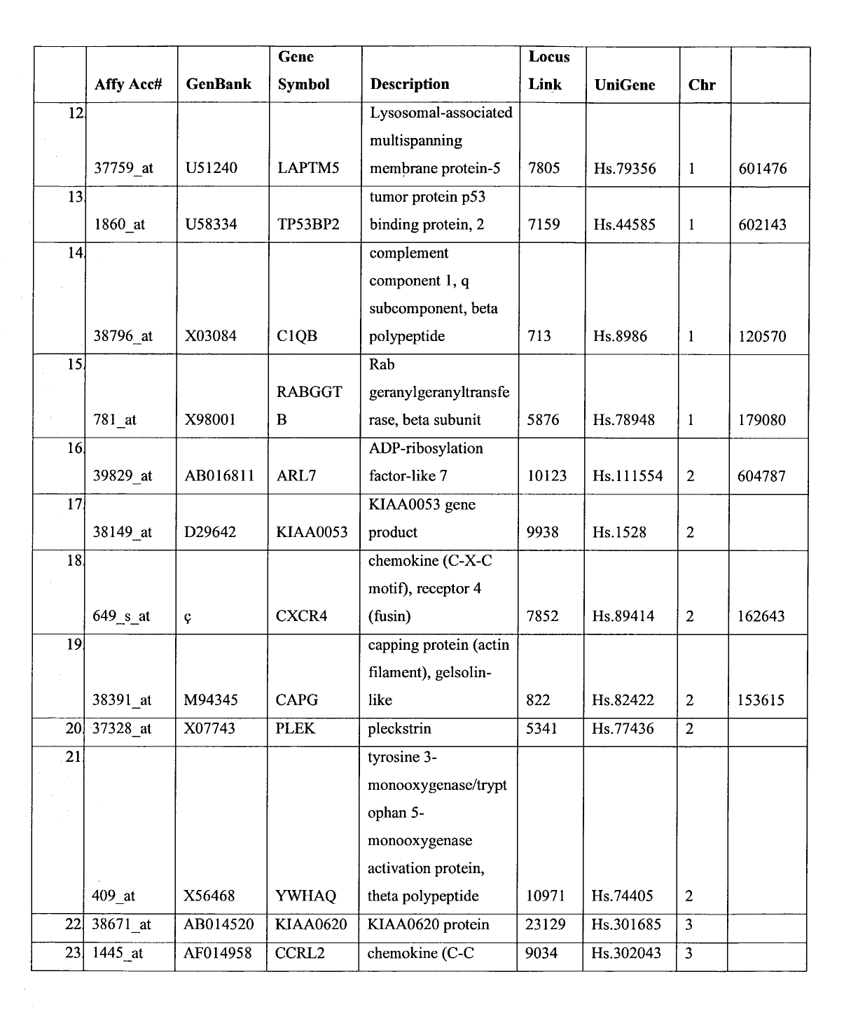

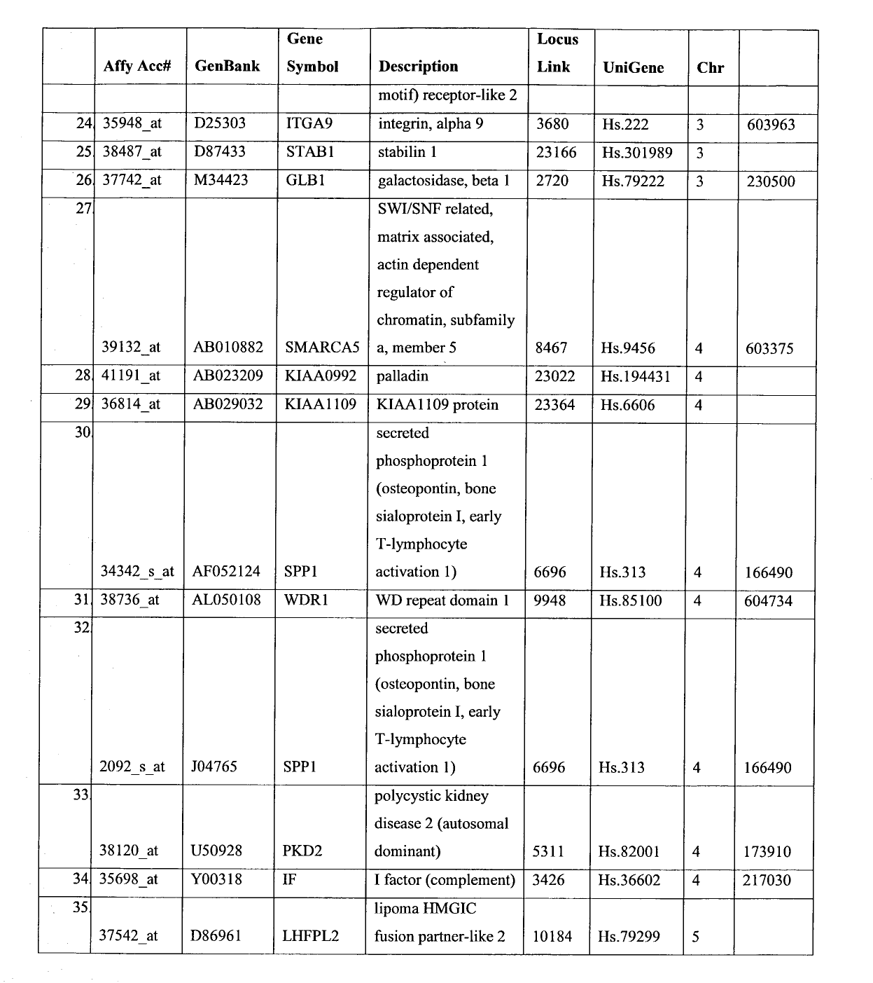

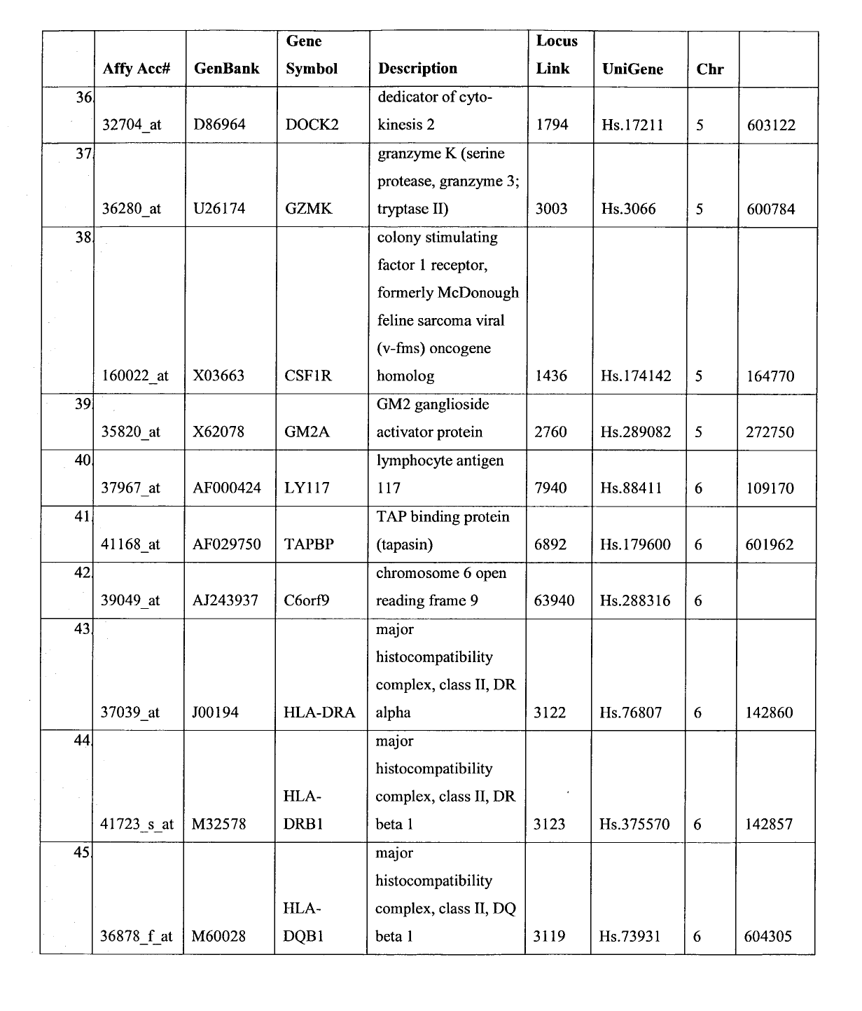

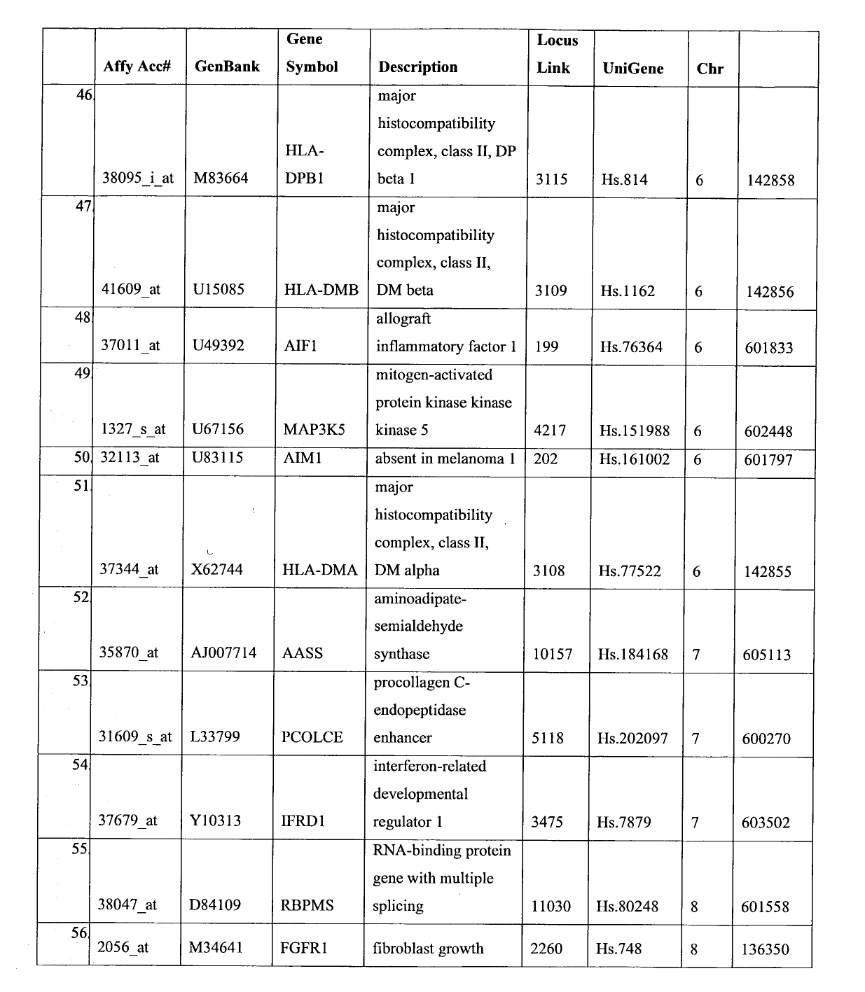

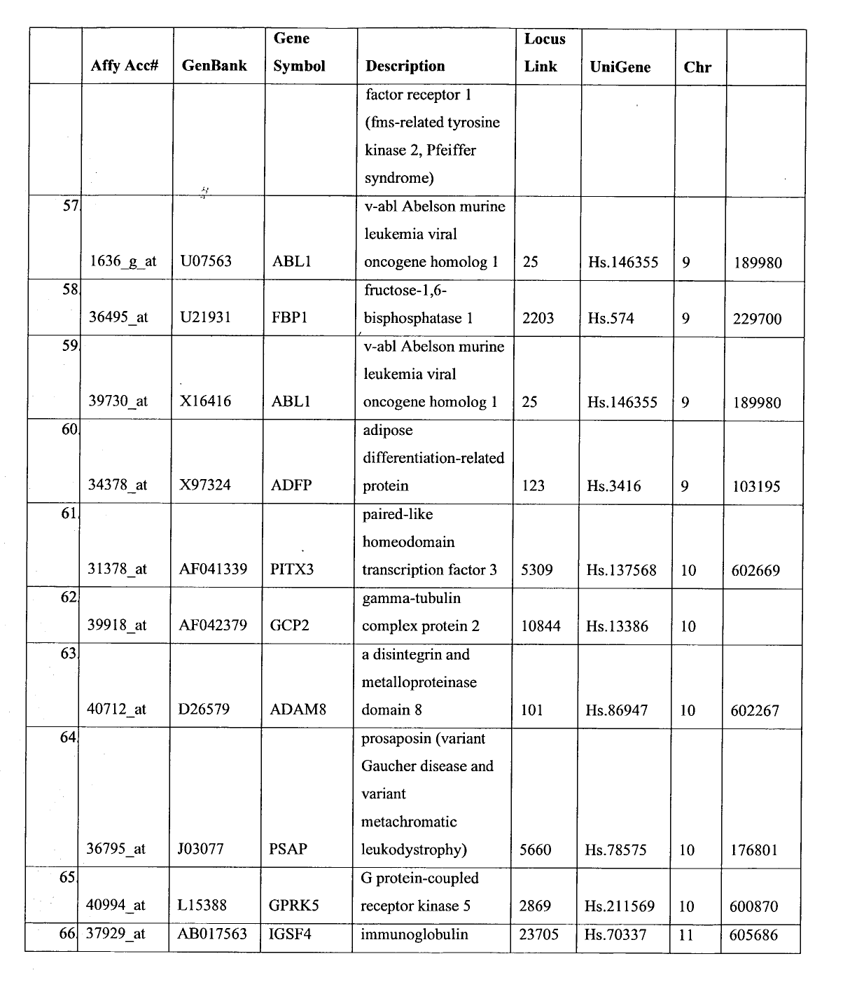

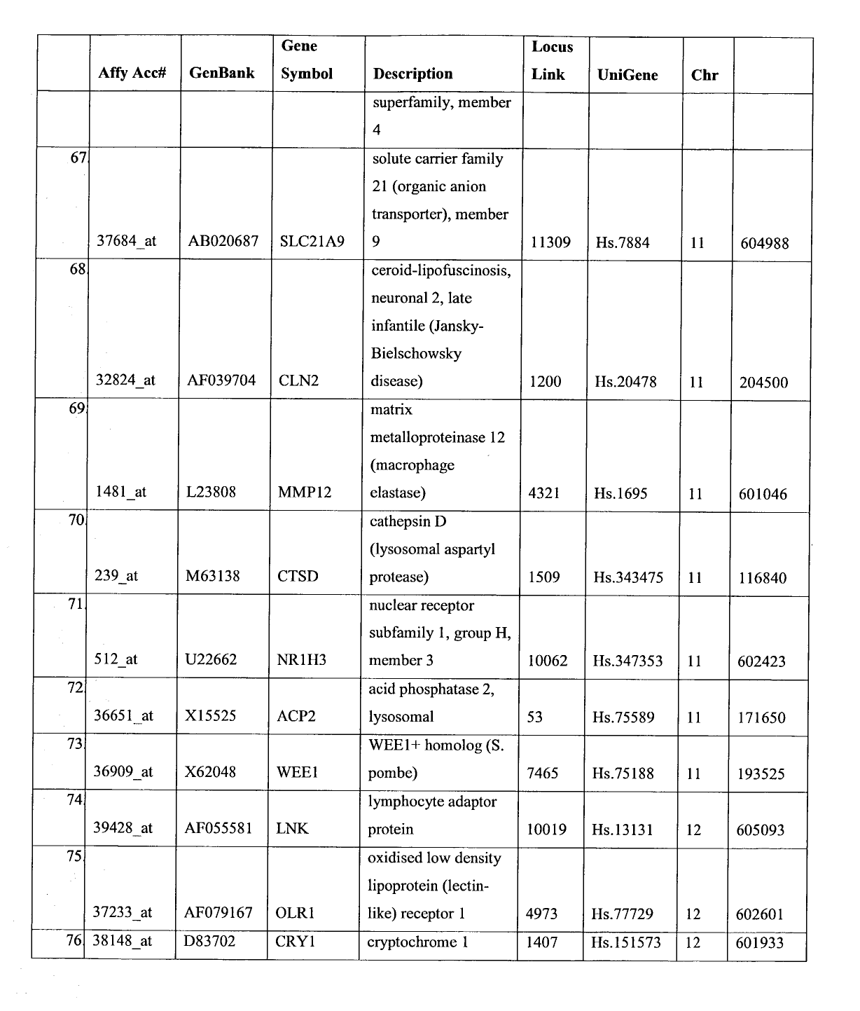

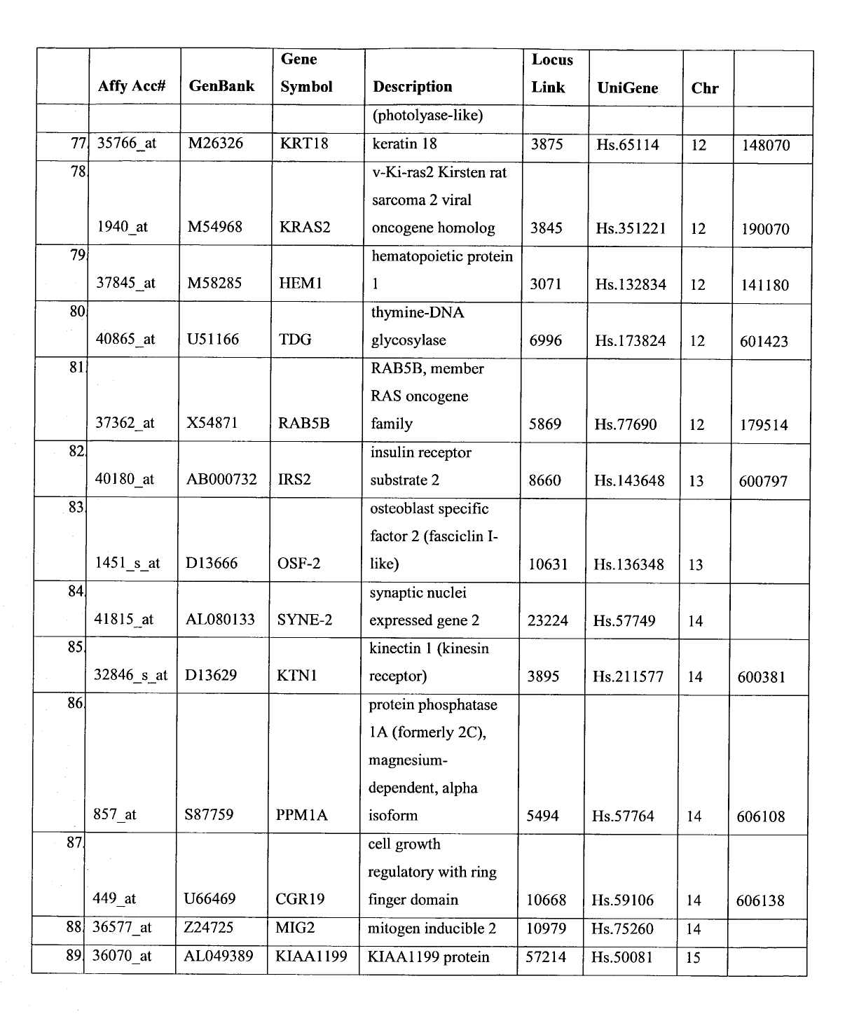

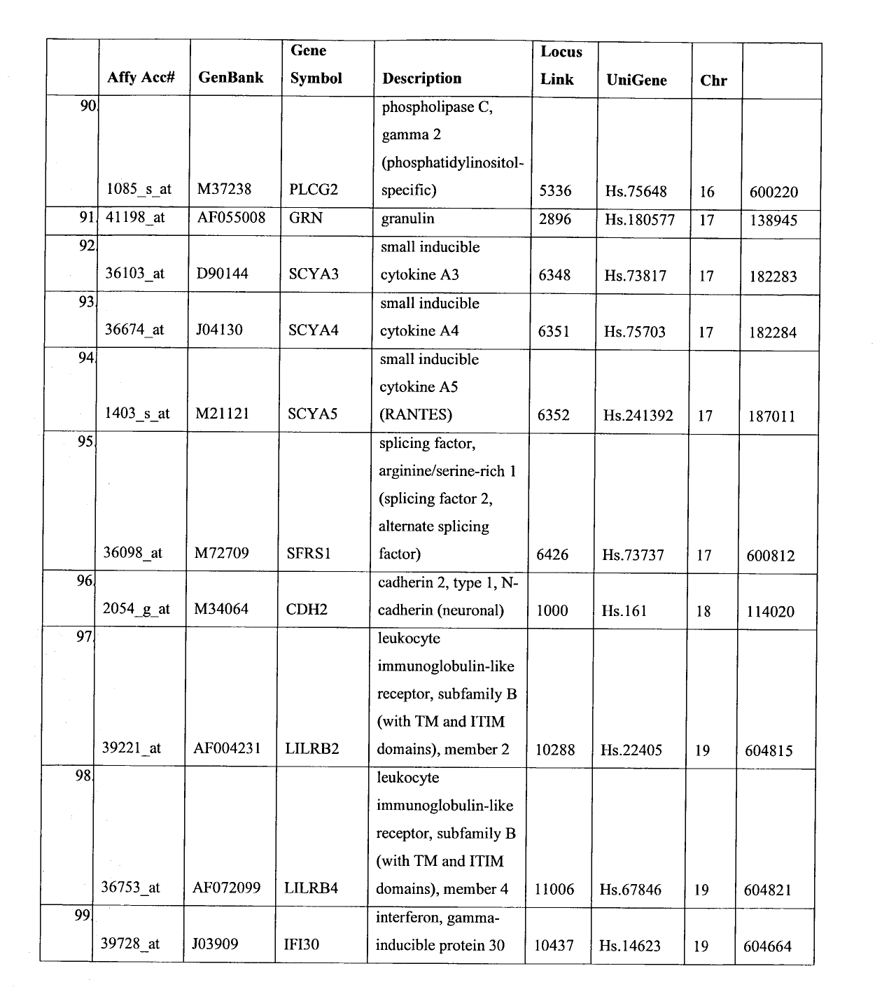

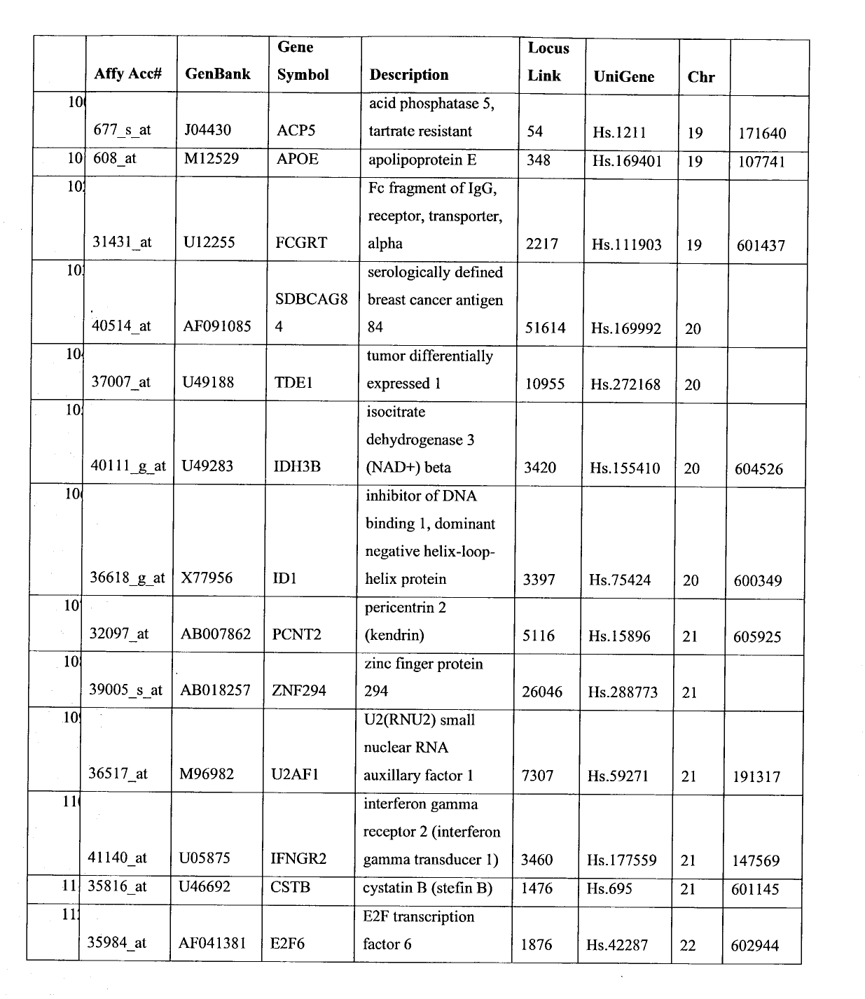

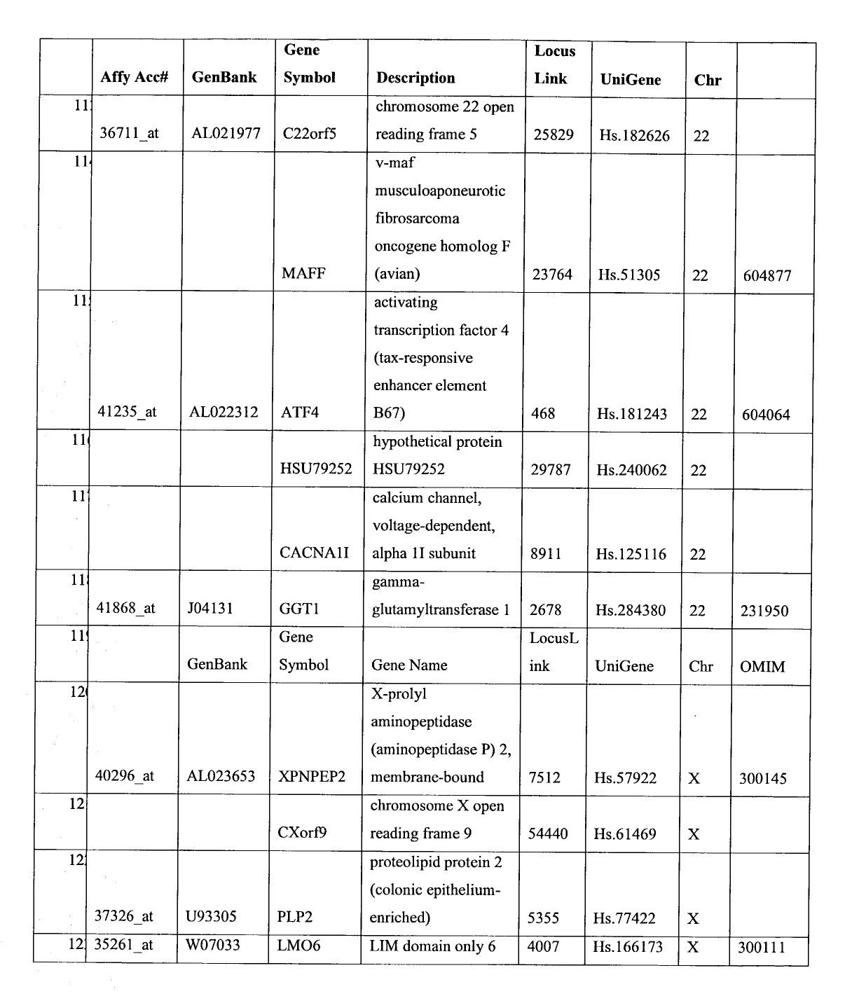

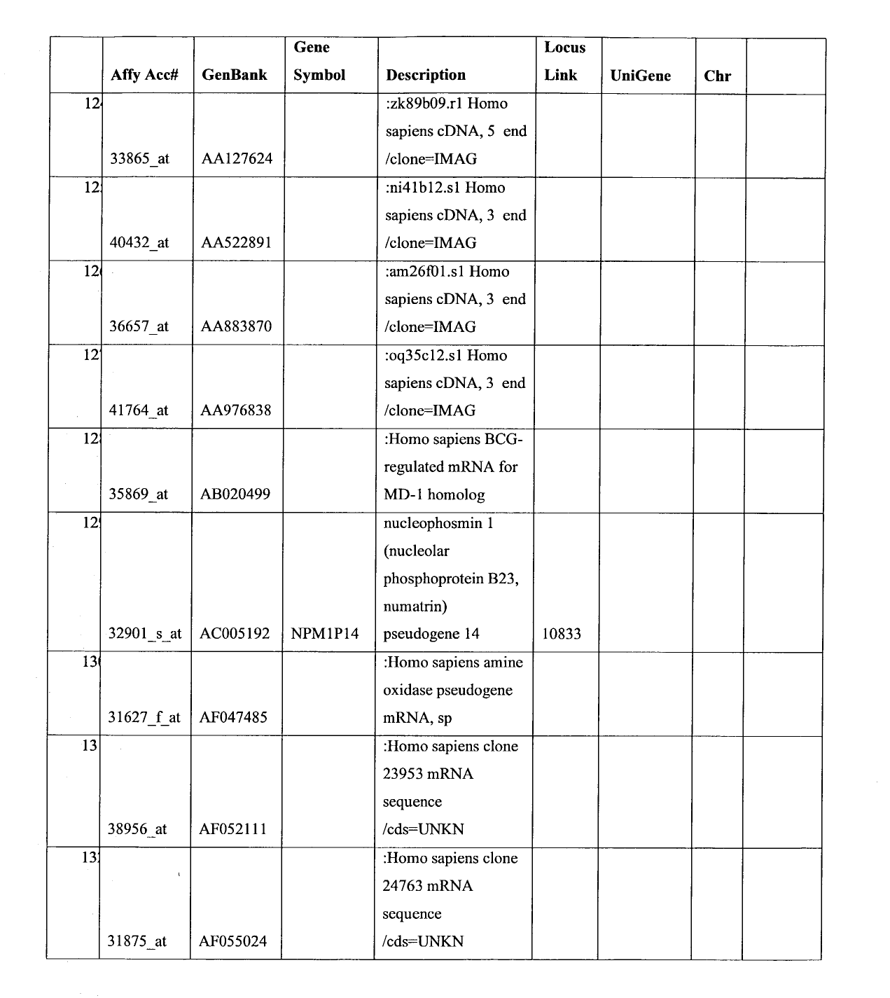

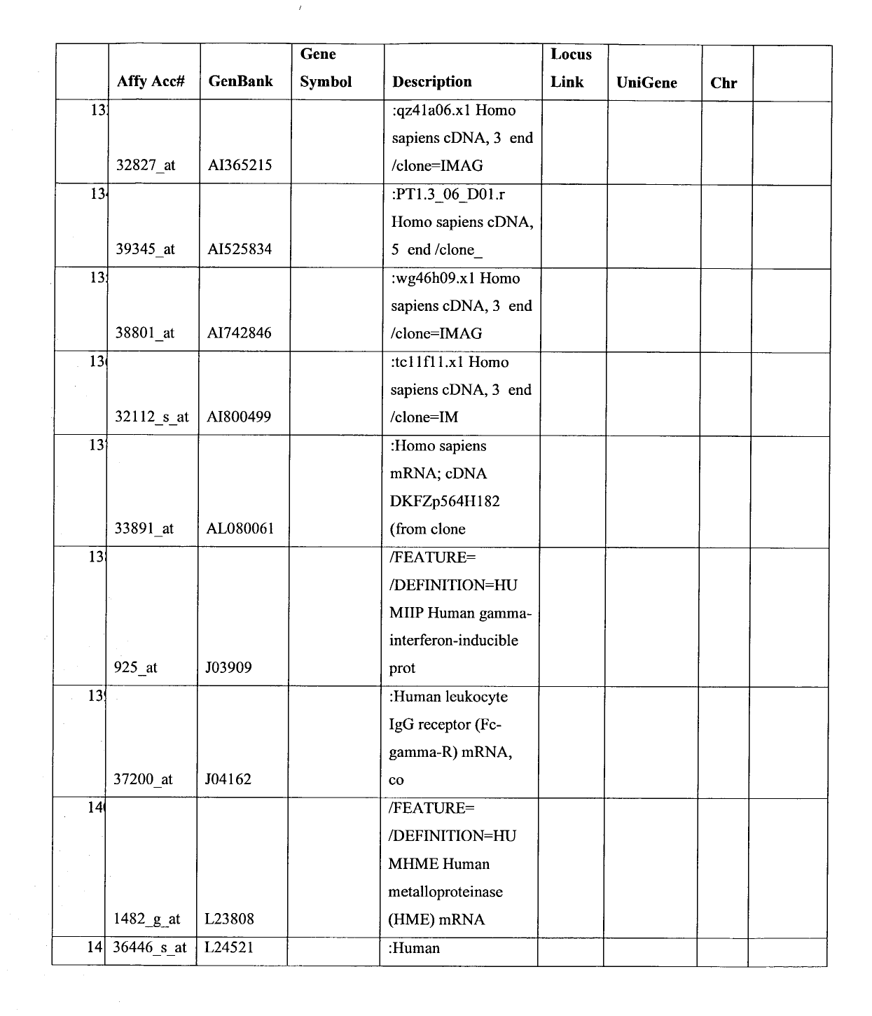

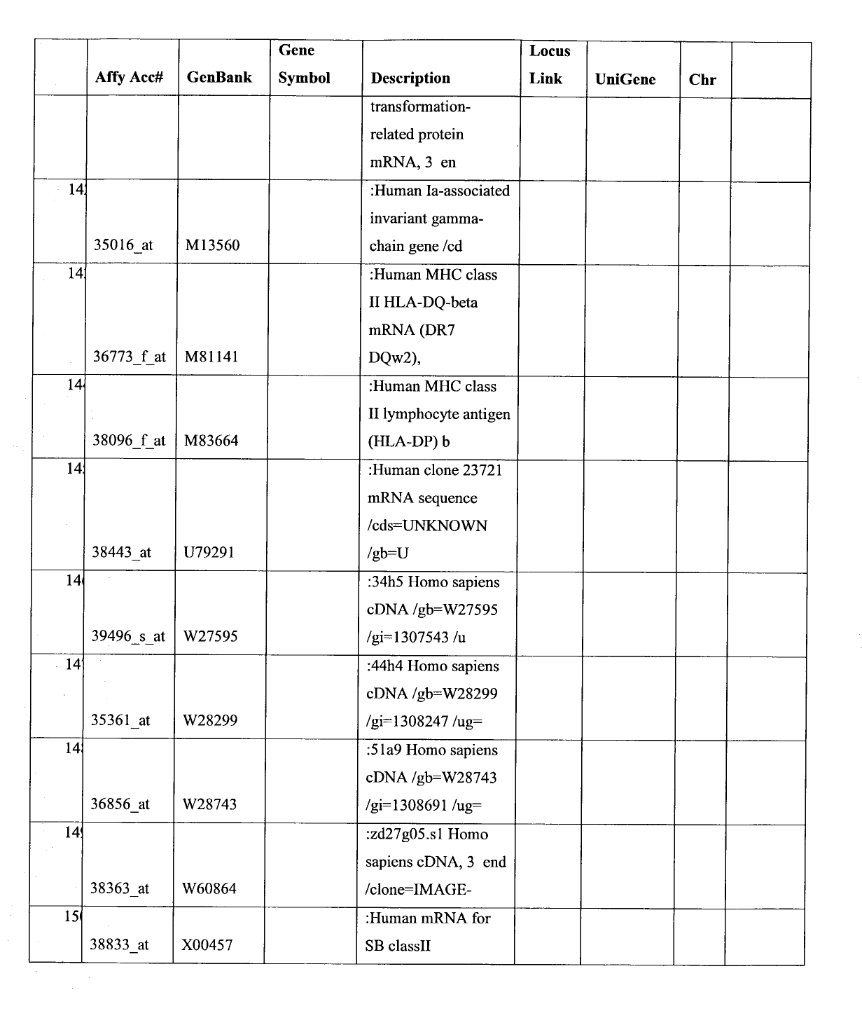

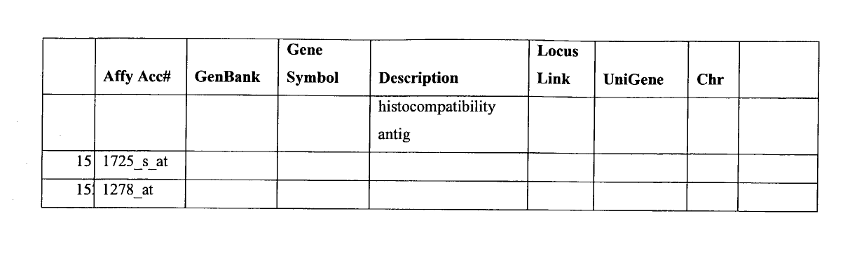

Comparison of minimally vs severely diseased sections. The second analysis identified genes associated with the minimal vs. severely diseased phenotype. The samples were grouped into the binary classification by Sudan IV and raised lesion quantification. There were 14 minimally diseased and 12 severely diseased sections. We identified 150 genes that contribute to this binary classification were identified (See table in FIG. 19). FIG. 14 shows the standardized expression levels of the 150 genes for the 26 samples with a qualitative display that clearly shows differential expression patterns between the minimally and severely diseased tissue samples.

The out of sample cross validation analysis was performed for each of the 26 samples to assess the predictive capacity of the model. The model accurately predicted the status of an unknown sample with 92% accuracy (24/26).

The genes identified as having discriminatory expression code for both known proteins and others whose annotation has not been established. Some of the genes, such apolipoprotein E (apoE) and osteopontin, have been previously associated with atherosclerosis. Another group of genes code for proteins that belong to categories that one would expect from a survey of atherosclerotic tissue but have not been directly linked to atherosclerosis. Genes in this category belong to inflammatory, growth signaling, and cell-cell communication pathways. Interesting and unexpected candidates include genes such as chemokine receptor (CXCR4) and E2F transcription factor 6 (E2F-6).

Additional Gene Lists

We have also considered statistical approaches that have lead to lists of discriminatory genes that are not entirely overlapping with those presented above. Thus, based on our discovery that, as it relates to gene expression and atherosclerosis, genes appear to be “on” or “off”. Hence, we have designed a “tree” approach, where a specific gene fits at each branching point (or node). Upon testing all genes for each node, we have identified another group of genes that is listed in the table in FIG. 18, which was based on the ability of these genes to discriminate perfectly fragment I and IV. For each node, the gene is either expressed or silenced.

Discussion

This novel, nonbiased tree model identifies genes associated with the susceptibility for atherosclerotic development as well as the extent of atherosclerotic burden in human aorta samples. Rather than merely identifying genes whose expression increased or decreased by some arbitrary amount for a given clinical phenotype, patterns of gene expression there were highly correlated with clinical phenotypes of interest were identified.

The characterization of the aorta samples showed that the binary classifications that were designated in our two analyses were valid. The first analysis identified genes associated with susceptibility to the development of atherosclerosis. The PDAY study and the data showed conclusively that atherosclerotic development progresses form the distal to the proximal aorta indicating differences in susceptibility to disease development among the different locations of the aorta. Thus, in this study the gene expression levels in the proximal (1A) and distal (4B) sections of the human aortas were compared. The characterization analysis of the 1A and 4B sections used in the analysis showed no differences in the Sudan IV and raised lesion analyses indicating that the differential gene expression patterns we found were indicative of disease susceptibility as designated by aortic location relation to earlier outcome of disease, rather than the presence of disease. The second analysis identified genes associated with the binary classification of minimal vs. severe atherosclerosis. There was a clear and significant difference between the amount of Sudan IV and raised lesions contained in the two groups of tissues that were studied. The samples included in the analysis were classified purely based upon the measures of atherosclerosis, therefore the genes identified reflect extent of disease.

It is believes that the statistical methodology described in this report is robust. The use of the metegenes in this statistical approach places the emphasis on the differential expression of multiple genes acting in concert which fits with the biological model of complex diseases. Thus this approach is applicable to the study of complex diseases such as atherosclerosis.

Second, the statistical tree models used in this study showed considerable predictive accuracy in honest, cross-validation analysis. In the cross validation analysis, the tree model was built using all of the samples except for one. The model was then used to predict the status of the held-out sample. Even with a limited sample size, our statistical model was able to correctly classify unknown samples with 87% (39/45) accuracy in analysis of susceptibility for disease and with 92% (24/26) accuracy in analysis of extent of disease severity. Even with the heterogeneous nature of human aorta tissues that are additionally affected by atherosclerosis, we were able to detect differences in gene expression patterns with a high degree of accuracy.

Finally, this methodology has identified a number of clinically relevant candidate genes that encode proteins whose function is consistent with a role in atherosclerosis, such as proteins belonging to inflammatory, growth signaling, and cell-cell communication pathways. Some of these genes such as apoE, ER-β and osteopontin have previously been directly associated with atherosclerosis. The apoE gene and particularly apoE gene variants, have been linked to the development of atherosclerosis in humans. See Ilveskoski E, Perola M, Lehtimaki T, et al. Agedependent association of apolipoprotein E genotype with coronary and aortic atherosclerosis in middle-aged men: an autopsy study. Circulation 1999; 100:608-13. In these studies, apoE gene expression was elevated in the high susceptibility sections. While primarily expressed in the liver and the brain, apoE is also expressed in monocytes and vascular smooth muscle cells where it may play a role in paracrine and autocrine cholesterol transport, and induce smooth muscle cell differentiation and proliferation. See Mahley R W. Apolipoprotein E: cholesterol transport protein with expanding role in cell biology. Science 1988; 240:622-30.

ERP is one of two receptors that mediates the effects of estrogen and is present in the vascular tissue of both females and male. See Savolainen H, Frosen J, Petrov L, Aavik E, Hayry P. Expression of estrogen receptor sub-types alpha and beta in acute and chronic cardiac allograft vasculopathy. J Heart Lung Transplant 2001; 20:1252-64. In our study, ERβ levels were decreased in the high susceptibility sections. The vasoprotective effects of estrogen have been well documented in human subjects and in animal models and is in part estrogen receptor mediated. See Bakir S, Mori T, Durand J, Chen Y F, Thompson J A, Oparil S. Estrogen-induced vasoprotection is estrogen receptor dependent: evidence from the balloon-injured rat carotid artery model. Circulation 2000; 101:2342-4. See Savolainen H, Frosen J, Petrov L, Hayry P. Expression of the vasculoprotective estrogen receptor subtype beta in rat and human cardiac allograft vasculopathy. Transplant Proc 2001; 33:1605. See Tolbert T, Oparil S. Cardiovascular effects of estrogen. Am J Hypertens 2001; 14:186S-193S. The binding of estrogen to ERα and ERβ induces the production of the Fas ligand (FasL) resulting in the inhibition of leukocyte invasion into the vascular wall. This may be one mechanism for decreasing susceptibility to atherosclerosis. See Sata M, Walsh K. TNFalpha regulation of Fas ligand expression on the vascular endothelium modulates leukocyte extravasation. Nat Med 1998; 4:415-20.In another report, ERβ-deficient mice exhibited significant systolic and diastolic hypertension which are primary risk factors for atherosclerosis. See Zhu Y, Bian Z, Lu P, et al. Abnormal vascular function and hypertension in mice deficient in estrogen receptor beta. Science 2002; 295:505-8. Osteopontin expression was also elevated in the high susceptibility sections. It is a noncollagenous bone matrix protein highly expressed in calcified atheromas and is secreted by smooth muscle cells and macrophages. See Bini A, Mann K G, Kudryk B J, Schoen F J. Noncollagenous bone matrix proteins, calcification, and thrombosis in carotid artery atherosclerosis. Arterioscler Thromb Vasc Biol 1999; 19:1852-61. See Canfield A E, Farrington C, Dziobon M D, et al. The involvement of matrix glycoproteins in vascular calcification and fibrosis: an immunohistochemical study. J Pathol 2002; 196:228-34. See Dhore C R, Cleutjens J P, Lutgens E, et al. Differential expression of bone matrix regulatory proteins in human atherosclerotic plaques. Arterioscler Thromb Vasc Biol 2001; 21:1998-2003. See Kwon H M, Hong B K, Kang T S, et al. Expression of osteopontin in calcified coronary atherosclerotic plaques. J Korean Med Sci 2000; 15:485-93. See Moses S, Franzen A, Lovdahl C, Hultgardh-Nilsson A. Injury-induced osteopontin gene expression in rat arterial smooth muscle cells is dependent on mitogen-activated protein kinases ERK1/ERK2. Arch Biochem Biophys 2001; 396:133-7. There is evidence that osteopontin plays a role in crystallization and mineralization of vascular tissues and may influence the pathologic calcification seen in mature atheromas. Osteopontin also modulates tissue remodeling by inducing smooth muscle cell proliferation and migration. See Chaulet H, Desgranges C, Renault M A, et al. Extracellular nucleotides induce arterial smooth muscle cell migration via osteopontin. Circ Res 2001; 89:772-8.

Perhaps the most intriguing finding of this study was the identification of candidate genes without a previous association to atherosclerosis. They do, however, participate in cellular processes essential to atherogenesis. These include TSP-2 and immunoglobulins. TSP-2 is an extracellular matrix protein that has been implicated in cardiovascular disease whose expression was elevated in the high susceptibility sections. TSP-2 has a myriad of effects including inhibiting angiogenesis, modulating cell adhesion, and facilitating platelet aggregation. See Bornstein P, Armstrong L C, Hankenson K D, Kyriakides T R, Yang Z. Thrombospondin 2, a matricellular protein with diverse functions. Matrix Biol 2000; 19:557-68. See Hawighorst T, Velasco P, Streit M, et al. Thrombospondin-2 plays a protective role in multistep carcinogenesis: a novel host anti-tumor defense mechanism. Embo J 2001; 20:2631-40. See Noji Y, Kajinami K, Kawashiri M A, et al. Circulating matrix metalloproteinases and their inhibitors in premature coronary atherosclerosis. Clin Chem Lab Med 2001; 39:380-4. TSP-2 null mice display accelerated wound healing and markedly decreased scar formation. Thus, TSP-2 may influence atherogenesis through its deleterious effect on complex tissue repair mechanisms. See Bornstein P, Kyriakides TR, Yang Z, Armstrong LC, Birk DE. Thrombospondin 2 modulates collagen fibrillogenesis and angiogenesis. J Investig Dermatol Symp Proc 2000; 5:61-6. Expression of immunoglobulins was increased in the high susceptibility tissue sections. Immunoglobulin G (IgG) has been shown to induce monocyte chemoattractant protein 1 (MCP-1) as well as monocyte colony stimulating factor (M-CSF) by crosslinking to the Fe region of monocytes^{26,27}. Thus, IgG may potentiate atherosclerosis by recruiting and activating monocytes to areas of injury. Oxidized low density lipoproteins and microorganisms could represent the source of antigens that trigger to production of IgGs in regions of aorta that are prone to atherosclerosis.

Identifying novel candidate genes is a major focus of this study as it may shed further light on the development of atherosclerosis. Thus, this approach may identify not only the initial steps in a pathway but the secondary and tertiary events as well. As such, the analysis provides a much richer dataset than merely identifying the immediate effectors of a process. Many of the genes identified are likely to be causative and may be applicable to future therapeutic interventions. As well, some of the genes may in fact be “innocent bystander” genes. These could still be interesting from the standpoint of developing new diagnostic and prognostic tools.

V. Summary

The identification of genes that play a role in the development of atherosclerosis, with an ultimate goal of identifying those gene variants that contribute quantitative variation to the disease process, is a goal of numerous academic and industrial groups. Although previous studies have linked gene variants with outcomes in various aspects of cardiovascular disease, these are quite limited in number and likely only touch the surface of the wide range of contributions to the disease process. A key aspect of the studies described in this invention is the capacity to identify not just highly expressed genes but genes whose expression highly correlates with the phenotype, regardless of level of expression. Perhaps most important, however, is the fact that these analyses identify not only genes expected to be involved in the phenotype, thus validating the process, but also genes for which a connection is not immediately clear. It is precisely these genes that are the focus of this invention—the use of expression analysis to identify candidate genes that might not have been identified by other approaches.

It is evident that subject invention provides valuable new atherosclerotic phenotype determinative genes that find use in a variety of different applications, including diagnostic, therapeutic and research applications. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of determining whether a sample is from tissue having an atherosclerotic phenotype, said method comprising:
(a) obtaining an expression profile for said sample for at least two of said genes listed in Table 1; and
(b) comparing said obtained expression profile to a reference expression profile to determine whether said sample is from tissue having an atherosclerotic phenotype.

2. The method according to claim 1, wherein said expression profile is for at least one of said genes listed in Table 1 but not in Table 2.

3. The method according to claim 1, wherein said tissue is a vascular tissue.

4. The method according to claim 3, wherein said vascular tissue is aortic tissue.

5. A method of diagnosing whether a host has an atherosclerotic phenotype, said method comprising:
(a) obtaining a sample from said host;
(b) obtaining an expression profile for said sample for at least two of said genes listed in Table 1; and
(c) comparing said obtained expression profile to a reference expression profile to determine whether said host has an atherosclerotic phenotype.

6. The method according to claim 5, wherein said expression profile is for at least one of said genes listed in Table 1 but not in Table 2.

7. The method according to claim 5, wherein said sample is obtained from vascular tissue.

8. The method according to claim 7, wherein said vascular tissue is aortic tissue.

9. A method of treating a host suffering from atherosclerosis, said method comprising:
(a) determining an atherosclerotic phenotype for said host by:
(i) obtaining a sample from said host;
(ii) obtaining an expression profile for said sample for at least two of said genes listed in Table 1; and
(iii) comparing said obtained expression profile to a reference expression profile to determine an atherosclerotic phenotype for said host;
(b) determining a treatment protocol based on said determined atherosclerotic phenotype; and
(c) treating said host according to said determined treatment protocol.

10. The method according to claim 9, wherein said expression profile is for at least one of said genes listed in Table 1 but not in Table 2.

11. The method according to claim 9, wherein said sample is obtained from vascular tissue.

12. The method according to claim 11, wherein said vascular tissue is aortic tissue.

13. A method of screening a candidate agent for atherosclerotic modulatory activity, said method comprising:
(a) contacting a cell from tissue having an atherosclerotic phenotype with said candidate agent;
(b) obtaining an expression profile for said cell for at least two of said genes listed in Table 1; and
(c) comparing said obtained expression profile to a reference expression profile to determine whether said candidate agent has atherosclerotic modulatory activity.

14. The method according to claim 13, wherein said expression profile is for at least one of said genes listed in Table 1 but not in Table 2.

15. The method according to claim 13, wherein said method is in vitro.

16. The method according to claim 13, wherein said method is in vivo.

17. A method of identifying a gene whose expression is associated with a disease phenotype, said method comprising:
(a) preparing an expression profile for a nucleic acid sample obtained from a source having said disease phenotype;
(b) comparing said expression profile to a control profile; and
(c) identifying genes whose expression correlates with said disease phenotype, where correlation is based on at least one parameter that is other than expression level.

18. The method according to claim 17, wherein said disease phenotype is atherosclerosis.

19. The method according claim 18, wherein said correlation is determined using a Bayesian analysis.

20. The method according to claim 19, wherein said Bayesian analysis comprises use of binary regression models combined with singular value decompositions and stochastic regularization.

21. A reference expression profile for an atherosclerotic phenotype that includes expression data for at least two of the genes of Table 1, wherein said expression profile is recorded on a computer readable medium.

22. The expression profile according to claim 21, wherein said expression profile includes at least one of the genes from Table 1 but not from Table 2.

23. A collection of gene specific primers, said collection comprising:
gene specific primers specific for at least two of the genes of Table 1.

24. The collection according to claim 23, wherein said collection comprises at least one specific primer specific for at least one gene from Table 1 but not from Table 2.

25. A array of probe nucleic acids immobilized on a solid support, said array comprising:
a plurality of probe nucleic acid compositions, wherein each probe nucleic acid composition is specific for a gene whose expression profile is correlated with an atherosclerotic phenotype, wherein at least two of said probe nucleic acid compositions correspond to genes listed in Table 1.

26. The array according to claim 25, wherein said array further comprises at least one control nucleic acid composition.

27. The array according to claim 26, wherein said array includes at least one probe composition corresponding to a gene listed in Table 1 but not from Table 2.

28. A kit for use in determining the atherosclerotic phenotype of a source of a nucleic acid sample, said kit comprising:
at least one of:
(a) an array according to claim 25; and
(b) a collection of gene specific primers according to claim 23.

29. The kit according to claim 28, wherein said kit comprises both said array and said collection of gene specific primers.
